(19) United States
(12) Patent Application Publication
(10) Pub. No.: US 2025/0361514 A1
(43) Pub. Date: Nov. 27, 2025

(54) ENGINEERING CASSAVA FOR IMPROVED GROWTH AND YIELD

(71) Applicants: University of Kaiserslautern-Landau, Kaiserslautern (DE); ETH Zürich, Zürich (CH); Friedrich-Alexander-Universität Erlangen-Nürnberg in Vertretung des Freistaates Bayem, Erlangen (DE); National Chung Hsing University, Taichung City (TW)

(72) Inventors: Ekkehard NEUHAUS, Kaiserslautern (DE); Leo BELLIN, Kaiserslautern (DE); Uwe SONNEWALD, Möhrendorf (DE); Wolfgang ZIERER, Haiming (DE); Wilhelm GRUISSEM, Forch (CH)

(73) Assignees: University of Kaiserslautern-Landau, Kaiserslautern (DE); ETH Zürich, Zürich (CH); Friedrich-Alexander-Universität Erlangen-Nürnberg in Vertretung des Freistaates Bayem, Erlangen (DE); National Chung Hsing University, Taichung City (TW)

(21) Appl. No.: 19/216,373

(22) Filed: May 22, 2025

Related U.S. Application Data
(60) Provisional application No. 63/651,899, filed on May 24, 2024.

(51) Int. Cl. C12N 15/82

(52) U.S. Cl. CPC C12N 15/8227; C12N 15/8205; C12N 15/8245; C12N 15/8262; C12N 15/8271

(57) ABSTRACT

The present disclosure relates to genetically modified plants including a modified POTASSIUM TRANSPORTER 2 (AKT2) protein or an overexpressed AKT2 protein. The present disclosure further relates to methods of producing genetically modified plants including the modified AKT2 protein or the overexpressed AKT2 protein. In addition, the present disclosure relates to genetically modified plants with improved photosynthesis, higher rate of CO₂ fixation and/or electron transport rate, improved yield under different growing conditions, and improved storage root or tuber growth.

### CROSS-REFERENCE TO RELATED APPLICATIONS

This application claims the benefit of U.S. Provisional Application No. 63/651,899, filed May 24, 2024, hereby incorporated by reference in its entirety.

### REFERENCE TO AN ELECTRONIC SEQUENCE LISTING

The content of the electronic sequence listing (794542003100SEQLIST.xml; Size: 81,516 bytes; and Date of Creation: Apr. 21, 2025) is herein incorporated by reference in its entirety.

### TECHNICAL FIELD

The present disclosure relates to genetically modified plants or plant parts thereof including a modified POTASSIUM TRANSPORTER 2 (AKT2) protein or an overexpressed AKT2 protein. The present disclosure further relates to methods of producing genetically modified plants or plant parts thereof including the modified AKT2 protein or the overexpressed AKT2 protein. In addition, the present disclosure relates to genetically modified plants or plant parts thereof with improved phloem transport, improved photosynthesis, higher rate of CO₂ fixation and/or electron transport rate, improved yield under different growing conditions, and improved storage root or tuber growth.

### BACKGROUND

Potassium is a major plant nutrient and a key factor for crop yield. In particular, the cation (K⁺) is a major active solute in plants with a key function in maintaining turgor pressure and driving changes in cell volume. In addition, potassium is involved in many metabolic processes and also serves as an important enzymatic cofactor. More importantly, potassium is the primary cation in the phloem, and is increasingly recognized as a key factor for influencing phloem mass flow.

The importance of potassium fertilization for cassava storage root yield has been demonstrated in numerous studies (to cite just a few recent studies: Chua et al., 2020. Potassium Fertilisation Is Required to Sustain Cassava Yield and Soil Fertility. Agronomy [Online], 10; Fernandes et al., 2017. Yield and nutritional requirements of cassava in response to potassium fertilizer in the second cycle. Journal of Plant Nutrition, 40, 2785-2796.; Gazola et al., 2022. Potassium management effects on yield and quality of cassava varieties in tropical sandy soils. Crop and Pasture Science, 73, 285-299.; Sukkaew et al., 2022. Response of cassava (Manihot esculenta Crantz) to calcium and potassium in a humid tropical upland loamy sand soil. Annals of Agricultural Sciences, 67, 204-210.). Van Laere et al. (Van Laere et al., 2023. Carbon allocation in cassava is affected by water deficit and potassium application—A (13) C—CO(2) pulse labelling assessment. Rapid Commun Mass Spectrom, 37, e9426.) recently also showed that potassium application can improve carbon allocation to storage roots. It has been hypothesized that K⁺ circulating in phloem serves as a decentralized energy storage which may be used to overcome local energy limitations (Gajdanowicz et al., 2011. Potassium (K+) gradients serve as a mobile energy source in plant vascular tissues. Proc Natl Acad Sci USA, 108, 864-9.). Additional reports have shown that potassium plays a role in maintaining phloem pressure flow in maize, an active phloem loader (Babst et al., 2022. Sugar loading is not required for phloem sap flow in maize plants. Nat Plants 8(2): 171-180.).

Potassium channels are important facilitators of K⁺ uptake from the soil and K⁺ movement within the plant. Broadly, voltage-gated potassium channels can be divided into inward-rectifying K⁺ channels (Kᵢₙ) and outward-rectifying K⁺ channels (Kₒᵤₜ). One such potassium channel is POTASSIUM TRANSPORTER 2 (AKT2). Wild type AKT2 has two modes, namely mode 1, where AKT2 acts as an inward-rectifying K⁺ channel (Kᵢₙ), and mode 2, where AKT2 acts as a nonrectifying channel (both Kᵢₙ and Kₒᵤₜ; i.e., mediating both K⁺ uptake and release) (Dreyer et al., 2017, The potassium battery: a mobile energy source for transport processes in plant vascular tissues. New Phytologist 216: 1049-1053). Modification of AKT2 can result in AKT2 being biased toward or locked in mode 2, such that it acts as a nonrectifying channel with both Kᵢₙ and Kₒᵤₜ functions (Gajdanowicz et al., 2011. Potassium (K+) gradients serve as a mobile energy source in plant vascular tissues. Proc Natl Acad Sci USA, 108, 864-9.). Posttranslational modifications of the AKT2 channel can allow the plant to tap into the circulating K⁺ energy storage by efficiently assisting the plasma membrane H⁺-ATPase in energizing the transmembrane phloem loading process (Gajdanowicz et al., 2011. Potassium (K+) gradients serve as a mobile energy source in plant vascular tissues. Proc Natl Acad Sci USA, 108, 864-9.). Following this hypothesis, AKT2 and the “potassium battery” would be most relevant in apoplasmic phloem loaders that actively transport assimilates against a concentration gradient.

Phloem transport in cassava is nuanced and dynamic. While cassava is a mostly symplasmic phloem loader in its leaves, and a mostly symplasmic phloem unloader in its lower stem and storage roots (Rüscher et al., (2024) Symplasmic phloem loading and subcellular transport in storage roots are key factors for carbon allocation in cassava. bioRxiv.), it still exhibits active transport, which may be especially important for the long-distance transport required in the cassava stem.

### BRIEF SUMMARY

Increasing potassium in the soil, facilitating potassium uptake from the soil, and improving potassium transport in the plant represent promising approaches for improving growth and yield of plants. These approaches may be particularly beneficial for plants with long transport distances between source and sink and/or plants that produce tubers, such as cassava. There exists a need for genetic engineering approaches to improve phloem loading and transport in such plants. One way in which this could be achieved is through increased AKT2 activity. These approaches could increase the delivery of assimilates to, e.g., cassava storage roots, thereby improving storage root yield.

In order to meet these needs, the present disclosure provides modified AKT2 proteins and overexpressed AKT2 proteins. In particular, the present disclosure provides modified Arabidopsis thaliana POTASSIUM TRANSPORTER 2 (AtAKT2) proteins, modified first Manihot esculenta AKT2 proteins (MeAKT2a), and modified second Manihot esculenta AKT2 proteins (MeAKT2b), as well as promoters suitable for overexpression of AKT2 proteins. The present disclosure further provides genetically modified plants, plant parts thereof, methods of producing genetically modified plants, and expression vectors including modified AKT2 proteins and overexpressed AKT2 proteins.

An aspect of the disclosure includes a genetically modified plant or plant part thereof including one or more nucleotide sequences encoding a modified POTASSIUM TRANSPORTER 2 (AKT2) protein. In an additional embodiment of this aspect, the modified AKT2 protein is selected from the group of a modified Arabidopsis thaliana POTASSIUM TRANSPORTER 2 (AtAKT2) protein, a modified first Manihot esculenta AKT2 protein (MeAKT2a), and/or a modified second Manihot esculenta AKT2 protein (MeAKT2b). One aspect of the present disclosure provides a genetically modified plant, plant part thereof, or plant cell thereof, including one or more nucleotide sequences encoding a modified POTASSIUM TRANSPORTER 2 (AKT2) protein, wherein the modified AKT2 protein is selected from the group of a modified plant AKT2 protein, a modified Arabidopsis thaliana AKT2 (AtAKT2) protein, a modified first Manihot esculenta AKT2 protein (MeAKT2a), and/or a modified second Manihot esculenta AKT2 protein (MeAKT2b), or a homolog thereof. In some embodiments, a wild-type AKT2 protein has phloem potassium transport activity and the modified AKT2 protein has phloem potassium transport activity. In a further embodiment of this aspect, an AKT2 protein includes: (a) mode 1, wherein the AKT2 protein acts as an inward-rectifying K+ channel (Kᵢₙ); and (b) mode 2, wherein the AKT2 protein acts as a nonrectifying channel; wherein the wild-type AKT2 protein comprises mode 1; and wherein the modified AKT2 protein comprises modifications that bias the modified AKT2 toward mode 2 or lock the modified AKT2 in mode 2. In another embodiment, which may be combined with any preceding embodiment, the modified AKT2 protein, the modified plant AKT2 protein, the modified AtAKT2 protein, the modified MeAKT2a protein, and/or the modified MeAKT2b protein (a) includes one or more amino acid substitutions, insertions, or deletions in the Ion_trans (PF00520, Ion transport protein) motif (corresponding to amino acids 75-323 of SEQ ID NO: 17) and/or one or more amino acid substitutions, insertions, or deletions in the Ion_trans_2 (PF07885, Ion channel) motif (corresponding to amino acids 225-317 of SEQ ID NO: 17), wherein the one or more substitutions, insertions, or deletions modulate or increase the ion transport activity; (b) includes one or more amino acid substitutions, insertions, or deletions, wherein the one or more substitutions, insertions, or deletions modulate or increase the ion transport activity; (c) includes one or both of the amino acid substitutions corresponding to S210N and S329N when aligned to SEQ ID NO: 17; (d) includes one or both of the amino acid substitutions corresponding to S199N and S139N when aligned to SEQ ID NO: 19; or (e) includes one or both of the amino acid substitutions corresponding to S216N and S319N when aligned to SEQ ID NO: 20. In still another embodiment of this aspect, the wild-type AKT2 protein includes a protein comprising at least 70% identity, at least 80% identity, at least 90% identity, at least 95% identity, or at least 99% identity to one of SEQ ID NOs: 17, 19, and 20, the wild-type AtAKT2 protein includes a protein including at least 70% identity, at least 80% identity, at least 90% identity, at least 95% identity, or at least 99% identity to SEQ ID NO: 17, the wild-type MeAKT2a protein includes a protein including at least 70% identity, at least 80% identity, at least 90% identity, at least 95% identity, or at least 99% identity to SEQ ID NO: 19, and the wild-type MeAKT2b protein includes a protein including at least 70% identity, at least 80% identity, at least 90% identity, at least 95% identity, or at least 99% identity to SEQ ID NO: 20; or a functional fragment of one of the foregoing. In still another embodiment of this aspect, which may be combined with any preceding embodiment, the wild-type plant AKT2 protein includes a protein including at least 70% identity, at least 80% identity, at least 90% identity, at least 95% identity, or at least 99% identity to one of SEQ ID NOs: 17, 19, and 20, the wild-type AtAKT2 protein includes a protein including at least 70% identity, at least 80% identity, at least 90% identity, at least 95% identity, or at least 99% identity to SEQ ID NO: 17, the wild-type MeAKT2a protein includes a protein including at least 70% identity, at least 80% identity, at least 90% identity, at least 95% identity, or at least 99% identity to SEQ ID NO: 19, and the wild-type MeAKT2b protein includes a protein including at least 70% identity, at least 80% identity, at least 90% identity, at least 95% identity, or at least 99% identity to SEQ ID NO: 20; or a functional fragment of one of the foregoing. In still another embodiment of this aspect, which may be combined with any preceding embodiment, the modified plant AKT2 protein includes a protein including at least 70% identity, at least 80% identity, at least 90% identity, at least 95% identity, or at least 99% identity to one of SEQ ID NOs: 18, 25, and 26 the modified AtAKT2 protein includes a protein including at least 70% identity, at least 80% identity, at least 90% identity, at least 95% identity, or at least 99% identity to SEQ ID NO: 18, wherein the modified MeAKT2a protein includes a protein including at least 70% identity, at least 80% identity, at least 90% identity, at least 95% identity, or at least 99% identity to SEQ ID NO: 25, and wherein the modified MeAKT2b protein includes a protein including at least 70% identity, at least 80% identity, at least 90% identity, at least 95% identity, or at least 99% identity to SEQ ID NO: 26; or a functional fragment of one of the foregoing. In still another embodiment of this aspect, which may be combined with any preceding embodiment, the one or more nucleotide sequences encoding the modified AtAKT2 protein includes a nucleotide sequence including at least 70% identity, at least 80% identity, at least 90% identity, at least 95% identity, or at least 99% identity to SEQ ID NO: 3.

In still another embodiment of this aspect, which may be combined with any of the preceding embodiments, the modified plant AKT2 protein, the modified AtAKT2 protein, the modified MeAKT2a protein, and/or the modified MeAKT2b protein (a) includes one or more amino acid substitutions, insertions, or deletions in the Ion_trans (PF00520, Ion transport protein) motif (corresponding to amino acids 75-323 of SEQ ID NO: 17) and/or one or more amino acid substitutions, insertions, or deletions in the Ion_trans_2 (PF07885, Ion channel) motif (corresponding to amino acids 225-317 of SEQ ID NO: 17), wherein the one or more substitutions, insertions, or deletions modulate or increase the ion transport activity; (b) includes one or more amino acid substitutions, insertions, or deletions, wherein the one or more substitutions, insertions, or deletions modulate or increase the ion transport activity; or (c) includes one or both of the amino acid substitutions corresponding to S210N and S329N when aligned to SEQ ID NO: 17. In yet another embodiment of this aspect, which may be combined with any of the preceding embodiments, the modified plant AKT2 protein, the modified AtAKT2 protein, the modified MeAKT2a protein, and/or the modified MeAKT2b protein includes one or both of the amino acid substitutions corresponding to S210N and S329N when aligned to SEQ ID NO: 17. In an additional embodiment of this aspect, which may be combined with any of the preceding embodiments, the modified plant AKT2 protein includes a protein comprising at least 70% identity, at least 80% identity, at least 90% identity, at least 95% identity, or at least 99% identity to one of SEQ ID NOs: 18, 25, and 26, the modified AtAKT2 protein includes a protein including at least 70% identity, at least 80% identity, at least 90% identity, at least 95% identity, or at least 99% identity to SEQ ID NO: 18, wherein the modified MeAKT2a protein includes a protein comprising at least 70% identity, at least 80% identity, at least 90% identity, at least 95% identity, or at least 99% identity to SEQ ID NO: 25, and wherein the modified MeAKT2b protein includes a protein comprising at least 70% identity, at least 80% identity, at least 90% identity, at least 95% identity, or at least 99% identity to SEQ ID NO: 26; or a functional fragment of one of the foregoing. In a further embodiment of this aspect, which may be combined with any of the preceding embodiments, the one or more nucleotide sequences encoding the modified AtAKT2 protein includes a nucleotide sequence including at least 70% identity, at least 80% identity, at least 90% identity, at least 95% identity, or at least 99% identity to SEQ ID NO: 3. In some embodiments, which may be combined with any of the preceding embodiments, the wild-type plant AKT2 protein comprises a protein comprising at least 70% identity, at least 80% identity, at least 90% identity, at least 95% identity, or at least 99% identity to one of SEQ ID NOs: 17, 19, and 20, the wild-type plant AtAKT2 protein comprises a protein comprising at least 70% identity, at least 80% identity, at least 90% identity, at least 95% identity, or at least 99% identity to SEQ ID NO: 17, the wild-type MeAKT2a protein comprises a protein comprising at least 70% identity, at least 80% identity, at least 90% identity, at least 95% identity, or at least 99% identity to SEQ ID NO: 19, and the wild-type MeAKT2b protein comprises a protein comprising at least 70% identity, at least 80% identity, at least 90% identity, at least 95% identity, or at least 99% identity to SEQ ID NO: 20; or a functional fragment of one of the foregoing; the modified plant AKT2 protein comprises a protein comprising at least 70% identity, at least 80% identity, at least 90% identity, at least 95% identity, or at least 99% identity to one of SEQ ID NOs: 18, 25, and 26, the modified AtAKT2 protein comprises a protein comprising at least 70% identity, at least 80% identity, at least 90% identity, at least 95% identity, or at least 99% identity to SEQ ID NO: 18, wherein the modified MeAKT2a protein comprises a protein comprising at least 70% identity, at least 80% identity, at least 90% identity, at least 95% identity, or at least 99% identity to SEQ ID NO: 25, and wherein the modified MeAKT2b protein comprises a protein comprising at least 70% identity, at least 80% identity, at least 90% identity, at least 95% identity, or at least 99% identity to SEQ ID NO: 26; or a functional fragment of one of the foregoing; and/or the one or more nucleotide sequences encoding the modified AtAKT2 protein includes a nucleotide sequence including at least 70% identity, at least 80% identity, at least 90% identity, at least 95% identity, or at least 99% identity to SEQ ID NO: 3. In still another embodiment of this aspect, which may be combined with any of the preceding embodiments, the one or more nucleotide sequences encoding the modified plant AKT2 protein, the modified AtAKT2 protein, the modified MeAKT2a protein, and/or the modified MeAKT2b protein is operably linked to an expression control sequence. In an additional embodiment of this aspect, the expression control sequence includes an overexpression promoter, a phloem-specific promoter, and/or a xylem-specific promoter. In still another embodiment of this aspect, the promoter includes an Arabidopsis thaliana POTASSIUM TRANSPORTER 2 (pAtAKT2) promoter, an Arabidopsis thaliana SUCROSE TRANSPORTER 1 promoter (pAtSUC1), a COMMELINA YELLOW MOTTLE VIRUS promoter (pCoYMV), a Rice tungro bacilliform virus promoter (pRTBV), a Solanum tuberosum KST1 promoter (pStKST1), a cassava MeAKT2a promoter, a cassava MeAKT2b promoter, or a proIC promoter. In still another embodiment of this aspect, which may be combined with any of the preceding embodiments, the one or more nucleotide sequences encoding the modified plant AKT2 protein, the modified AtAKT2 protein, the modified MeAKT2a protein, and/or the modified MeAKT2b protein is operably linked to an expression control sequence; wherein the expression control sequence includes an overexpression promoter, a phloem-specific promoter, and/or a xylem-specific promoter; and optionally wherein the promoter includes an Arabidopsis thaliana POTASSIUM TRANSPORTER 2 (pAtAKT2) promoter, an Arabidopsis thaliana SUCROSE TRANSPORTER 2 promoter (pAtSUC2), a COMMELINA YELLOW MOTTLE VIRUS promoter (pCoYMV), a Rice tungro bacilliform virus promoter (pRTBV), a Solanum tuberosum KST1 promoter (pStKST1), a cassava MeAKT2a promoter, a cassava MeAKT2b promoter, or a proIC promoter. In a further embodiment of this aspect, the promoter includes the pAtAKT2 promoter, the cassava MeAKT2a promoter, or the cassava MeAKT2b promoter. In yet another embodiment of this aspect, the promoter includes the pAtAKT2 promoter, and wherein the promoter includes a nucleotide sequence including at least 70% identity, at least 80% identity, at least 90% identity, at least 95% identity, or at least 99% identity to SEQ ID NO: 2.

An additional aspect of the disclosure includes a genetically modified plant or plant part thereof including one or more nucleotide sequences encoding a POTASSIUM TRANSPORTER 2 (AKT2) protein operably linked to an expression control sequence, wherein the expression control sequence includes an overexpression promoter, optionally wherein the AKT2 protein is a wild-type protein. In a further embodiment of this aspect, the AKT2 protein is selected from the group of an Arabidopsis thaliana POTASSIUM TRANSPORTER 2 (AtAKT2) protein, a first Manihot esculenta AKT2 protein (MeAKT2a), and/or a second Manihot esculenta AKT2 protein (MeAKT2b). In another embodiment of this aspect, the AKT2 protein comprises a protein comprising at least 70% identity, at least 80% identity, at least 90% identity, at least 95% identity, or at least 99% identity to one of SEQ ID NOs: 17-20, 25, and 26, the AtAKT2 protein includes a protein including at least 70% identity, at least 80% identity, at least 90% identity, at least 95% identity, or at least 99% identity to SEQ ID NO: 17 or SEQ ID NO: 18, the MeAKT2a protein comprises a protein comprising at least 70% identity, at least 80% identity, at least 90% identity, at least 95% identity, or at least 99% identity to SEQ ID NO: 19 or SEQ ID NO: 25, and the MeAKT2b protein comprises a protein comprising at least 70% identity, at least 80% identity, at least 90% identity, at least 95% identity, or at least 99% identity to SEQ ID NO: 20 or SEQ ID NO: 26; or a functional fragment of one of the foregoing. In still another embodiment of this aspect, which may be combined with any of the preceding embodiments, the overexpression promoter additionally includes tissue-specific expression, and wherein the tissue-specific expression is selected from the group of phloem-specific expression, xylem-specific expression, root-specific expression, or stomata-specific expression. In yet another embodiment of this aspect, which may be combined with any of the preceding embodiments, the overexpression promoter includes an Arabidopsis thaliana POTASSIUM TRANSPORTER 2 (pAtAKT2) promoter, an Arabidopsis thaliana SUCROSE TRANSPORTER 2 promoter (pAtSUC2), a COMMELINA YELLOW MOTTLE VIRUS promoter (pCoYMV), a Rice tungro bacilliform virus promoter (pRTBV), a Solanum tuberosum KST1 promoter (pStKST1), a cassava MeAKT2a promoter, or a cassava MeAKT2b promoter. In a further embodiment of this aspect, the promoter includes the pAtAKT2 promoter, the cassava MeAKT2a promoter, or the cassava MeAKT2b promoter. In an additional embodiment of this aspect, the promoter is the pAtAKT2 promoter, and wherein the promoter includes a nucleotide sequence including at least 70% identity, at least 80% identity, at least 90% identity, at least 95% identity, or at least 99% identity to SEQ ID NO: 2.

In a further embodiment of this aspect, which may be combined with any of the preceding embodiments, the plant is a dicot and/or the plant produces storage roots or tubers. In one embodiment of this aspect, the plant produces storage roots. In another embodiment of this aspect, the plant produces tubers. In an additional embodiment of this aspect, the plant is selected from the group of cassava, potato, sweet potato, yam, ube, yacón, taro, konjac, ginger, radish, turnip, rutabaga, parsnip, jicama, Jerusalem artichoke, turmeric, horseradish, beet, lotus, maca, celeriac, skirret, or wasabi. In yet another embodiment, the plant can be a crop that benefits from potassium fertilization. For example, some crops that can benefit from potassium fertilization can be cassava, yams, potatoes, tomatoes, citrus fruits (e.g., oranges, lemons, limes, etc.), bananas, grains (e.g., wheat, rice, barley, sorghum, etc.), cotton, sugar beets, soybeans, cowpea, alfalfa, grapes, peppers, apples, and melons (e.g., watermelon, cantaloupe, etc.). In a further embodiment of this aspect, which may be combined with any of the preceding embodiments, the plant is a dicot; the plant produces storage roots or tubers and/or benefits from potassium fertilization, optionally wherein the plant is selected from the group consisting of cassava, potato, sweet potato, yam, ube, yacón, taro, konjac, ginger, radish, turnip, rutabaga, parsnip, jicama, Jerusalem artichoke, turmeric, horseradish, beet, lotus, maca, celeriac, skirret, wasabi, citrus fruits, bananas, grains, tomatoes, sorghum, cotton, sugar beets, soybeans, cowpea, alfalfa, grapes, peppers, apples, and melons; and/or the plant has a large transport distance between a storage organ and a photosynthetic leaf. In still another embodiment of this aspect, which may be combined with any of the preceding embodiments, the genetically modified plant has improved phloem transport, improved phloem mass flow, improved source-sink delivery, increased fibrous root formation, higher photosynthetic efficiency, improved photosynthesis, higher rate of CO₂ fixation and/or electron transport rate, earlier maximum growth rate, increased yield under field conditions, increased yield under drought conditions, increased drought stress resistance, improved drought tolerance, and/or increased yield under potassium deficiency as compared to a control plant grown under the same conditions, optionally wherein the genetically modified plant or a progenitor thereof was selected for improved phloem transport, improved phloem mass flow, improved source-sink delivery, increased fibrous root formation, increased growth, improved photosynthesis, higher rate of CO₂ fixation, and/or electron transport rate when grown under non-limiting energy conditions, earlier maximum growth rate, increased yield, increased plant growth, increased plant height, increased shoot growth, increased total shoot dry matter, increased storage root or tuber biomass, increased number of storage roots or tubers per plant, and/or increased total storage root or tuber dry matter. In yet another embodiment of this aspect, which may be combined with any of the preceding embodiments, the genetically modified plant or plant part thereof is a cassava plant, and wherein the genetically modified cassava plant has increased plant growth, increased plant height, increased shoot growth, increased total shoot dry matter, improved storage root growth, improved number of storage roots per plant, and/or increased total storage root dry matter as compared to a control cassava plant grown under the same conditions.

In yet another embodiment of this aspect, which may be combined with any of the preceding embodiments, the genetically modified plant is a cassava plant, wherein the genetically modified cassava plant has improved phloem transport, increased plant growth, increased plant height, increased shoot growth, increased total shoot dry matter (TSDM), increased storage root growth, increased drought stress resistance, increased drought tolerance, improved photosynthetic performance, lower proline and/or serine levels in drought conditions, increased number of storage root per plant, and/or increased total storage root dry matter (TRDM) as compared to a control cassava plant grown under the same conditions, the genetically modified plant includes (a) at least one of the following shoot traits: increased height, increased concentrations of sodium (Na+), increased concentrations of calcium (Ca2+), increased concentrations of magnesium (Mg2+), increased concentrations of potassium (K+), reduced sucrose concentration or level in aboveground plant parts, increased starch concentration or level, increased shoot fresh weight, increased TSDM, and increased phloem transport rate; and/or (b) at least one of the following root traits: reduced concentrations of K+, reduced sucrose concentration, increased glucose concentration, increased fructose concentration, increased starch concentration, increased root fresh weight, and increased TRDM as compared to a control plant grown under the same conditions. In a further embodiment of this aspect, the genetically modified cassava plant has elevated concentrations of sodium (Na⁺), calcium (Ca²⁺), magnesium (Mg²⁺), and/or potassium (K⁺) in shoot tissue, reduced concentrations of sodium (Na⁺), calcium (Ca²⁺), magnesium (Mg²⁺), and/or potassium (K⁺) in root tissue, reduced sucrose concentration in shoot and/or root tissue, increased glucose and/or fructose concentration in root tissue, and/or increased starch concentration in root tissue as compared to a control cassava plant grown under the same conditions. In an additional embodiment of this aspect, which may be combined with any of the preceding embodiments that has a cassava plant, the genetically modified cassava plant includes cultivar TMS60444. In another embodiment of this aspect, which may be combined with any of the preceding embodiments, the genetically modified plant comprises (a) at least one of the following shoot traits: increased height, increased concentrations of sodium (Na⁺), increased concentrations of calcium (Ca²⁺), increased concentrations of magnesium (Mg²⁺), increased concentrations of potassium (K⁺), reduced sucrose concentration or level in aboveground plant parts, increased starch concentration or level, increased shoot fresh weight, increased TSDM, and increased phloem transport rate; and/or (b) at least one of the following root traits: reduced concentrations of K⁺, reduced sucrose concentration, increased glucose concentration, increased fructose concentration, increased starch concentration, increased root fresh weight, and increased TRDM as compared to a control plant grown under the same conditions. In yet another embodiment of this aspect, which may be combined with any of the preceding embodiments, the genetically modified plant (a) reaches the maximum relative growth rate (RGR) faster, (b) has an increased harvest index (HI), (c) has increased yield, (d) has a higher maximum electron transport rate (ETR), (e) has an increased tracer transport velocity; and/or (f) has an increased CO2 assimilation rate as compared to a control plant grown under the same conditions. In yet another embodiment of this aspect, which may be combined with any of the preceding embodiments, the genetically modified plant grown under drought conditions has (a) increased relative yield, (b) elevated sucrose concentrations; (c) elevated glucose concentrations; (d) elevated fructose concentrations; (e) elevated starch concentrations; (f) increased TSDM; (g) increased TRDM; and/or (h) reduced serine and/or proline concentrations as compared to a control plant grown under drought conditions. In yet another embodiment of this aspect, which may be combined with any of the preceding embodiments, the genetically modified plant exhibits increased drought stress resistance and/or increased drought tolerance as compared to a control plant grown under the same conditions, and wherein the increased drought stress resistance and/or increased drought tolerance is indicated by reduced proline concentrations, reduced serine concentrations, and/or increased relative yield as compared to a control plant grown under the same conditions.

A further aspect of the disclosure includes methods of producing the genetically modified plant or plant part thereof of any one of the preceding embodiments that has a modified AKT2 protein, including introducing one or more nucleotide sequences encoding the modified plant AKT2 protein, modified AtAKT2 protein, the modified MeAKT2a protein, and/or the modified MeAKT2b protein. In some embodiments of this aspect, the method includes introducing one or more nucleotide sequences encoding the modified plant AKT2 protein, the modified AtAKT2 protein, the modified MeAKT2a protein, and/or the modified MeAKT2b protein, optionally wherein the one or more nucleotide sequences are operably linked to the expression control sequence comprising the overexpression promoter. In a further embodiment of this aspect, which may be combined with any previous embodiment including the one or more nucleotide sequences, the one or more nucleotide sequences encoding the modified plant AKT2 protein, the modified AtAKT2 protein, the modified MeAKT2a protein, and/or the modified MeAKT2b protein is operably linked to an expression control sequence; wherein the expression control sequence comprises an overexpression promoter and/or a phloem-specific promoter; and optionally wherein the promoter comprises a plant AKT2 promoter, an Arabidopsis thaliana POTASSIUM TRANSPORTER 2 (pAtAKT2) promoter, an Arabidopsis thaliana SUCROSE TRANSPORTER 1 promoter (pAtSUC2), a cassava MeAKT2a promoter, a cassava MeAKT2b promoter, or a proIC promoter. In still another embodiment of this aspect, the modified plant AKT2 protein, the modified AtAKT2 protein, the modified MeAKT2a protein, and/or the modified MeAKT2b protein (a) comprises one or more amino acid substitutions, insertions, or deletions in the Ion_trans (PF00520, Ion transport protein) motif (corresponding to amino acids 75-323 of SEQ ID NO: 17) and/or one or more amino acid substitutions, insertions, or deletions in the Ion_trans_2 (PF07885, Ion channel) motif (corresponding to amino acids 225-317 of SEQ ID NO: 17), wherein the one or more substitutions, insertions, or deletions modulate or increase the ion transport activity; or (b) comprises one or more amino acid substitutions, insertions, or deletions, wherein the one or more substitutions, insertions, or deletions modulate or increase the ion transport activity. In an additional embodiment of this aspect, the modified plant AKT2 protein, the modified AtAKT2 protein, the modified MeAKT2a protein, and/or the modified MeAKT2b protein includes one or both of the amino acid substitutions corresponding to S210N and S329N when aligned to SEQ ID NO: 17. In yet another embodiment of this aspect, which may be combined with any of the preceding embodiments, the modified plant AKT2 protein comprises a protein comprising at least 70% identity, at least 80% identity, at least 90% identity, at least 95% identity, or at least 99% identity to one of SEQ ID NOs: 18, 25, and 26, the modified AtAKT2 protein includes a protein including at least 70% identity, at least 80% identity, at least 90% identity, at least 95% identity, or at least 99% identity to SEQ ID NO: 18, wherein the modified MeAKT2a protein includes a protein comprising at least 70% identity, at least 80% identity, at least 90% identity, at least 95% identity, or at least 99% identity to SEQ ID NO: 25, and wherein the modified MeAKT2b protein includes a protein comprising at least 70% identity, at least 80% identity, at least 90% identity, at least 95% identity, or at least 99% identity to SEQ ID NO: 26; or a functional fragment of one of the foregoing. In still another embodiment of this aspect, which may be combined with any of the preceding embodiments, the one or more nucleotide sequences encoding the modified AtAKT2 protein includes a nucleotide sequence including at least 70% identity, at least 80% identity, at least 90% identity, at least 95% identity, or at least 99% identity to SEQ ID NO: 3. In a further embodiment of this aspect, which may be combined with any of the preceding embodiments, the one or more nucleotide sequences encoding the modified plant AKT2 protein, the modified AtAKT2 protein, the modified MeAKT2a protein, and/or the modified MeAKT2b protein is operably linked to an expression control sequence. In yet another embodiment of this aspect, the expression control sequence includes an overexpression promoter and/or a phloem-specific promoter. In still another embodiment of this aspect, the promoter includes an Arabidopsis thaliana POTASSIUM TRANSPORTER 2 (pAtAKT2) promoter, an Arabidopsis thaliana SUCROSE TRANSPORTER 1 promoter (pAtSUC1), a COMMELINA YELLOW MOTTLE VIRUS promoter (pCoYMV), a Rice tungro bacilliform virus promoter (pRTBV), a Solanum tuberosum KST1 promoter (pStKST1), a cassava MeAKT2a promoter, or a cassava MeAKT2b promoter. In an additional embodiment of this aspect, the promoter includes the pAtAKT2 promoter, the cassava MeAKT2a promoter, or the cassava MeAKT2b promoter. In a further embodiment of this aspect, the promoter includes the pAtAKT2 promoter, and wherein the promoter includes a nucleotide sequence including at least 70% identity, at least 80% identity, at least 90% identity, at least 95% identity, or at least 99% identity to SEQ ID NO: 2.

In some embodiments of this aspect, the one or more nucleotide sequences encoding the modified plant AKT2 protein, the modified AtAKT2 protein, the modified MeAKT2a protein, and/or the modified MeAKT2b protein is operably linked to an expression control sequence; wherein the expression control sequence includes an overexpression promoter and/or a phloem-specific promoter; and optionally wherein the promoter comprises a plant AKT2 promoter, an Arabidopsis thaliana POTASSIUM TRANSPORTER 2 (pAtAKT2) promoter, an Arabidopsis thaliana SUCROSE TRANSPORTER 1 promoter (pAtSUC2), a cassava MeAKT2a promoter, a cassava MeAKT2b promoter, or a proIC promoter.

Yet another aspect of the disclosure includes methods of producing the genetically modified plant or plant part thereof of any one of the preceding embodiments that has a modified plant AKT2 protein, including genetically modifying a plant by transforming the plant with one or more gene editing components that target an endogenous nuclear genome sequence encoding the wild-type plant ATK2 protein, wild-type AtAKT2 protein, the wild-type MeAKT2a protein, and/or the wild-type MeAKT2b protein to produce the modified plant AKT2 protein, modified AtAKT2 protein, the modified MeAKT2a protein, and/or the modified MeAKT2b protein. In a further embodiment of this aspect, the one or more gene editing components include a ribonucleoprotein complex that targets the nuclear genome sequence; a vector including a TALEN protein encoding sequence, wherein the TALEN protein targets the nuclear genome sequence; a vector including a ZFN protein encoding sequence, wherein the ZFN protein targets the nuclear genome sequence; an oligonucleotide donor (OND), wherein the OND targets the nuclear genome sequence; or a vector CRISPR/Cas enzyme encoding sequence and a targeting sequence, wherein the targeting sequence targets the nuclear genome sequence. In a further embodiment of the aspect including a method of producing the genetically modified plant or plant part thereof, the method includes genetically modifying a plant by transforming the plant with one or more gene editing components that target an endogenous nuclear genome sequence encoding a promoter of an endogenous plant AKT2 protein, a promoter of an endogenous AtAKT2 protein, a promoter of an endogenous MeAKT2a protein, and/or a promoter of an endogenous MeAKT2b protein to produce a modified AKT2 promoter, a modified AtAKT2 promoter, a modified MeAKT2a promoter, and/or a modified MeAKT2b promoter, wherein the modified AKT2 promoter, the modified AtAKT2 promoter, the modified MeAKT2a promoter, and/or the modified MeAKT2b promoter has increased expression and/or altered tissue-specific expression as compared to the unmodified promoter, wherein the one or more gene editing components comprise a ribonucleoprotein complex that targets the nuclear genome sequence; a vector including a TALEN protein encoding sequence, wherein the TALEN protein targets the nuclear genome sequence; a vector including a ZFN protein encoding sequence, wherein the ZFN protein targets the nuclear genome sequence; an oligonucleotide donor (OND), wherein the OND targets the nuclear genome sequence; or a vector CRISPR/Cas enzyme encoding sequence and a targeting sequence, wherein the targeting sequence targets the nuclear genome sequence. In an additional embodiment of this aspect, which may be combined with any one of the preceding embodiments, the wild-type plant AKT2 protein includes a protein including at least 70% identity, at least 80% identity, at least 90% identity, at least 95% identity, or at least 99% identity to one of SEQ ID NOs: 17, 19, and 20, the wild-type AtAKT2 protein includes a protein including at least 70% identity, at least 80% identity, at least 90% identity, at least 95% identity, or at least 99% identity to SEQ ID NO: 17, the wild-type MeAKT2a protein includes a protein including at least 70% identity, at least 80% identity, at least 90% identity, at least 95% identity, or at least 99% identity to SEQ ID NO: 19, and the wild-type MeAKT2b protein includes a protein including at least 70% identity, at least 80% identity, at least 90% identity, at least 95% identity, or at least 99% identity to SEQ ID NO: 20; or a functional fragment of one of the foregoing. In an additional embodiment of this aspect, which may be combined with any one of the preceding embodiments, the modified plant AKT2 protein includes a protein including at least 70% identity, at least 80% identity, at least 90% identity, at least 95% identity, or at least 99% identity to one of SEQ ID NOs: 18, 25, and 26 the modified AtAKT2 protein includes a protein including at least 70% identity, at least 80% identity, at least 90% identity, at least 95% identity, or at least 99% identity to SEQ ID NO: 18, wherein the modified MeAKT2a protein includes a protein including at least 70% identity, at least 80% identity, at least 90% identity, at least 95% identity, or at least 99% identity to SEQ ID NO: 25, and wherein the modified MeAKT2b protein includes a protein including at least 70% identity, at least 80% identity, at least 90% identity, at least 95% identity, or at least 99% identity to SEQ ID NO: 26; or a functional fragment of one of the foregoing; and/or (iii) the one or more nucleotide sequences encoding the modified AtAKT2 protein includes a nucleotide sequence including at least 70% identity, at least 80% identity, at least 90% identity, at least 95% identity, or at least 99% identity to SEQ ID NO: 3. In still another embodiment of this aspect, which may be combined with any of the preceding embodiments, the modified plant AKT2 protein, the modified AtAKT2 protein, the modified MeAKT2a protein, and/or the modified MeAKT2b protein (a) includes one or more amino acid substitutions, insertions, or deletions in the Ion_trans (PF00520, Ion transport protein) motif (corresponding to amino acids 75-323 of SEQ ID NO: 17) and/or one or more amino acid substitutions, insertions, or deletions in the Ion_trans_2 (PF07885, Ion channel) motif (corresponding to amino acids 225-317 of SEQ ID NO: 17), wherein the one or more substitutions, insertions, or deletions modulate or increase the ion transport activity; (b) includes one or more amino acid substitutions, insertions, or deletions, wherein the one or more substitutions, insertions, or deletions modulate or increase the ion transport activity; or (c) includes one or both of the amino acid substitutions corresponding to S210N and S329N when aligned to SEQ ID NO: 17. In yet another embodiment of this aspect, which may be combined with any of the preceding embodiments, the modified plant AKT2 protein, modified AtAKT2 protein, the modified MeAKT2a protein, and/or the modified MeAKT2b protein includes one or both of the amino acid substitutions corresponding to S210N and S329N when aligned to SEQ ID NO: 17. In still another embodiment of this aspect, which may be combined with any of the preceding embodiments, the modified plant AKT2 protein comprises a protein comprising at least 70% identity, at least 80% identity, at least 90% identity, at least 95% identity, or at least 99% identity to one of SEQ ID NOs: 18, 25, and 26, the modified AtAKT2 protein includes a protein including at least 70% identity, at least 80% identity, at least 90% identity, at least 95% identity, or at least 99% identity to SEQ ID NO: 18, wherein the modified MeAKT2a protein includes a protein comprising at least 70% identity, at least 80% identity, at least 90% identity, at least 95% identity, or at least 99% identity to SEQ ID NO: 25, and wherein the modified MeAKT2b protein includes a protein comprising at least 70% identity, at least 80% identity, at least 90% identity, at least 95% identity, or at least 99% identity to SEQ ID NO: 26; or a functional fragment of one of the foregoing.

Still another aspect of the disclosure includes methods of producing the genetically modified plant or plant part thereof of any one of the preceding embodiments that has a plant AKT2 protein operably linked to an overexpression promoter, including introducing one or more nucleotide sequences encoding the plant AKT2 protein, the AtAKT2 protein, the MeAKT2a protein, and/or the MeAKT2b protein operably linked to the expression control sequence including the overexpression promoter. In a further embodiment of this aspect, the AKT2 protein comprises a protein comprising at least 70% identity, at least 80% identity, at least 90% identity, at least 95% identity, or at least 99% identity to one of SEQ ID NOs: 17-19, 25, and 26, the AtAKT2 protein includes a protein including at least 70% identity, at least 80% identity, at least 90% identity, at least 95% identity, or at least 99% identity to SEQ ID NO: 17 or SEQ ID NO: 18, the MeAKT2a protein comprises a protein comprising at least 70% identity, at least 80% identity, at least 90% identity, at least 95% identity, or at least 99% identity to SEQ ID NO: 19 or SEQ ID NO: 25, and the MeAKT2b protein comprises a protein comprising at least 70% identity, at least 80% identity, at least 90% identity, at least 95% identity, or at least 99% identity to SEQ ID NO: 20 or SEQ ID NO: 26; or a functional fragment of one of the foregoing. In an additional embodiment of this aspect, which may be combined with any of the preceding embodiments, the overexpression promoter includes the Arabidopsis thaliana POTASSIUM TRANSPORTER 2 (pAtAKT2) promoter, the Arabidopsis thaliana SUCROSE TRANSPORTER 2 promoter (pAtSUC2), the COMMELINA YELLOW MOT TLE VIRUS promoter (pCoYMV), the Rice tungro bacilliform virus promoter (pRTBV), the Solanum tuberosum KST1 promoter (pStKST1), the cassava MeAKT2a promoter, or the cassava MeAKT2b promoter. In yet another embodiment of this aspect, the promoter includes the pAtAKT2 promoter, the cassava MeAKT2a promoter, the cassava MeAKT2b promoter, or a proIC promoter.

Yet another aspect of the disclosure includes methods of producing the genetically modified plant or plant part thereof of any one of the preceding embodiments that has an AKT2 protein operably linked to an overexpression promoter, including genetically modifying a plant by transforming the plant with one or more gene editing components that target an endogenous nuclear genome sequence encoding a promoter of an endogenous plant AKT2 protein, a promoter of an endogenous AtAKT2 protein, a promoter of an endogenous MeAKT2a protein, and/or a promoter of an endogenous MeAKT2b protein to produce a modified plant AKT2 promoter, a modified AtAKT2 promoter, a modified MeAKT2a promoter, and/or a modified MeAKT2b promoter, wherein the modified plant AKT2 promoter, the modified AtAKT2 promoter, the modified MeAKT2a promoter, and/or the modified MeAKT2b promoter has increased expression and/or altered tissue-specific expression as compared to the unmodified promoter. In an additional embodiment of this aspect, the one or more gene editing components include a ribonucleoprotein complex that targets the nuclear genome sequence; a vector including a TALEN protein encoding sequence, wherein the TALEN protein targets the nuclear genome sequence; a vector including a ZFN protein encoding sequence, wherein the ZFN protein targets the nuclear genome sequence; an oligonucleotide donor (OND), wherein the OND targets the nuclear genome sequence; or a vector CRISPR/Cas enzyme encoding sequence and a targeting sequence, wherein the targeting sequence targets the nuclear genome sequence.

In a further embodiment of this aspect, which may be combined with any of the preceding embodiments that have methods, the method further includes selecting a genetically modified plant or plant part thereof with improved growth, improved photosynthesis, higher rate of CO₂ fixation, and/or higher electron transport rate when the genetically modified plant or plant part thereof is grown under non-limiting energy conditions, earlier maximum growth rate, improved yield, increased plant growth, increased plant height, increased shoot growth, increased total shoot dry matter, improved storage root or tuber growth, increased number of storage roots or tubers per plant, and/or increased total storage root or tuber dry matter as compared to a control plant. In a further embodiment of this aspect, which may be combined with any of the preceding embodiments that have methods, the method further includes selecting a genetically modified plant or plant part thereof with improved growth, improved photosynthesis, higher rate of CO₂ fixation and/or higher electron transport rate when the genetically modified plant is grown under non-limiting energy conditions, earlier maximum growth rate, increased yield, increased plant growth, increased plant height, increased shoot growth, increased total shoot dry matter, increased storage root or tuber biomass, increased number of storage roots or tubers per plant, and/or increased total storage root or tuber dry matter as compared to a control plant.

Some aspects of the present disclosure relate to a genetically modified plant or plant part thereof produced by the method of any one of the preceding embodiments. In an additional embodiment of this aspect, the plant produces storage roots or tubers. In a further embodiment of this aspect, the plant is selected from the group of cassava, potato, sweet potato, yam, ube, yacón, taro, konjac, ginger, radish, turnip, rutabaga, parsnip, jicama, Jerusalem artichoke, turmeric, horseradish, beet, lotus, maca, celeriac, skirret, or wasabi. In yet another embodiment, the plant can be a crop that benefits from potassium fertilization. For example, some crops that can benefit from potassium fertilization can be cassava, yams, potatoes, tomatoes, citrus fruits (e.g., oranges, lemons, limes, etc.), bananas, grains (e.g., wheat, rice, barley, sorghum, etc.), cotton, sugar beets, soybeans, cowpea, alfalfa, grapes, peppers, apples, melons (e.g., watermelon, cantaloupe, etc.). In an additional embodiment of this aspect, which may be combined with any of the preceding embodiments, the genetically modified plant or plant part thereof has higher photosynthetic efficiency, improved photosynthesis, higher rate of CO₂ fixation and/or electron transport rate, earlier maximum growth rate, improved yield under field conditions, improved yield under drought conditions, and/or improved yield under potassium deficiency as compared to a control plant grown under the same conditions. In an additional embodiment of this aspect, which may be combined with any of the preceding embodiments, the genetically modified plant or plant part thereof has improved phloem transport, improved phloem mass flow, improved source-sink delivery, increased fibrous root formation, higher photosynthetic efficiency, improved photosynthesis, higher rate of CO2 fixation and/or electron transport rate, earlier maximum growth rate, increased yield under field conditions, increased yield under drought conditions, increased drought stress resistance, increased drought tolerance, and/or increased yield under potassium deficiency as compared to a control plant grown under the same conditions, and wherein: (i) the plant produces storage roots or tubers and/or benefits from potassium fertilization, optionally wherein the plant is selected from the group consisting of cassava, potato, sweet potato, yam, ube, yacón, taro, konjac, ginger, radish, turnip, rutabaga, parsnip, jicama, Jerusalem artichoke, turmeric, horseradish, beet, lotus, maca, celeriac, skirret, wasabi, citrus fruits, bananas, grains, tomatoes, sorghum, cotton, sugar beets, soybeans, cowpea, alfalfa, grapes, peppers, apples, and melons; and/or (ii) wherein the plant is a passive symplasmic phloem loader.

An additional aspect of the disclosure includes an expression vector or isolated DNA molecule including one or more nucleotide sequences encoding a modified POTASSIUM TRANSPORTER 2 (AKT2) protein operably linked to an expression control sequence. Further embodiments of this aspect include the modified plant AKT2 protein being selected from the group of a modified Arabidopsis thaliana POTASSIUM TRANSPORTER 2 (AtAKT2) protein, a modified first Manihot esculenta AKT2 protein (MeAKT2a), and/or a modified second Manihot esculenta AKT2 protein (MeAKT2b). In an additional embodiment of this aspect, which may be combined with any of the preceding embodiments, the expression vector or isolated DNA molecule includes one or more gene editing components of preceding embodiments. In yet another embodiment of this aspect, the modified plant AKT2 protein comprises a protein comprising at least 70% identity, at least 80% identity, at least 90% identity, at least 95% identity, or at least 99% identity to one of SEQ ID NOs: 18, 25, and 26, the modified AtAKT2 protein includes a protein including at least 70% identity, at least 80% identity, at least 90% identity, at least 95% identity, or at least 99% identity to SEQ ID NO: 18, wherein the modified MeAKT2a protein includes a protein comprising at least 70% identity, at least 80% identity, at least 90% identity, at least 95% identity, or at least 99% identity to SEQ ID NO: 25, and wherein the modified MeAKT2b protein includes a protein comprising at least 70% identity, at least 80% identity, at least 90% identity, at least 95% identity, or at least 99% identity to SEQ ID NO: 26; or a functional fragment of one of the foregoing. In still another embodiment of this aspect, which may be combined with any one of the preceding embodiments, the one or more nucleotide sequences encoding the modified AtAKT2 protein includes a nucleotide sequence including at least 70% identity, at least 80% identity, at least 90% identity, at least 95% identity, or at least 99% identity to SEQ ID NO: 3.

A further aspect of the disclosure includes an expression vector or isolated DNA molecule including one or more nucleotide sequences encoding a wild-type POTASSIUM TRANSPORTER 2 (AKT2) protein operably linked to an expression control sequence. Additional embodiments of this aspect include the wild-type AKT2 protein being selected from the group of an Arabidopsis thaliana POTASSIUM TRANSPORTER 2 (AtAKT2) protein, a first Manihot esculenta AKT2 protein (MeAKT2a), and/or a second Manihot esculenta AKT2 protein (MeAKT2b). In still another embodiment of this aspect, the plant AKT2 protein comprises a protein comprising at least 70% identity, at least 80% identity, at least 90% identity, at least 95% identity, or at least 99% identity to one of SEQ ID NOs: 17, 19, and 20, the AtAKT2 protein includes a protein including at least 70% identity, at least 80% identity, at least 90% identity, at least 95% identity, or at least 99% identity to SEQ ID NO: 17, the MeAKT2a protein includes a protein including at least 70% identity, at least 80% identity, at least 90% identity, at least 95% identity, or at least 99% identity to SEQ ID NO: 19, and the MeAKT2b protein includes a protein including at least 70% identity, at least 80% identity, at least 90% identity, at least 95% identity, or at least 99% identity to SEQ ID NO: 20; or a functional fragment of one of the foregoing.

In yet another embodiment of this aspect, which may be combined with any of the preceding embodiments that has an expression vector or isolated DNA molecule, the expression control sequence includes an overexpression promoter, a phloem-specific promoter, a xylem-specific promoter, a root-specific promoter, and/or a stomata-specific promoter. In still another embodiment of this aspect, the promoter includes an Arabidopsis thaliana POTASSIUM TRANSPORTER 2 (pAtAKT2) promoter, an Arabidopsis thaliana SUCROSE TRANSPORTER 2 promoter (pAtSUC2), a COMMELINA YELLOW MOTTLE VIRUS promoter (pCoYMV), a Rice tungro bacilliform virus promoter (pRTBV), a Solanum tuberosum KST1 promoter (pStKST1), a cassava MeAKT2a promoter, a cassava MeAKT2b promoter, or a proIC promoter. In a further embodiment of this aspect, the promoter includes the pAtAKT2 promoter, the cassava MeAKT2a promoter, or the cassava MeAKT2b promoter. In an additional embodiment of this aspect, the promoter includes the pAtAKT2 promoter, and wherein the promoter includes a nucleotide sequence including at least 70% identity, at least 80% identity, at least 90% identity, at least 95% identity, or at least 99% identity to SEQ ID NO: 2.

Some aspects of the present disclosure relate to a bacterial cell or an Agrobacterium cell including the expression vector or isolated DNA molecule of any of the preceding embodiments.

Further aspects of the present disclosure relate to a composition or kit including the expression vector or isolated DNA molecule of any of the preceding embodiments, or the bacterial cell or the Agrobacterium cell of the preceding embodiment.

Additional aspects of the present disclosure relate to a genetically modified plant, plant part, plant cell, or seed including the expression vector or isolated DNA molecule of any of the preceding embodiments.

Further aspects of the present disclosure relate to a composition or kit including the genetically modified plant or plant part thereof of any of the preceding embodiments, the genetically modified plant, plant part, plant cell, or seed of the preceding embodiment, or the genetically modified plant or plant part thereof produced by the method of any of the preceding embodiments.

Still further aspects of the present disclosure relate to methods of increasing photosynthetic efficiency, improving photosynthesis, producing earlier maximum growth rate, improving yield under field conditions, improving yield under drought conditions, improving yield under potassium deficiency, increasing plant growth, increasing plant height, increasing shoot growth, increasing total shoot dry matter, improving storage root or tuber growth, increasing number of storage roots or tubers per plant, and/or increasing total storage root or tuber dry matter including: introducing a genetic alteration via the expression vector or isolated DNA molecule of any of the preceding embodiments to a cell, wherein the cell is a plant cell.

Further aspects of the present disclosure relate to a method of improving phloem transport, improving phloem mass flow, improving source-sink delivery, increasing fibrous root formation, increasing photosynthetic efficiency, improving photosynthesis, producing earlier maximum growth rate, increasing yield under field conditions, increasing yield under drought conditions, increasing drought stress resistance, increasing drought tolerance, increasing yield under potassium deficiency, increasing plant growth, increasing plant height, increasing shoot growth, increasing total shoot dry matter, increasing storage root or tuber biomass, increasing number of storage roots or tubers per plant, and/or increasing total storage root or tuber dry matter including: introducing a genetic alteration via the expression vector or isolated DNA molecule of any preceding embodiment including expression vectors or isolated DNA molecules to a cell, wherein the cell is a plant cell.

Further aspects of the present disclosure relate to a genetically altered plant genome including (i) the one or more edited endogenous nucleotide sequences in the genetically modified plant or plant part thereof of any one of the preceding embodiments, or (ii) the one or more edited endogenous nucleotide sequences in the genetically modified plant or plant part thereof produced by the method of any one of the preceding embodiments including a method.

Additional aspects of the present disclosure relate to a non-regenerable part or cell of the genetically modified plant or plant part thereof of any one of the preceding embodiments.

Still another aspect of the present disclosure relates to cassava plant or plant part thereof including (a) one or more nucleotide sequences encoding a modified AtAKT2 protein, a modified MeAKT2a protein, and/or a modified MeAKT2b protein, and (b) improved phloem transport, improved phloem mass flow, improved source-sink delivery, increased fibrous root formation, increased growth, improved photosynthesis, higher rate of CO2 fixation and/or higher electron transport rate when the genetically modified plant is grown under non-limiting energy conditions, earlier maximum growth rate, increased yield, increased plant growth, increased plant height, increased shoot growth, increased total shoot dry matter, increased storage root or tuber biomass, increased number of storage roots or tubers per plant, and/or increased total storage root or tuber dry matter as compared to a control plant.

### ENUMERATED EMBODIMENTS

1. A genetically modified plant comprising one or more nucleotide sequences encoding a modified POTASSIUM TRANSPORTER 2 (AKT2) protein.
2. The genetically modified plant of embodiment 1, wherein the modified AKT2 protein is selected from the group of a modified Arabidopsis thaliana POTASSIUM TRANSPORTER 2 (AtAKT2) protein, a modified first Manihot esculenta AKT2 protein (MeAKT2a), and/or a modified second Manihot esculenta AKT2 protein (MeAKT2b).
3. The genetically modified plant of embodiment 1 or embodiment 2, wherein a wild-type AKT2 protein comprises mode 1, wherein the wild-type AKT2 acts as an inward-rectifying K⁺ channel (Kᵢₙ), and mode 2, wherein the AKT2 acts as a nonrectifying channel, and wherein the modified AKT2 protein comprises modifications that bias the modified AKT2 toward mode 2 or lock the modified AKT2 in mode 2.
4. The genetically modified plant of embodiment 1 or embodiment 2, wherein the wild-type AKT2 protein comprises a protein comprising at least 70% identity, at least 80% identity, at least 90% identity, at least 95% identity, or at least 99% identity to one of SEQ ID NOs: 17, 19, and 20, the wild-type AtAKT2 protein comprises a protein comprising at least 70% identity, at least 80% identity, at least 90% identity, at least 95% identity, or at least 99% identity to SEQ ID NO: 17, the wild-type MeAKT2a protein comprises a protein comprising at least 70% identity, at least 80% identity, at least 90% identity, at least 95% identity, or at least 99% identity to SEQ ID NO: 19, and the wild-type MeAKT2b protein comprises a protein comprising at least 70% identity, at least 80% identity, at least 90% identity, at least 95% identity, or at least 99% identity to SEQ ID NO: 20; or a functional fragment of one of the foregoing.
5. The genetically modified plant of any one of embodiments 1-4, wherein the modified AKT (a) comprises one or more amino acid substitutions, insertions, or deletions in the Ion_trans (PF00520, Ion transport protein) motif (corresponding to amino acids 75-323 of SEQ ID NO: 17) and/or one or more amino acid substitutions, insertions, or deletions in the Ion_trans_2 (PF07885, Ion channel) motif (corresponding to amino acids 225-317 of SEQ ID NO: 17), wherein the one or more substitutions, insertions, or deletions increase the ion transport activity; or (b) comprises one or more amino acid substitutions, insertions, or deletions, wherein the one or more substitutions, insertions, or deletions increase the ion transport activity.
6. The genetically modified plant of any one of embodiments 1-5, wherein the modified AKT2 protein, the modified AtAKT2 protein, the modified MeAKT2a protein, and/or the modified MeAKT2b protein comprises one or both of the amino acid substitutions corresponding to S210N and S329N when aligned to SEQ ID NO: 17.
7. The genetically modified plant of any one of embodiments 1-6, wherein the modified AKT2 protein comprises a protein comprising at least 70% identity, at least 80% identity, at least 90% identity, at least 95% identity, or at least 99% identity to one of SEQ ID NOs: 18, 25, and 26 the modified AtAKT2 protein comprises a protein comprising at least 70% identity, at least 80% identity, at least 90% identity, at least 95% identity, or at least 99% identity to SEQ ID NO: 18, wherein the modified MeAKT2a protein comprises a protein comprising at least 70% identity, at least 80% identity, at least 90% identity, at least 95% identity, or at least 99% identity to SEQ ID NO: 25, and wherein the modified MeAKT2b protein comprises a protein comprising at least 70% identity, at least 80% identity, at least 90% identity, at least 95% identity, or at least 99% identity to SEQ ID NO: 26; or a functional fragment of one of the foregoing.
8. The genetically modified plant of any one of embodiments 1-7, wherein the one or more nucleotide sequences encoding the modified AtAKT2 protein comprises a nucleotide sequence comprising at least 70% identity, at least 80% identity, at least 90% identity, at least 95% identity, or at least 99% identity to SEQ ID NO: 3.
9. The genetically modified plant of any one of embodiments 2-8, wherein the one or more nucleotide sequences encoding the modified AtAKT2 protein, the modified MeAKT2a protein, and/or the modified MeAKT2b protein is operably linked to an expression control sequence.
10. The genetically modified plant of embodiment 9, wherein the expression control sequence comprises an overexpression promoter and/or a phloem-specific promoter.
11. The genetically modified plant of embodiment 10, wherein the promoter comprises an Arabidopsis thaliana POTASSIUM TRANSPORTER 2 (pAtAKT2) promoter, an Arabidopsis thaliana SUCROSE TRANSPORTER 2 promoter (pAtSUC2), a COMMELINA YELLOW MOTTLE VIRUS promoter (pCoYMV), a Rice tungro bacilliform virus promoter (pRTBV), a Solanum tuberosum KST1 promoter (pStKST1), a cassava MeAKT2a promoter, or a cassava MeAKT2b promoter.
12. The genetically modified plant of embodiment 11, wherein the promoter comprises the pAtAKT2 promoter, the cassava MeAKT2a promoter, or the cassava MeAKT2b promoter.
13. The genetically modified plant of embodiment 12, wherein the promoter comprises the pAtAKT2 promoter, and wherein the promoter comprises a nucleotide sequence comprising at least 70% identity, at least 80% identity, at least 90% identity, at least 95% identity, or at least 99% identity to SEQ ID NO: 2.
14. A genetically modified plant comprising one or more nucleotide sequences encoding a POTASSIUM TRANSPORTER 2 (AKT2) protein operably linked to an expression control sequence, wherein the expression control sequence comprises an overexpression promoter, optionally wherein the AKT2 protein is a wild-type protein.
15. The genetically modified plant of embodiment 14, wherein the AKT2 protein is selected from the group of an Arabidopsis thaliana POTASSIUM TRANSPORTER 2 (AtAKT2) protein, a first Manihot esculenta AKT2 protein (MeAKT2a), and/or a second Manihot esculenta AKT2 protein (MeAKT2b).
16. The genetically modified plant of embodiment 14 or embodiment 15, wherein the AKT2 protein comprises a protein comprising at least 70% identity, at least 80% identity, at least 90% identity, at least 95% identity, or at least 99% identity to one of SEQ ID NOs: 17-20, 25, and 26, the AtAKT2 protein comprises a protein comprising at least 70% identity, at least 80% identity, at least 90% identity, at least 95% identity, or at least 99% identity to SEQ ID NO: 17 or SEQ ID NO: 18, the MeAKT2a protein comprises a protein comprising at least 70% identity, at least 80% identity, at least 90% identity, at least 95% identity, or at least 99% identity to SEQ ID NO: 19 or SEQ ID NO: 25, and the MeAKT2b protein comprises a protein comprising at least 70% identity, at least 80% identity, at least 90% identity, at least 95% identity, or at least 99% identity to SEQ ID NO: 20 or SEQ ID NO: 26; or a functional fragment of one of the foregoing.
17. The genetically modified plant of any one of embodiments 14-16, wherein the overexpression promoter additionally comprises tissue-specific expression, and wherein the tissue-specific expression is selected from the group of phloem-specific expression, xylem-specific expression, root-specific expression, and/or stomata-specific expression.
18. The genetically modified plant of any one of embodiments 14-17, wherein the overexpression promoter comprises an Arabidopsis thaliana POTASSIUM TRANSPORTER 2 (pAtAKT2) promoter, an Arabidopsis thaliana SUCROSE TRANSPORTER 2 promoter (pAtSUC2), a COMMELINA YELLOW MOTTLE VIRUS promoter (pCoYMV), a Rice tungro bacilliform virus promoter (pRTBV), a Solanum tuberosum KST1 promoter (pStKST1), a cassava MeAKT2a promoter, or a cassava MeAKT2b promoter.
19. The genetically modified plant of embodiment 18, wherein the promoter comprises the pAtAKT2 promoter, the cassava MeAKT2a promoter, or the cassava MeAKT2b promoter.
20. The genetically modified plant of embodiment 19, wherein the promoter is the pAtAKT2 promoter, and wherein the promoter comprises a nucleotide sequence comprising at least 70% identity, at least 80% identity, at least 90% identity, at least 95% identity, or at least 99% identity to SEQ ID NO: 2.
21. The genetically modified plant of any one of embodiments 1-20, wherein the plant produces storage roots or tubers, optionally wherein the plant produces storage roots.
22. The genetically modified plant of embodiment 21, wherein the plant is selected from the group of cassava, potato, sweet potato, yam, ube, yacón, taro, konjac, ginger, radish, turnip, rutabaga, parsnip, jicama, Jerusalem artichoke, turmeric, horseradish, beet, lotus, maca, celeriac, skirret, or wasabi.
23. The genetically modified plant of any one of embodiments 1-22, wherein the plant is a crop that benefits from potassium fertilization, for example, cassava, yams, potatoes, tomatoes, citrus fruits (e.g., oranges, lemons, limes, etc.), bananas, grains (e.g., wheat, rice, barley, sorghum, etc.), cotton, sugar beets, soybeans, cowpea, alfalfa, grapes, peppers, apples, melons (e.g., watermelon, cantaloupe, etc.).
24. The genetically modified plant of any one of embodiments 1-23, wherein the plant is a passive symplasmic phloem loader.
25. The genetically modified plant of any one of embodiments 1-24, wherein the genetically modified plant has higher photosynthetic efficiency, improved photosynthesis, higher rate of CO₂ fixation and/or electron transport rate, earlier maximum growth rate, improved yield under field conditions, improved yield under drought conditions, and/or improved yield under potassium deficiency as compared to a control plant grown under the same conditions, optionally wherein the genetically modified plant or a progenitor thereof was selected for improved growth, improved photosynthesis, higher rate of CO₂ fixation, and/or electron transport rate when grown under non-limiting energy conditions, earlier maximum growth rate, improved yield, increased plant growth, increased plant height, increased shoot growth, increased total shoot dry matter, improved storage root or tuber growth, increased number of storage roots or tubers per plant, and/or increased total storage root or tuber dry matter.
26. The genetically modified plant of any one of embodiments 1-25, wherein the genetically modified plant is a cassava plant, and wherein the genetically modified cassava plant has increased plant growth, increased plant height, increased shoot growth, increased total shoot dry matter, improved storage root growth, improved number of storage root per plant, and/or increased total storage root dry matter as compared to a control cassava plant grown under the same conditions.
27. The genetically modified plant of embodiment 26, wherein the genetically modified cassava plant has elevated concentrations of sodium (Na⁺), calcium (Ca²⁺), magnesium (Mg²⁺), and/or potassium (K⁺) in shoot tissue, reduced concentrations of sodium (Na⁺), calcium (Ca²⁺), magnesium (Mg²⁺), and/or potassium (K⁺) in root tissue, reduced sucrose concentration in shoot and/or root tissue, increased glucose and/or fructose concentration in root tissue, and/or increased starch concentration in root tissue as compared to a control cassava plant grown under the same conditions.
28. The genetically modified plant of embodiment 26 or embodiment 27, wherein the genetically modified cassava plant comprises cultivar TMS60444.
29. A method of producing the genetically modified plant of any one of embodiments 1-13 and 21-28, comprising introducing one or more nucleotide sequences encoding the modified AKT2 protein, the modified AtAKT2 protein, the modified MeAKT2a protein, and/or the modified MeAKT2b protein.
30. The method of embodiment 29, wherein the modified AKT, the modified AtAKT2 protein, the modified MeAKT2a protein, and/or the modified MeAKT2b protein (a) comprises one or more amino acid substitutions, insertions, or deletions in the Ion_trans (PF00520, Ion transport protein) motif (corresponding to amino acids 75-323 of SEQ ID NO: 17) and/or one or more amino acid substitutions, insertions, or deletions in the Ion_trans_2 (PF07885, Ion channel) motif (corresponding to amino acids 225-317 of SEQ ID NO: 17), wherein the one or more substitutions, insertions, or deletions increase the ion transport activity; or (b) comprises one or more amino acid substitutions, insertions, or deletions, wherein the one or more substitutions, insertions, or deletions increase the ion transport activity.
31. The method of embodiment 29, wherein the modified AKT2 protein, the modified AtAKT2 protein, the modified MeAKT2a protein, and/or the modified MeAKT2b protein comprises one or both of the amino acid substitutions corresponding to S210N and S329N when aligned to SEQ ID NO: 17.
32. The method of any one of embodiments 29-31, wherein the modified AKT2 protein comprises a protein comprising at least 70% identity, at least 80% identity, at least 90% identity, at least 95% identity, or at least 99% identity to one of SEQ ID NOs: 18, 25, and 26, the modified AtAKT2N protein comprises a protein comprising at least 70% identity, at least 80% identity, at least 90% identity, at least 95% identity, or at least 99% identity to SEQ ID NO: 18, wherein the modified MeAKT2a protein comprises a protein comprising at least 70% identity, at least 80% identity, at least 90% identity, at least 95% identity, or at least 99% identity to SEQ ID NO: 25, and wherein the modified MeAKT2b protein comprises a protein comprising at least 70% identity, at least 80% identity, at least 90% identity, at least 95% identity, or at least 99% identity to SEQ ID NO: 26; or a functional fragment of one of the foregoing.
33. The method of any one of embodiments 29-32, wherein the one or more nucleotide sequences encoding the modified AtAKT2 protein comprises a nucleotide sequence comprising at least 70% identity, at least 80% identity, at least 90% identity, at least 95% identity, or at least 99% identity to SEQ ID NO: 3.
34. The method of any one of embodiments 29-33, wherein the one or more nucleotide sequences encoding the modified AKT2 protein, the modified AtAKT2 protein, the modified MeAKT2a protein, and/or the modified MeAKT2b protein is operably linked to an expression control sequence.
35. The method of embodiment 34, wherein the expression control sequence comprises an overexpression promoter and/or a phloem-specific promoter.
36. The method of embodiment 35, wherein the promoter comprises an Arabidopsis thaliana POTASSIUM TRANSPORTER 2 (pAtAKT2) promoter, an Arabidopsis thaliana SUCROSE TRANSPORTER 1 promoter (pAtSUC1), a COMMELINA YELLOW MOTTLE VIRUS promoter (pCoYMV), a Rice tungro bacilliform virus promoter (pRTBV), a Solanum tuberosum KST1 promoter (pStKST1), a cassava MeAKT2a promoter, or a cassava MeAKT2b promoter.
37. The method of embodiment 36, wherein the promoter comprises the pAtAKT2 promoter, the cassava MeAKT2a promoter, or the cassava MeAKT2b promoter.
38. The method of embodiment 37, wherein the promoter comprises the pAtAKT2 promoter, and wherein the promoter comprises a nucleotide sequence comprising at least 70% identity, at least 80% identity, at least 90% identity, at least 95% identity, or at least 99% identity to SEQ ID NO: 2.
39. A method of producing the genetically modified plant of any one of embodiments 1-13 and 21-28, comprising genetically modifying a plant by transforming the plant with one or more gene editing components that target an endogenous nuclear genome sequence encoding the wild-type AKT2 protein, the wild-type AtAKT2 protein, the wild-type MeAKT2a protein, and/or the wild-type MeAKT2b protein to produce the modified AKT2 protein, modified AtAKT2 protein, the modified MeAKT2a protein, and/or the modified MeAKT2b protein.
40. The method of embodiment 39, wherein the one or more gene editing components comprise a ribonucleoprotein complex that targets the nuclear genome sequence; a vector including a TALEN protein encoding sequence, wherein the TALEN protein targets the nuclear genome sequence; a vector including a ZFN protein encoding sequence, wherein the ZFN protein targets the nuclear genome sequence; an oligonucleotide donor (OND), wherein the OND targets the nuclear genome sequence; or a vector CRISPR/Cas enzyme encoding sequence and a targeting sequence, wherein the targeting sequence targets the nuclear genome sequence.
41. The method of embodiment 39 or embodiment 40, wherein the wild-type AKT2 protein comprises a protein comprising at least 70% identity, at least 80% identity, at least 90% identity, at least 95% identity, or at least 99% identity to one of SEQ ID NOs: 17, 19, and 20, the wild-type AtAKT2 protein comprises a protein comprising at least 70% identity, at least 80% identity, at least 90% identity, at least 95% identity, or at least 99% identity to SEQ ID NO: 17, the wild-type MeAKT2a protein comprises a protein comprising at least 70% identity, at least 80% identity, at least 90% identity, at least 95% identity, or at least 99% identity to SEQ ID NO: 19, and the wild-type MeAKT2b protein comprises a protein comprising at least 70% identity, at least 80% identity, at least 90% identity, at least 95% identity, or at least 99% identity to SEQ ID NO: 20; or a functional fragment of one of the foregoing.
42. The genetically modified plant of any one of embodiments 39-41, wherein the modified AKT2 protein, the modified AtAKT2 protein, the modified MeAKT2a protein, and/or the modified MeAKT2b protein (a) comprises one or more amino acid substitutions, insertions, or deletions in the Ion_trans (PF00520, Ion transport protein) motif (corresponding to amino acids 75-323 of SEQ ID NO: 17) and/or one or more amino acid substitutions, insertions, or deletions in the Ion_trans_2 (PF07885, Ion channel) motif (corresponding to amino acids 225-317 of SEQ ID NO: 17), wherein the one or more substitutions, insertions, or deletions increase the ion transport activity; or (b) comprises one or more amino acid substitutions, insertions, or deletions, wherein the one or more substitutions, insertions, or deletions increase the ion transport activity.
43. The method of any one of embodiments 39-41, wherein the modified AKT2 protein, the modified AtAKT2 protein, the modified MeAKT2a protein, and/or the modified MeAKT2b protein comprises one or both of the amino acid substitutions corresponding to S210N and S329N when aligned to SEQ ID NO: 17.
44. The method of any one of embodiments 39-43, wherein the modified AKT2 protein comprises a protein comprising at least 70% identity, at least 80% identity, at least 90% identity, at least 95% identity, or at least 99% identity to one of SEQ ID NOs: 18, 25, and 26, the modified AtAKT2N protein comprises a protein comprising at least 70% identity, at least 80% identity, at least 90% identity, at least 95% identity, or at least 99% identity to SEQ ID NO: 18, wherein the modified MeAKT2a protein comprises a protein comprising at least 70% identity, at least 80% identity, at least 90% identity, at least 95% identity, or at least 99% identity to SEQ ID NO: 25, and wherein the modified MeAKT2b protein comprises a protein comprising at least 70% identity, at least 80% identity, at least 90% identity, at least 95% identity, or at least 99% identity to SEQ ID NO: 26; or a functional fragment of one of the foregoing.
45. A method of producing the genetically modified plant of any one of embodiments 14-28, comprising introducing one or more nucleotide sequences encoding the AKT2 protein, the AtAKT2 protein, the MeAKT2a protein, and/or the MeAKT2b protein operably linked to the expression control sequence comprising the overexpression promoter.
46. The method of embodiment 45, wherein the AKT2 protein comprises a protein comprising at least 70% identity, at least 80% identity, at least 90% identity, at least 95% identity, or at least 99% identity to one of SEQ ID NOs: 17-19, 25, and 26, the AtAKT2 protein comprises a protein comprising at least 70% identity, at least 80% identity, at least 90% identity, at least 95% identity, or at least 99% identity to SEQ ID NO: 17 or SEQ ID NO: 18, the MeAKT2a protein comprises a protein comprising at least 70% identity, at least 80% identity, at least 90% identity, at least 95% identity, or at least 99% identity to SEQ ID NO: 19 or SEQ ID NO: 25, and the MeAKT2b protein comprises a protein comprising at least 70% identity, at least 80% identity, at least 90% identity, at least 95% identity, or at least 99% identity to SEQ ID NO: 20 or SEQ ID NO: 26; or a functional fragment of one of the foregoing.
47. The method of embodiment 45 or embodiment 46, wherein the overexpression promoter comprises the Arabidopsis thaliana POTASSIUM TRANSPORTER 2 (pAtAKT2) promoter, the Arabidopsis thaliana SUCROSE TRANSPORTER 1 promoter (pAtSUC1), the COMMELINA YELLOW MOT TLE VIRUS promoter (pCoYMV), the Rice tungro bacilliform virus promoter (pRTBV), the Solanum tuberosum KST1 promoter (pStKST1), the cassava MeAKT2a promoter, or the cassava MeAKT2b promoter.
48. The method of embodiment 47, wherein the promoter comprises the pAtAKT2 promoter, the cassava MeAKT2a promoter, or the cassava MeAKT2b promoter.
49. A method of producing the genetically modified plant of any one of embodiments 14-28, comprising genetically modifying a plant by transforming the plant with one or more gene editing components that target an endogenous nuclear genome sequence encoding a promoter of an endogenous AKT2 protein, a promoter of an endogenous AtAKT2 protein, a promoter of an endogenous MeAKT2a protein, and/or a promoter of an endogenous MeAKT2b protein to produce a modified AKT2 promoter, a modified AtAKT2 promoter, a modified MeAKT2a promoter, and/or a modified MeAKT2b promoter, wherein the modified AKT2 promoter, the modified AtAKT2 promoter, the modified MeAKT2a promoter, and/or the modified MeAKT2b promoter has increased expression and/or altered tissue-specific expression as compared to the unmodified promoter.
50. The method of embodiment 49, wherein the one or more gene editing components comprise a ribonucleoprotein complex that targets the nuclear genome sequence; a vector including a TALEN protein encoding sequence, wherein the TALEN protein targets the nuclear genome sequence; a vector including a ZFN protein encoding sequence, wherein the ZFN protein targets the nuclear genome sequence; an oligonucleotide donor (OND), wherein the OND targets the nuclear genome sequence; or a vector CRISPR/Cas enzyme encoding sequence and a targeting sequence, wherein the targeting sequence targets the nuclear genome sequence.
51. The method of any one of embodiments 29-50, further comprising selecting a genetically modified plant with improved growth, improved photosynthesis, higher rate of CO₂ fixation and/or higher electron transport rate when the genetically modified plant is grown under non-limiting energy conditions, earlier maximum growth rate, improved yield, increased plant growth, increased plant height, increased shoot growth, increased total shoot dry matter, improved storage root or tuber growth, increased number of storage roots or tubers per plant, and/or increased total storage root or tuber dry matter as compared to a control plant.
52. A genetically modified plant produced by the method of any one of embodiments 29-51.
53. The genetically modified plant of embodiment 52, wherein the plant produces storage roots or tubers, and/or is a passive symplasmic phloem loader.
54. The genetically modified plant of embodiment 53, wherein the plant is selected from the group of cassava, potato, sweet potato, yam, ube, yacón, taro, konjac, ginger, radish, turnip, rutabaga, parsnip, jicama, Jerusalem artichoke, turmeric, horseradish, beet, lotus, maca, celeriac, skirret, or wasabi.
55. The genetically modified plant of embodiment 52, wherein the plant is a crop that benefits from potassium fertilization, for example, cassava, yams, potatoes, tomatoes, citrus fruits (e.g., oranges, lemons, limes, etc.), bananas, grains (e.g., wheat, rice, barley, sorghum, etc.), cotton, sugar beets, soybeans, cowpea, alfalfa, grapes, peppers, apples, melons (e.g., watermelon, cantaloupe, etc.).
56. The genetically modified plant of any one of embodiments 52-55, wherein the genetically modified plant has higher photosynthetic efficiency, improved photosynthesis, higher rate of CO₂ fixation and/or electron transport rate, earlier maximum growth rate, improved yield under field conditions, improved yield under drought conditions, and/or improved yield under potassium deficiency as compared to a control plant grown under the same conditions.
57. An expression vector or isolated DNA molecule comprising one or more nucleotide sequences encoding a modified POTASSIUM TRANSPORTER 2 (AKT2) protein operably linked to an expression control sequence.
58. The expression vector or isolated DNA molecule of embodiment 57, wherein the modified AKT2 protein is selected from the group of a modified Arabidopsis thaliana POTASSIUM TRANSPORTER 2 (AtAKT2) protein, a modified first Manihot esculenta AKT2 protein (MeAKT2a), and/or a modified second Manihot esculenta AKT2 protein (MeAKT2b).
59. The expression vector or isolated DNA molecule of embodiment 58, wherein the modified AKT2 protein comprises a protein comprising at least 70% identity, at least 80% identity, at least 90% identity, at least 95% identity, or at least 99% identity to one of SEQ ID NOs: 18, 25, and 26, the modified AtAKT2 protein comprises a protein comprising at least 70% identity, at least 80% identity, at least 90% identity, at least 95% identity, or at least 99% identity to SEQ ID NO: 18, wherein the modified MeAKT2a protein comprises a protein comprising at least 70% identity, at least 80% identity, at least 90% identity, at least 95% identity, or at least 99% identity to SEQ ID NO: 25, and wherein the modified MeAKT2b protein comprises a protein comprising at least 70% identity, at least 80% identity, at least 90% identity, at least 95% identity, or at least 99% identity to SEQ ID NO: 26; or a functional fragment of one of the foregoing.
60. The expression vector or isolated DNA molecule of 58 or embodiment 59, wherein the one or more nucleotide sequences encoding the modified AtAKT2 protein comprises a nucleotide sequence comprising at least 70% identity, at least 80% identity, at least 90% identity, at least 95% identity, or at least 99% identity to SEQ ID NO: 3.
61. An expression vector or isolated DNA molecule comprising one or more nucleotide sequences encoding a wild-type POTASSIUM TRANSPORTER 2 (AKT2) protein operably linked to an expression control sequence.
62. The expression vector or isolated DNA molecule of embodiment 61, wherein the wild-type AKT2 protein is selected from the group of an Arabidopsis thaliana POTASSIUM TRANSPORTER 2 (AtAKT2) protein, a first Manihot esculenta AKT2 protein (MeAKT2a), and/or a second Manihot esculenta AKT2 protein (MeAKT2b).
63. The expression vector or isolated DNA molecule of embodiment 61 or embodiment 62, wherein the AKT2 protein comprises a protein comprising at least 70% identity, at least 80% identity, at least 90% identity, at least 95% identity, or at least 99% identity to one of SEQ ID NOs: 17, 19, and 20, the AtAKT2 protein comprises a protein comprising at least 70% identity, at least 80% identity, at least 90% identity, at least 95% identity, or at least 99% identity to SEQ ID NO: 17, the MeAKT2a protein comprises a protein comprising at least 70% identity, at least 80% identity, at least 90% identity, at least 95% identity, or at least 99% identity to SEQ ID NO: 19, and the MeAKT2b protein comprises a protein comprising at least 70% identity, at least 80% identity, at least 90% identity, at least 95% identity, or at least 99% identity to SEQ ID NO: 20; or a functional fragment of one of the foregoing.
64. The expression vector or isolated DNA molecule of any one of embodiments 57-63, wherein the expression control sequence comprises an overexpression promoter, a phloem-specific promoter, a xylem-specific promoter, a root-specific promoter, and/or a stomata-specific promoter.
65. The expression vector or isolated DNA molecule of embodiment 64 wherein the promoter comprises an Arabidopsis thaliana POTASSIUM TRANSPORTER 2 (pAtAKT2) promoter, an Arabidopsis thaliana SUCROSE TRANSPORTER 1 promoter (pAtSUC1), a COMMELINA YELLOW MOTTLE VIRUS promoter (pCoYMV), a Rice tungro bacilliform virus promoter (pRTBV), a Solanum tuberosum KST1 promoter (pStKST1), a cassava MeAKT2a promoter, or a cassava MeAKT2b promoter.
66. The expression vector or isolated DNA molecule of embodiment 65, wherein the promoter comprises the pAtAKT2 promoter, the cassava MeAKT2a promoter, or the cassava MeAKT2b promoter.
67. The expression vector or isolated DNA molecule of embodiment 66, wherein the promoter comprises the pAtAKT2 promoter, and wherein the promoter comprises a nucleotide sequence comprising at least 70% identity, at least 80% identity, at least 90% identity, at least 95% identity, or at least 99% identity to SEQ ID NO: 2.
68. A bacterial cell or an Agrobacterium cell comprising the expression vector or isolated DNA molecule of any one of embodiments 57-67.
69. A composition or kit comprising the expression vector or isolated DNA molecule of embodiment any one of embodiments 57-67 or the bacterial cell or the Agrobacterium cell of embodiment 68.
70. A genetically modified plant, plant part, plant cell, or seed including the expression vector or isolated DNA molecule of any one of embodiments 57-67.
71. A composition or kit comprising the genetically modified plant of any one of embodiments 1-28, the genetically modified plant, plant part, plant cell, or seed of embodiment 70, or the genetically modified plant produced by the method of any one of embodiments 29-51.
72. A method of increasing photosynthetic efficiency, improving photosynthesis, producing earlier maximum growth rate, improving yield under field conditions, improving yield under drought conditions, improving yield under potassium deficiency, increasing plant growth, increasing plant height, increasing shoot growth, increasing total shoot dry matter, improving storage root or tuber growth, increasing number of storage roots or tubers per plant, and/or increasing total storage root or tuber dry matter comprising: introducing a genetic alteration via the expression vector or isolated DNA molecule of any one of embodiments 57-67 to a cell, wherein the cell is a plant cell.
73. A genetically altered plant genome comprising (i) the one or more edited endogenous nucleotide sequences in the genetically modified plant of any one of embodiments 1-28, or (ii) the one or more edited endogenous nucleotide sequences in the genetically modified plant produced by the method of any one of embodiments 29-51.
74. A non-regenerable part or cell of the genetically modified plant of any one of embodiments 1-28.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIGS. 1A-1H show that fertilization with potassium leads to improved shoot and storage root growth in cassava. FIG. 1A shows a bar graph of cassava shoot height measured nine months after planting and growth under greenhouse conditions in soil with different concentrations of potassium. FIG. 1B shows a bar graph of cassava shoot fresh weight measured nine months after planting and growth under greenhouse conditions in soil with different concentrations of potassium. FIG. 1C shows representative storage roots grown under greenhouse conditions in soil with different concentrations of potassium. FIG. 1D shows a bar graph of storage root fresh weight (g) for storage roots grown under greenhouse conditions in soil with different concentrations of potassium. FIG. 1E shows a bar graph of the number of storage roots per plant for plants grown under greenhouse conditions in soil with different concentrations of potassium. FIG. 1F shows a bar graph of storage root weight (g) per plant for plants grown under greenhouse conditions in soil with different concentrations of potassium. FIG. 1G shows bar graphs of the quantification in μmol per gram fresh weight of potassium (K⁺; top left graph), sodium (Na⁺, top right graph), chlorine (Cl⁻, bottom left graph), and phosphoric acid ions (PO₄³⁻, bottom right graph) of leaf (left half of each graph) and stem (right half of each graph) tissue of plants grown under greenhouse conditions in soil with different concentrations of potassium. FIG. 1H shows bar graphs of the quantification in μmol per gram fresh weight of glucose (top row), fructose (middle row), and sucrose (bottom row) in leaf tissue (left column), petioles (middle column), and storage roots (right column) of plants grown under greenhouse conditions in soil with different concentrations of potassium. For FIGS. 1A-1H, K1=27 mg K⁺/kg soil; K2=142 mg K⁺/kg soil; K3=500 mg K⁺/kg soil; and K4=2000 mg K⁺/kg soil. For FIGS. 1A, 1B, and 1D-1H, bars represent mean+/−standard deviation, and letters indicate the same level of significance calculated via one-way ANOVA with post-hoc Tukey HSD test with p<0.05.

FIGS. 2A-2B show that fertilization with potassium leads to altered ion distributions in cassava. FIG. 2A shows bar graphs of the quantification in μmol per gram fresh weight of ammonium (NH₄⁺, top left graph), magnesium (Mg²⁺, top right graph), fluoride (F⁻, bottom left graph), and sulphate ions (SO₄²⁻, bottom right graph) of leaf (left half of each graph) and stem (right half of each graph) tissue of plants grown under greenhouse conditions in soil with different concentrations of potassium. FIG. 2B shows bar graphs of the quantification in μmol per gram fresh weight of starch in leaf tissue (left graph), petioles (middle graph), and storage roots (right graph) of plants grown under greenhouse conditions in soil with different concentrations of potassium. For FIGS. 2A-2B, K1=27 mg K⁺/kg soil; K2=142 mg K⁺/kg soil; K3=500 mg K⁺/kg soil; and K4=2000 mg K⁺/kg soil. Bars represent mean+/−standard deviation, and letters indicate the same level of significance calculated via one-way ANOVA with post-hoc Tukey HSD test with p<0.05.

FIGS. 3A-3J show that overexpression of AtAKT2var in cassava leads to alteration in shoot and growth. FIG. 3A shows a diagram of the mutagenized Arabidopsis thaliana potassium channel AKT2 (S210N-S329N; AtAKT2var; SEQ ID NO: 3, represented by the dark grey box) that was overexpressed under the Arabidopsis thaliana promoter (“pAtAKT2”, SEQ ID NO: 2, represented by the arrow) in cassava. Mutagenized residues are indicated below (“S210N” and “S329N”). FIG. 3B shows a Southern Blot analysis of AtAKT2var overexpression events compared to wild type (“WT”). From left to right, lanes represent: Std=standard; WT=wild type; 4255=AKT2var-4255 overexpression line; 4261=AKT2var-4261 overexpression line; 4262=AKT2var-4262 overexpression line; 4264=AKT2var-4264 overexpression line; 4265=AKT2var-4265 overexpression line; 4266=AKT2var-4266 overexpression line. FIG. 3C shows the legend for FIGS. 3G and 3I. Wild type plants are indicated by black squares. In addition to the wild type, six different friable embryonic calli events (FEC) are indicated by diamonds, six different empty vector controls (EV) are indicated by circles, and six different AtAKT2var overexpression events (AKT2) are indicated by triangles. FIG. 3D shows dot plots of the relative transcript levels of AtAKT2var in the overexpression lines indicated on the x-axis in the following tissues, from leftmost graph to rightmost graph: leaf tissue, petiole, and upper stem (top row of panels); middle stem, lower stem, and root (bottom row of panels). Transcript levels were normalized to GAPDH. FIG. 3E shows pictures of typical examples of shoot tissue approximately 9 months after planting from wild type (top left), empty vector controls (top rightmost three pictures), and selected AtAKT2var overexpression events (bottom row). Pictures were taken during final harvest. Scale bar=10 cm. FIG. 3F shows dot plots of the determination of plant height in meters (top) and total shoot dry matter (TSDM) in kilograms (bottom) of the same plants as in FIG. 3D. The measurements are grouped along the horizontal axes by wild type, friable embryonic calli events (FEC), empty vector control events (EV), and AtAKT2var overexpression events (AKT2). FIG. 3G shows pictures of representative storage roots of harvested plants approximately 9 months after planting which were used for quantification in FIG. 3H. Pictures were taken during final harvest. Scale bar=10 cm. FIG. 3H shows dot plots of total root dry matter (TRDM) in kilograms of storage roots of harvested plants of the genotypes indicated on the x-axis. FIG. 3I shows a vector map of the transformation plasmid p134GG_pAtAKT2::AtAKT2mut. FIG. 3J shows a vector map of the transformation plasmid p134GG_Vector control. For FIGS. 3D, 3F, and 3H, bars show means of biological replicates+/−standard deviation. Letters indicate the same level of significance calculated via one-way ANOVA with the post-hoc Tukey HSD test with p<0.05.

FIGS. 4A-4B show that overexpression of AtAKT2var in cassava leads to alteration in shoot and root growth. FIG. 4A shows pictures of typical examples of shoot tissue from wild type (top row), empty vector control (bottom row, leftmost three columns), and overexpression lines (bottom row, rightmost three columns), approximately 9 months after planting; pictures were taken during final harvest. FIG. 4B shows pictures of typical examples of storage roots from wild type (top row), empty vector control (bottom row, leftmost three columns), and overexpression lines (bottom row, rightmost three columns), approximately 9 months after planting; pictures were taken during final harvest.

FIGS. 5A-5D show that overexpression of AtAKT2var in cassava leads to alterations in ion content. FIG. 5A shows, for shoots, dot plots of anion concentrations of fluoride (F⁻, first row), chloride (Cl⁻, second row), nitrate (NO₃⁻, third row), phosphate (PO₄³⁻, fourth row) and sulphate (SO₄²⁻, fifth row) in shoot tissue of friable embryonic calli (FEC), empty vector control (EV) and AtAKT2var overexpression (AKT2) lines. FIG. 5B shows, for roots, dot plots of anion concentrations of fluoride (F⁻, first row), chloride (Cl⁻, second row), nitrate (NO₃⁻, third row), phosphate (PO₄³⁻, fourth row) and sulphate (SO₄²⁻, fifth row) in root tissue of friable embryonic calli (FEC), empty vector control (EV) and AtAKT2var overexpression (AKT2) lines. FIG. 5C shows dot plots of cation concentrations of sodium (Na⁺, first row), calcium (Ca²⁺, second row), magnesium (Mg²⁺, third row), and potassium (K⁺, fourth row) in shoot tissue of friable embryonic calli (FEC), empty vector control (EV) and AtAKT2var overexpression (AKT2) lines. FIG. 5D shows dot plots of cation concentrations of sodium (Na⁺, first row), calcium (Ca²⁺, second row), magnesium (Mg²⁺, third row), and potassium (K⁺, fourth row) in root tissue of friable embryonic calli (FEC), empty vector control (EV) and AtAKT2var overexpression (AKT2) lines. For FIGS. 5A-5D, bars show means of biological replicates+/−standard deviation. Letters indicate the same level of significance calculated via one-way ANOVA with the post-hoc Tukey HSD test with p<0.05.

FIGS. 6A-6G show that overexpression of AtAKT2var in cassava leads to increased photosynthetic performance. FIG. 6A shows volume-based seasonal growth in 2022 field trials in cubic meters (left), and the respective sigmoidal fitting and its derivative (right) as a reference for the relative growth rate (RGR) across 250 days after planting (DAP) for AtAKT2var overexpression events (AKT2, dotted line) and empty vector control events (VC, dashed line). FIG. 6B shows plots of electron transport rate (ETR) as a reference for the photosynthetic efficiency under varying natural daylight conditions, for AtAKT2var overexpression events (“AKT2var”, right graph) and empty vector control events (“EV”, left graph) in 2022 field trials. PAR stands for photosynthetically active radiation. Raw data downloaded from the MoniPAM was filtered based on the visual (not automated) comparison between the diurnal cycles of PAR and the quantum efficiency of photosystem II, Y (II). FIG. 6C shows volume-based seasonal growth in 2023 field trials in cubic meters (left), and the respective sigmoidal fitting and its derivative (right) as a reference for the relative growth rate (RGR) across 160 days after planting (DAP) for AtAKT2var overexpression events (AKT2, dotted line) and empty vector control events (VC, dashed line). FIG. 6D shows plots of electron transport rate (ETR) as a reference for the photosynthetic efficiency under varying natural daylight conditions, for AtAKT2var overexpression events (AKT2, left graph) and empty vector control events (VC, right graph) in 2022 field trials at intermediate harvest. PAR stands for photosynthetically active radiation. Raw data downloaded from the MoniPAM was filtered based on the automated process aiming to remove data points that met one of the following conditions: Y (II)>0.6 & PAR>800 (shaded leaf). Y (II)<0.2 & PAR<200 (shaded PAR sensor); Y (II)<0.15 (loose leaf). Y (II) stands for the quantum efficiency of photosystem II. In the left panel, line AKT2var-4261 is indicated by white circles with a solid outline, line AKT2var-4262 is indicated by banded circles, and line AKT2var-4266 is indicated by white circles with a dashed outline. In the right panel, line EV-4220 is indicated by white circles with a solid outline, and line EV-4234 is indicated by banded circles. FIG. 6E shows plots of electron transport rate (ETR) as a reference for the photosynthetic efficiency under varying natural daylight conditions, for AtAKT2var overexpression events (AKT2, left graph) and empty vector control events (VC, right graph) in 2022 field trials at final harvest. PAR stands for photosynthetically active radiation. Raw data downloaded from the MoniPAM was filtered based on the automated process aiming to remove data points that met one of the following conditions: Y (II)>0.6 & PAR>800 (shaded leaf); Y (II)<0.2 & PAR<200 (shaded PAR sensor); Y (II)<0.15 (loose leaf). Y (II) stands for the quantum efficiency of photosystem II. FIG. 6F shows dot plots of quantification of sugar concentrations in μmol per gram dry weight of glucose (Glc, top row), fructose (Frc, second row), sucrose (Suc, third row) and starch (bottom row) contents in shoot tissue of friable embryonic calli (FEC), empty vector control (EV) and AtAKT2var overexpression (AKT2) lines. In the left panel, line AKT2var-4262 is indicated by dotted circles with a solid outline, and line AKT2var-4261 is indicated by shaded and dashed circles with a solid outline. In the right panel, line AKT2var-4234 is indicated by dotted circles with a solid outline, and line AKT2var-4220 is indicated by shaded and dashed circles with a solid outline. FIG. 6G shows dot plots of quantification of sugar concentrations in μmol per gram dry weight of glucose (Glc, top row), fructose (Frc, second row), sucrose (Suc, third row) and starch (bottom row) contents in root tissue of friable embryonic calli (FEC), empty vector control (EV) and AtAKT2var overexpression (AKT2) lines. For FIGS. 6F-6G, bars show means of biological replicates (horizontal bars)+/−standard deviation (vertical bars). Letters indicate the same level of significance calculated via one-way ANOVA with the post-hoc Tukey HSD test with p<0.05.

FIGS. 7A-7I show intermediate harvest results. FIGS. 7A-7C show pictures of typical examples of shoot tissue from wild type (WT, or WT-FEC for friable embryonic calli; FIG. 7A), empty vector control (VC or “Vector Control”; FIG. 7B), and AtAKT2var overexpression (“AKT2 Ox”, FIG. 7C) lines taken approximately four months after planting. FIG. 7D shows plots of determination of plant height in centimeters of wild type (leftmost section), friable embryonic calli (WT-FEC, second section), empty vector control (EN; third section), and AtAKT2var overexpression (AKT2; rightmost section) lines. FIG. 7E shows plots of determination of shoot fresh weight in kilograms of wild type (leftmost section), friable embryonic calli (WT-FEC, second section), empty vector control (EN; third section), and AtAKT2var overexpression (AKT2; rightmost section). FIGS. 7F-7H shows pictures of representative storage roots of harvested plants from wild type (leftmost panel of FIG. 7F), friable embryonic calli (WT-FEC; FIG. 7F), empty vector control (VC; FIG. 7G), and AtAKT2var overexpression (AKT2; FIG. 7H) lines which were then used for quantification in FIG. 7I. FIG. 7I shows plots of storage root fresh weight measured in kilograms of storage roots of harvested plants from wild type (leftmost section), friable embryonic calli (WT-FEC, second section), empty vector control (EN; third section), and AtAKT2var overexpression (AKT2; rightmost section) lines. For FIGS. 7D, 7E, and 7I, bars show means of biological replicates+/−standard deviation. Experimental lines with results that are commented on in the Examples are marked by distinguishing patterns in their bar plots. Letters indicate the same level of significance calculated via one-way ANOVA with the post-hoc Tukey HSD test with p<0.05.

FIGS. 8A-8H show that overexpression of AtAKT2var in cassava leads to alteration in shoot and root growth. FIG. 8A shows the legend for FIGS. 8B, 8C, and 8F-8H. Wild type plants are indicated by black squares. In addition to the wild type, empty vector controls (EV) are indicated by circles in various shades of grey, and AtAKT2var overexpression lines (AKT2) are indicated by triangles in various shades of grey. C1 and C2 refer to two independent greenhouse cultivations. FIG. 8B shows dot plots of relative transcript levels of atATK2 in leaf tissue (left column), shoot tissue (middle column), and root tissue (right column) of overexpression lines after normalization to GAPDH for greenhouse cultivation C1 (top row) and greenhouse cultivation C2 (bottom row). FIG. 8C shows quantification of growth of plant height in centimeters over the course of 14 weeks of plants of empty vector (circles) and AtAKT2var overexpression (triangles) lines for greenhouse cultivation C1 (top row) and greenhouse cultivation C2 (bottom row). Shown are means of biological replications+/−standard deviation. FIGS. 8D-8E show images of shoot tissue (FIG. 8D) and harvested storage roots (FIG. 8E) for wild type (top left panel), empty vector (EV, remaining top row of panels), and AtAKT2var overexpression (AKT2, bottom row of panels) lines of greenhouse cultivation C2. In FIG. 8E, the scale bar=2 cm. FIG. 8F shows dot plots of the determination of plant height in centimeters for greenhouse cultivation C1 (left) and greenhouse cultivation C2 (right) for WT (greenhouse cultivation C2 only, squares), empty vector (EV, circles), and AtAKT2var overexpression (AKT, triangles) lines. FIG. 8G shows dot plots of total shoot dry matter (TSDM) in grams for greenhouse cultivation C1 (left) and greenhouse cultivation C2 (right) for WT (greenhouse cultivation C2 only, squares), empty vector (EV, circles), and AtAKT2var overexpression (AKT, triangles) lines. FIG. 8H shows dot plots of total root dry matter (TRDM) in grams for greenhouse cultivation C1 (left) and greenhouse cultivation C2 (right) for WT (greenhouse cultivation C2 only, squares), empty vector (EV, circles), and AtAKT2var overexpression (AKT, triangles) lines. For FIGS. 8B, 8F, 8G, and 8H, bars show means of biological replicates+/−standard deviation. Letters indicate the same level of significance calculated via one-way ANOVA with the post-hoc Tukey HSD test with p<0.05.

FIGS. 9A-9I show, for confined greenhouse trials, effects of AtAKT2var expression in cassava on shoot and root growth. Results are shown for empty vector (EV) control lines “EV-4234” and “EV-4243”, as well as AKT2var overexpression lines “AKT2var-4261”, “AKT2var-4262”, and “AKT2var-4264”. FIGS. 9A-9B show results of a first set of cultivation trials (“Cultivation 1”), FIGS. 9C-9D show results of a second set of cultivation trials (“Cultivation II”), and FIGS. 9E-9F show results of a third set of cultivation trials (“Cultivation III”). FIGS. 9A, 9C, and 9E show exemplary phenotypes of shoot tissues (top panels) and root tissues (bottom panels) from the first cultivation (FIG. 9A), the second cultivation (FIG. 9C), and the third cultivation (FIG. 9E). Scale bars=20 cm. FIGS. 9B, 9D, and 9F show results of sampled plants' TSDM (“Shoot dry weight”, plotted in grams (g) along the top panel's vertical axis), and TRDM (“Root dry weight”, plotted in grams (g) along the bottom panel's vertical axis) for the first cultivation (FIG. 9B), the second cultivation (FIG. 9D), and the third cultivation (FIG. 9F). FIG. 9G shows cation levels, specifically calcium (Ca²⁺, top row of panels), magnesium (Mg²⁺, middle row of panels), and sodium (Na⁺, bottom row of panels), plotted in mol gDW⁻¹ along the vertical axes for leaf tissue from source leaves (left column), shoot tissue from the lower stem (middle column), and root tissue from storage roots (right column). FIG. 9H shows anion levels, specifically phosphate (PO₄³⁻, top row of panels), sulphate (SO₄²⁻, middle row of panels), and chloride (Cl⁻, bottom row of panels), plotted in mol gDW⁻¹ along the vertical axes for leaf tissue from source leaves (left column), shoot tissue from the lower stem (middle column), and root tissue from storage roots (right column). FIG. 9I shows sugar levels, specifically glucose (Glc, top row of panels) and fructose (Frc, bottom row of panels), plotted in mol gDW⁻¹ along the vertical axes for leaf tissue from source leaves (left column), shoot tissue from the lower stem (middle column), and root tissue from storage roots (right column). In FIGS. 9B, 9D, 9F, 9G, 9H, and 9I, for each cassava line, the central horizontal line represents the median, plus (+) indicates the mean; box edges delineate first and third quartiles, whiskers extend to maximum and minimum values and dots show individual values. Different lowercase letters indicate statistical significance, as calculated by one-way ANOVA with a post-hoc Tukey HSD test (p<0.05). In FIGS. 9A and 9C, scale bars=20 cm for the shoot tissues (top panels) and scale bars=5 cm for the root tissues (bottom panels). In FIG. 9E, scale bars=10 cm.

FIG. 10 shows scatterplots of shoot dry weight in grams (horizontal axes) versus root dry weight in grams (vertical axes) for greenhouse cultivation C1 (top row) and greenhouse cultivation C2 (bottom row), for wild type (WT, C2 only, cross-hatched squares), empty vector control (“EV”, circles), and AtAKT2var overexpression lines (AKT2 or “AKT2-Ox”, triangles), for five-week-old plants (left column) and twelve-week-old plants (right column) grown in greenhouse trials. Solid lines encompass all data points for each experimental condition.

FIGS. 11A-11H show additional analysis of AtAKT2var overexpression in cassava grown in greenhouse trials. FIG. 11A shows a diagram of the mutagenized Arabidopsis thaliana potassium channel AKT2 (S210N-S329N; SEQ ID NO: 3, represented by the dark grey box) that was overexpressed under the Arabidopsis thaliana promoter (AtAKT2, SEQ ID NO: 2, represented by the arrow) in cassava. Mutagenized residues are indicated below. The construct also included an HA-Tag, indicated on the right. FIG. 11B shows dot plots of the relative transcript levels of AtAKT2var in empty vector control (left two columns of each graph, circles) and AtAKT2var overexpression (right two columns of each graph, triangles) in the following tissues, from leftmost graph to rightmost graph: leaf tissue, stem tissue, and root tissue. Transcript levels were normalized to GAPDH. ‘n.d.’ indicates that no expression was detectable. FIG. 11C shows quantification of the growth of plant height in centimeters over the course of 16 weeks of empty vector (circles) and AtAKT2var overexpression (triangles) lines. Shown are means of biological replications+/−standard deviation. Line EV-4234 (“EN4234”) is shown as barred circles, line EV-4243 (“EN4243”) is shown as dotted circles, line AKT2var-4262 (“AKT2-4262”) is shown as barred triangles, and line AKT2var-4264 (“AKT2-4264”) is shown as inverted triangles with grid patterns. FIG. 11D shows images of shoot tissue (top row) and harvested storage roots (bottom row) for 19-week-old plants of empty vector (“EN”, first and second columns), and AtAKT2var overexpression (“AKT2”, third and fourth columns) lines. Scale bar in top row=11 cm. Scale bar in bottom row=2.5 cm. FIG. 11E shows images of shoot tissue (top row) and harvested storage roots (bottom row) for 28-week-old plants of empty vector (EN, left column), and AtAKT2var overexpression (AKT2, right column) lines. Scale bar in top row=11 cm. Scale bar in bottom row=2.5 cm. FIG. 11F shows plots of plant height in centimeters for 19-week-old plants (left) and 28-week-old plants of empty vector (EN, left half of columns, circles), and AtAKT2var overexpression (AKT2, right half of columns, triangles) lines. FIG. 11G shows plots of shoot dry matter in grams for 19-week-old plants (left) and 28-week-old plants of empty vector (EN, left half of columns, circles), and AtAKT2var overexpression (AKT2, right half of columns, triangles) lines. FIG. 11H shows plots of root dry matter in grams for 19-week-old plants (left) and 28-week-old plants of empty vector (EN, left half of columns, circles), and AtAKT2var overexpression (AKT2, right half of columns, triangles) lines. For FIGS. 11B and 11F-11H, bars show means of biological replicates+/−standard deviation. Letters indicate the same level of significance calculated via one-way ANOVA with the post-hoc Tukey HSD test with p<0.05.

FIGS. 12A-12G show analysis of the impact on growth and yield of AtAKT2var overexpression in cassava grown in greenhouse conditions. FIG. 12A shows images of shoots of 19-week-old plants of wild type (WT, first image), empty vector (EV, second and third image), and AtAKT2var overexpression (AKT2, fourth through sixth images) lines. Scale bar=10 cm. FIG. 12B shows images of harvested storage roots of 19-week-old plants of wild type (WT, first image), empty vector (EV, second and third image), and AtAKT2var overexpression (AKT2, fourth through sixth images) lines. Scale bar=5 cm. FIG. 12C shows plots of plant height in centimeters of 19-week-old plants of wild type (WT, squares), empty vector (EV, circles), and AtAKT2var overexpression (AKT2, triangles) lines. FIG. 12D shows plots of shoot fresh weight in grams of 19-week-old plants of wild type (WT, squares), empty vector (EV, circles), and AtAKT2var overexpression (AKT2, triangles) lines. FIG. 12E shows plots of root fresh weight in grams of 19-week-old plants of wild type (WT, squares), empty vector (EV, circles), and AtAKT2var overexpression (AKT2, triangles) lines. FIG. 12F shows plots of harvest index of 19-week-old plants of wild type (WT, squares), empty vector (EV, circles), and AtAKT2var overexpression (AKT2, triangles) lines. FIG. 12G shows a scatterplot of shoot weight in grams against root weight in grams for 19-week-old plants of wild type (squares), empty vector (circles), and AtAKT2var overexpression (triangles) lines. For FIGS. 12C-12F, bars show means of biological replicates+/−standard deviation. Letters indicate the same level of significance calculated via one-way ANOVA with the post-hoc Tukey HSD test with p<0.05.

FIG. 13 shows a plot of plant height in centimeters over the course of 19 weeks for wild type (WT, squares), empty vector (EV, circles), and AtAKT2var overexpression (AKT2, triangles) lines grown in greenhouse conditions. Bars show means of biological replicates+/−standard deviation.

FIGS. 14A-14H show the identification of putative AKT2 homologs in cassava. FIG. 14A shows a phylogenetic tree of AKT family genes in Arabidopsis thaliana (gene names beginning with “AT”), Populus trichocarpa (gene names beginning with “Potri”), and cassava (Manihot esculenta, gene names beginning with “Manes”). The enclosed clade includes AtAKT2 and two putative cassava AKT2 homologs, MeAKT2a (Manes.07G018900, SEQ ID NO: 19) and MeAKT2b (Manes.10G122000, SEQ ID NO: 20). FIG. 14B shows a sequence alignment of AtAKT2 (second sequence, SEQ ID NO: 21) with putative cassava AKT2 homologs MeAKT2a (third sequence, SEQ ID NO: 22) and MeAKT2b (fourth sequence, SEQ ID NO: 23). At the top, a consensus identity sequence (SEQ ID NO: 24) is shown with a plot quantifying consensus identity. Regulatory serines S210 and S329 are indicated by dash-segmented boxes between the Arabidopsis and cassava sequences, and are conserved. FIGS. 14C-14D show mean tissue-specific expression for MeAKT2a (Manes.07G018900) according to data from Rüscher et al. (Rüscher et al., (2024) Symplasmic phloem loading and subcellular transport in storage roots are key factors for carbon allocation in cassava. bioRxiv.). In FIG. 14C, mean expression of MeAKT2a is shown for, from left to right, stem phloem, stem cambium 1, stem cambium 2, stem xylem, source root phloem, source root cambium 1, source root cambium 2, and source root xylem. In FIG. 14D, mean expression of MeAKT2a is shown for, from left to right, source leaves, upper stem, middle stem peel, middle stem core, lower stem peel, lower stem core, storage root, and fibrous root. FIGS. 14E-14F show mean tissue-specific expression for MeAKT2b (Manes.10G122000) according to data from Rüscher et al. (Rüscher et al., (2024) Symplasmic phloem loading and subcellular transport in storage roots are key factors for carbon allocation in cassava. bioRxiv.). In FIG. 14E, mean expression of MeAKT2b is shown for, from left to right, stem phloem, stem cambium 1, stem cambium 2, stem xylem, source root phloem, source root cambium 1, source root cambium 2, and source root xylem. In FIG. 14F, mean expression of MeAKT2b is shown for, from left to right, source leaves, upper stem, middle stem peel, middle stem core, lower stem peel, lower stem core, storage root, and fibrous root. FIG. 14G shows normalized counts (left) of the data shown in FIGS. 14C-14D and a whisker-and-box plot of the data shown in FIGS. 14C-14D (right). FIG. 14H shows normalized counts (left) of the data shown in FIGS. 14E-14F and a whisker-and-box plot of the data shown in FIGS. 14E-14F (right). For normalized counts, each tissue type has results shown for pre-bulking (“PB”, approximately 30 days after planting, dotted circles), early bulking (“EB”, approximately 45 days after planting, barred circles), and during bulking (“DB”, approximately 60 days after planting, black circles segmented by white dashed lines). Normalized counts are shown, from left to right, for sink leaves (“SiL”), source leaves (“SoL”), petioles (“Pet”), upper stem (“US”), peel tissue of the middle of the stem (“MS peel”), core tissue of the middle of the stem (“MS core”), peel tissue of the lower stem (“LS peel”), core tissue of the lower stem (“LS core”), storage root (“SR”), and fibrous roots (“FR”).

FIGS. 15A-15D show expression patterns of AtAKT2var under the native Arabidopsis promoter. FIGS. 15A-15B show quantitative real time PCR measurements of relative AtAKT2var mRNA expression levels in different tissues after normalization to MeGAPDH (with unitless ratio of relative expression to MeGAPDH plotted along the vertical axis), for five different AtAKT2var lines (plotted along the horizontal axis from left to right, from lines designated 4255, 4261, 4262, 4265, and 4266). For each cassava line, the central horizontal line represents the median, box edges delineate first and third quartiles, whiskers extend to maximum and minimum values and dots show individual values. Different lowercase letters indicate statistical significance, as calculated by one-way ANOVA with a post-hoc Tukey HSD test (p<0.05). From left to right, FIG. 15A shows results from source leaf tissue, petiole tissue, storage root peel tissue, and storage root core tissue. From left to right, FIG. 15B shows results from upper stem peel tissue, upper stem core tissue, lower stem peel tissue, and lower stem core tissue. FIG. 15C-15D show histochemical GUS staining patterns of representative pAtAKT2::GUS transgenic cassava lines. Arrows with asterisks mark phloem companion cells (dotted structures), black arrows mark xylem parenchyma cells closely associated with xylem vessels, and light grey arrows mark xylem ray cells connecting phloem and xylem. FIG. 15C shows results from source and sink leaf tissue (top panel, left scale bar=750 μm and right scale bar=500 μm), petiole tissue (middle panel, left scale bar=500 μm and right scale bar=200 μm), and storage and fibrous roots (bottom panel, left scale bar=500 μm, middle and right scale bars=750 μm). FIG. 15D shows results from the upper stem tissue about 5 cm below the apex (top panel, left scale bar=750 μm and right scale bar=500 μm), middle stem tissue in the transition zone between green stem and browning/greying stem (middle panel, left scale bar=750 μm and right scale bar=500 μm), and lower stem tissue at the base part of the stem (bottom panel, left scale bar=1 mm and right scale bar=500 μm).

FIGS. 16A-16D show, for greenhouse trials, AKT2var overexpression boosting phloem tracer transport velocities and CO₂ assimilation alongside growth rates. Different lowercase letters indicate statistical significance, as calculated by one-way ANOVA with a post-hoc Tukey HSD test (p<0.05). FIG. 16A shows a schematic view of the experimental setup for the analysis using ¹¹C-labelling and positron emission tomography (PET) scanning. Leaf petiole and stem are aligned between the figure's two panels. Visually segmented portions along the stem are designated as regions of interest (ROI) 1-12, each a small detector for radioactivity placed in sequence. FIG. 16B shows phloem flow velocities (left panel, plotted along the vertical axis as tracer transport velocity in mm min⁻¹) and carbon dioxide assimilation rates (right panel, plotted along the vertical axis as mol CO₂ m⁻²s⁻¹) for ¹¹CO₂-labelled leaves twelve weeks after planting in the greenhouse. Results were measured for a control empty vector line designated EV-4234 and AKT2var lines designated 4261 and 4262. Each triangle represents an individual AKT2var biological replicate, and each circle represents an individual empty vector line 4234 biological replicate. FIG. 16C shows images of harvested shoots and storage roots of 19-week-old plants of empty vector lines (EV-4218, EV-4234, and EV-4243, top panels left to right) and AKT2 overexpression (AKT2var4261, AKT2var4262, and AKT2var4264, bottom panels left to right) lines. Scale bar=20 cm for the shoots and 5 cm for the storage roots. FIG. 16D shows results for total shoot dry matter (TSDM, “Shoot dry weight”, plotted along the vertical axis in grams, left panel), total root dry matter (TRDM, “Root dry weight” plotted along the vertical axis in grams, middle panel), and harvest index dry weight (plotted along the vertical axis, right panel). Results are shown for, from left to right, empty vector lines 4218, 4234, and 4243 and AKT2var lines 4261, 4262, and 4264, each grouped by horizontal bars below each plot. For each cassava line, the central horizontal line represents the median, plus (+) indicates the mean; box edges delineate first and third quartiles, whiskers extend to maximum and minimum values and dots show individual values. Different lowercase letters indicate statistical significance, as calculated by one-way ANOVA with a post-hoc Tukey HSD test (p<0.05).

FIGS. 17A-17D show, for greenhouse trials, results of measuring relevant nutrients and compounds across AKT2var-expressing cassava. Results are measured for empty vector lines 4218, 4234, and 4243, and AKT2var lines 4261, 4262, and 4264. The left column in each figure shows results from leaf tissue (source leaves), the middle column shows results from the shoot (lower stem region), and the right column shows results from the root tissue (storage root). For each cassava line, the central horizontal line represents the median, plus (+) indicates the mean; box edges delineate first and third quartiles, whiskers extend to maximum and minimum values and dots show individual values. Different lowercase letters indicate statistical significance, as calculated by one-way ANOVA with a post-hoc Tukey HSD test (p<0.05). FIG. 17A shows results for cations potassium (K⁺, plotted in mol gDW⁻¹ along the vertical axes of the top row), calcium (CA²⁺, plotted in mol gDW⁻¹ along the vertical axes of the middle row), and magnesium (Mg²⁺, plotted in mol gDW⁻¹ along the vertical axes of the bottom row). FIG. 17B shows results for anions phosphate (PO₄³⁻, top row of panels), sulphate (SO₄²⁻, middle row of panels), and chloride (Cl⁻, bottom row of panels), plotted in mol gDW⁻¹ along the vertical axes. FIG. 17C shows results for glucose (top row of panels) and fructose (bottom row of panels), plotted in mol gDW⁻¹ along the vertical axes. FIG. 17D shows results for sucrose (top row of panels) and starch (bottom row of panels), plotted in mol gDW⁻¹ along the vertical axes.

FIGS. 18A-18D show, for confined field trials, performance metrics of AKT2var lines of cassava compared to empty vector controls. FIG. 18A shows aboveground plant growth (top panels) and storage roots (bottom panels) showing representative growth of both plant groups across three years (2022 in left two panels, 2023 in middle two panels, and 2024 in right two panels). Scale bar for each image is 20 cm. For FIGS. 18B-18D, significance values are indicated as follows: non-significant (p>0.05, ns), significant (p<0.05, *), highly significant (p<0.01, **), and very highly significant (p<0.001, ***), calculated by one-way ANOVA with a post-hoc Dunnetts test compared to mean vector control plant lines. “TRDM”=total root dry matter; “HI”=harvest index; “TFW”=total fresh weight; “RFW”=root fresh weight; “SFW”=shoot fresh weight; and “DMC”=dry matter content. FIG. 18B shows Pearson correlations between selected agronomic traits per year. Positive and negative correlations are depicted in dotted shades and barred shades, respectively, with darker coloration indicating a stronger positive or negative correlation. The top panel depicts comparison for 2022 results, the middle panel depicts comparisons for 2023 results, and the bottom panel depicts comparisons for 2024 results. FIG. 18C shows performance for various agronomic traits, plotted along the vertical axes, for empty vector lines 4218, 4220, 4221, 4234, and 4243, and AKT2var lines 4255, 4261, 4262, 4265, and 4266, plotted from left to right along the horizontal axes. The columns marked by a white triangle plot results from 2022, the light grey dots plot results from 2023, and the black dots plot results from 2024. From top to bottom, the agronomic traits plotted in each panel are SFW in kilograms, RFW in kilograms, HI (unitless), and TRDM in kilograms. FIG. 18D shows performance after spatial and temporal correction of raw data, wherein genotypic best linear unbiased estimates (BLUEs) and standard errors are shown per agronomic trait. The agronomic traits plotted in each panel are, from top to bottom, SFW, RFW, HI, and TRDM, all in unitless normalized measurements. The horizontal dotted line indicates the mean value of all vector control lines, and the shaded bar flanking the dotted line in each graph indicates the standard deviation of all vector control lines. Whiskers above and below each plotted point indicate standard error. As in FIG. 18C, the results in FIG. 18D are depicted for empty vector lines 4218, 4220, 4221, 4234, and 4243, and AKT2var lines 4255, 4261, 4262, 4265, and 4266, plotted from left to right along the horizontal axes.

FIGS. 19A-19D show, for field trials, results of measuring metrics of photosynthesis and various nutrients for empty vector lines and AKT2var expression lines in cassava. FIG. 19A shows results of measuring electron transport rate (ETR, plotted along the vertical axes as ETR (II)) across increasing light intensities (measured in photosynthetically active radiation (PAR), plotted along the horizontal axes as mol m⁻²s⁻¹). The left panel shows results for empty vector lines (“EV”), and the right panel shows results for AKT2var lines. FIGS. 19B-19D show results of measuring relevant nutrients and compounds across AKT2var-expressing cassava. Results are measured for empty vector lines 4218, 4220, 4221, 4234, and 4243, and AKT2var lines 4255, 4261, 4262, 4265, and 4266. The top panel in each figure shows results from shoot tissue (lower stem region), and the bottom panel shows results from the root tissue (storage root). For each cassava line, the central horizontal line represents the median, plus (+) indicates the mean; box edges delineate first and third quartiles, whiskers extend to maximum and minimum values and dots show individual values. Different lowercase letters indicate statistical significance, as calculated by one-way ANOVA with a post-hoc Tukey HSD test (p<0.05). FIG. 19B shows results for potassium (K+, plotted in mol gDW⁻¹ along the vertical axes), FIG. 19C shows results for sucrose (Suc, plotted in mol gDW⁻¹ along the vertical axes), and FIG. 19D shows results for starch (plotted in mol gDW⁻¹ along the vertical axes).

FIGS. 20A-20N show, for both standard conditions and drought conditions in greenhouse trials, results demonstrating enhanced growth and improved drought stress response. Standard conditions are indicated as “Control”, drought conditions are indicated as “Drought”, intermediate harvest measurements are indicated with “IH”, and final harvest measurements are indicated with “FIT”. FIGS. 20A and 20C respectively show representative phenotypes of shoot and root tissues from three empty vector (EV) controls (lines EV-4218, EV-4234, and EV-4243) and three AKT2var transgenic lines (AKT2var-4261, AKT2var-4262, and AKT2va-r4264) for growth under standard conditions. FIGS. 20B and 20D shows these results for shoot (FIG. 20B) and root (FIG. 20D) growth under drought conditions. FIGS. 20A and 20B's scale bars=20 cm; FIGS. 20C, 20D, 20H, and 20I's scale bars=5 cm. FIGS. 20E-20F show, for a set of controlled standard conditions, results of measuring plant height (in centimeters, top left panel), leaf dry weight (“leaf DW” in grams, top right panel), stem dry weight (“SDW” in grams, bottom left panel), and root dry weight (“RDW” in grams, bottom right panel). The lines selected for the tests of FIGS. 20A-20N are the same used in FIGS. 16A-16D. FIG. 20E shows results for plants exposed to standard conditions and measured at final harvest (“Control—FH”), and FIG. 20F shows results for plants exposed to drought conditions when measured at final harvest (“Drought—FH). FIG. 20G shows a direct comparison of root dry weight (in grams, plotted along the vertical axis) for these same lines, comparing plants grown under standard conditions (left graph) to plants grown under drought conditions (right graph), with measurements taken at final harvest (FH). FIGS. 20H-20I show results for root growth of these plant lines under standard growth conditions (FIG. 20H) and drought conditions (FIG. 20I) when measured at intermediate harvest (IH). FIGS. 20J and 20K show, for standard growth conditions (FIG. 20J) and drought conditions (FIG. 20K), indicators of drought stress for measurements taken at intermediate harvest. Leaf tissue sample results are shown in the top row of panels, and fibrous root sample results are shown in the bottom row of panels. The left column shows results of measuring proline, the middle column shows results of measuring serine, and the right column shows results of measuring glutamate, each plotted in mol gDW⁻¹ along each respective vertical axis. FIGS. 20L-20M show a corresponding set of measurements taken at intermediate harvest in standard conditions (FIG. 20L, “Control—IH”) and drought conditions (FIG. 20M, “Drought IH”), utilizing the same cassava lines. In both FIGS. 20L and 20M, the top panel shows results for samples taken from leaf tissue of source leaves (“leaf tissue (source)—IH”), the middle panel shows results for samples taken from lower stem tissue (“shoot (lower stem)—IH”), and the bottom panel shows results for samples taken from fibrous roots (“root tissue (storage root)—IH”). For FIGS. 20L-20M, results are shown for sucrose (“Suc”, plotted in mol gDW⁻¹ along the vertical axes of the left panels) and starch (plotted in mol gDW⁻¹ along the vertical axes of the right panels). FIG. 20N shows a direct comparison of root dry weight (in grams, plotted along the vertical axis) for these same lines, comparing plants grown under standard conditions (“Control”, left graph) to plants grown under drought conditions (“Drought”, right graph), with measurements taken at intermediate harvest (IH). In FIGS. 20E-20G and 20J-20N, for each cassava line, the central horizontal line represents the median, plus (+) indicates the mean; box edges delineate first and third quartiles, whiskers extend to maximum and minimum values and dots show individual values. Different lowercase letters indicate statistical significance, as calculated by one-way ANOVA with a post-hoc Tukey HSD test (p<0.05).

FIG. 21 shows a working model of ATK2var expressed in cassava to enhance phloem transport and increase drought tolerance. Empty vector (EV) lines and AKT2var lines are labeled. A representative model of a cassava plant, complete with storage roots, are to the left. The types of tissue portrayed in the rest of the model are indicated along the top, with xylem to the left and various relevant phloem components to the right. A magnified inset of the model shows a close-up schematic of the loading mechanism tied to voltage-gated potassium channel AKT2 between companion cells and apoplast (for which K⁺ indicates potassium ions), also showing the movement of sucrose across phloem complex components. “Suc”=sucrose, which is also illustrated being transported to the storage root for starch production. “H+”=hydrogen ions, “ATP”=adenosine triphosphate; “ADP”=adenosine diphosphate. “TP”=triose phosphates. The bidirectional movement of potassium at the bottom of the inset image of the model indicates the alteration in the mechanism present in AKT2var lines (“AKT2var”) that results in AKT2 acting as a rectifying channel.

FIGS. 22A-22D show, for greenhouse trials, the expression pattern of AKT2var under the native Arabidopsis promoter, and expression of AKT2var in various cassava tissues. FIG. 22A shows semi-quantitative real time polymerase chain reaction (PCR) measurements of relative AKT2var mRNA expression levels in source leaf tissue (“SoL”), petioles, upper stem peel tissue (“USP”), upper stem core tissue (USC), lower stem core tissue (“LSC”), lower stem peel tissue (“LSP”), root core tissue (“RC”), and root peel tissue (“RP”); results from empty vector plants are on the left (“Control”), and results from transformed pAtAKT2::AKT2var line 4266 are on the right. “H₂O” indicates a reaction run with water instead of a tissue sample. A molecular ladder is aligned between the control and transformed results. The top panel shows results for loading control MeGAPDH, and the bottom panel shows results for AtAKT2var. FIG. 22B shows a comparison of tracer transport velocity (in mm min⁻¹, plotted along the vertical axes) for up to four individual measurements (M01-M04) of biological triplicates (#1, #2, and #3) of empty vector (EV) line EV-4237 (left panel, circular plotted points), transformed line AKT2var-4261 (middle panel, triangular plotted points), and transformed line AKT2var-4262 (right panel, triangular plotted points). Shown are means+/−standard deviation for each individual measurement. Level of significance was calculated via one-way ANOVA with the post-hoc Tukey HSD test (** p<0.01, * p<0.05). In FIG. 22C, the same lines were sampled and displayed the same way as in FIG. 22B for CO₂ assimilation levels of ¹¹C-labelled leaves. For each trait, the average value and standard deviation (“SD”) of each line is listed to the right of the plots. FIG. 22D shows comparisons of levels of quantitative real time PCR measurements of relative AKT2var mRNA expression levels, compared between leaf tissue from source leaves (“leaf tissue (source)”, left panel), shoot tissue from the lower stem (“shoot (lower stem)”, middle panel), and root tissue from storage roots (“root tissue (storage root)”, right panel). Different lowercase letters indicate statistical significance, as calculated by one-way ANOVA with a post-hoc Tukey HSD test (p<0.05).

FIGS. 23A-23D show, for confined field trials in 2022, effects of expression of AKT2var on shoot and root growth. FIG. 23A shows representative lines' shoot and root tissues for empty vector (EV) control lines, from left to right of the top row of panels, “EV-4218”, “EV-4220”, “EV-4221”, “EV-4234”, and “EV-4243”; and for AKT2var lines, from left to right along the bottom row of panels, “AKT2var-4255”, “AKT2var-4261”, “AKT2var-4262”, “AKT2var-4265”, and “AKT2var-4266”. Scale bar=20 cm. FIG. 23B shows a plot of plant height (plotted in meters along the vertical axis) across plant growth (plotted in days after planting along the horizontal axis) for the same series of EV and AKT2var lines represented in FIG. 23A. FIG. 23C's left panel shows a plot of plant height (plotted in meters along the vertical axis) across plant growth (plotted in days after planting (DAP) along the horizontal axis) for the same series of EV and AKT2var lines represented in FIGS. 23A-23B. FIG. 23C's right panel shows a plot of plant volume (plotted in cubic meters along the vertical axis) across DAP for the same series of EV and AKT2var lines represented in FIGS. 23A-23B. FIG. 23D shows, for the same series of lines represented in FIGS. 23A-23B, shoot fresh weight (plotted in grams (g) along the vertical axis of the left panel), root fresh weight (plotted in g along the vertical axis of the middle panel), and dry matter content (plotted in % dry matter content along the vertical axis of the right panel). For each cassava line, the central horizontal line represents the median, plus (+) indicates the mean; box edges delineate first and third quartiles, whiskers extend to maximum and minimum values and dots show individual values. Different lowercase letters indicate statistical significance, as calculated by one-way ANOVA with a post-hoc Tukey HSD test (p<0.05).

FIGS. 24A-24D show, for confined field trials in 2023, effects of expression of AKT2var on shoot and root growth. FIG. 24A shows representative lines' shoot and root tissues for empty vector (EV) control lines, from left to right, “EV-4218”, “EV-4220”, “EV-4221”, “EV-4234”, and “EV-4243”. Scale bar=20 cm. FIG. 24B shows representative lines' shoot and root tissues for AKT2var lines, from left to right, “AKT2var-4255”, “AKT2var-4261”, “AKT2var-4262”, “AKT2var-4265”, and “AKT2var-4266”. Scale bar=20 cm. FIG. 24C's left panel shows a plot of plant height (plotted in meters along the vertical axis) across plant growth (plotted in days after planting along the horizontal axis) for the same series of EV and AKT2var lines represented in FIGS. 24A-24B. FIG. 24C's right panel shows a plot of plant volume (plotted in cubic meters along the vertical axis) across DAP for the same series of EV and AKT2var lines represented in FIGS. 24A-24B. FIG. 24D shows, for the same series of lines represented in FIGS. 24A-24C, shoot fresh weight (plotted in grams (g) along the vertical axis of the left panel), root fresh weight (plotted in g along the vertical axis of the middle panel), and dry matter content (plotted in % dry matter content along the vertical axis of the right panel). For each cassava line, the central horizontal line represents the median, plus (+) indicates the mean; box edges delineate first and third quartiles, whiskers extend to maximum and minimum values and dots show individual values. Different lowercase letters indicate statistical significance, as calculated by one-way ANOVA with a post-hoc Tukey HSD test (p<0.05).

FIGS. 25A-25H show, for confined field trials in 2022, effects of expression of AKT2var on shoot and root growth. FIG. 25A shows, from left to right, representative shoot and root tissues for empty vector (EV) control lines “EV-4218”, “EV-4220”, “EV-4221”, “EV-4234”, and “EV-4243”. Scale bar=20 cm. FIG. 25B shows, from left to right, representative shoot and root tissues for AKT2var lines, from left to right, “AKT2var-4255”, “AKT2var-4261”, “AKT2var-4262”, “AKT2var-4265”, and “AKT2var-4266”. Scale bar=20 cm. FIG. 25C's left panel shows a plot of plant height (plotted in meters along the vertical axis) across plant growth (plotted in days after planting along the horizontal axis) for the same series of EV and AKT2var lines represented in FIGS. 25A-25B. FIG. 25C's right panel shows a plot of plant volume (plotted in cubic meters along the vertical axis) across DAP for the same series of EV and AKT2var lines represented in FIGS. 25A-25B. FIG. 25D shows, for the same series of lines represented in FIGS. 25A-25C, shoot fresh weight (plotted in grams (g) along the vertical axis of the left panel), root fresh weight (plotted in g along the vertical axis of the middle panel), and dry matter content (plotted in % dry matter content along the vertical axis of the right panel). For each cassava line, the central horizontal line represents the median, plus (+) indicates the mean; box edges delineate first and third quartiles, whiskers extend to maximum and minimum values and dots show individual values. Different lowercase letters indicate statistical significance, as calculated by one-way ANOVA with a post-hoc Tukey HSD test (p<0.05). FIG. 25E shows patterns of precipitation (left panels) and temperature (right panels) for field trial sites across 2022-2024 (top row to bottom row), plotted across months of the year (horizontal axes), with the time of final harvest (“FH”) indicated in each plot. FIG. 25F shows cation levels, specifically potassium (K⁺, left column), calcium (Ca²⁺, middle column), and magnesium (Mg²⁺, right column), plotted in mol gDW⁻¹ along the vertical axes for shoot tissue from the lower stem (top row of panels), and root tissue from storage roots (bottom row of panels). FIG. 25G shows anion levels, specifically phosphate (PO₄³⁻, left column), sulphate (SO₄²⁻, middle column), and chloride (Cl⁻, right column), plotted in mol gDW⁻¹ along the vertical axes for shoot tissue from the lower stem (top row of panels), and root tissue from storage roots (bottom row of panels). FIG. 25H shows sugar levels, specifically (in rows of panels from top to bottom) glucose, fructose, and sucrose, as well as starch (bottom row of panels), plotted in mol gDW⁻¹ along the vertical axes for leaf tissue from shoot tissue from the lower stem (left column), and root tissue from storage roots (right column). In FIGS. 25D and 25F-25H, for each cassava line, the central horizontal line represents the median, plus (+) indicates the mean; box edges delineate first and third quartiles, whiskers extend to maximum and minimum values and dots show individual values. Different lowercase letters indicate statistical significance, as calculated by one-way ANOVA with a post-hoc Tukey HSD test (p<0.05).

FIG. 26 shows, for greenhouse conditions, a schematic representation of the cultivation process to apply drought stress to cassava lines. Proceeding from left to right, all plants are grown under “standard” conditions, wherein 300-400 mL of water is supplied every one to two days. For one group of these plants, water is then supplied in either 200 or 100 mL amounts as indicated, across 5 weeks (followed by an intermediate harvest). During this time, the other group of plants are still exposed to only standard conditions as initially described and at the end of the 5 weeks also undergo an intermediate harvest (IH). Following the 5 weeks of differing treatments, all plants are then exposed to only standard conditions for an additional 4-6 weeks, followed by a final harvest (FH).

FIGS. 27A-27J show, for both standard conditions and drought conditions of greenhouse trials, effects of expression of AKT2var on shoot and root growth as well as tolerance of drought stress. FIGS. 27A-27B show results from measurements taken at intermediate harvest. FIG. 27A shows, for standard conditions, from left to right, representative shoot and root tissues for empty vector (EV) control lines “EV-4218”, “EV-4234”, and “EV-4243”; and AKT2var lines “AKT2-4261”, “AKT2-4262”, and “AKT2-4264”. Scale bar=20 cm for the top row of panels, and scale bar=5 cm for the bottom row of panels. FIG. 27B shows, for drought conditions, from left to right, representative shoot and root tissues for empty vector (EV) control lines “EV-4218”, “EV-4234”, and “EV-4243”; and AKT2var lines “AKT2-4261”, “AKT2-4262”, and “AKT2-4264”. Scale bar=20 cm for the top row of panels, and scale bar=5 cm for the bottom row of panels. FIG. 27C shows, for the same lines and standard conditions used in FIG. 27A, results of measuring stem dry weight (in grams, left column of panels) and leaf dry weight (in grams, right column of panels), for measurements taken at final harvest (bottom row) or intermediate harvest (top row). FIG. 27D shows, the measurements corresponding to FIG. 27C taken for plants grown in drought conditions. FIGS. 27E-27J show results taken from leaf tissue (top row of panels), lower stem shoot tissue (middle row of panels), and fibrous root tissue (bottom row of panels). FIGS. 27E and 27G show, for standard conditions, effects of AKT2var expression on cassava lines' cation contents (FIG. 27E) and anion contents (FIG. 27G). FIGS. 27F and 27H show measurements corresponding to FIGS. 27E and 27G, respectively, for plants grown in drought conditions. For FIGS. 27E-27F, from left to right, the cations compared are potassium (K⁺ ions), calcium (Ca²⁺ ions), and magnesium (Mg²⁺ ions), plotted in mol gDW⁻¹ along the vertical axes. For each of FIGS. 27G-27H, from left to right, the anions compared are phosphate (PO₄³⁻ ions), sulfate (SO₄²⁻ ions), and chloride (Cl⁻ ions), plotted in mol gDW⁻¹ along the vertical axes. From the leftmost column to rightmost column, FIGS. 27I-27J show sugar and starch levels, specifically glucose, fructose, sucrose, and starch, plotted in mol gDW⁻¹ along the vertical axes. For each cassava line in FIGS. 27C-27J, the central horizontal line represents the median, plus (+) indicates the mean; box edges delineate first and third quartiles, whiskers extend to maximum and minimum values and dots show individual values. Different lowercase letters indicate statistical significance, as calculated by one-way ANOVA with a post-hoc Tukey HSD test (p<0.05).

FIGS. 28A-28B show locations and resultant canopy profiles generated through field examinations of photosynthetic performance. FIG. 28A shows a generalized experimental setup. The left panel shows an aerial view of a field's locations of AKT2var and empty vector (EV) control plants in 2022. The field is oriented with north-south (measured in latitude) plotted from top to bottom along the vertical axis and west to east (measured in longitude) plotted from left to right along the horizontal axis. Accession names of each sampled plant is listed next to the image of the field. This selection of accession plant locations is the first step. The right panel shows a generalized set of following steps, where the top row of panels each show a measuring head attached to leaves, with the right panel showing brighter light conditions and the left panel showing darker light conditions. The bottom right panel shows a labeled white reflectance pane used in the experiment for measuring photosynthetically active radiation (PAR). FIG. 28B shows results of analyzing canopy structure for cassava. The left panel shows a generalized schematic of a cassava canopy profile, with digital elevation (DEM) marked as the top continuous line and digital terrain (DTM) marked as a dotted line. The lower, sinusoidal continuous line represents crop surface models (CSM) showing different aspects of the canopy, with CSM at each measured point=DEM value−DTM value, plotted as meters above ground level (mAGL). The barred box along the CSM's line marks the value of the 95^{th} quantile of CSM, and the lowest continuous line represents ground level. CSM is plotted along each panel's left vertical axis. DEM and DTM in the meters above the mean sea level (mAMSL) is plotted along the right side of the vertical axis for the left panel. The middle panel and right panel represent a closer look at the shaded bars under the CSM line of the left panel. The middle panel (taken from the left panel's dotted box region) has a set of individual pixel values plotted along the horizontal axis; the solid horizontal line represents the 95^{th} quantile, the value of CSM below which 95% of the data fall—which is also the value used for height estimation. Counterpart to the middle panel's height estimation, the right panel shows the corresponding volume estimation, wherein the volume is the area under the left panel's CSM curve, or the pixel area multiplied by the pixel (CSM) value. Distance in meters is plotted along the horizontal axis. The continuous line marked with the barred triangle represents the ground level (“Ground representation”).

### DETAILED DESCRIPTION

The following description sets forth exemplary methods, parameters, and the like. It should be recognized, however, that such description is not intended as a limitation on the scope of the present disclosure but is instead provided as a description of exemplary embodiments.

### Genetically Modified Plants, Plant Parts, Plant Cells, and Related Methods

An aspect of the disclosure includes a genetically modified plant or part thereof including one or more nucleotide sequences encoding a modified POTASSIUM TRANSPORTER 2 (AKT2) protein. In an additional embodiment of this aspect, the modified AKT2 protein is selected from the group of a modified Arabidopsis thaliana POTASSIUM TRANSPORTER 2 (AtAKT2) protein, a modified first Manihot esculenta AKT2 protein (MeAKT2a), and/or a modified second Manihot esculenta AKT2 protein (MeAKT2b). One aspect of the present disclosure provides a genetically modified plant, plant part thereof, or plant cell thereof, comprising one or more nucleotide sequences encoding a modified POTASSIUM TRANSPORTER 2 (AKT2) protein, wherein the modified AKT2 protein is selected from the group of a modified plant AKT2 protein, a modified Arabidopsis thaliana AKT2 (AtAKT2) protein, a modified first Manihot esculenta AKT2 protein (MeAKT2a), and/or a modified second Manihot esculenta AKT2 protein (MeAKT2b), or a homolog thereof. In some embodiments, a wild-type AKT2 protein has phloem potassium transport activity and the modified AKT2 protein has phloem potassium transport activity. In a further embodiment of this aspect, an AKT2 protein includes: (a) mode 1, wherein the AKT2 protein acts as an inward-rectifying K+ channel (Kin); and (b) mode 2, wherein the AKT2 protein acts as a nonrectifying channel; wherein the wild-type AKT2 protein comprises mode 1; and wherein the modified AKT2 protein comprises modifications that bias the modified AKT2 toward mode 2 or lock the modified AKT2 in mode 2. In a further embodiment of this aspect, a wild-type AKT2 protein includes mode 1, wherein the wild-type AKT2 acts as an inward-rectifying K⁺ channel (Kᵢₙ), and mode 2, wherein the AKT2 acts as a nonrectifying channel, and wherein the modified AKT2 protein includes modifications that bias the modified AKT2 toward mode 2 or lock the modified AKT2 in mode 2. In still another embodiment of this aspect, the wild-type AtAKT2 protein includes a protein including at least 70% identity, at least 80% identity, at least 90% identity, at least 95% identity, or at least 99% identity to SEQ ID NO: 17, the wild-type MeAKT2a protein includes a protein including at least 70% identity, at least 80% identity, at least 90% identity, at least 95% identity, or at least 99% identity to SEQ ID NO: 19, and the wild-type MeAKT2b protein includes a protein including at least 70% identity, at least 80% identity, at least 90% identity, at least 95% identity, or at least 99% identity to SEQ ID NO: 20; or a functional fragment of one of the foregoing. In still another embodiment of this aspect, which may be combined with any of the preceding embodiments, the modified AKT2 protein comprises one or more amino acid substitutions, insertions, or deletions in the Ion_trans (PF00520, Ion transport protein) motif (corresponding to amino acids 75-323 of SEQ ID NO: 17) and/or one or more amino acid substitutions, insertions, or deletions in the Ion_trans_2 (PF07885, Ion channel) motif (corresponding to amino acids 225-317 of SEQ ID NO: 17), wherein the one or more substitutions, insertions, or deletions modulate or increase the ion transport activity; or the modified AKT2 protein comprises one or more amino acid substitutions, insertions, or deletions, wherein the one or more substitutions, insertions, or deletions modulate, alter, or increase the ion transport activity. In yet another embodiment of this aspect, which may be combined with any of the preceding embodiments, the modified plant AKT2 protein, the modified AtAKT2 protein, the modified MeAKT2a protein, and/or the modified MeAKT2b protein includes one or both of the amino acid substitutions corresponding to S210N and S329N when aligned to SEQ ID NO: 17. In an additional embodiment of this aspect, which may be combined with any of the preceding embodiments, the modified AtAKT2 protein includes a protein including at least 70% identity, at least 80% identity, at least 90% identity, at least 95% identity, or at least 99% identity to SEQ ID NO: 18, wherein the modified MeAKT2a protein includes a protein comprising at least 70% identity, at least 80% identity, at least 90% identity, at least 95% identity, or at least 99% identity to SEQ ID NO: 25, and wherein the modified MeAKT2b protein includes a protein comprising at least 70% identity, at least 80% identity, at least 90% identity, at least 95% identity, or at least 99% identity to SEQ ID NO: 26. In a further embodiment of this aspect, which may be combined with any of the preceding embodiments, the one or more nucleotide sequences encoding the modified AtAKT2 protein includes a nucleotide sequence including at least 70% identity, at least 80% identity, at least 90% identity, at least 95% identity, or at least 99% identity to SEQ ID NO: 3. In still another embodiment of this aspect, which may be combined with any of the preceding embodiments, the one or more nucleotide sequences encoding the modified AtAKT2 protein, the modified MeAKT2a protein, and/or the modified MeAKT2b protein is operably linked to an expression control sequence. In an additional embodiment of this aspect, the expression control sequence includes an overexpression promoter and/or a phloem-specific promoter. In an additional embodiment of this aspect, the expression control sequence includes an overexpression promoter, a phloem-specific promoter, and/or a xylem-specific promoter. In still another embodiment of this aspect, the promoter includes an Arabidopsis thaliana POTASSIUM TRANSPORTER 2 (pAtAKT2) promoter, an Arabidopsis thaliana SUCROSE TRANSPORTER 2 promoter (pAtSUC2), a COMMELINA YELLOW MOTTLE VIRUS promoter (pCoYMV), a Rice tungro bacilliform virus promoter (pRTBV), a Solanum tuberosum KST1 promoter (pStKST1), a cassava MeAKT2a promoter, a cassava MeAKT2b promoter, or a proIC promoter. In a further embodiment of this aspect, the promoter includes the pAtAKT2 promoter, the cassava MeAKT2a promoter, or the cassava MeAKT2b promoter. In yet another embodiment of this aspect, the promoter includes the pAtAKT2 promoter, and wherein the promoter includes a nucleotide sequence including at least 70% identity, at least 80% identity, at least 90% identity, at least 95% identity, or at least 99% identity to SEQ ID NO: 2.

In some embodiments, the genetically modified plant is a plant part or a plant cell. For example, cassava plant parts include cassava leaves, flowers, carpels, ovaries, ovules, stamens, anthers, pollen, extracted juices, fruit, calli, phloem, xylem, seeds, shoots, roots, cassava storage roots, parts of cassava storage roots, cassava tubers, fibrous roots, cells, and the like. In one embodiment, the present disclosure is directed to cassava leaves, xylem, phloem, shoots, roots, storage roots, seeds, and/or cells isolated from AtAKT2var cassava plants. In another embodiment, the present disclosure is further directed to tissue culture of AtAKT2var cassava plants, and to cassava plants regenerated from the tissue culture, where the plant has all of the morphological and physiological characteristics of the parent AtAKT2var cassava plant. In certain embodiments, tissue culture of AtAKT2var cassava plants is produced from a plant part selected from leaf, anther, pistil, stem, petiole, root, root tip, fruit, seed, flower, cotyledon, hypocotyl, embryo, and meristematic cell. In some embodiments, the plant part is a non-reproducible plant cell of an AtAKT2var cassava plant. In some embodiments, the genetically modified plant is an aboveground plant part. Aboveground plant parts include any plant tissue above soil level. Aboveground plant parts include leaves, flowers, carpels, ovaries, ovules, stamens, anthers, pollen, fruit, calli of aboveground tissue(s), phloem, xylem, seeds, shoots, aboveground cells, and the like. In cassava, aboveground plant parts do not include cassava roots, fibrous roots, storage roots, and the like.

An additional aspect of the disclosure includes a genetically modified plant or part thereof including one or more nucleotide sequences encoding a POTASSIUM TRANSPORTER 2 (AKT2) protein operably linked to an expression control sequence, wherein the expression control sequence includes an overexpression promoter, optionally wherein the AKT2 protein is a wild-type protein. In a further embodiment of this aspect, the AKT2 protein is selected from the group of an Arabidopsis thaliana POTASSIUM TRANSPORTER 2 (AtAKT2) protein, a first Manihot esculenta AKT2 protein (MeAKT2a), and/or a second Manihot esculenta AKT2 protein (MeAKT2b). In another embodiment, which may be combined with any preceding embodiment, the modified AKT2 protein, the modified plant AKT2 protein, the modified AtAKT2 protein, the modified MeAKT2a protein, and/or the modified MeAKT2b protein (a) includes one or more amino acid substitutions, insertions, or deletions in the Ion_trans (PF00520, Ion transport protein) motif (corresponding to amino acids 75-323 of SEQ ID NO: 17) and/or one or more amino acid substitutions, insertions, or deletions in the Ion_trans_2 (PF07885, Ion channel) motif (corresponding to amino acids 225-317 of SEQ ID NO: 17), wherein the one or more substitutions, insertions, or deletions modulate or increase the ion transport activity; (b) includes one or more amino acid substitutions, insertions, or deletions, wherein the one or more substitutions, insertions, or deletions modulate or increase the ion transport activity; (c) includes one or both of the amino acid substitutions corresponding to S210N and S329N when aligned to SEQ ID NO: 17; (d) includes one or both of the amino acid substitutions corresponding to S199N and S139N when aligned to SEQ ID NO: 19; or (e) includes one or both of the amino acid substitutions corresponding to S216N and S319N when aligned to SEQ ID NO: 20. In another embodiment of this aspect, the AtAKT2 protein includes a protein including at least 70% identity, at least 80% identity, at least 90% identity, at least 95% identity, or at least 99% identity to SEQ ID NO: 17 or SEQ ID NO: 18, the MeAKT2a protein comprises a protein comprising at least 70% identity, at least 80% identity, at least 90% identity, at least 95% identity, or at least 99% identity to SEQ ID NO: 19 or SEQ ID NO: 25, and the MeAKT2b protein comprises a protein comprising at least 70% identity, at least 80% identity, at least 90% identity, at least 95% identity, or at least 99% identity to SEQ ID NO: 20 or SEQ ID NO: 26; or a functional fragment of one of the foregoing. In still another embodiment of this aspect, which may be combined with any of the preceding embodiments, the overexpression promoter additionally includes tissue-specific expression, and wherein the tissue-specific expression is selected from the group of phloem-specific expression, xylem-specific expression, root-specific expression, or stomata-specific expression. In yet another embodiment of this aspect, which may be combined with any of the preceding embodiments, the overexpression promoter includes an Arabidopsis thaliana POTASSIUM TRANSPORTER 2 (pAtAKT2) promoter, an Arabidopsis thaliana SUCROSE TRANSPORTER 2 promoter (pAtSUC2), a COMMELINA YELLOW MOTTLE VIRUS promoter (pCoYMV), a Rice tungro bacilliform virus promoter (pRTBV), a Solanum tuberosum KST1 promoter (pStKST1), a cassava MeAKT2a promoter, or a cassava MeAKT2b promoter. In a further embodiment of this aspect, the promoter includes the pAtAKT2 promoter, the cassava MeAKT2a promoter, or the cassava MeAKT2b promoter. In an additional embodiment of this aspect, the promoter is the pAtAKT2 promoter, and wherein the promoter includes a nucleotide sequence including at least 70% identity, at least 80% identity, at least 90% identity, at least 95% identity, or at least 99% identity to SEQ ID NO: 2.

In a further embodiment of this aspect, which may be combined with any of the preceding embodiments, the plant produces a root vegetable, storage roots, or tubers. For example, a root vegetable can be an edible underground plant part. In one embodiment of this aspect, the plant produces storage roots. In another embodiment of this aspect, the plant produces tubers. In some embodiments, the plant that produces roots or tuberous roots can be cassava, sweet potato, jicama, or yacon. In some embodiments, the plant that produces tubers can be potato, yam, oca, mashua, ulluco, Jerusalem artichoke, or tiger nut. In some embodiments, the plant that produces stem tuber can be potato. In some embodiments, the plant that produces corms can be taro, water chestnut, elephant foot yam (suran), eddoe, or arrowhead. In some embodiments, the plant that produces rhizomes can be ginger, turmeric, galangal, lotus root, wasabi, arrowroot, canna, or bamboo shoots. In some embodiments, the plant that produces bulbs can be onion or garlic. In an additional embodiment of this aspect, the plant is selected from the group of cassava (Manihot esculenta), potato (Solanum tuberosum), sweet potato (Ipomoea batatas), yam (Dioscorea spp.), ube (Dioscorea alata), yacón (Smallanthus sonchifolius), taro (Colocasia esculenta), konjac (Amorphophallus konjac), ginger (Zingiber officinale), radish (Raphanus raphanistrum subsp. Sativus), turnip (Brassica rapa subsp. Rapa), rutabaga (Brassica napus), parsnip (Pastinaca sativa), jicama (Pachyrhizus erosus), Jerusalem artichoke (Helianthus tuberosus), turmeric (Curcuma longa), horseradish (Armoracia rusticana), beet (Beta vulgaris subsp. Vulgaris), lotus (Nelumbo nucifera), maca (Lepidium meyenii), celeriac (Apium graveolens var. rapaceum), skirret (Sium sisarum), or wasabi (Eutrema japonicum). In yet another embodiment, the genetically modified plant or part thereof can be a crop that benefits from potassium fertilization. For example, some crops that can benefit from potassium fertilization can be cassava, yams, potatoes, tomatoes, citrus fruits (e.g., oranges, lemons, limes, etc.), bananas, grains (e.g., wheat, rice, barley, sorghum, etc.), cotton, sugar beets, soybeans, cowpea, alfalfa, grapes, peppers, apples, melons (e.g., watermelon, cantaloupe, etc.). Accordingly, in some embodiments, the plant is a dicot; the plant produces storage roots or tubers and/or benefits from potassium fertilization, optionally wherein the plant is selected from the group consisting of cassava, potato, sweet potato, yam, ube, yacón, taro, konjac, ginger, radish, turnip, rutabaga, parsnip, jicama, Jerusalem artichoke, turmeric, horseradish, beet, lotus, maca, celeriac, skirret, wasabi, citrus fruits, bananas, grains, tomatoes, sorghum, cotton, sugar beets, soybeans, cowpea, alfalfa, grapes, peppers, apples, and melons; and/or the plant has a large transport distance between a storage organ and a photosynthetic leaf. In yet another embodiment of this aspect, which may be combined with any of the preceding embodiments, the plant is a passive symplasmic phloem loader. In still another embodiment of this aspect, which may be combined with any of the preceding embodiments, the genetically modified plant or part thereof has higher photosynthetic efficiency, improved photosynthesis, higher rate of CO₂ fixation and/or electron transport rate, earlier maximum growth rate, improved yield under field conditions, improved yield under drought conditions, and/or improved yield under potassium deficiency as compared to a control plant grown under the same conditions, optionally wherein the genetically modified plant or part thereof or a progenitor thereof was selected for improved growth, improved photosynthesis, higher rate of CO₂ fixation, and/or higher electron transport rate when grown under non-limiting energy conditions, earlier maximum growth rate, improved yield, increased plant growth, increased plant height, increased shoot growth, increased total shoot dry matter, improved storage root or tuber growth, increased number of storage roots or tubers per plant, and/or increased total storage root or tuber dry matter. In still another aspect, the genetically modified plant has improved phloem transport, improved phloem mass flow, improved source-sink delivery, increased fibrous root formation, higher photosynthetic efficiency, improved photosynthesis, higher rate of CO₂ fixation and/or electron transport rate, earlier maximum growth rate, increased yield under field conditions, increased yield under drought conditions, increased drought stress resistance, improved drought tolerance, and/or increased yield under potassium deficiency as compared to a control plant grown under the same conditions, optionally wherein the genetically modified plant or a progenitor thereof was selected for improved phloem transport, improved phloem mass flow, improved source-sink delivery, increased fibrous root formation, increased growth, improved photosynthesis, higher rate of CO₂ fixation, and/or electron transport rate when grown under non-limiting energy conditions, earlier maximum growth rate, increased yield, increased plant growth, increased plant height, increased shoot growth, increased total shoot dry matter, increased storage root or tuber biomass, increased number of storage roots or tubers per plant, and/or increased total storage root or tuber dry matter. In an additional embodiment of this aspect, which may be combined with any of the preceding embodiments, the genetically modified plant or plant part thereof has improved phloem transport, improved phloem mass flow, improved source-sink delivery, increased fibrous root formation, higher photosynthetic efficiency, improved photosynthesis, higher rate of CO2 fixation and/or electron transport rate, earlier maximum growth rate, increased yield under field conditions, increased yield under drought conditions, increased drought stress resistance, increased drought tolerance, and/or increased yield under potassium deficiency as compared to a control plant grown under the same conditions, and wherein: (i) the plant produces storage roots or tubers and/or benefits from potassium fertilization, optionally wherein the plant is selected from the group consisting of cassava, potato, sweet potato, yam, ube, yacón, taro, konjac, ginger, radish, turnip, rutabaga, parsnip, jicama, Jerusalem artichoke, turmeric, horseradish, beet, lotus, maca, celeriac, skirret, wasabi, citrus fruits, bananas, grains, tomatoes, sorghum, cotton, sugar beets, soybeans, cowpea, alfalfa, grapes, peppers, apples, and melons; and/or (ii) wherein the plant is a passive symplasmic phloem loader.

In yet another embodiment of this aspect, which may be combined with any of the preceding embodiments, the genetically modified plant or part thereof is a cassava plant, and wherein the genetically modified cassava plant has increased plant growth, increased plant height, increased shoot growth, increased total shoot dry matter, improved storage root growth, improved number of storage roots per plant, and/or increased total root dry matter as compared to a control cassava plant grown under the same conditions. In yet another embodiment of this aspect, which may be combined with any of the preceding embodiments, the genetically modified plant is a cassava plant, wherein the genetically modified cassava plant has improved phloem transport, increased plant growth, increased plant height, increased shoot growth, increased total shoot dry matter (TSDM), increased storage root growth, increased drought stress resistance, increased drought tolerance, improved photosynthetic performance, lower proline and/or serine levels in drought conditions, increased number of storage root per plant, and/or increased total storage root dry matter (TRDM) as compared to a control cassava plant grown under the same conditions, the genetically modified plant includes (a) at least one of the following shoot traits: increased height, increased concentrations of sodium (Na+), increased concentrations of calcium (Ca2+), increased concentrations of magnesium (Mg2+), increased concentrations of potassium (K+), reduced sucrose concentration or level in aboveground plant parts, increased starch concentration or level, increased shoot fresh weight, increased TSDM, and increased phloem transport rate; and/or (b) at least one of the following root traits: reduced concentrations of K+, reduced sucrose concentration, increased glucose concentration, increased fructose concentration, increased starch concentration, increased root fresh weight, and increased TRDM as compared to a control plant grown under the same conditions. In a further embodiment of this aspect, the genetically modified cassava plant has elevated concentrations of sodium (Na⁺), calcium (Ca²⁺), magnesium (Mg²⁺), and/or potassium (K⁺) in shoot tissue, reduced concentrations of sodium (Na⁺), calcium (Ca²⁺), magnesium (Mg²⁺), and/or potassium (K⁺) in root tissue, reduced sucrose concentration in shoot and/or root tissue, increased glucose and/or fructose concentration in root tissue, and/or increased starch concentration in root tissue as compared to a control cassava plant grown under the same conditions. In an additional embodiment of this aspect, which may be combined with any of the preceding embodiments that has a cassava plant, the genetically modified cassava plant includes cultivar TMS60444. In yet another embodiment, the genetically modified plant (a) reaches the maximum relative growth rate (RGR) faster, (b) has an increased harvest index (HI), (c) has increased yield, (d) has a higher maximum electron transport rate (ETR), (e) has an increased tracer transport velocity; and/or (f) has an increased CO2 assimilation rate as compared to a control plant grown under the same conditions. In yet another embodiment of this aspect, which may be combined with any of the preceding embodiments, the genetically modified plant grown under drought conditions has (a) increased relative yield, (b) elevated sucrose concentrations; (c) elevated glucose concentrations; (d) elevated fructose concentrations; (e) elevated starch concentrations; (f) increased TSDM; (g) increased TRDM; and/or (h) reduced serine and/or proline concentrations as compared to a control plant grown under drought conditions. In yet another embodiment of this aspect, which may be combined with any of the preceding embodiments, the genetically modified plant exhibits increased drought stress resistance and/or increased drought tolerance as compared to a control plant grown under the same conditions, and wherein the increased drought stress resistance and/or increased drought tolerance is indicated by reduced proline concentrations, reduced serine concentrations, and/or increased relative yield as compared to a control plant grown under the same conditions. Without wishing to be bound by theory, it is thought that the same effects would be observed in any cassava cultivars that have been modified according to the embodiments of the present disclosure.

The compositions and methods described herein are contemplated to be beneficial for seed plants generally, and are considered to be advantageous both for plants with symplasmic phloem loading and plants exhibiting active phloem loading. In particular, it is believed that plants characterized by extended source-to-sink transport distances may derive the greatest benefit. For example, although cassava (Manihot esculenta) primarily employs symplasmic phloem loading in its foliar tissues and symplasmic unloading in its lower stem and storage roots, active transport mechanisms are nonetheless utilized. It is further believed that such active transport is of particular importance in cassava due to the substantial long-distance assimilate translocation that occurs along the stem. As such, it believed that increased assimilate delivery to sink organs can also improve the size of the fibrous root network, improving the plants ability to take up nutrients or withstand drought conditions.

In a further embodiment of this aspect, which may be combined with any of the preceding embodiments, the plant can be a dicot or a monocot. For example, the plant can be soy, cowpea, brassica, or canola because this technology can be relevant to any harvestable organ far apart from the producing leaves. Dicots include a wide set of angiosperm clades that, among some typical traits, exhibit two cotyledons rather than one (which occurs in monocots). Exemplary dicots include sweet potato, radish, turnip, rutabaga, parsnip, jicama, Jerusalem artichoke, horseradish, beet, lotus, maca, celeriac, skirret, wasabi, tomatoes, cotton, sugar beets, soybeans, cowpea, alfalfa, grapes, peppers, apples, and melons.

A further aspect of the disclosure includes methods of producing the genetically modified plant or part thereof of any one of the preceding embodiments that has a modified AKT2 protein, including introducing one or more nucleotide sequences encoding the modified plant AKT2 protein, the modified AtAKT2 protein, the modified MeAKT2a protein, and/or the modified MeAKT2b protein. In an additional embodiment of this aspect, the modified AtAKT2 protein, the modified MeAKT2a protein, and/or the modified MeAKT2b protein includes one or both of the amino acid substitutions corresponding to S210N and S329N when aligned to SEQ ID NO: 17. In yet another embodiment of this aspect, which may be combined with any of the preceding embodiments, the modified AtAKT2 protein includes a protein including at least 70% identity, at least 80% identity, at least 90% identity, at least 95% identity, or at least 99% identity to SEQ ID NO: 18, wherein the modified MeAKT2a protein includes a protein comprising at least 70% identity, at least 80% identity, at least 90% identity, at least 95% identity, or at least 99% identity to SEQ ID NO: 25, and wherein the modified MeAKT2b protein includes a protein comprising at least 70% identity, at least 80% identity, at least 90% identity, at least 95% identity, or at least 99% identity to SEQ ID NO: 26. In still another embodiment of this aspect, which may be combined with any of the preceding embodiments, the one or more nucleotide sequences encoding the modified AtAKT2 protein includes a nucleotide sequence including at least 70% identity, at least 80% identity, at least 90% identity, at least 95% identity, or at least 99% identity to SEQ ID NO: 3. In a further embodiment of this aspect, which may be combined with any of the preceding embodiments, the one or more nucleotide sequences encoding the modified AtAKT2 protein, the modified MeAKT2a protein, and/or the modified MeAKT2b protein is operably linked to an expression control sequence. In yet another embodiment of this aspect, the expression control sequence includes an overexpression promoter and/or a phloem-specific promoter. In still another embodiment of this aspect, which may be combined with any of the preceding embodiments, the one or more nucleotide sequences encoding the modified plant AKT2 protein, the modified AtAKT2 protein, the modified MeAKT2a protein, and/or the modified MeAKT2b protein is operably linked to an expression control sequence. In an additional embodiment of this aspect, the expression control sequence includes an overexpression promoter, a phloem-specific promoter, and/or a xylem-specific promoter. In still another embodiment of this aspect, the promoter includes an Arabidopsis thaliana POTASSIUM TRANSPORTER 2 (pAtAKT2) promoter, an Arabidopsis thaliana SUCROSE TRANSPORTER 1 promoter (pAtSUC1), a COMMELINA YELLOW MOTTLE VIRUS promoter (pCoYMV), a Rice tungro bacilliform virus promoter (pRTBV), a Solanum tuberosum KST1 promoter (pStKST1), a cassava MeAKT2a promoter, or a cassava MeAKT2b promoter. In an additional embodiment of this aspect, the promoter includes the pAtAKT2 promoter, the cassava MeAKT2a promoter, a cassava MeAKT2b promoter, or a proIC promoter. In a further embodiment of this aspect, the one or more nucleotide sequences encoding the modified plant AKT2 protein, the modified AtAKT2 protein, the modified MeAKT2a protein, and/or the modified MeAKT2b protein is operably linked to an expression control sequence; wherein the expression control sequence includes an overexpression promoter and/or a phloem-specific promoter; and optionally wherein the promoter comprises a plant AKT2 promoter, an Arabidopsis thaliana POTASSIUM TRANSPORTER 2 (pAtAKT2) promoter, an Arabidopsis thaliana SUCROSE TRANSPORTER 1 promoter (pAtSUC2), a cassava MeAKT2a promoter, a cassava MeAKT2b promoter, or a proIC promoter. In a further embodiment of this aspect, the promoter includes the pAtAKT2 promoter, and wherein the promoter includes a nucleotide sequence including at least 70% identity, at least 80% identity, at least 90% identity, at least 95% identity, or at least 99% identity to SEQ ID NO: 2.

Yet another aspect of the disclosure includes methods of producing the genetically modified plant or part thereof of any one of the preceding embodiments that has a modified AKT2 protein, including genetically modifying a plant by transforming the plant with one or more gene editing components that target an endogenous nuclear genome sequence encoding the wild-type plant AKT2 protein, the wild-type AtAKT2 protein, the wild-type MeAKT2a protein, and/or the wild-type MeAKT2b protein to produce the modified plant AKT2 protein, the modified AtAKT2 protein, the modified MeAKT2a protein, and/or the modified MeAKT2b protein. In a further embodiment of this aspect, the one or more gene editing components include a ribonucleoprotein complex that targets the nuclear genome sequence; a vector including a TALEN protein encoding sequence, wherein the TALEN protein targets the nuclear genome sequence; a vector including a ZFN protein encoding sequence, wherein the ZFN protein targets the nuclear genome sequence; an oligonucleotide donor (OND), wherein the OND targets the nuclear genome sequence; or a vector CRISPR/Cas enzyme encoding sequence and a targeting sequence, wherein the targeting sequence targets the nuclear genome sequence. In an additional embodiment of this aspect, which may be combined with any one of the preceding embodiments, the wild-type AtAKT2 protein includes a protein including at least 70% identity, at least 80% identity, at least 90% identity, at least 95% identity, or at least 99% identity to SEQ ID NO: 17, the wild-type MeAKT2a protein includes a protein including at least 70% identity, at least 80% identity, at least 90% identity, at least 95% identity, or at least 99% identity to SEQ ID NO: 19, and the wild-type MeAKT2b protein includes a protein including at least 70% identity, at least 80% identity, at least 90% identity, at least 95% identity, or at least 99% identity to SEQ ID NO: 20; or a functional fragment of one of the foregoing. In still another embodiment of this aspect, the wild-type plant AKT2 protein includes a protein including at least 70% identity, at least 80% identity, at least 90% identity, at least 95% identity, or at least 99% identity to one of SEQ ID NOs: 17, 19, and 20, the wild-type AtAKT2 protein includes a protein including at least 70% identity, at least 80% identity, at least 90% identity, at least 95% identity, or at least 99% identity to SEQ ID NO: 17, the wild-type MeAKT2a protein includes a protein including at least 70% identity, at least 80% identity, at least 90% identity, at least 95% identity, or at least 99% identity to SEQ ID NO: 19, and the wild-type MeAKT2b protein includes a protein including at least 70% identity, at least 80% identity, at least 90% identity, at least 95% identity, or at least 99% identity to SEQ ID NO: 20; or a functional fragment of one of the foregoing. In still another embodiment of this aspect, which may be combined with any of the preceding embodiments, the modified plant AKT2 protein comprises one or more amino acid substitutions, insertions, or deletions in the Ion_trans (PF00520, Ion transport protein) motif (corresponding to amino acids 75-323 of SEQ ID NO: 17) and/or one or more amino acid substitutions, insertions, or deletions in the Ion_trans_2 (PF07885, Ion channel) motif (corresponding to amino acids 225-317 of SEQ ID NO: 17), wherein the one or more substitutions, insertions, or deletions modulate or increase the ion transport activity; or the modified AKT2 protein comprises one or more amino acid substitutions, insertions, or deletions, wherein the one or more substitutions, insertions, or deletions modulate or increase the ion transport activity. In yet another embodiment of this aspect, which may be combined with any of the preceding embodiments, the modified AtAKT2 protein, the modified MeAKT2a protein, and/or the modified MeAKT2b protein includes one or both of the amino acid substitutions corresponding to S210N and S329N when aligned to SEQ ID NO: 17. In still another embodiment of this aspect, which may be combined with any of the preceding embodiments, the modified AtAKT2 protein includes a protein including at least 70% identity, at least 80% identity, at least 90% identity, at least 95% identity, or at least 99% identity to SEQ ID NO: 18, wherein the modified MeAKT2a protein includes a protein comprising at least 70% identity, at least 80% identity, at least 90% identity, at least 95% identity, or at least 99% identity to SEQ ID NO: 25, and wherein the modified MeAKT2b protein includes a protein comprising at least 70% identity, at least 80% identity, at least 90% identity, at least 95% identity, or at least 99% identity to SEQ ID NO: 26. In some embodiments, which may be combined with any of the preceding embodiments, the wild-type plant AKT2 protein comprises a protein comprising at least 70% identity, at least 80% identity, at least 90% identity, at least 95% identity, or at least 99% identity to one of SEQ ID NOs: 17, 19, and 20, the wild-type plant AtAKT2 protein comprises a protein comprising at least 70% identity, at least 80% identity, at least 90% identity, at least 95% identity, or at least 99% identity to SEQ ID NO: 17, the wild-type MeAKT2a protein comprises a protein comprising at least 70% identity, at least 80% identity, at least 90% identity, at least 95% identity, or at least 99% identity to SEQ ID NO: 19, and the wild-type MeAKT2b protein comprises a protein comprising at least 70% identity, at least 80% identity, at least 90% identity, at least 95% identity, or at least 99% identity to SEQ ID NO: 20; or a functional fragment of one of the foregoing; the modified plant AKT2 protein comprises a protein comprising at least 70% identity, at least 80% identity, at least 90% identity, at least 95% identity, or at least 99% identity to one of SEQ ID NOs: 18, 25, and 26, the modified AtAKT2 protein comprises a protein comprising at least 70% identity, at least 80% identity, at least 90% identity, at least 95% identity, or at least 99% identity to SEQ ID NO: 18, wherein the modified MeAKT2a protein comprises a protein comprising at least 70% identity, at least 80% identity, at least 90% identity, at least 95% identity, or at least 99% identity to SEQ ID NO: 25, and wherein the modified MeAKT2b protein comprises a protein comprising at least 70% identity, at least 80% identity, at least 90% identity, at least 95% identity, or at least 99% identity to SEQ ID NO: 26; or a functional fragment of one of the foregoing; and/or the one or more nucleotide sequences encoding the modified AtAKT2 protein includes a nucleotide sequence including at least 70% identity, at least 80% identity, at least 90% identity, at least 95% identity, or at least 99% identity to SEQ ID NO: 3.

Still another aspect of the disclosure includes methods of producing the genetically modified plant or part thereof of any one of the preceding embodiments that has an AKT2 protein operably linked to an overexpression promoter, including introducing one or more nucleotide sequences encoding the AtAKT2 protein, the MeAKT2a protein, and/or the MeAKT2b protein operably linked to the expression control sequence including the overexpression promoter. In a further embodiment of this aspect, the AtAKT2 protein includes a protein including at least 70% identity, at least 80% identity, at least 90% identity, at least 95% identity, or at least 99% identity to SEQ ID NO: 17 or SEQ ID NO: 18, the MeAKT2a protein comprises a protein comprising at least 70% identity, at least 80% identity, at least 90% identity, at least 95% identity, or at least 99% identity to SEQ ID NO: 19 or SEQ ID NO: 25, and the MeAKT2b protein comprises a protein comprising at least 70% identity, at least 80% identity, at least 90% identity, at least 95% identity, or at least 99% identity to SEQ ID NO: 20 or SEQ ID NO: 26; or a functional fragment of one of the foregoing. In an additional embodiment of this aspect, which may be combined with any of the preceding embodiments, the overexpression promoter includes the Arabidopsis thaliana POTASSIUM TRANSPORTER 2 (pAtAKT2) promoter, the Arabidopsis thaliana SUCROSE TRANSPORTER 2 promoter (pAtSUC2), the COMMELINA YELLOW MOTTLE VIRUS promoter (pCoYMV), the Rice tungro bacilliform virus promoter (pRTBV), the Solanum tuberosum KST1 promoter (pStKST1), the cassava MeAKT2a promoter, or the cassava MeAKT2b promoter. In yet another embodiment of this aspect, the promoter includes the pAtAKT2 promoter, the cassava MeAKT2a promoter, or the cassava MeAKT2b promoter.

In some embodiments, methods of producing the genetically modified plant or plant part thereof as disclosed herein that has a modified plant AKT2 protein include genetically modifying a plant by transforming the plant with one or more gene editing components that target an endogenous nuclear genome sequence encoding the wild-type plant ATK2 protein, wild-type AtAKT2 protein, the wild-type MeAKT2a protein, and/or the wild-type MeAKT2b protein to produce the modified AKT2 protein, modified AtAKT2 protein, the modified MeAKT2a protein, and/or the modified MeAKT2b protein. In a further embodiment of this aspect, the one or more gene editing components include a ribonucleoprotein complex that targets the nuclear genome sequence; a vector including a TALEN protein encoding sequence, wherein the TALEN protein targets the nuclear genome sequence; a vector including a ZFN protein encoding sequence, wherein the ZFN protein targets the nuclear genome sequence; an oligonucleotide donor (OND), wherein the OND targets the nuclear genome sequence; or a vector CRISPR/Cas enzyme encoding sequence and a targeting sequence, wherein the targeting sequence targets the nuclear genome sequence. In an additional embodiment of this aspect, the wild-type plant AKT2 protein includes a protein including at least 70% identity, at least 80% identity, at least 90% identity, at least 95% identity, or at least 99% identity to one of SEQ ID NOs: 17, 19, and 20, the wild-type AtAKT2 protein includes a protein including at least 70% identity, at least 80% identity, at least 90% identity, at least 95% identity, or at least 99% identity to SEQ ID NO: 17, the wild-type MeAKT2a protein includes a protein including at least 70% identity, at least 80% identity, at least 90% identity, at least 95% identity, or at least 99% identity to SEQ ID NO: 19, and the wild-type MeAKT2b protein includes a protein including at least 70% identity, at least 80% identity, at least 90% identity, at least 95% identity, or at least 99% identity to SEQ ID NO: 20; or a functional fragment of one of the foregoing. In still another embodiment of this aspect, which may be combined with any of the preceding embodiments, the modified plant AKT2 protein, the modified AtAKT2 protein, the modified MeAKT2a protein, and/or the modified MeAKT2b protein (a) comprises one or more amino acid substitutions, insertions, or deletions in the Ion_trans (PF00520, Ion transport protein) motif (corresponding to amino acids 75-323 of SEQ ID NO: 17) and/or one or more amino acid substitutions, insertions, or deletions in the Ion_trans_2 (PF07885, Ion channel) motif (corresponding to amino acids 225-317 of SEQ ID NO: 17), wherein the one or more substitutions, insertions, or deletions modulate or increase the ion transport activity; or (b) comprises one or more amino acid substitutions, insertions, or deletions, wherein the one or more substitutions, insertions, or deletions modulate or increase the ion transport activity. In yet another embodiment of this aspect, which may be combined with any of the preceding embodiments, the modified plant AKT2 protein, modified AtAKT2 protein, the modified MeAKT2a protein, and/or the modified MeAKT2b protein includes one or both of the amino acid substitutions corresponding to S210N and S329N when aligned to SEQ ID NO: 17. In still another embodiment of this aspect, which may be combined with any of the preceding embodiments, the modified plant AKT2 protein comprises a protein comprising at least 70% identity, at least 80% identity, at least 90% identity, at least 95% identity, or at least 99% identity to one of SEQ ID NOs: 18, 25, and 26, the modified AtAKT2 protein includes a protein including at least 70% identity, at least 80% identity, at least 90% identity, at least 95% identity, or at least 99% identity to SEQ ID NO: 18, wherein the modified MeAKT2a protein includes a protein comprising at least 70% identity, at least 80% identity, at least 90% identity, at least 95% identity, or at least 99% identity to SEQ ID NO: 25, and wherein the modified MeAKT2b protein includes a protein comprising at least 70% identity, at least 80% identity, at least 90% identity, at least 95% identity, or at least 99% identity to SEQ ID NO: 26; or a functional fragment of one of the foregoing.

Yet another aspect of the disclosure includes methods of producing the genetically modified plant or part thereof of any one of the preceding embodiments that has an AKT2 protein operably linked to an overexpression promoter, including genetically modifying a plant by transforming the plant with one or more gene editing components that target an endogenous nuclear genome sequence encoding a promoter of an endogenous AtAKT2 protein, a promoter of an endogenous MeAKT2a protein, and/or a promoter of an endogenous MeAKT2b protein to produce a modified AtAKT2 promoter, a modified MeAKT2a promoter, and/or a modified MeAKT2b promoter, wherein the modified AtAKT2 promoter, the modified MeAKT2a promoter, and/or the modified MeAKT2b promoter has increased expression and/or altered tissue-specific expression as compared to the unmodified promoter. In an additional embodiment of this aspect, the one or more gene editing components include a ribonucleoprotein complex that targets the nuclear genome sequence; a vector including a TALEN protein encoding sequence, wherein the TALEN protein targets the nuclear genome sequence; a vector including a ZFN protein encoding sequence, wherein the ZFN protein targets the nuclear genome sequence; an oligonucleotide donor (OND), wherein the OND targets the nuclear genome sequence; or a vector CRISPR/Cas enzyme encoding sequence and a targeting sequence, wherein the targeting sequence targets the nuclear genome sequence. Yet another aspect of the disclosure includes methods of producing the genetically modified plant or part thereof of any one of the preceding embodiments that has an AKT2 protein operably linked to an overexpression promoter, including genetically modifying a plant by transforming the plant with one or more gene editing components that target an endogenous nuclear genome sequence encoding a promoter of an endogenous plant AKT2 protein, a promoter of an endogenous AtAKT2 protein, a promoter of an endogenous MeAKT2a protein, and/or a promoter of an endogenous MeAKT2b protein to produce a modified AKT2 promoter, a modified AtAKT2 promoter, a modified MeAKT2a promoter, and/or a modified MeAKT2b promoter, wherein the modified plant AKT2 promoter, the modified AtAKT2 promoter, the modified MeAKT2a promoter, and/or the modified MeAKT2b promoter has increased expression and/or altered tissue-specific expression as compared to the unmodified promoter. In an additional embodiment of this aspect, the one or more gene editing components include a ribonucleoprotein complex that targets the nuclear genome sequence; a vector including a TALEN protein encoding sequence, wherein the TALEN protein targets the nuclear genome sequence; a vector including a ZFN protein encoding sequence, wherein the ZFN protein targets the nuclear genome sequence; an oligonucleotide donor (OND), wherein the OND targets the nuclear genome sequence; or a vector CRISPR/Cas enzyme encoding sequence and a targeting sequence, wherein the targeting sequence targets the nuclear genome sequence.

In a further embodiment of this aspect, which may be combined with any of the preceding embodiments that have methods, the method further includes selecting a genetically modified plant or part thereof with improved growth, improved photosynthesis, higher rate of CO₂ fixation and/or higher electron transport rate when the genetically modified plant or part thereof is grown under non-limiting energy conditions, earlier maximum growth rate, improved yield, increased plant growth, increased plant height, increased shoot growth, increased total shoot dry matter, improved storage root or tuber growth, increased number of storage roots or tubers per plant, and/or increased total storage root or tuber dry matter as compared to a control plant. In a further embodiment of this aspect, which may be combined with any of the preceding embodiments that have methods, the method further includes selecting a genetically modified plant or plant part thereof with improved growth, improved photosynthesis, higher rate of CO₂ fixation and/or higher electron transport rate when the genetically modified plant is grown under non-limiting energy conditions, earlier maximum growth rate, increased yield, increased plant growth, increased plant height, increased shoot growth, increased total shoot dry matter, increased storage root or tuber biomass, increased number of storage roots or tubers per plant, and/or increased total storage root or tuber dry matter as compared to a control plant.

Some aspects of the present disclosure relate to a genetically modified plant or plant part thereof produced by the method of any one of the preceding embodiments. In an additional embodiment of this aspect, the plant produces storage roots or tubers, and/or is a passive symplasmic phloem loader. In a further embodiment of this aspect, the plant is selected from the group of cassava, potato, sweet potato, yam, yacón, taro, yuca, konjac, ginger, radish, turnip, rutabaga, parsnip, jicama, Jerusalem artichoke, arrowroot, turmeric, horseradish, beet, water chestnut, lotus root, maca root, celeriac, malanga, ube, skirret, or wasabi. In yet another embodiment, the plant can be a crop that benefits from potassium fertilization. For example, some crops that can benefit from potassium fertilization can be cassava, yams, potatoes, tomatoes, citrus fruits (e.g., oranges, lemons, limes, etc.), bananas, grains (e.g., wheat, rice, barley, sorghum, etc.), cotton, sugar beets, soybeans, cowpea, alfalfa, grapes, peppers, apples, melons (e.g., watermelon, cantaloupe, etc.). In an additional embodiment of this aspect, which may be combined with any of the preceding embodiments, the genetically modified plant or part thereof has higher photosynthetic efficiency, improved photosynthesis, higher rate of CO₂ fixation and/or electron transport rate, earlier maximum growth rate, improved yield under field conditions, improved yield under drought conditions, and/or improved yield under potassium deficiency as compared to a control plant grown under the same conditions.

Further aspects of the present disclosure relate to a genetically altered plant genome including (i) the one or more edited endogenous nucleotide sequences in the genetically modified plant or part thereof of any one of the preceding embodiments, or (ii) the one or more edited endogenous nucleotide sequences in the genetically modified plant or part thereof produced by the method of any one of the preceding embodiments.

Additional aspects of the present disclosure relate to a non-regenerable part or cell of the genetically modified plant or part thereof of any one of the preceding embodiments.

In certain embodiments, the plant part may be a seed, pod, fruit, leaf, flower, stem, root, storage root, tuber, root tuber, stem tuber, storage organ, any part of the foregoing or a cell thereof, or a non-regenerable part or cell of a genetically modified plant or part thereof part. As used in this context, a “non-regenerable” part or cell of a genetically modified plant or part thereof or part thereof is a part or cell that itself cannot be induced to form a whole plant or cannot be induced to form a whole plant capable of sexual and/or asexual reproduction. In certain embodiments, the non-regenerable part or cell of the plant part is a part of a transgenic seed, pod, fruit, leaf, flower, stem, root, storage root, tuber, root tuber, stem tuber, storage organ or is a cell thereof.

Processed plant products that contain a detectable amount of a nucleotide segment, expressed RNA, and/or protein comprising a genetic modification disclosed herein are also provided. Such processed products include, but are not limited to, plant biomass, oil, meal, animal feed, flour, flakes, bran, lint, hulls, and processed seed. The processed product may be non-regenerable. The plant product can comprise commodity or other products of commerce derived from a transgenic plant or transgenic plant part, where the commodity or other products can be tracked through commerce by detecting a nucleotide segment, expressed RNA, and/or protein that comprises distinguishing portions of a genetic modification disclosed herein.

A control as described herein can be a control sample or a reference sample from a wild-type, an azygous, or a null-segregant plant, species, or sample or from populations thereof. A reference value can be used in place of a control or reference sample, which was previously obtained from a wild-type, azygous, or null-segregant plant, species, or sample or from populations thereof or a group of a wild-type, azygous, or null-segregant plant, species, or sample. A control sample or a reference sample can also be a sample with a known amount of a detectable composition or a spiked sample.

### Isolated Constructs or Expression Cassettes, Cells or Kits Including the Same, and Related Methods

An additional aspect of the disclosure includes an expression vector or isolated DNA molecule including one or more nucleotide sequences encoding a modified POTASSIUM TRANSPORTER 2 (AKT2) protein operably linked to an expression control sequence. Further embodiments of this aspect include the modified AKT2 protein being selected from the group of a modified Arabidopsis thaliana POTASSIUM TRANSPORTER 2 (AtAKT2) protein, a modified first Manihot esculenta AKT2 protein (MeAKT2a), and/or a modified second Manihot esculenta AKT2 protein (MeAKT2b). In yet another embodiment of this aspect, the modified AtAKT2 protein includes a protein including at least 70% identity, at least 80% identity, at least 90% identity, at least 95% identity, or at least 99% identity to SEQ ID NO: 18, wherein the modified MeAKT2a protein includes a protein comprising at least 70% identity, at least 80% identity, at least 90% identity, at least 95% identity, or at least 99% identity to SEQ ID NO: 25, and wherein the modified MeAKT2b protein includes a protein comprising at least 70% identity, at least 80% identity, at least 90% identity, at least 95% identity, or at least 99% identity to SEQ ID NO: 26. In still another embodiment of this aspect, which may be combined with any one of the preceding embodiments, the one or more nucleotide sequences encoding the modified AtAKT2 protein includes a nucleotide sequence including at least 70% identity, at least 80% identity, at least 90% identity, at least 95% identity, or at least 99% identity to SEQ ID NO: 3.

A further aspect of the disclosure includes an expression vector or isolated DNA molecule including one or more nucleotide sequences encoding a wild-type POTASSIUM TRANSPORTER 2 (AKT2) protein operably linked to an expression control sequence. Additional embodiments of this aspect include the wild-type AKT2 protein being selected from the group of an Arabidopsis thaliana POTASSIUM TRANSPORTER 2 (AtAKT2) protein, a first Manihot esculenta AKT2 protein (MeAKT2a), and/or a second Manihot esculenta AKT2 protein (MeAKT2b). In still another embodiment of this aspect, the AtAKT2 protein includes a protein including at least 70% identity, at least 80% identity, at least 90% identity, at least 95% identity, or at least 99% identity to SEQ ID NO: 17, the MeAKT2a protein includes a protein including at least 70% identity, at least 80% identity, at least 90% identity, at least 95% identity, or at least 99% identity to SEQ ID NO: 19, and the MeAKT2b protein includes a protein including at least 70% identity, at least 80% identity, at least 90% identity, at least 95% identity, or at least 99% identity to SEQ ID NO: 20; or a functional fragment of one of the foregoing. An additional aspect of the disclosure includes an expression vector or isolated DNA molecule including one or more nucleotide sequences encoding a modified POTASSIUM TRANSPORTER 2 (AKT2) protein operably linked to an expression control sequence. Further embodiments of this aspect include the modified plant AKT2 protein being selected from the group of a modified Arabidopsis thaliana POTASSIUM TRANSPORTER 2 (AtAKT2) protein, a modified first Manihot esculenta AKT2 protein (MeAKT2a), and/or a modified second Manihot esculenta AKT2 protein (MeAKT2b). In yet another embodiment of this aspect, the modified AKT2 protein comprises a protein comprising at least 70% identity, at least 80% identity, at least 90% identity, at least 95% identity, or at least 99% identity to one of SEQ ID NOs: 18, 25, and 26, the modified AtAKT2 protein includes a protein including at least 70% identity, at least 80% identity, at least 90% identity, at least 95% identity, or at least 99% identity to SEQ ID NO: 18, wherein the modified MeAKT2a protein includes a protein comprising at least 70% identity, at least 80% identity, at least 90% identity, at least 95% identity, or at least 99% identity to SEQ ID NO: 25, and wherein the modified MeAKT2b protein includes a protein comprising at least 70% identity, at least 80% identity, at least 90% identity, at least 95% identity, or at least 99% identity to SEQ ID NO: 26; or a functional fragment of one of the foregoing. In still another embodiment of this aspect, which may be combined with any one of the preceding embodiments, the one or more nucleotide sequences encoding the modified AtAKT2 protein includes a nucleotide sequence including at least 70% identity, at least 80% identity, at least 90% identity, at least 95% identity, or at least 99% identity to SEQ ID NO: 3.

A further aspect of the disclosure includes an expression vector or isolated DNA molecule including one or more nucleotide sequences encoding a wild-type POTASSIUM TRANSPORTER 2 (AKT2) protein operably linked to an expression control sequence. Additional embodiments of this aspect include the wild-type AKT2 protein being selected from the group of an Arabidopsis thaliana POTASSIUM TRANSPORTER 2 (AtAKT2) protein, a first Manihot esculenta AKT2 protein (MeAKT2a), and/or a second Manihot esculenta AKT2 protein (MeAKT2b). In still another embodiment of this aspect, the plant AKT2 protein comprises a protein comprising at least 70% identity, at least 80% identity, at least 90% identity, at least 95% identity, or at least 99% identity to one of SEQ ID NOs: 17, 19, and 20, the AtAKT2 protein includes a protein including at least 70% identity, at least 80% identity, at least 90% identity, at least 95% identity, or at least 99% identity to SEQ ID NO: 17, the MeAKT2a protein includes a protein including at least 70% identity, at least 80% identity, at least 90% identity, at least 95% identity, or at least 99% identity to SEQ ID NO: 19, and the MeAKT2b protein includes a protein including at least 70% identity, at least 80% identity, at least 90% identity, at least 95% identity, or at least 99% identity to SEQ ID NO: 20; or a functional fragment of one of the foregoing.

In yet another embodiment of this aspect, which may be combined with any of the preceding embodiments that has an expression vector or isolated DNA molecule, the expression control sequence includes an overexpression promoter, a phloem-specific promoter, a xylem-specific promoter, a root-specific promoter, and/or a stomata-specific promoter. In still another embodiment of this aspect, the promoter includes an Arabidopsis thaliana POTASSIUM TRANSPORTER 2 (pAtAKT2) promoter, an Arabidopsis thaliana SUCROSE TRANSPORTER 2 promoter (pAtSUC2), a COMMELINA YELLOW MOTTLE VIRUS promoter (pCoYMV), a Rice tungro bacilliform virus promoter (pRTBV), a Solanum tuberosum KST1 promoter (pStKST1), a cassava MeAKT2a promoter, or a cassava MeAKT2b promoter. In a further embodiment of this aspect, the promoter includes the pAtAKT2 promoter, the cassava MeAKT2a promoter, or the cassava MeAKT2b promoter. In an additional embodiment of this aspect, the promoter includes the pAtAKT2 promoter, and wherein the promoter includes a nucleotide sequence including at least 70% identity, at least 80% identity, at least 90% identity, at least 95% identity, or at least 99% identity to SEQ ID NO: 2. In yet another embodiment of this aspect, which may be combined with any of the preceding embodiments that has an expression vector or isolated DNA molecule, the expression control sequence includes an overexpression promoter, a phloem-specific promoter, a xylem-specific promoter, a root-specific promoter, and/or a stomata-specific promoter. In still another embodiment of this aspect, the promoter includes an Arabidopsis thaliana POTASSIUM TRANSPORTER 2 (pAtAKT2) promoter, an Arabidopsis thaliana SUCROSE TRANSPORTER 2 promoter (pAtSUC2), a COMMELINA YELLOW MOTTLE VIRUS promoter (pCoYMV), a Rice tungro bacilliform virus promoter (pRTBV), a Solanum tuberosum KST1 promoter (pStKST1), a cassava MeAKT2a promoter, a cassava MeAKT2b promoter, or a proIC promoter. In a further embodiment of this aspect, the promoter includes the pAtAKT2 promoter, the cassava MeAKT2a promoter, or the cassava MeAKT2b promoter. In an additional embodiment of this aspect, the promoter includes the pAtAKT2 promoter, and wherein the promoter includes a nucleotide sequence including at least 70% identity, at least 80% identity, at least 90% identity, at least 95% identity, or at least 99% identity to SEQ ID NO: 2.

Some aspects of the present disclosure relate to a bacterial cell or an Agrobacterium cell including the expression vector or isolated DNA molecule of any of the preceding embodiments.

Further aspects of the present disclosure relate to a composition or kit including the expression vector or isolated DNA molecule of any of the preceding embodiments, or the bacterial cell or the Agrobacterium cell of the preceding embodiment.

Additional aspects of the present disclosure relate to a genetically modified plant, plant part, aboveground plant part, plant cell, or seed including the expression vector or isolated DNA molecule of any of the preceding embodiments.

Further aspects of the present disclosure relate to a composition or kit including the genetically modified plant or part thereof of any of the preceding embodiments, the genetically modified plant, plant part, aboveground plant part, plant cell, or seed of the preceding embodiment, or the genetically modified plant or part thereof produced by the method of any of the preceding embodiments. Further aspects of the present disclosure relate to a composition or kit including the genetically modified plant or plant part thereof of any of the preceding embodiments, the genetically modified plant, plant part, plant cell, or seed of the preceding embodiment, or the genetically modified plant or plant part thereof produced by the method of any of the preceding embodiments.

Still further aspects of the present disclosure relate to methods of increasing photosynthetic efficiency, improving photosynthesis, producing earlier maximum growth rate, improving yield under field conditions, improving yield under drought conditions, improving yield under potassium deficiency, increasing plant growth, increasing plant height, increasing shoot growth, increasing total shoot dry matter, improving storage root or tuber growth, increasing number of storage roots or tubers per plant, and/or increasing total storage root or tuber dry matter including: introducing a genetic alteration via the expression vector or isolated DNA molecule of any of the preceding embodiments to a cell, wherein the cell is a plant cell.

Further aspects of the present disclosure relate to a method of improving phloem transport, improving phloem mass flow, improving source-sink delivery, increasing fibrous root formation, increasing photosynthetic efficiency, improving photosynthesis, producing earlier maximum growth rate, increasing yield under field conditions, increasing yield under drought conditions, increasing drought stress resistance, increasing drought tolerance, increasing yield under potassium deficiency, increasing plant growth, increasing plant height, increasing shoot growth, increasing total shoot dry matter, increasing storage root or tuber biomass, increasing number of storage roots or tubers per plant, and/or increasing total storage root or tuber dry matter including: introducing a genetic alteration via the expression vector or isolated DNA molecule of any preceding embodiment including expression vectors or isolated DNA molecules to a cell, wherein the cell is a plant cell.

Further aspects of the present disclosure relate to a genetically altered plant genome including (i) the one or more edited endogenous nucleotide sequences in the genetically modified plant or plant part thereof of any one of the preceding embodiments, or (ii) the one or more edited endogenous nucleotide sequences in the genetically modified plant or plant part thereof produced by the method of any one of the preceding embodiments.

Additional aspects of the present disclosure relate to a non-regenerable part or cell of the genetically modified plant or plant part thereof of any one of the preceding embodiments.

Still another aspect of the present disclosure relates to cassava plant or plant part thereof including (a) one or more nucleotide sequences encoding a modified AtAKT2 protein, a modified MeAKT2a protein, and/or a modified MeAKT2b protein, and (b) improved phloem transport, improved phloem mass flow, improved source-sink delivery, increased fibrous root formation, increased growth, improved photosynthesis, higher rate of CO2 fixation and/or higher electron transport rate when the genetically modified plant is grown under non-limiting energy conditions, earlier maximum growth rate, increased yield, increased plant growth, increased plant height, increased shoot growth, increased total shoot dry matter, increased storage root or tuber biomass, increased number of storage roots or tubers per plant, and/or increased total storage root or tuber dry matter as compared to a control plant.

“Improved photosynthesis” may, for example, denote increased carbon dioxide assimilation rates, increased electron transport rates, improved phloem transport, increased plant yield, increased plant height, increased plant biomass, increased total shoot dry mass, increased total root dry mass, and other metrics known in the art. “Improved photosynthesis” may also denote any known aspect of increased plant growth or productivity.

“Improved phloem transport” may, for example, denote increased phloem transport velocity, improved phloem mass flow (which may include increased phloem mass flow), and improved source-sink delivery (which may include faster source-sink delivery). Phloem transport velocity may be measured via tracers, such as in ¹¹C-transport velocity, or other methods known in the art.

### Phloem Transport

Vascular plants rely on phloem tissue for multidirectional transport of compounds that are critical to plant growth and development. These compounds are primarily photoassimilates: soluble organic compounds or, generally, carbohydrate nutrients such as glucose. Transport through the phloem, also called translocation, carries the nutrients produced by photosynthesis to many plant tissues as part of carbon allocation. Phloem “loading” and “unloading” refer to the movement of the photoassimilates from leaf mesophyll to phloem (“loading”) and the movement of photoassimilates from phloem to sink tissues (“unloading”). Translocation through the phloem generally relies on an osmotic pressure gradient moving from “source” to “sink” tissues, and phloem loading and unloading may be characterized as either symplasmic (transport through plasmodesmata, remaining in the cytoplasm) or apoplastic (in which the apoplasts, or cell walls outside the protoplasts, are entered). Apoplastic loading is characterized as active transport, as energy is required to drive it (Rüscher et al., (2024) Symplasmic phloem loading and subcellular transport in storage roots are key factors for carbon allocation in cassava. bioRxiv).

Plant species vary in the utilization of symplasmic vs. active phloem loading, and the type of sugar being transported may also determine the type of loading used (De Schepper, Veerle & Swaef, Tom & Bauweraerts, Ingvar & Steppe, Kathy. (2013). Phloem transport: A review of mechanisms and controls. Journal of experimental botany. 64). Additionally, within a single plant, the maturity of tissue may additionally determine whether symplasmic or active phloem unloading is exhibited: phloem unloading is typically symplasmic in growing and respiring sinks such as meristems, roots, and young leaves, where sucrose can be rapidly metabolized. Young leaves typically act as sink tissues until their photosynthetic machinery is fully developed, at which point they become source tissues. This explanation is not exhaustive, as there can be many determinants for the type of loading/unloading exhibited in a vascular plant.

Speaking to this complexity, phloem transport in cassava is nuanced and dynamic. While cassava is a mostly symplasmic phloem loader in its leaves, and a mostly symplasmic phloem unloader in its lower stem and storage roots (Rüscher et al., (2024) Symplasmic phloem loading and subcellular transport in storage roots are key factors for carbon allocation in cassava. bioRxiv), it still exhibits active transport, which may be especially important for the long-distance transport required in the cassava stem. Although cassava is characterized by symplasmic phloem loading in leaves and symplasmic phloem unloading in storage roots (Mehdi et al. (2019) Symplasmic phloem unloading and radial post-phloem transport via vascular rays in tuberous roots of Manihot esculenta, Journal of Experimental Botany, Volume 70, Issue 20, 15 Oct. 2019, Pages 5559-5573), the transport phloem still requires active sugar transport to retrieve leaked sucrose during long-distance transport (also referred to as a “leakage-retrieval mechanism”; as in De Schepper et al. (2013) (De Schepper et al. (2013). Phloem transport: A review of mechanisms and controls. Journal of experimental botany. 64)).

In part because of the nuance in this crucial system, active phloem reloading is believed to be important in any plant species, including active and/or passive phloem loaders. Reloading into the high-sugar vascular system would always require active transport due to the reloading acting against the concentration gradient that largely governs the direction of phloem transport. Accordingly, the present disclosure may be utilized for any vascular plant species, especially species requiring significant transport distances, such as those with storage tissues (e.g., harvestable organs of interest to humans) that are significantly separated from the leaves (as seen in storage roots). In some embodiments, the plant is cassava. In some embodiments, the plant is not cassava. In some embodiments, the plant is potato, sweet potato, yam, ube, yacón, taro, konjac, ginger, radish, turnip, rutabaga, parsnip, jicama, Jerusalem artichoke, turmeric, horseradish, beet, lotus, maca, celeriac, skirret, wasabi, grains, sorghum, or sugar beets.

### POTASSIUM TRANSPORTER 2 (AKT2) Proteins

POTASSIUM TRANSPORTER 2 (AKT2) is a voltage-gated potassium (K⁺) channel. Wild type AKT2 has two modes, namely mode 1, where AKT2 acts as an inward-rectifying K⁺ channel (Kᵢₙ), and mode 2, where AKT2 acts as a nonrectifying channel (both Kᵢₙ and Kₒᵤₜ; i.e., mediating both K⁺ uptake and release) (Dreyer et al., 2017, The potassium battery: a mobile energy source for transport processes in plant vascular tissues. New Phytologist 216: 1049-1053). Modification of AKT2 can result in AKT2 being biased toward or locked in mode 2, such that it acts as a nonrectifying channel with both Kᵢₙ and Kₒᵤₜ functions (Gajdanowicz et al., 2011. Potassium (K+) gradients serve as a mobile energy source in plant vascular tissues. Proc Natl Acad Sci USA, 108, 864-9.). Posttranslational modifications of the AKT2 channel can allow the plant to tap into the circulating K⁺ energy storage by efficiently assisting the plasma membrane H⁺-ATPase in energizing the transmembrane phloem loading process (Gajdanowicz et al., 2011. Potassium (K+) gradients serve as a mobile energy source in plant vascular tissues. Proc Natl Acad Sci USA, 108, 864-9.).

Homologs of AKT2 can be identified through methods known in the art. Functional differences in AKT1 and AKT2 proteins are thought to be that AKT1 is important for potassium uptake from soil, especially under low K⁺ conditions. Based on this, it is believed that knockout of AKT1 leads to K⁺ deficiency symptoms, even if soil K⁺ is sufficient. AKT2 is thought to function in long-distance K⁺ transport (particularly in the phloem), can switch between inward and outward conductance (as described above), and is important for K⁺ recycling from shoots to roots and phloem loading. AKT2 activity can switch between two activity modes: (i) electrogenic mode: allows K⁺ to move with a net charge transfer across the membrane (described herein as mode 1, a rectifying channel); or (ii) electrically silent mode: functions as a K⁺ leak channel, balancing osmotic gradients without significantly altering membrane voltage (described herein as mode 2, a non-rectifying channel). The physiological role or activity of AKT2 can include: (i) phloem loading and unloading: AKT2 plays a critical role in K⁺ transport within phloem tissues, especially in companion cells and helps maintain osmotic balance required for sugar transport (via pressure-driven flow in the sieve tubes); (ii) K⁺ recycling and redistribution: facilitates long-distance K⁺ transport between leaves and roots and is important for recycling K⁺ from shoot tissues back to the root via the phloem; and (iii) adaptation to environmental conditions: because of its ability to switch modes, AKT2 helps plants adapt to fluctuating energy and ion availability, especially under sugar or osmotic stress. Modifying AKT2 can modulate the activity and/or modes of the AKT2 channel. In other embodiments, the present disclosure provides for improved phloem transport. It is presently believed that improved phloem transport improves source-sink delivery, photosynthesis parameters, and growth parameters.

AKT2 proteins of the present disclosure include a plant AKT2 protein, an Arabidopsis thaliana AKT2 (AtAKT2) protein, a first Manihot esculenta AKT2 protein (MeAKT2a), and/or a second Manihot esculenta AKT2 protein (MeAKT2b) or AKT2 homologs thereof, such as AKT2 orthologs or AKT2 paralogs. MeAKT2a and MeAKT2b may be expressed wholly or in part within different plant tissues, as in cassava, for which the endogenous MeAKT2a and MeAKT2b are phloem- and xylem-sided, respectively, indicating a xylem function for AKT2 as well. The wild-type AtAKT2 protein includes a protein including at least 70% identity, at least 80% identity, at least 90% identity, at least 95% identity, or at least 99% identity to SEQ ID NO: 17, the wild-type MeAKT2a protein includes a protein including at least 70% identity, at least 80% identity, at least 90% identity, at least 95% identity, or at least 99% identity to SEQ ID NO: 19, and the wild-type MeAKT2b protein includes a protein including at least 70% identity, at least 80% identity, at least 90% identity, at least 95% identity, or at least 99% identity to SEQ ID NO: 20; or a functional fragment of one of the foregoing. The coding sequence of the wild-type AtAKT2 protein (i.e., the wild-type gene) is written as AtAKT2.

Modified AKT2 proteins of the present disclosure include a modified Arabidopsis thaliana POTASSIUM TRANSPORTER 2 (AtAKT2) protein, a modified first Manihot esculenta AKT2 protein (MeAKT2a), and/or a modified second Manihot esculenta AKT2 protein (MeAKT2b). The modified AtAKT2 protein includes a protein including at least 70% identity, at least 80% identity, at least 90% identity, at least 95% identity, or at least 99% identity to SEQ ID NO: 18, in which the amino acid substitutions S210N and S329N are present as compared to the wild-type AtAKT2 protein. The modified MeAKT2a protein includes a protein comprising at least 70% identity, at least 80% identity, at least 90% identity, at least 95% identity, or at least 99% identity to SEQ ID NO: 25, in which the amino acid substitutions S199N and S319N are present as compared to the wild-type MeAKT2a protein. The modified MeAKT2b protein includes a protein comprising at least 70% identity, at least 80% identity, at least 90% identity, at least 95% identity, or at least 99% identity to SEQ ID NO: 26, in which the amino acid substitutions S216N and S336N are present as compared to the wild-type MeAKT2b protein. The coding sequence of the modified AtAKT2 protein (i.e., the modified gene) is written as AtAKT2var, and includes a nucleotide sequence including at least 70% identity, at least 80% identity, at least 90% identity, at least 95% identity, or at least 99% identity to SEQ ID NO: 3. “Atakt2”, “AKT2var”, and “AtAKT2var” can all be used to mean an AtAKT2 protein with substitutions S210N and S329N. S210 is a mutation in a cytosolic part of the AKT2 protein that belongs to the PF00520 Ion transport protein domain (Pfam). S329 is a mutation in a cytosolic part of the AKT2 protein that is not assigned to a specific Pfam domain.

Without wishing to be bound by theory, the present disclosure may indicate that, for the non-rectifying mode of AKT2 enacted herein, mediating potassium release from the phloem and providing local energy for sucrose (re)-loading causes improved phloem transport, improved photosynthesis, and other desirable effects. The inward-rectifying mode of AKT2 may exhibit similar effects with relation to potassium phloem loading rather than potassium phloem unloading. The WT version of the AKT2 protein may also have a positive effect, for example, when the WT version of the AKT2 protein is overexpressed. Either mode of AKT2 may also have beneficial effects on sugar transport.

### Plant Breeding Methods

Plant breeding begins with the analysis of the current germplasm, the definition of problems and weaknesses of the current germplasm, the establishment of program goals, and the definition of specific breeding objectives. The next step is the selection of germplasm that possess the traits to meet the program goals. The selected germplasm is crossed in order to recombine the desired traits and through selection, varieties or parent lines are developed. The goal is to combine in a single variety or hybrid an improved combination of desirable traits from the parental germplasm. These important traits may include higher yield, field performance, improved fruit and agronomic quality, resistance to biological stresses, such as diseases and pests, and tolerance to environmental stresses, such as drought and heat.

Each breeding program should include a periodic, objective evaluation of the efficiency of the breeding procedure. Evaluation criteria vary depending on the goal and objectives, but should include gain from selection per year based on comparisons to an appropriate standard, overall value of the advanced breeding lines, and number of successful cultivars produced per unit of input (e.g., per year, per dollar expended, etc.). Promising advanced breeding lines are thoroughly tested and compared to appropriate standards in environments representative of the commercial target area(s) for three years at least. The best lines are candidates for new commercial cultivars; those still deficient in a few traits are used as parents to produce new populations for further selection. These processes, which lead to the final step of marketing and distribution, usually take five to ten years from the time the first cross or selection is made.

The choice of breeding or selection methods depends on the mode of plant reproduction, the heritability of the trait(s) being improved, and the type of cultivar used commercially (e.g., F₁ hybrid cultivar, inbred cultivar, etc.). For highly heritable traits, a choice of superior individual plants evaluated at a single location will be effective, whereas for traits with low heritability, selection should be based on mean values obtained from replicated evaluations of families of related plants. The complexity of inheritance also influences the choice of the breeding method. Backcross breeding is used to transfer one or a few genes for a highly heritable trait into a desirable cultivar (e.g., for breeding disease-resistant cultivars), while recurrent selection techniques are used for quantitatively inherited traits controlled by numerous genes, various recurrent selection techniques are used. Commonly used selection methods include pedigree selection, modified pedigree selection, mass selection, and recurrent selection.

Pedigree selection is generally used for the improvement of self-pollinating crops or inbred lines of cross-pollinating crops. Two parents which possess favorable, complementary traits are crossed to produce an F₁. An F₂ population is produced by selfing one or several F₁s or by intercrossing two F₁s (sib mating). Selection of the best individuals is usually begun in the F₂ population; then, beginning in the F₃, the best individuals in the best families are selected. Replicated testing of families, or hybrid combinations involving individuals of these families, often follows in the F₄ generation to improve the effectiveness of selection for traits with low heritability. At an advanced stage of inbreeding (i.e., F₆ and F₇), the best lines or mixtures of phenotypically similar lines are tested for potential release as new cultivars.

Mass and recurrent selections can be used to improve populations of either self- or cross-pollinating crops. A genetically variable population of heterozygous individuals is either identified or created by intercrossing several different parents. The best plants are selected based on individual superiority, outstanding progeny, or excellent combining ability. The selected plants are intercrossed to produce a new population in which further cycles of selection are continued.

Backcross breeding (i.e., recurrent selection) may be used to transfer genes for a simply inherited, highly heritable trait into a desirable homozygous cultivar or line that is the recurrent parent. The source of the trait to be transferred is called the donor parent. The resulting plant is expected to have the attributes of the recurrent parent (e.g., cultivar) and the desirable trait transferred from the donor parent. After the initial cross, individuals possessing the phenotype of the donor parent are selected and repeatedly crossed (backcrossed) to the recurrent parent. The resulting plant is expected to have the attributes of the recurrent parent (e.g., cultivar) and the desirable trait transferred from the donor parent.

The single-seed descent procedure in the strict sense refers to planting a segregating population, harvesting a sample of one seed per plant, and using the one-seed sample to plant the next generation. When the population has been advanced from the F₂ to the desired level of inbreeding, the plants from which lines are derived will each trace to different F₂ individuals. The number of plants in a population declines each generation due to failure of some seeds to germinate or some plants to produce at least one seed. As a result, not all of the F₂ plants originally sampled in the population will be represented by a progeny when generation advance is completed.

In addition to phenotypic observations, the genotype of a plant can also be examined. There are many laboratory-based techniques available for the analysis, comparison and characterization of plant genotype; among these are Isozyme Electrophoresis, Restriction Fragment Length Polymorphisms (RFLPs), Randomly Amplified Polymorphic DNAs (RAPDs), Arbitrarily Primed Polymerase Chain Reaction (AP-PCR), DNA Amplification Fingerprinting (DAF), Sequence Characterized Amplified Regions (SCARs), Amplified Fragment Length Polymorphisms (AFLPs), Simple Sequence Repeats (SSRs, which are also referred to as Microsatellites), Fluorescently Tagged Inter-simple Sequence Repeats (ISSRs), Single Nucleotide Polymorphisms (SNPs), Genotyping by Sequencing (GbS), and Next-generation Sequencing (NGS).

Molecular markers, or “markers”, can also be used during the breeding process for the selection of qualitative traits. For example, markers closely linked to alleles or markers containing sequences within the actual alleles of interest can be used to select plants that contain the alleles of interest. The use of markers in the selection process is often called genetic marker enhanced selection or marker-assisted selection. Methods of performing marker analysis are generally known to those of skill in the art.

Mutation breeding may also be used to introduce new traits into plant varieties. Mutations that occur spontaneously or are artificially induced can be useful sources of variability for a plant breeder. The goal of artificial mutagenesis is to increase the rate of mutation for a desired characteristic. Mutation rates can be increased by many different means including temperature, long-term seed storage, tissue culture conditions, radiation (such as X-rays, Gamma rays, neutrons, Beta radiation, or ultraviolet radiation), chemical mutagens (such as base analogs like 5-bromo-uracil), antibiotics, alkylating agents (such as sulfur mustards, nitrogen mustards, epoxides, ethyleneamines, sulfates, sulfonates, sulfones, or lactones), azide, hydroxylamine, nitrous acid or acridines. Once a desired trait is observed through mutagenesis the trait may then be incorporated into existing germplasm by traditional breeding techniques. Details of mutation breeding can be found in Principles of Cultivar Development: Theory and Technique, Walter Fehr (1991), Agronomy Books, 1 (lib [DOT] dr [DOT] iastate [DOT] edu [FORWARD SLASH] agron_books [FORWARD SLASH] 1).

The production of double haploids can also be used for the development of homozygous lines in a breeding program. Double haploids are produced by the doubling of a set of chromosomes from a heterozygous plant to produce a completely homozygous individual. For example, see Wan, et al., Theor. Appl. Genet., 77:889-892, 1989.

Additional non-limiting examples of breeding methods that may be used include, without limitation, those found in Principles of Plant Breeding, John Wiley and Son, pp. 115-161 (1960); Principles of Cultivar Development: Theory and Technique, Walter Fehr (1991), Agronomy Books, 1 (lib [DOT] dr [DOT] iastate [DOT] edu [FORWARD SLASH] agron_books [FORWARD SLASH]1), which are herewith incorporated by reference.

### Molecular Biological Methods to Produce Genetically Modified Plant Cells, Plant Parts, and Plants

Transformation and generation of genetically altered monocotyledonous and dicotyledonous plant cells is well known in the art. See, e.g., Weising, et al., Ann. Rev. Genet. 22:421-477 (1988); U.S. Pat. No. 5,679,558; Agrobacterium Protocols, ed: Gartland, Humana Press Inc. (1995); Wang, et al. Acta Hort. 461:401-408 (1998), and Broothaerts, et al. Nature 433:629-633 (2005). The choice of method varies with the type of plant to be transformed, the particular application, and/or the desired result. The appropriate transformation technique is readily chosen by the skilled practitioner.

Any methodology known in the art to delete, insert or otherwise modify the cellular DNA (e.g., genomic DNA and organelle DNA) can be used in practicing the compositions, methods, and processes disclosed herein. As an example, the CRISPR/Cas-9 system and related systems (e.g., TALEN, ZFN, ODN, etc.) may be used to insert a heterologous gene to a targeted site in the genomic DNA or substantially edit an endogenous gene to express the heterologous gene or to modify the promoter to increase or otherwise alter expression of an endogenous gene through, for example, removal of repressor binding sites or introduction of enhancer binding sites. For example, a disarmed Ti plasmid, containing a genetic construct for deletion or insertion of a target gene, in Agrobacterium tumefaciens can be used to transform a plant cell, and thereafter, a transformed plant can be regenerated from the transformed plant cell using procedures described in the art, for example, in EP 0116718, EP 0270822, PCT publication WO 84/02913 and published European Patent application (“EP”) 0242246. Ti-plasmid vectors each contain the gene between the border sequences, or at least located to the left of the right border sequence, of the T-DNA of the Ti-plasmid. Of course, other types of vectors can be used to transform the plant cell, using procedures such as direct gene transfer (as described, for example in EP 0233247), pollen mediated transformation (as described, for example in EP 0270356, PCT publication WO 85/01856, and U.S. Pat. No. 4,684,611), plant RNA virus-mediated transformation (as described, for example in EP 0 067 553 and U.S. Pat. No. 4,407,956), liposome-mediated transformation (as described, for example in U.S. Pat. No. 4,536,475), and other methods such as the methods for transforming certain lines of corn (e.g., U.S. Pat. No. 6,140,553; Fromm et al., Bio/Technology (1990) 8, 833-839); Gordon-Kamm et al., The Plant Cell, (1990) 2, 603-618), rice (Shimamoto et al., Nature, (1989) 338, 274-276; Datta et al., Bio/Technology, (1990) 8, 736-740), and the method for transforming monocots generally (PCT publication WO 92/09696). For cotton transformation, the method described in PCT patent publication WO 00/71733 can be used. For soybean transformation, reference is made to methods known in the art, e.g., Hinchee et al. (Bio/Technology, (1988) 6, 915) and Christou et al. (Trends Biotech, (1990) 8, 145) or the method of WO 00/42207.

Genetically altered plants of the present disclosure can be used in a conventional plant breeding scheme to produce more genetically altered plants with the same characteristics, or to introduce the genetic alteration(s) in other varieties of the same or related plant species. Seeds, which are obtained from the altered plants, preferably contain the genetic alteration(s) as a stable insert in chromosomal DNA or as modifications to an endogenous gene or promoter. Plants including the genetic alteration(s) in accordance with this disclosure include plants including, or derived from, root stocks of plants including the genetic alteration(s) of this disclosure, e.g., fruit trees or ornamental plants. Hence, any non-transgenic grafted plant parts inserted on a transformed plant or plant part are included in this disclosure.

Genetic alterations of the disclosure, including in an expression vector or expression cassette, which result in the expression of an introduced gene or altered expression of an endogenous gene will typically utilize a plant-expressible promoter. A ‘plant-expressible promoter’ as used herein refers to a promoter that ensures expression of the genetic alteration(s) of this disclosure in a plant cell. Examples of constitutive promoters that are often used in plant cells are the cauliflower mosaic (CaMV) 35S promoter (KAY et al. Science, 236, 4805, 1987), the minimal CaMV 35S promoter (Benfey & Chua, Science, (1990) 250, 959-966), various other derivatives of the CaMV 35S promoter, the figwort mosaic virus (FMV) promoter (Richins, et al., Nucleic Acids Res. (1987) 15:8451-8466), the maize ubiquitin promoter (CHRISTENSEN & QUAIL, Transgenic Res, 5, 213-8, 1996), the polyubiquitin promoter (Ljubql, MAEKAWA et al. Mol Plant Microbe Interact. 21, 375-82, 2008), the vein mosaic cassava virus promoter (International Application WO 97/48819), and the Arabidopsis UBQ10 promoter, Norris et al. Plant Mol. Biol. 21, 895-906, 1993).

Additional examples of promoters directing constitutive expression in plants are known in the art and include: the strong constitutive 35S promoters (the “35S promoters”) of the cauliflower mosaic virus (CaMV), e.g., of isolates CM 1841 (Gardner et al., Nucleic Acids Res, (1981) 9, 2871-2887), CabbB S (Franck et al., Cell (1980) 21, 285-294) and CabbB JI (Hull and Howell, Virology, (1987) 86, 482-493); promoters from the ubiquitin family (e.g., the maize ubiquitin promoter of Christensen et al., Plant Mol Biol, (1992) 18, 675-689), the gos2 promoter (de Pater et al., The Plant J (1992) 2, 834-844), the emu promoter (Last et al., Theor Appl Genet, (1990) 81, 581-588), actin promoters such as the promoter described by An et al. (The Plant J, (1996) 10, 107), the rice actin promoter described by Zhang et al. (The Plant Cell, (1991) 3, 1155-1165); promoters of the figwort mosaic virus (FMV) (Richins, et al., Nucleic Acids Res. (1987) 15:8451-8466), promoters of the Cassava vein mosaic virus (WO 97/48819; Verdaguer et al., Plant Mol Biol, (1998) 37, 1055-1067), the pPLEX series of promoters from Subterranean Clover Stunt Virus (WO 96/06932, particularly the S4 or S7 promoter), an alcohol dehydrogenase promoter, e.g., pAdh1S (GenBank accession numbers X04049, X00581), and the TR1′ promoter and the TR2′ promoter (the “TR1′ promoter” and “TR2′ promoter”, respectively) which drive the expression of the 1′ and 2′ genes, respectively, of the T DNA (Velten et al., EMBO J, (1984) 3, 2723-2730).

Alternatively, a plant-expressible promoter can be a tissue-specific promoter, i.e., a promoter directing a higher level of expression in some cells or tissues of the plant, e.g., in phloem tissues, in vasculature tissues, in companion cells, in guard cells, etc., and is useful in one embodiment of the current disclosure. A plant-expressible promoter can be a phloem-specific promoter, a xylem-specific promoter, or both a phloem-specific and a xylem-specific promoter. In one embodiment of the present disclosure, the phloem-specific promoter Arabidopsis thaliana POTASSIUM TRANSPORTER 2 (pAtAKT2) is used. In a separate embodiment, the companion cell-specific Arabidopsis thaliana SUCROSE TRANSPORTER 2 promoter (pAtSUC2) is used. In another embodiment, the vasculature-specific COMMELINA YELLOW MOT TLE VIRUS promoter (pCoYMV) is used (Zierer et al., 2022. A promoter toolbox for tissue-specific expression supporting translational research in cassava (Manihot esculenta). Front Plant Sci 13:1042379.). In an additional embodiment, the phloem-specific Rice tungro bacilliform virus promoter (pRTBV) is used (Dutt et al., 2012. Evaluation of four phloem-specific promoters in vegetative tissues of transgenic citrus plants. Tree Physiology. 32(1):83-93). In still another embodiment, the guard cell-specific Solanum tuberosum KST1 promoter (pStKST1) is used (Kelly et al., 2017. The Solanum tuberosum KST1 partial promoter as a tool for guard cell expression in multiple plant species. J Exp Bot 68(11): 2885-2897). In yet another embodiment, the cassava MeAKT2a promoter is used. In an alternative embodiment, the cassava MeAKT2b promoter is used. These plant-expressible promoters can be combined with enhancer elements, they can be combined with minimal promoter elements, or can include repeated elements to ensure the expression profile desired.

Additional non-limiting examples of tissue-specific promoters include the maize metallothionein promoter (DE FRAMOND et al, FEBS 290, 103-106, 1991; Application EP 452269), the chitinase promoter (SAMAC et al. Plant Physiol 93, 907-914, 1990), the maize ZRP2 promoter (U.S. Pat. No. 5,633,363), the tomato LeExtl promoter (Bucher et al. Plant Physiol. 128, 911-923, 2002), the glutamine synthetase soybean root promoter (HIREL et al. Plant Mol. Biol. 20, 207-218, 1992), the RCC3 promoter (PCT Application WO 2009/016104), the rice antiquitin promoter (PCT Application WO 2007/076115), the LRR receptor kinase promoter (PCT application WO 02/46439), and the Arabidopsis pCO2 promoter (HEIDSTRA et al, Genes Dev. 18, 1964-1969, 2004). Further non-limiting examples of tissue-specific promoters include the RbcS2B promoter, RbcS1B promoter, RbcS3B promoter, LHB1B1 promoter, LHB1B2 promoter, cab1 promoter, and other promoters described in Engler et al., ACS Synthetic Biology, DOI: 10.1021/sb4001504, 2014. These plant promoters can be combined with enhancer elements, they can be combined with minimal promoter elements, or can include repeated elements to ensure the expression profile desired.

In some embodiments, further genetic alterations to increase expression in plant cells can be utilized. For example, an intron at the 5′ end or 3′ end of an introduced gene, or in the coding sequence of the introduced gene, e.g., the hsp70 intron. Other such genetic elements can include, but are not limited to, promoter enhancer elements, duplicated or triplicated promoter regions, 5′ leader sequences different from another transgene or different from an endogenous (plant host) gene leader sequence, 3′ trailer sequences different from another transgene used in the same plant or different from an endogenous (plant host) trailer sequence.

An introduced gene of the present disclosure can be inserted in host cell DNA so that the inserted gene part is upstream (i.e., 5′) of suitable 3′ end transcription regulation signals (i.e., transcript formation and polyadenylation signals). This is preferably accomplished by inserting the gene in the plant cell genome (nuclear or chloroplast). Preferred polyadenylation and transcript formation signals include those of the nopaline synthase gene (Depicker et al., J. Molec Appl Gen, (1982) 1, 561-573), the octopine synthase gene (Gielen et al., EMBO J, (1984) 3:835-845), the SCSV or the Malic enzyme terminators (Schunmann et al., Plant Funct Biol, (2003) 30:453-460), and the T DNA gene 7 (Velten and Schell, Nucleic Acids Res, (1985) 13, 6981-6998), which act as 3′ untranslated DNA sequences in transformed plant cells. In some embodiments, one or more of the introduced genes are stably integrated into the nuclear genome. Stable integration is present when the nucleic acid sequence remains integrated into the nuclear genome and continues to be expressed (i.e., detectable mRNA transcript or protein is produced) throughout subsequent plant generations. Stable integration into the nuclear genome can be accomplished by any known method in the art (e.g., microparticle bombardment, Agrobacterium-mediated transformation, CRISPR/Cas9, electroporation of protoplasts, microinjection, etc.).

The term recombinant or modified nucleic acids refers to polynucleotides which are made by the combination of two otherwise separated segments of sequence accomplished by the artificial manipulation of isolated segments of polynucleotides by genetic engineering techniques or by chemical synthesis. In so doing one may join together polynucleotide segments of desired functions to generate a desired combination of functions.

As used herein, the term “overexpression” refers to increased expression (e.g., of mRNA, polypeptides, etc.) relative to expression in a wild type organism (e.g., plant) as a result of genetic modification and can refer to expression of heterologous genes at a sufficient level to achieve the desired result such as increased yield. In some embodiments, the increase in expression is a slight increase of about 10% more than expression in wild type. In some embodiments, the increase in expression is an increase of 50% or more (e.g., 60%, 70%, 80%, 100%, etc.) relative to expression in wild type. In some embodiments, an endogenous gene is upregulated. In some embodiments, an exogenous gene is upregulated by virtue of being expressed. Upregulation of a gene in plants can be achieved through any known method in the art, including but not limited to, the use of constitutive promoters with inducible response elements added, inducible promoters, high expression promoters (e.g., PsaD promoter) with inducible response elements added, enhancers, transcriptional and/or translational regulatory sequences, codon optimization, modified transcription factors, and/or mutant or modified genes that control expression of the gene to be upregulated in response to a stimulus such as cytokinin signaling. The term “overexpression” includes constitutive expression as well as increased expression in specific tissues.

Where a recombinant nucleic acid is intended for expression, cloning, or replication of a particular sequence, DNA constructs prepared for introduction into a host cell will typically include a replication system (e.g., vector) recognized by the host, including the intended DNA fragment encoding a desired polypeptide, and can also include transcription and translational initiation regulatory sequences operably linked to the polypeptide-encoding segment. Additionally, such constructs can include cellular localization signals (e.g., plasma membrane localization signals). In preferred embodiments, such DNA constructs are introduced into a host cell's genomic DNA, chloroplast DNA or mitochondrial DNA.

In some embodiments, a non-integrated expression system can be used to induce expression of one or more introduced genes. Expression systems (expression vectors) can include, for example, an origin of replication or autonomously replicating sequence (ARS) and expression control sequences, a promoter, an enhancer and necessary processing information sites, such as ribosome-binding sites, RNA splice sites, polyadenylation sites, transcriptional terminator sequences, and mRNA stabilizing sequences. Signal peptides can also be included where appropriate from secreted polypeptides of the same or related species, which allow the protein to cross and/or lodge in cell membranes, cell wall, or be secreted from the cell.

Selectable markers useful in practicing the methodologies disclosed herein can be positive selectable markers. Typically, positive selection refers to the case in which a genetically altered cell can survive in the presence of a toxic substance only if the recombinant polynucleotide of interest is present within the cell. Negative selectable markers and screenable markers are also well known in the art and are contemplated by the present disclosure. One of skill in the art will recognize that any relevant markers available can be utilized in practicing the compositions, methods, and processes disclosed herein.

Screening and molecular analysis of recombinant strains of the present disclosure can be performed utilizing nucleic acid hybridization techniques. Hybridization procedures are useful for identifying polynucleotides, such as those modified using the techniques described herein, with sufficient homology to the subject regulatory sequences to be useful as taught herein. The particular hybridization techniques are not essential to this disclosure. As improvements are made in hybridization techniques, they can be readily applied by one of skill in the art. Hybridization probes can be labeled with any appropriate label known to those of skill in the art. Hybridization conditions and washing conditions, for example temperature and salt concentration, can be altered to change the stringency of the detection threshold. See, e.g., Sambrook et al. (1989) vide infra or Ausubel et al. (1995) Current Protocols in Molecular Biology, John Wiley & Sons, NY, N.Y., for further guidance on hybridization conditions.

Additionally, screening and molecular analysis of genetically altered strains, as well as creation of desired isolated nucleic acids can be performed using Polymerase Chain Reaction (PCR). PCR is a repetitive, enzymatic, primed synthesis of a nucleic acid sequence. This procedure is well known and commonly used by those skilled in this art (see Mullis, U.S. Pat. Nos. 4,683,195, 4,683,202, and 4,800,159; Saiki et al. (1985) Science 230:1350-1354). PCR is based on the enzymatic amplification of a DNA fragment of interest that is flanked by two oligonucleotide primers that hybridize to opposite strands of the target sequence. The primers are oriented with the 3′ ends pointing towards each other. Repeated cycles of heat denaturation of the template, annealing of the primers to their complementary sequences, and extension of the annealed primers with a DNA polymerase result in the amplification of the segment defined by the 5′ ends of the PCR primers. Because the extension product of each primer can serve as a template for the other primer, each cycle essentially doubles the amount of DNA template produced in the previous cycle. This results in the exponential accumulation of the specific target fragment, up to several million-fold in a few hours. By using a thermostable DNA polymerase such as the Taq polymerase, which is isolated from the thermophilic bacterium Thermus aquaticus, the amplification process can be completely automated. Other enzymes which can be used are known to those skilled in the art.

Nucleic acids and proteins of the present disclosure can also encompass homologues of the specifically disclosed sequences. Homology (e.g., sequence identity) can be 50%-100%. In some instances, such homology is greater than 80%, greater than 85%, greater than 90%, or greater than 95%. The degree of homology or identity needed for any intended use of the sequence(s) is readily identified by one of skill in the art. As used herein, percent sequence identity of two nucleic acids is determined using an algorithm known in the art, such as that disclosed by Karlin and Altschul (1990) Proc. Natl. Acad. Sci. USA 87:2264-2268, modified as in Karlin and Altschul (1993) Proc. Natl. Acad. Sci. USA 90:5873-5877. Such an algorithm is incorporated into the BLASTN, BLASTP, and BLASTX, programs of Altschul et al. (1990) J. Mol. Biol. 215:402-410. BLAST nucleotide searches are performed with the BLASTN program, score=100, wordlength=12, to obtain nucleotide sequences with the desired percent sequence identity. To obtain gapped alignments for comparison purposes, Gapped BLAST is used as described in Altschul et al. (1997) Nucl. Acids. Res. 25:3389-3402. When utilizing BLAST and Gapped BLAST programs, the default parameters of the respective programs (BLASTN and BLASTX) are used. See www.ncbi.nih.gov. One of skill in the art can readily determine in a sequence of interest where a position corresponding to amino acid or nucleic acid in a reference sequence occurs by aligning the sequence of interest with the reference sequence using the suitable BLAST program with the default settings (e.g., for BLASTP: Gap opening penalty: 11, Gap extension penalty: 1, Expectation value: 10, Word size: 3, Max scores: 25, Max alignments: 15, and Matrix: blosum62; and for BLASTN: Gap opening penalty: 5, Gap extension penalty: 2, Nucleic match: 1, Nucleic mismatch −3, Expectation value: 10, Word size: 11, Max scores: 25, and Max alignments: 15).

Preferred host cells are plant cells. Recombinant host cells, in the present context, are those which have been genetically modified to contain an isolated nucleic molecule, contain one or more deleted or otherwise non-functional genes normally present and functional in the host cell, or contain one or more genes to produce at least one recombinant protein. The nucleic acid(s) encoding the protein(s) of the present disclosure can be introduced by any means known to the art which is appropriate for the particular type of cell, including without limitation, transformation, lipofection, electroporation or any other methodology known by those skilled in the art.

“Isolated”, “isolated DNA molecule” or an equivalent term or phrase is intended to mean that the DNA molecule or other moiety is one that is present alone or in combination with other compositions, but altered from or not within its natural environment. For example, nucleic acid elements such as a coding sequence, intron sequence, untranslated leader sequence, promoter sequence, transcriptional termination sequence, and the like, that are naturally found within the DNA of the genome of an organism are not considered to be “isolated” so long as the element is within the genome of the organism and at the location within the genome in which it is naturally found. However, each of these elements, and subparts of these elements, would be “isolated” from its natural setting within the scope of this disclosure so long as the element is not within the genome of the organism in which it is naturally found, the element is altered from its natural form, or the element is not at the location within the genome in which it is naturally found. Similarly, a nucleotide sequence encoding a protein or any naturally occurring variant of that protein would be an isolated nucleotide sequence so long as the nucleotide sequence was not within the DNA of the organism from which the sequence encoding the protein is naturally found in its natural location or if that nucleotide sequence was altered from its natural form. A synthetic nucleotide sequence encoding the amino acid sequence of the naturally occurring protein would be considered to be isolated for the purposes of this disclosure. For the purposes of this disclosure, any transgenic nucleotide sequence, i.e., the nucleotide sequence of the DNA inserted into the genome of the cells of a plant, alga, fungus, or bacterium, or present in an extrachromosomal vector, would be considered to be an isolated nucleotide sequence whether it is present within the plasmid or similar structure used to transform the cells, within the genome of the plant or bacterium, or present in detectable amounts in tissues, progeny, biological samples or commodity products derived from the plant or bacterium.

The sequences in this application are provided in Table 1 below.

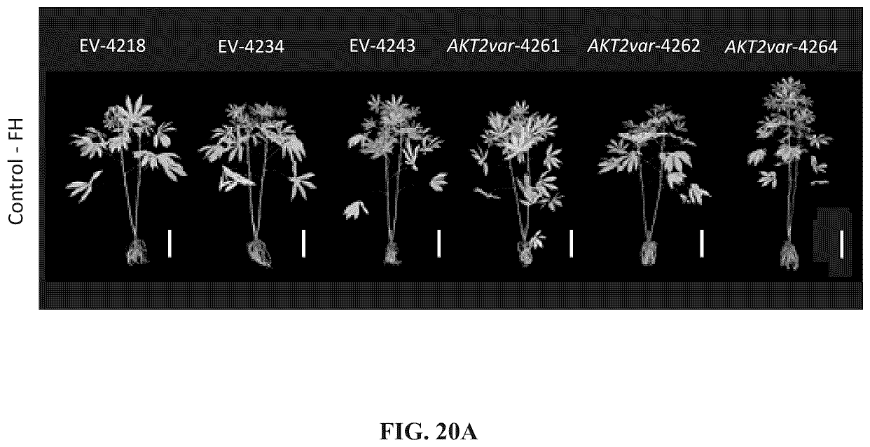

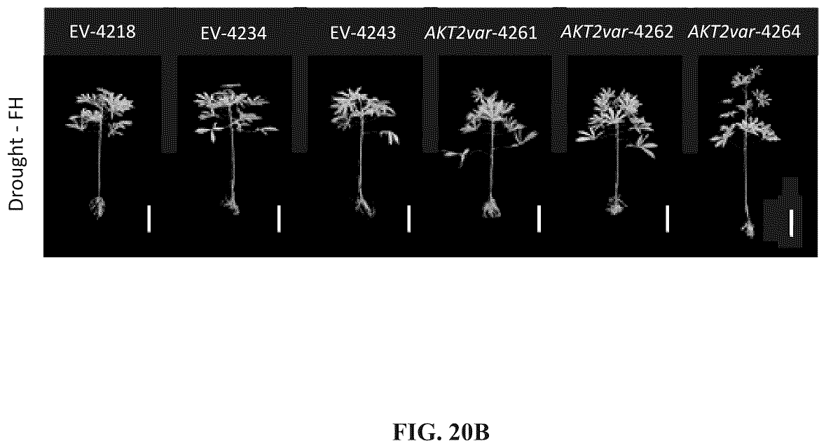

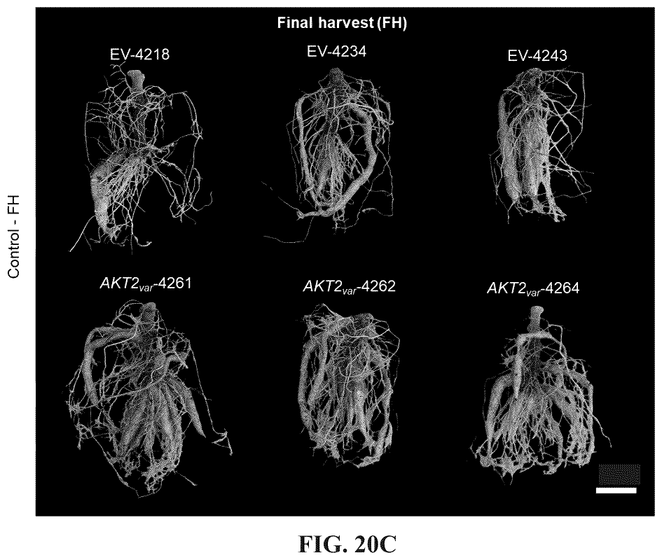

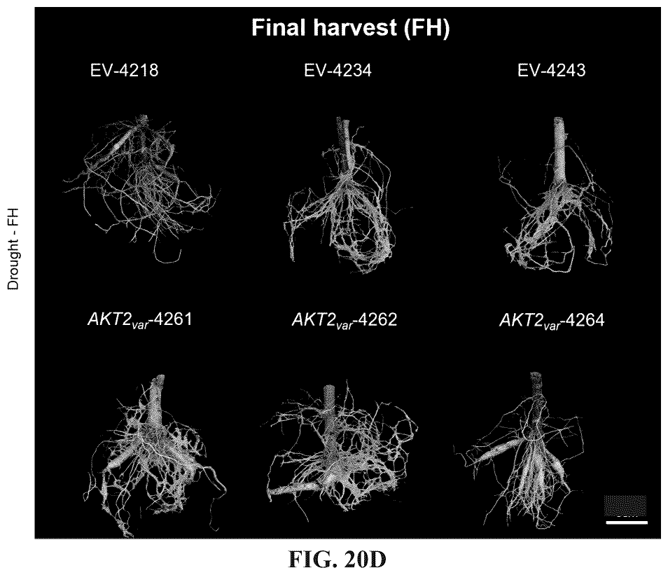

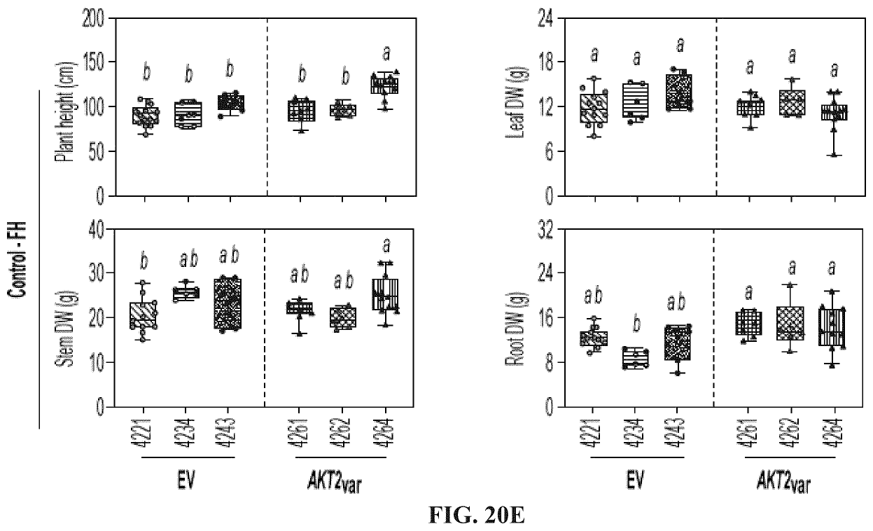

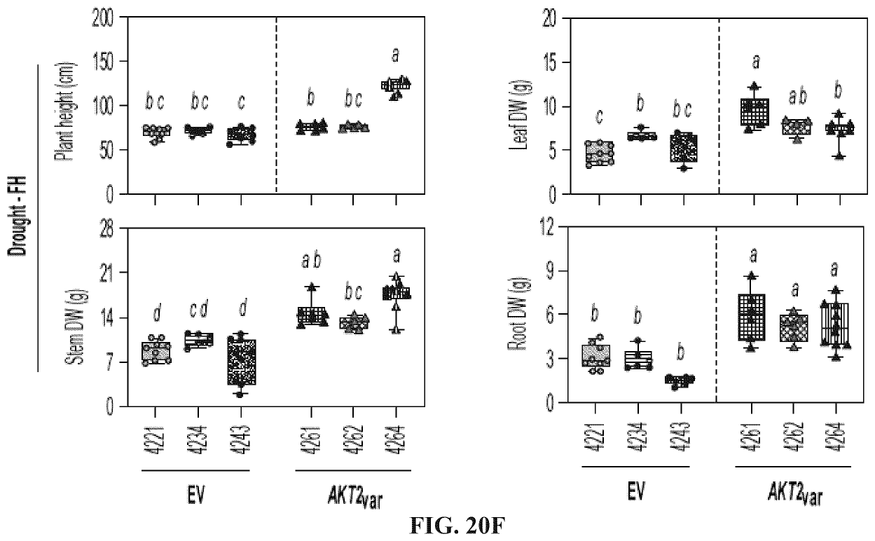

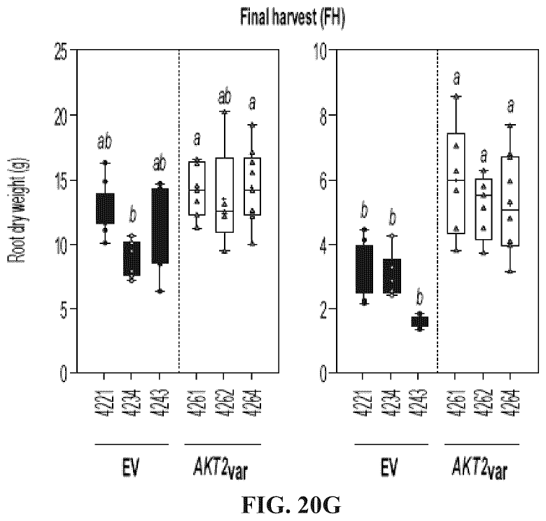

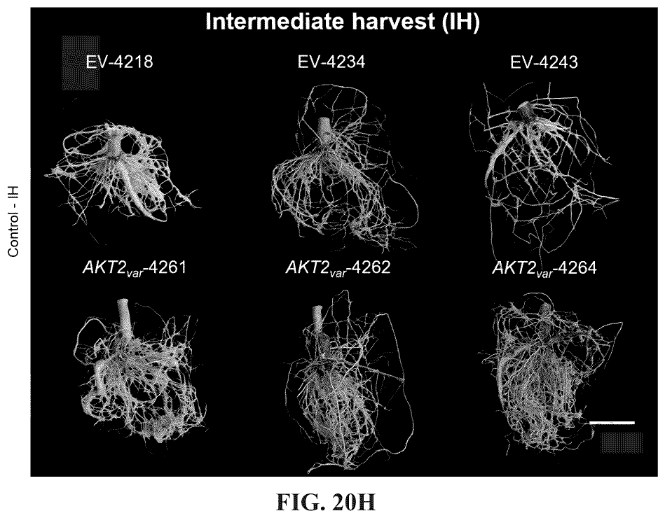

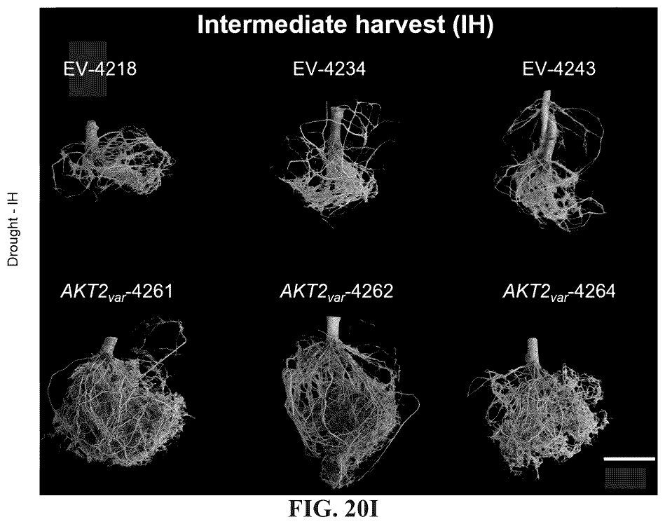

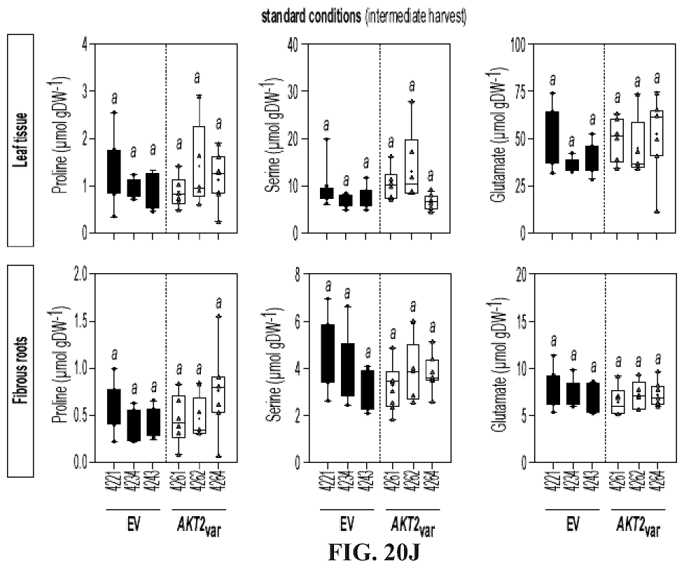

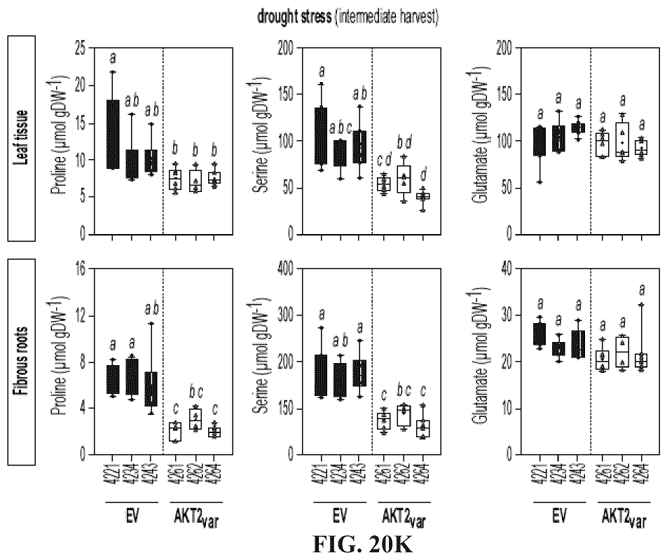

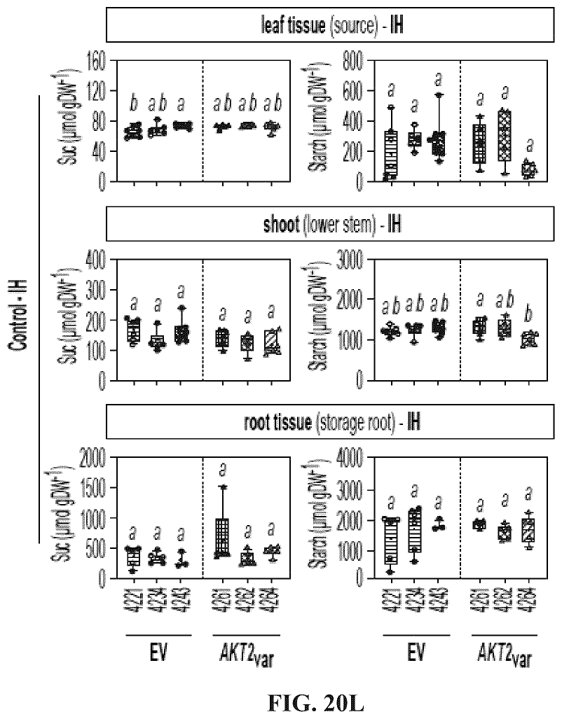

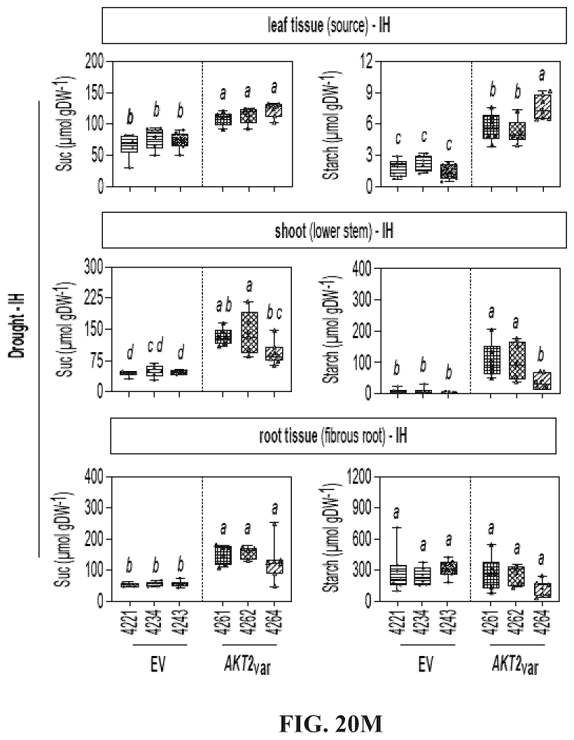

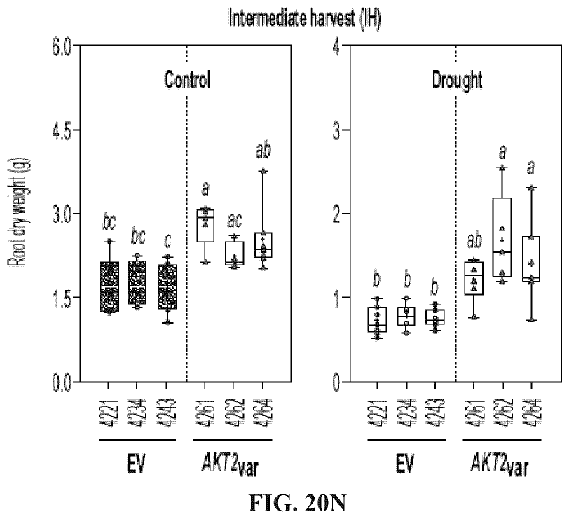

TABLE 1
Sequences

| SEQ ID | | |
|---|---|---|
| NO | Description | Sequence |
| 1 | binary vector p134GG_ | tgccgaattcggatccggagattgagtctaaaatattgaaaatactcact |
| | pAtAKT2: AtAKT2mut | atcacatatgtcaacgataacactaatttttttttttgtataatatacgt |
| | | aacgacacctgcattagatctaatttttaatcgattagagcttaaatgac |
| | | atcgttttggtttgactaagagaatcgttaaaaatgttagaaaatctgtt |
| | | aaagcttaataactgtttgtttgttttttttctttctttttttgtcaatc |
| | | gaaaattagactattaaatgacaattttccgtttaaaatggcaagtccta |
| | | gatttgaaaaggtcttttgaaatctttttaagagaattaagggtgaatta |
| | | aatatattcagaaattaaagggtcgaattgcaaactttcttggttcatac |
| | | ttggtctgcctttgtccctttcttatcttcttcgcgcgcttttgaaaaaa |
| | | atagaaacccgccactgaacgtcagtcatccacgtgtcctctcctgtgct |
| | | ctgtttccgatcgtcggagacatcaatttgcctgcttgtagggtgattcg |
| | | tcaaatctattatcaggttttaaatatactcgaattgacttccaaattct |
| | | tagtctctagtgtaatgattttgagaatcacttaactccaaaaatataat |
| | | ccacgatcccgtgttaattattgaagaatcaatcgtttttaatttctcac |
| | | caatagatgttgctcttattacttaaaacaaattgtttagacaaatgtag |
| | | caagtgtgatacttagtgggatcttaaagacgatttctcctataacagag |
| | | gacaaacaggtcggtcaattacaatgtcatccctctttaccctgtctttt |
| | | tttttcttcttaaaacctaaccatttgattgtttctaaaggtatttcaag |
| | | aatatatgatcaatctagatgaatactataccgacgatgactacacacac |
| | | aaggaaatatatatatcagctttcttttcacctaaaagtggtcccggttt |
| | | agaatctaattcctttatctctcattttcttctgcttcacattcccgcta |
| | | gtcaaatgttaataagtgcacacaacgttttctcgaagcattagaatgtc |
| | | ctcctcttaattaatctccttctgattagattctcaatagagtttaaatt |
| | | tgttaatggagagatatattgggaccctcaaggcttctaattataccacg |
| | | tttggcataattctctatcgtttggggccacatctttcacacttcattac |
| | | cttatcaccaaaacataaaatcaatcaacttttttttgccttattgattg |
| | | tgttggatccctccaaaattaaaacttgtgttccccacaaaagcttaccc |
| | | aatttcacttcaatcttaacaaataggaccaccactaccacgtacggttt |
| | | gcatcatacaaaccacaaactccttcttcattacaattattatatcatct |
| | | actaaaacctctttctccctctctctttcttgttcttagtgctaaatttt |
| | | ctttgttcaggagaaatatatactgaattcggatcctctagaccatgtac |
| | | ccatacgatgttcctgactatgcgggctatccctatgacgtcccggacta |
| | | tgcaggattgtatccatatgacgttccagattacgccactagagctgctt |
| | | acccatacgatgttcctgactatgcgggctatccctatgacgtcccggac |
| | | tatgcaggattgtatccatatgacgttccagattacgcaatggacctcaa |
| | | gtattcagcatctcattgcaacttatcctcagacatgaagctcaggcgtt |
| | | ttcatcagcatcgtggaaaaggaagagaagaagagtatgatgcttcttct |
| | | ctcagcttgaacaatctgtcaaaacttattcttcctccacttggtgttgc |
| | | tagctataaccagaatcacatcaggtctagtggatggatcatctcaccta |
| | | tggactcaagatacaggtgctgggaattttatatggtgcttttagtggca |
| | | tactctgcttgggtttacccttttgaagttgcatttctgaattcatcacc |
| | | aaagagaaacctttgtatcgctgacaacatcgtagacttgttcttcgctg |
| | | ttgacattgtcttgacttttttcgttgcttacattgacgaaagaacacag |
| | | cttcttgtccgtgaacctaaacagattgcagtgaggtacctatcaacatg |
| | | gttcttgatggatgttgcatcaactattccatttgacgctattggatact |
| | | taatcactggcacatccactttaaatatcacttgtaatctcttgggatta |
| | | cttagattttggagacttcgtagagttaaacacctcttcactaggctcga |
| | | gaaggacattagatataactatttctggattcgctgctttagacttctat |
| | | cagtgacattgtttctagtgcactgtgctggatgcagttattacctaatt |
| | | gcagacagatatccacaccaaggaaagacatggactgatgctatccctaa |
| | | tttcacagagacaagtctttccatcagatacattgcagctatttattggt |
| | | ctatcactacaatgaccacagtgggatatggagatcttcatgcaagcaac |
| | | actattgaaatggtattcattacagtctacatgttattcaatcttggcct |
| | | cactgcttaccttattggtaacatgactaatttggtcgtggaagggactc |
| | | gtcgtaccatggaatttaggaataacattgaagcagcttcaaactttgtt |
| | | aacagaaacagattgcctcctagattaaaagaccagattttagcttacat |
| | | gtgtttaaggtttaaagcagagagcttaaatcagcaacatcttattgacc |
| | | agctcccaaaatctatctacaaaagcatttgtcaacatctttttcttcca |
| | | tctgttgaaaaagtttacctcttcaaaggcgtctcaagagaaattcttct |
| | | tcttctggtttcaaaaatgaaggctgagtatattccaccaagagaggatg |
| | | tcattatgcagaacgaagctccagatgatgtttacattattgtgtcagga |
| | | gaagttgagatcattgattcagagatggagagagagtctgttttaggcac |
| | | tctacgttgtggagacatttttggagaagttggagcactttgttgcagac |
| | | cacaaagctacacttttcaaactaagtctttatcacagcttctccgtctc |
| | | aaaacatctttccttattgagacaatgcagattaaacaacaagacaatgc |
| | | cacaatgctcaagaacttcttgcagcatcacaaaaagctgagtaatttag |
| | | acattggtgatctaaaggcacaacaaaatggcgaaaacaccgatgttgtt |
| | | cctcctaacattgcctcaaatctcatcgctgtggtgactacaggcaatgc |
| | | agctcttcttgatgagctacttaaggctaagttaagccctgacattacag |
| | | attccaaaggaaaaactccattgcatgtagcagcttctagaggatatgaa |
| | | gattgtgttttagtactcttaaagcacggttgcaacatccacattagaga |
| | | tgtgaatggtaatagtgctctatgggaagcaattatttctaagcattacg |
| | | agattttcagaatcctttatcatttcgcagccatttctgatccacacatt |
| | | gctggagatcttctatgtgaagcagctaaacagaacaatgtagaagtcat |
| | | gaaggctcttttaaaacaggggcttaacgtcgacacagaggatcaccatg |
| | | gcgtcacagctttacaggtcgctatggctgaggatcagatggacatggtg |
| | | aatctcctggctactaacggtgcagatgtagtttgtgtgaatacacataa |
| | | tgaattcacaccattggagaagttaagagttgtggaagaagaagaagaag |
| | | aagaacgtggaagagtgagtatttacagaggacatccattggagaggaga |
| | | gaaagaagttgcaatgaagctgggaagcttattcttcttcctccttcact |
| | | tgatgacctcaagaaaattgcaggagagaagtttgggtttgatggaagtg |
| | | agactatggtgactaatgaagatggagctgagattgacagtattgaagtg |
| | | attagagataatgacaaactctactttgtcgtaaacaagattatttaggc |
| | | ttatatgaagatgaagatgaaatatttggtgtgtcaaataaaaagcttgt |
| | | gtgcttaagtttgtgtttttttcttggcttgttgtgttatgaatttgtgg |
| | | ctttttctaatattaaatgaatgtaagatctcattataatgaataaacaa |
| | | atgtttctataatccattgtgaatgttttgttggatctcttctgcagcat |
| | | ataactactgtatgtgctatggtatggactatggaattccgctgcaagag |
| | | gatgcacatgtgaccgagggaatcgggaattaaactatcagtgtttgaca |
| | | ggatatattggcgggtaaacctaagagaaaagagcgtttattagaataac |
| | | ggatatttaaaagggcgtgaaaaggtttatccgttcgtccatttgtatgt |
| | | gcatgccaaccacagggttcccctcgggatcaaagtactttgatccaacc |
| | | cctccgctgctatagtgcagtcggcttctgacgttcagtgcagccgtgtt |
| | | ctgaaaacgacatgtcgcacaagtcctaagttacgcgacaggctgccgcc |
| | | ctgcccttttcctggcgttttcttgtcgcgtgttttagtcgcataaagta |
| | | gaatacttgcgactagaaccggagacattacgccatgaacaagagcgccg |
| | | ccgctggcctgctgggctatgcccgcgtcagcaccgacgaccaggacttg |
| | | accaaccaacgggccgaactgcacgcggccggctgcaccaagctgttttc |
| | | cgagaagatcaccggcaccaggcgcgaccgcccggagctggccaggatgc |
| | | ttgaccacctacgccctggcgacgttgtgacagtgaccaggctagaccgc |
| | | ctggcccgcagcacccgcgacctactggacattgccgagcgcatccagga |
| | | ggccggcgcgggcctgcgtagcctggcagagccgtgggccgacaccacca |
| | | cgccggccggccgcatggtgttgaccgtgttcgccggcattgccgagttc |
| | | gagcgttccctaatcatcgaccgcacccggagcgggcgcgaggccgccaa |
| | | ggcccgaggcgtgaagtttggcccccgccctaccctcaccccggcacaga |
| | | tcgcgcacgcccgcgagctgatcgaccaggaaggccgcaccgtgaaagag |
| | | gcggctgcactgcttggcgtgcatcgctcgaccctgtaccgcgcacttga |
| | | gcgcagcgaggaagtgacgcccaccgaggccaggcggcgcggtgccttcc |
| | | gtgaggacgcattgaccgaggccgacgccctggcggccgccgagaatgaa |
| | | cgccaagaggaacaagcatgaaaccgcaccaggacggccaggacgaaccg |
| | | tttttcattaccgaagagatcgaggcggagatgatcgcggccgggtacgt |
| | | gttcgagccgcccgcgcacgtctcaaccgtgcggctgcatgaaatcctgg |
| | | ccggtttgtctgatgccaagctggcggcctggccggccagcttggccgct |
| | | gaagaaaccgagcgccgccgtctaaaaaggtgatgtgtatttgagtaaaa |
| | | cagcttgcgtcatgcggtcgctgcgtatatgatgcgatgagtaaataaac |
| | | aaatacgcaaggggaacgcatgaaggttatcgctgtacttaaccagaaag |
| | | gcgggtcaggcaagacgaccatcgcaacccatctagcccgcgccctgcaa |
| | | ctcgccggggccgatgttctgttagtcgattccgatccccagggcagtgc |
| | | ccgcgattgggcggccgtgcgggaagatcaaccgctaaccgttgtcggca |
| | | tcgaccgcccgacgattgaccgcgacgtgaaggccatcggccggcgcgac |
| | | ttcgtagtgatcgacggagcgccccaggcggcggacttggctgtgtccgc |
| | | gatcaaggcagccgacttcgtgctgattccggtgcagccaagcccttacg |
| | | acatatgggccaccgccgacctggtggagctggttaagcagcgcattgag |
| | | gtcacggatggaaggctacaagcggcctttgtcgtgtcgcgggcgatcaa |
| | | aggcacgcgcatcggcggtgaggttgccgaggcgctggccgggtacgagc |
| | | tgcccattcttgagtcccgtatcacgcagcgcgtgagctacccaggcact |
| | | gccgccgccggcacaaccgttcttgaatcagaacccgagggcgacgctgc |
| | | ccgcgaggtccaggcgctggccgctgaaattaaatcaaaactcatttgag |
| | | ttaatgaggtaaagagaaaatgagcaaaagcacaaacacgctaagtgccg |
| | | gccgtccgagcgcacgcagcagcaaggctgcaacgttggccagcctggca |
| | | gacacgccagccatgaagcgggtcaactttcagttgccggcggaggatca |
| | | caccaagctgaagatgtacgcggtacgccaaggcaagaccattaccgagc |
| | | tgctatctgaatacatcgcgcagctaccagagtaaatgagcaaatgaata |
| | | aatgagtagatgaattttagcggctaaaggaggcggcatggaaaatcaag |
| | | aacaaccaggcaccgacgccgtggaatgccccatgtgtggaggaacgggc |
| | | ggttggccaggcgtaagcggctgggttgtctgccggccctgcaatggcac |
| | | tggaacccccaagcccgaggaatcggcgtgacggtcgcaaaccatccggc |
| | | ccggtacaaatcggcgcggcgctgggtgatgacctggtggagaagttgaa |
| | | ggccgcgcaggccgcccagcggcaacgcatcgaggcagaagcacgccccg |
| | | gtgaatcgtggcaagcggccgctgatcgaatccgcaaagaatcccggcaa |
| | | ccgccggcagccggtgcgccgtcgattaggaagccgcccaagggcgacga |
| | | gcaaccagattttttcgttccgatgctctatgacgtgggcacccgcgata |
| | | gtcgcagcatcatggacgtggccgttttccgtctgtcgaagcgtgaccga |
| | | cgagctggcgaggtgatccgctacgagcttccagacgggcacgtagaggt |
| | | ttccgcagggccggccggcatggccagtgtgtgggattacgacctggtac |
| | | tgatggcggtttcccatctaaccgaatccatgaaccgataccgggaaggg |
| | | aagggagacaagcccggccgcgtgttccgtccacacgttgcggacgtact |
| | | caagttctgccggcgagccgatggcggaaagcagaaagacgacctggtag |
| | | aaacctgcattcggttaaacaccacgcacgttgccatgcagcgtacgaag |
| | | aaggccaagaacggccgcctggtgacggtatccgagggtgaagccttgat |
| | | tagccgctacaagatcgtaaagagcgaaaccgggcggccggagtacatcg |
| | | agatcgagctagctgattggatgtaccgcgagatcacagaaggcaagaac |
| | | ccggacgtgctgacggttcaccccgattactttttgatcgatcccggcat |
| | | cggccgttttctctaccgcctggcacgccgcgccgcaggcaaggcagaag |
| | | ccagatggttgttcaagacgatctacgaacgcagtggcagcgccggagag |
| | | ttcaagaagttctgtttcaccgtgcgcaagctgatcgggtcaaatgacct |
| | | gccggagtacgatttgaaggaggaggcggggcaggctggcccgatcctag |
| | | tcatgcgctaccgcaacctgatcgagggcgaagcatccgccggttcctaa |
| | | tgtacggagcagatgctagggcaaattgccctagcaggggaaaaaggtcg |
| | | aaaaggtctctttcctgtggatagcacgtacattgggaacccaaagccgt |
| | | acattgggaaccggaacccgtacattgggaacccaaagccgtacattggg |
| | | aaccggtcacacatgtaagtgactgatataaaagagaaaaaaggcgattt |
| | | ttccgcctaaaactctttaaaacttattaaaactcttaaaacccgcctgg |
| | | cctgtgcataactgtctggccagcgcacagccgaagagctgcaaaaagcg |
| | | cctacccttcggtcgctgcgctccctacgccccgccgcttcgcgtcggcc |
| | | tatcgcggccgctggccgctcaaaaatggctggcctacggccaggcaatc |
| | | taccagggcgcggacaagccgcgccgtcgccactcgaccgccggcgccca |
| | | catcaaggcaccctgcctcgcgcgtttcggtgatgacggtgaaaacctct |
| | | gacacatgcagctcccggagacggtcacagcttgtctgtaagcggatgcc |
| | | gggagcagacaagcccgtcagggcgcgtcagcgggtgttggcgggtgtcg |
| | | gggcgcagccatgacccagtcacgtagcgatagcggagtgtatactggct |
| | | taactatgcggcatcagagcagattgtactgagagtgcaccatatgcggt |
| | | gtgaaataccgcacagatgcgtaaggagaaaataccgcatcaggcgctct |
| | | tccgcttcctcgctcactgactcgctgcgctcggtcgttcggctgcggcg |
| | | agcggtatcagctcactcaaaggcggtaatacggttatccacagaatcag |
| | | gggataacgcaggaaagaacatgtgagcaaaaggccagcaaaaggccagg |
| | | aaccgtaaaaaggccgcgttgctggcgtttttccataggctccgcccccc |
| | | tgacgagcatcacaaaaatcgacgctcaagtcagaggtggcgaaacccga |
| | | caggactataaagataccaggcgtttccccctggaagctccctcgtgcgc |
| | | tctcctgttccgaccctgccgcttaccggatacctgtccgcctttctccc |
| | | ttcgggaagcgtggcgctttctcatagctcacgctgtaggtatctcagtt |
| | | cggtgtaggtcgttcgctccaagctgggctgtgtgcacgaaccccccgtt |
| | | cagcccgaccgctgcgccttatccggtaactatcgtcttgagtccaaccc |
| | | ggtaagacacgacttatcgccactggcagcagccactggtaacaggatta |
| | | gcagagcgaggtatgtaggcggtgctacagagttcttgaagtggtggcct |
| | | aactacggctacactagaaggacagtatttggtatctgcgctctgctgaa |
| | | gccagttaccttcggaaaaagagttggtagctcttgatccggcaaacaaa |
| | | ccaccgctggtagcggtggtttttttgtttgcaagcagcagattacgcgc |
| | | agaaaaaaaggatctcaagaagatcctttgatcttttctacggggtctga |
| | | cgctcagtggaacgaaaactcacgttaagggattttggtcatgcattcta |
| | | ggtactaaaacaattcatccagtaaaatataatattttattttctcccaa |
| | | tcaggcttgatccccagtaagtcaaaaaatagctcgacatactgttcttc |
| | | cccgatatcctccctgatcgaccggacgcagaaggcaatgtcataccact |
| | | tgtccgccctgccgcttctcccaagatcaataaagccacttactttgcca |
| | | tctttcacaaagatgttgctgtctcccaggtcgccgtgggaaaagacaag |
| | | ttcctcttcgggcttttccgtctttaaaaaatcatacagctcgcgcggat |
| | | ctttaaatggagtatcttcttcccagttttcgcaatccacatcggccaga |
| | | tcgttattcagtaagtaatccaattcggctaagcggctgtctaagctatt |
| | | cgtatagggacaatccgatatgtcgatggagtgaaagagcctgatgcact |
| | | ccgcatacagctcgataatcttttcagggctttgttcatcttcatactct |
| | | tccgagcaaaggacgccatcggcctcactcatgagcagattgctccagcc |
| | | atcatgccgttcaaagtgcaggacctttggaacaggcagctttccttcca |
| | | gccatagcatcatgtccttttcccgttccacatcataggtggtcccttta |
| | | taccggctgtccgtcatttttaaatataggttttcattttctcccaccag |
| | | cttatataccttagcctgggcctcatgggccttcctttcactgcccgctt |
| | | tccagtcgggaaacctgtcgtgccaggcccacatcaaggcaccctgcctc |
| | | gcgcgtttcggtgatgacggtgaaaacctctgacacatgcagctcccgga |
| | | gacggtcacagcttgtctgtaagcggatgccgggagcagacaagcccgtc |
| | | agggcgcgtcagcgggtgttggcgggtgtcggggcgcagccatgacccag |
| | | tcacgtagcgatagcggagtgtatactggcttaactatgcggcatcagag |
| | | cagattgtactgagagtgcaccatatgcggtgtgaaataccgcacagatg |
| | | cgtaaggagaaaataccgcatcaggcgctcttccgcttcctcgctcactg |
| | | actcgctgcgctcggtcgttcggctgcggcgagcggtatcagctcactca |
| | | aaggcggtaatacggttatccacagaatcaggggataacgcaggaaagaa |
| | | catgtgagcaaaaggccagcaaaaggccaggaaccgtaaaaaggccgcgt |
| | | tgctggcgtttttccataggctccgcccccctgacgagcatcacaaaaat |
| | | cgacgctcaagtcagaggtggcgaaacccgacaggactataaagatacca |
| | | ggcgtttccccctggaagctccctcgtgcgctctcctgttccgaccctgc |
| | | cgcttaccggatacctgtccgcctttctcccttcgggaagcgtggcgctt |
| | | tctcatagctcacgctgtaggtatctcagttcggtgtaggtcgttcgctc |
| | | caagctgggctgtgtgcacgaaccccccgttcagcccgaccgctgcgcct |
| | | tatccggtaactatcgtcttgagtccaacccggtaagacacgacttatcg |
| | | ccactggcagcagccactggtaacaggattagcagagcgaggtatgtagg |
| | | cggtgctacagagttcttgaagtggtggcctaactacggctacactagaa |
| | | ggacagtatttggtatctgcgctctgctgaagccagttaccttcggaaaa |
| | | agagttggtagctcttgatccggcaaacaaaccaccgctggtagcggtgg |
| | | tttttttgtttgcaagcagcagattacgcgcagaaaaaaaggatctcaag |
| | | aagatcctttgatcttttctacggggtctgacgctcagtggaacgaaaac |
| | | tcacgttaagggattttggtcatgcattctaggtactaaaacaattcatc |
| | | cagtaaaatataatattttattttctcccaatcaggcttgatccccagta |
| | | agtcaaaaaatagctcgacatactgttcttccccgatatcctccctgatc |
| | | gaccggacgcagaaggcaatgtcataccacttgtccgccctgccgcttct |
| | | cccaagatcaataaagccacttactttgccatctttcacaaagatgttgc |
| | | tgtctcccaggtcgccgtgggaaaagacaagttcctcttcgggcttttcc |
| | | gtctttaaaaaatcatacagctcgcgcggatctttaaatggagtatcttc |
| | | ttcccagttttcgcaatccacatcggccagatcgttattcagtaagtaat |
| | | ccaattcggctaagcggctgtctaagctattcgtatagggacaatccgat |
| | | atgtcgatggagtgaaagagcctgatgcactccgcatacagctcgataat |
| | | cttttcagggctttgttcatcttcatactcttccgagcaaaggacgccat |
| | | cggcctcactcatgagcagattgctccagccatcatgccgttcaaagtgc |
| | | aggacctttggaacaggcagctttccttccagccatagcatcatgtcctt |
| | | ttcccgttccacatcataggtggtccctttataccggctgtccgtcattt |
| | | ttaaatataggttttcattttctcccaccagcttatataccttagcagga |
| | | gacattccttccgtatcttttacgcagcggtatttttcgatcagtttttt |
| | | caattccggtgatattctcattttagccatttattatttccttcctcttt |
| | | tctacagtatttaaagataccccaagaagctaattataacaagacgaact |
| | | ccaattcactgttccttgcattctaaaaccttaaataccagaaaacagct |
| | | ttttcaaagttgttttcaaagttggcgtataacatagtatcgacggagcc |
| | | gattttgaaaccgcggtgatcacaggcagcaacgctctgtcatcgttaca |
| | | atcaacatgctaccctccgcgagatcatccgtgtttcaaacccggcagct |
| | | tagttgccgttcttccgaatagcatcggtaacatgagcaaagtctgccgc |
| | | cttacaacggctctcccgctgacgccgtcccggactgatgggctgcctgt |
| | | atcgagtggtgattttgtgccgagctgccggtcggggagctgttggctgg |
| | | ctggtggcaggatatattgtggtgtaaacaaattgacgcttagacaactt |
| | | aataacacattgcggacgtttttaatgtactgaattaacgccgaattaat |
| | | tcgggggatctggattttagtactggattttggttttaggaattagaaat |
| | | tttattgatagaagtattttacaaatacaaatacatactaagggtttctt |
| | | atatgctcaacacatgagcgaaaccctataggaaccctaattcccttatc |
| | | tgggaactactcacacattattatggagaaactcgagcttgtcgatcgac |
| | | agatcccggtcggcatctactctatttctttgccctcggacgagtgctgg |
| | | ggcgtcggtttccactatcggcgagtacttctacacagccatcggtccag |
| | | acggccgcgcttctgcgggcgatttgtgtacgcccgacagtcccggctcc |
| | | ggatcggacgattgcgtcgcatcgaccctgcgcccaagctgcatcatcga |
| | | aattgccgtcaaccaagctctgatagagttggtcaagaccaatgcggagc |
| | | atatacgcccggagtcgtggcgatcctgcaagctccggatgcctccgctc |
| | | gaagtagcgcgtctgctgctccatacaagccaaccacggcctccagaaga |
| | | agatgttggcgacctcgtattgggaatccccgaacatcgcctcgctccag |
| | | tcaatgaccgctgttatgcggccattgtccgtcaggacattgttggagcc |
| | | gaaatccgcgtgcacgaggtgccggacttcggggcagtcctcggcccaaa |
| | | gcatcagctcatcgagagcctgcgcgacggacgcactgacggtgtcgtcc |
| | | atcacagtttgccagtgatacacatggggatcagcaatcgcgcatatgaa |
| | | atcacgccatgtagtgtattgaccgattccttgcggtccgaatgggccga |
| | | acccgctcgtctggctaagatcggccgcagcgatcgcatccatagcctcc |
| | | gcgaccggttgtagaacagcgggcagttcggtttcaggcaggtcttgcaa |
| | | cgtgacaccctgtgcacggcgggagatgcaataggtcaggctctcgctaa |
| | | actccccaatgtcaagcacttccggaatcgggagcgcggccgatgcaaag |
| | | tgccgataaacataacgatctttgtagaaaccatcggcgcagctatttac |
| | | ccgcaggacatatccacgccctcctacatcgaagctgaaagcacgagatt |
| | | cttcgccctccgagagctgcatcaggtcggagacgctgtcgaacttttcg |
| | | atcagaaacttctcgacagacgtcgcggtgagttcaggctttttcatatc |
| | | cggtgcagattatttggattgagagtgaatatgagactctaattggatac |
| | | cgaggggaatttatggaacgtcagtggagcatttttgacaagaaatattt |
| | | gctagctgatagtgaccttaggcgacttttgaacgcgcaataatggtttc |
| | | tgacgtatgtgcttagctcattaaactccagaaacccgcggctgagtggc |
| | | tccttcaacgttgcggttctgtcagttccaaacgtaaaacggcttgtccc |
| | | gcgtcatcggcgggggtcataacgtgactcccttaattctccgctcatga |
| | | tcagattgtcgtttcccgccttcagtttaaactatcagtgtccaacatgt |
| | | tggcaagctgctctagccaatacgcaaaccgcctctccccgcgcgttggc |
| | | cgattcattaatgcagctggcacgacaggtttcccgactggaaagcgggc |
| | | agtgagcgcaacgcaattaatgtgagttagctcactcattaggcacccca |
| | | ggctttacactttatgcttccggctcgtatgttgtgtggaattgtgagcg |
| | | gataacaatttcacacaggaaacagctatgaccatgattacgaattc |
| 2 | Promoter-Arabidopsis | attgagtctaaaatattgaaaatactcactatcacatatgtcaacgataa |
| | thaliana POTASSIUM | cactaatttttttttttgtataatatacgtaacgacacctgcattagatc |
| | TRANSPORTER 2 | taatttttaatcgattagagcttaaatgacatcgttttggtttgactaag |
| | (pAtAKT2) | agaatcgttaaaaatgttagaaaatctgttaaagcttaataactgtttgt |
| | | ttgttttttttctttctttttttgtcaatcgaaaattagactattaaatg |
| | | acaattttccgtttaaaatggcaagtcctagatttgaaaaggtcttttga |
| | | aatctttttaagagaattaagggtgaattaaatatattcagaaattaaag |
| | | ggtcgaattgcaaactttcttggttcatacttggtctgcctttgtccctt |
| | | tcttatcttcttcgcgcgcttttgaaaaaaatagaaacccgccactgaac |
| | | gtcagtcatccacgtgtcctctcctgtgctctgtttccgatcgtcggaga |
| | | catcaatttgcctgcttgtagggtgattcgtcaaatctattatcaggttt |
| | | taaatatactcgaattgacttccaaattcttagtctctagtgtaatgatt |
| | | ttgagaatcacttaactccaaaaatataatccacgatcccgtgttaatta |
| | | ttgaagaatcaatcgtttttaatttctcaccaatagatgttgctcttatt |
| | | acttaaaacaaattgtttagacaaatgtagcaagtgtgatacttagtggg |
| | | atcttaaagacgatttctcctataacagaggacaaacaggtcggtcaatt |
| | | acaatgtcatccctctttaccctgtctttttttttcttcttaaaacctaa |
| | | ccatttgattgtttctaaaggtatttcaagaatatatgatcaatctagat |
| | | gaatactataccgacgatgactacacacacaaggaaatatatatatcagc |
| | | tttcttttcacctaaaagtggtcccggtttagaatctaattcctttatct |
| | | ctcattttcttctgcttcacattcccgctagtcaaatgttaataagtgca |
| | | cacaacgttttctcgaagcattagaatgtcctcctcttaattaatctcct |
| | | tctgattagattctcaatagagtttaaatttgttaatggagagatatatt |
| | | gggaccctcaaggcttctaattataccacgtttggcataattctctatcg |
| | | tttggggccacatctttcacacttcattaccttatcaccaaaacataaaa |
| | | tcaatcaacttttttttgccttattgattgtgttggatccctccaaaatt |
| | | aaaacttgtgttccccacaaaagcttacccaatttcacttcaatcttaac |
| | | aaataggaccaccactaccacgtacggtttgcatcatacaaaccacaaac |
| | | tccttcttcattacaattattatatcatctactaaaacctctttctccct |
| | | ctctctttcttgttcttagtgctaaattttctttgttcaggagaaatata |
| | | tactgaattcggatcctcta |
| 3 | CDS-Arabidopsis | gacctcaagtattcagcatctcattgcaacttatcctcagacatgaagct |
| | thaliana POTASSIUM | caggcgttttcatcagcatcgtggaaaaggaagagaagaagagtatgatg |
| | TRANSPORTER 2 | cttcttctctcagcttgaacaatctgtcaaaacttattcttcctccactt |
| | (AtAKT2) | ggtgttgctagctataaccagaatcacatcaggtctagtggatggatcat |
| | | ctcacctatggactcaagatacaggtgctgggaattttatatggtgcttt |
| | | tagtggcatactctgcttgggtttacccttttgaagttgcatttctgaat |
| | | tcatcaccaaagagaaacctttgtatcgctgacaacatcgtagacttgtt |
| | | cttcgctgttgacattgtcttgacttttttcgttgcttacattgacgaaa |
| | | gaacacagcttcttgtccgtgaacctaaacagattgcagtgaggtaccta |
| | | tcaacatggttcttgatggatgttgcatcaactattccatttgacgctat |
| | | tggatacttaatcactggcacatccactttaaatatcacttgtaatctct |
| | | tgggattacttagattttggagacttcgtagagttaaacacctcttcact |
| | | aggctcgagaaggacattagatataactatttctggattcgctgctttag |
| | | acttctatcagtgacattgtttctagtgcactgtgctggatgcagttatt |
| | | acctaattgcagacagatatccacaccaaggaaagacatggactgatgct |
| | | atccctaatttcacagagacaagtctttccatcagatacattgcagctat |
| | | ttattggtctatcactacaatgaccacagtgggatatggagatcttcatg |
| | | caagcaacactattgaaatggtattcattacagtctacatgttattcaat |
| | | cttggcctcactgcttaccttattggtaacatgactaatttggtcgtgga |
| | | agggactcgtcgtaccatggaatttaggaataacattgaagcagcttcaa |
| | | actttgttaacagaaacagattgcctcctagattaaaagaccagatttta |
| | | gcttacatgtgtttaaggtttaaagcagagagcttaaatcagcaacatct |
| | | tattgaccagctcccaaaatctatctacaaaagcatttgtcaacatcttt |
| | | ttcttccatctgttgaaaaagtttacctcttcaaaggcgtctcaagagaa |
| | | attcttcttcttctggtttcaaaaatgaaggctgagtatattccaccaag |
| | | agaggatgtcattatgcagaacgaagctccagatgatgtttacattattg |
| | | tgtcaggagaagttgagatcattgattcagagatggagagagagtctgtt |
| | | ttaggcactctacgttgtggagacatttttggagaagttggagcactttg |
| | | ttgcagaccacaaagctacacttttcaaactaagtctttatcacagcttc |
| | | tccgtctcaaaacatctttccttattgagacaatgcagattaaacaacaa |
| | | gacaatgccacaatgctcaagaacttcttgcagcatcacaaaaagctgag |
| | | taatttagacattggtgatctaaaggcacaacaaaatggcgaaaacaccg |
| | | atgttgttcctcctaacattgcctcaaatctcatcgctgtggtgactaca |
| | | ggcaatgcagctcttcttgatgagctacttaaggctaagttaagccctga |
| | | cattacagattccaaaggaaaaactccattgcatgtagcagcttctagag |
| | | gatatgaagattgtgttttagtactcttaaagcacggttgcaacatccac |
| | | attagagatgtgaatggtaatagtgctctatgggaagcaattatttctaa |
| | | gcattacgagattttcagaatcctttatcatttcgcagccatttctgatc |
| | | cacacattgctggagatcttctatgtgaagcagctaaacagaacaatgta |
| | | gaagtcatgaaggctcttttaaaacaggggcttaacgtcgacacagagga |
| | | tcaccatggcgtcacagctttacaggtcgctatggctgaggatcagatgg |
| | | acatggtgaatctcctggctactaacggtgcagatgtagtttgtgtgaat |
| | | acacataatgaattcacaccattggagaagttaagagttgtggaagaaga |
| | | agaagaagaagaacgtggaagagtgagtatttacagaggacatccattgg |
| | | agaggagagaaagaagttgcaatgaagctgggaagcttattcttcttcct |
| | | ccttcacttgatgacctcaagaaaattgcaggagagaagtttgggtttga |
| | | tggaagtgagactatggtgactaatgaagatggagctgagattgacagta |
| | | ttgaagtgattagagataatgacaaactctactttgtcgtaaacaagatt |
| | | atttaggcttatatgaagatgaagatgaaatatttggtgtgtcaaataaa |
| | | aagcttgtgtgcttaagtttgtgtttttttcttggcttgttgtgttatga |
| | | atttgtggctttttctaatattaaatgaatgtaagatctcattataatga |
| | | ataaacaaatgtttctataatccattgtgaatgttttgttggatctcttc |
| | | tgcagcatataactactgtatgtgctatggtatggactatggaattccgc |
| | | tgcaagaggatgcacatgtgaccgagggaatcgggaattaaactatcagt |
| | | gtttgacaggatatattggcgggtaaacctaagagaaaagagcgtttatt |
| | | agaataacggatatttaaaagggcgtgaaaaggtttatccgttcgtccat |
| | | ttgtatgtgcatgccaaccacagggttcccctcgggatcaaagtactttg |
| | | atccaacccctccgctgctatagtgcagtcggcttctgacgttcagtgca |
| | | gccgtgttctgaaaacgacatgtcgcacaagtcctaagttacgcgacagg |
| | | ctgccgccctgcccttttcctggcgttttcttgtcgcgtgttttagtcgc |
| | | ataaagtagaatacttgcgactagaaccggagacattacgccatgaacaa |
| | | gagcgccgccgctggcctgctgggctatgcccgcgtcagcaccgacgacc |
| | | aggacttgaccaaccaacgggccgaactgcacgcggccggctgcaccaag |
| | | ctgttttccgagaagatcaccggcaccaggcgcgaccgcccggagctggc |
| | | caggatgcttgaccacctacgccctggcgacgttgtgacagtgaccaggc |
| | | tagaccgcctggcccgcagcacccgcgacctactggacattgccgagcgc |
| | | atccaggaggccggcgcgggcctgcgtagcctggcagagccgtgggccga |
| | | caccaccacgccggccggccgcatggtgttgaccgtgttcgccggcattg |
| | | ccgagttcgagcgttccctaatcatcgaccgcacccggagcgggcgcgag |
| | | gccgccaaggcccgaggcgtgaagtttggcccccgccctaccctcacccc |
| | | ggcacagatcgcgcacgcccgcgagctgatcgaccaggaaggccgcaccg |
| | | tgaaagaggcggctgcactgcttggcgtgcatcgctcgaccctgtaccgc |
| | | gcacttgagcgcagcgaggaagtgacgcccaccgaggccaggcggcgcgg |
| | | tgccttccgtgaggacgcattgaccgaggccgacgccctggcggccgccg |
| | | agaatgaacgccaagaggaacaagcatgaaaccgcaccaggacggccagg |
| | | acgaaccgtttttcattaccgaagagatcgaggcggagatgatcgcggcc |
| | | gggtacgtgttcgagccgcccgcgcacgtctcaaccgtgcggctgcatga |
| | | aatcctggccggtttgtctgatgccaagctggcggcctggccggccagct |
| | | tggccgctgaagaaaccgagcgccgccgtctaaaaaggtgatgtgtattt |
| | | gagtaaaacagcttgcgtcatgcggtcgctgcgtatatgatgcgatgagt |
| | | aaataaacaaatacgcaaggggaacgcatgaaggttatcgctgtacttaa |
| | | ccagaaaggcgggtcaggcaagacgaccatcgcaacccatctagcccgcg |
| | | ccctgcaactcgccggggccgatgttctgttagtcgattccgatcccc |
| 4 | CDS-HYGROMYCIN | ctatttctttgccctcggacgagtgctggggcgtcggtttccactatcgg |
| | PHOSPHOTRANSFERASE | cgagtacttctacacagccatcggtccagacggccgcgcttctgcgggcg |
| | (Hpt2) | atttgtgtacgcccgacagtcccggctccggatcggacgattgcgtcgca |
| | | tcgaccctgcgcccaagctgcatcatcgaaattgccgtcaaccaagctct |
| | | gatagagttggtcaagaccaatgcggagcatatacgcccggagtcgtggc |
| | | gatcctgcaagctccggatgcctccgctcgaagtagcgcgtctgctgctc |
| | | catacaagccaaccacggcctccagaagaagatgttggcgacctcgtatt |
| | | gggaatccccgaacatcgcctcgctccagtcaatgaccgctgttatgcgg |
| | | ccattgtccgtcaggacattgttggagccgaaatccgcgtgcacgaggtg |
| | | ccggacttcggggcagtcctcggcccaaagcatcagctcatcgagagcct |
| | | gcgcgacggacgcactgacggtgtcgtccatcacagtttgccagtgatac |
| | | acatggggatcagcaatcgcgcatatgaaatcacgccatgtagtgtattg |
| | | accgattccttgcggtccgaatgggccgaacccgctcgtctggctaagat |
| | | cggccgcagcgatcgcatccatagcctccgcgaccggttgtagaacagcg |
| | | ggcagttcggtttcaggcaggtcttgcaacgtgacaccctgtgcacggcg |
| | | ggagatgcaataggtcaggctctcgctaaactccccaatgtcaagcactt |
| | | ccggaatcgggagcgcggccgatgcaaagtgccgataaacataacgatct |
| | | ttgtagaaaccatcggcgcagctatttacccgcaggacatatccacgccc |
| | | tcctacatcgaagctgaaagcacgagattcttcgccctccgagagctgca |
| | | tcaggtcggagacgctgtcgaacttttcgatcagaaacttctcgacagac |
| | | gtcgcggtgagttcaggctttttcatatccggtgcagattatttggattg |
| | | agagtgaatatgagactctaattggataccgaggggaatttatggaacgt |
| | | cagtggagcatttttgacaagaaatatttgctagctgatagtgaccttag |
| | | gcgacttttgaacgcgcaataatggtttctgacgtatgtgcttagctcat |
| | | taaactccagaaacccgcggctgagtggctccttcaacgttgcggttctg |
| | | tcagttccaaacgtaaaacggcttgtcccgcgtcatcggcgggggtcata |
| | | acgtgactcccttaattctccgctcatgatcagattgtcgtttcccgcct |
| | | tcagtttaaactatcagtgtccaacatgttggcaagctgctctagccaat |
| | | acgcaaaccgcctctccccgcgcgttggccgattcattaatgcagctggc |
| | | acgacaggtttcccgactggaaagcgggcagtgagcgcaacgcaattaat |
| | | gtgagttagctcactcattaggcaccccaggctttacactttatgcttcc |
| | | ggctcgtatgttgtgtggaattgtgagcggataacaatttcacacaggaa |
| | | acagctatgaccatgattacgaattc |
| 5 | Promoter-Agrobacterium | taattggataccgaggggaatttatggaacgtcagtggagcatttttgac |
| | tumefaciens NOPALINE | aagaaatatttgctagctgatagtgaccttaggcgacttttgaacgcgca |
| | SYNTHASE (AtuNOS) | ataatggtttctgacgtatgtgcttagctcattaaactccagaaacccgc |
| | | ggctgagtggctccttcaacgttgcggttctgtcagttccaaacgtaaaa |
| | | cggcttgtcccgcgtcatcggcgggggtcataacgtgactcccttaattc |
| | | tccgctcatgatcagattgtcgtttcccgccttcagtttaaactatcagt |
| | | gtccaacatgttggcaagctgctctagccaatacgcaaaccgcctctccc |
| | | cgcgcgttggccgattcattaatgcagctggcacgacaggtttcccgact |
| | | ggaaagcgggcagtgagcgcaacgcaattaatgtgagttagctcactcat |
| | | taggcaccccaggctttacactttatgcttccggctcgtatgttgtgtgg |
| | | aattgtgagcggataacaatttcacacaggaaacagctatgaccatgatt |
| | | acgaattc |
| 6 | p134GG Vector control | cgatccccagggcagtgcccgcgattgggcggccgtgcgggaagatcaac |
| | that lacked the | cgctaaccgttgtcggcatcgaccgcccgacgattgaccgcgacgtgaag |
| | AKT2 cassette | gccatcggccggcgcgacttcgtagtgatcgacggagcgccccaggcggc |
| | | ggacttggctgtgtccgcgatcaaggcagccgacttcgtgctgattccgg |
| | | tgcagccaagcccttacgacatatgggccaccgccgacctggtggagctg |
| | | gttaagcagcgcattgaggtcacggatggaaggctacaagcggcctttgt |
| | | cgtgtcgcgggcgatcaaaggcacgcgcatcggcggtgaggttgccgagg |
| | | cgctggccgggtacgagctgcccattcttgagtcccgtatcacgcagcgc |
| | | gtgagctacccaggcactgccgccgccggcacaaccgttcttgaatcaga |
| | | acccgagggcgacgctgcccgcgaggtccaggcgctggccgctgaaatta |
| | | aatcaaaactcatttgagttaatgaggtaaagagaaaatgagcaaaagca |
| | | caaacacgctaagtgccggccgtccgagcgcacgcagcagcaaggctgca |
| | | acgttggccagcctggcagacacgccagccatgaagcgggtcaactttca |
| | | gttgccggcggaggatcacaccaagctgaagatgtacgcggtacgccaag |
| | | gcaagaccattaccgagctgctatctgaatacatcgcgcagctaccagag |
| | | taaatgagcaaatgaataaatgagtagatgaattttagcggctaaaggag |
| | | gcggcatggaaaatcaagaacaaccaggcaccgacgccgtggaatgcccc |
| | | atgtgtggaggaacgggcggttggccaggcgtaagcggctgggttgtctg |
| | | ccggccctgcaatggcactggaacccccaagcccgaggaatcggcgtgac |
| | | ggtcgcaaaccatccggcccggtacaaatcggcgcggcgctgggtgatga |
| | | cctggtggagaagttgaaggccgcgcaggccgcccagcggcaacgcatcg |
| | | aggcagaagcacgccccggtgaatcgtggcaagcggccgctgatcgaatc |
| | | cgcaaagaatcccggcaaccgccggcagccggtgcgccgtcgattaggaa |
| | | gccgcccaagggcgacgagcaaccagattttttcgttccgatgctctatg |
| | | acgtgggcacccgcgatagtcgcagcatcatggacgtggccgttttccgt |
| | | ctgtcgaagcgtgaccgacgagctggcgaggtgatccgctacgagcttcc |
| | | agacgggcacgtagaggtttccgcagggccggccggcatggccagtgtgt |
| | | gggattacgacctggtactgatggcggtttcccatctaaccgaatccatg |
| | | aaccgataccgggaagggaagggagacaagcccggccgcgtgttccgtcc |
| | | acacgttgcggacgtactcaagttctgccggcgagccgatggcggaaagc |
| | | agaaagacgacctggtagaaacctgcattcggttaaacaccacgcacgtt |
| | | gccatgcagcgtacgaagaaggccaagaacggccgcctggtgacggtatc |
| | | cgagggtgaagccttgattagccgctacaagatcgtaaagagcgaaaccg |
| | | ggcggccggagtacatcgagatcgagctagctgattggatgtaccgcgag |
| | | atcacagaaggcaagaacccggacgtgctgacggttcaccccgattactt |
| | | tttgatcgatcccggcatcggccgttttctctaccgcctggcacgccgcg |
| | | ccgcaggcaaggcagaagccagatggttgttcaagacgatctacgaacgc |
| | | agtggcagcgccggagagttcaagaagttctgtttcaccgtgcgcaagct |
| | | gatcgggtcaaatgacctgccggagtacgatttgaaggaggaggcggggc |
| | | aggctggcccgatcctagtcatgcgctaccgcaacctgatcgagggcgaa |
| | | gcatccgccggttcctaatgtacggagcagatgctagggcaaattgccct |
| | | agcaggggaaaaaggtcgaaaaggtctctttcctgtggatagcacgtaca |
| | | ttgggaacccaaagccgtacattgggaaccggaacccgtacattgggaac |
| | | ccaaagccgtacattgggaaccggtcacacatgtaagtgactgatataaa |
| | | agagaaaaaaggcgatttttccgcctaaaactctttaaaacttattaaaa |
| | | ctcttaaaacccgcctggcctgtgcataactgtctggccagcgcacagcc |
| | | gaagagctgcaaaaagcgcctacccttcggtcgctgcgctccctacgccc |
| | | cgccgcttcgcgtcggcctatcgcggccgctggccgctcaaaaatggctg |
| | | gcctacggccaggcaatctaccagggcgcggacaagccgcgccgtcgcca |
| | | ctcgaccgccggcgcccacatcaaggcaccctgcctcgcgcgtttcggtg |
| | | atgacggtgaaaacctctgacacatgcagctcccggagacggtcacagct |
| | | tgtctgtaagcggatgccgggagcagacaagcccgtcagggcgcgtcagc |
| | | gggtgttggcgggtgtcggggcgcagccatgacccagtcacgtagcgata |
| | | gcggagtgtatactggcttaactatgcggcatcagagcagattgtactga |
| | | gagtgcaccatatgcggtgtgaaataccgcacagatgcgtaaggagaaaa |
| | | taccgcatcaggcgctcttccgcttcctcgctcactgactcgctgcgctc |
| | | ggtcgttcggctgcggcgagcggtatcagctcactcaaaggcggtaatac |
| | | ggttatccacagaatcaggggataacgcaggaaagaacatgtgagcaaaa |
| | | ggccagcaaaaggccaggaaccgtaaaaaggccgcgttgctggcgttttt |
| | | ccataggctccgcccccctgacgagcatcacaaaaatcgacgctcaagtc |
| | | agaggtggcgaaacccgacaggactataaagataccaggcgtttccccct |
| | | ggaagctccctcgtgcgctctcctgttccgaccctgccgcttaccggata |
| | | cctgtccgcctttctcccttcgggaagcgtggcgctttctcatagctcac |
| | | gctgtaggtatctcagttcggtgtaggtcgttcgctccaagctgggctgt |
| | | gtgcacgaaccccccgttcagcccgaccgctgcgccttatccggtaacta |
| | | tcgtcttgagtccaacccggtaagacacgacttatcgccactggcagcag |
| | | ccactggtaacaggattagcagagcgaggtatgtaggcggtgctacagag |
| | | ttcttgaagtggtggcctaactacggctacactagaaggacagtatttgg |
| | | tatctgcgctctgctgaagccagttaccttcggaaaaagagttggtagct |
| | | cttgatccggcaaacaaaccaccgctggtagcggtggtttttttgtttgc |
| | | aagcagcagattacgcgcagaaaaaaaggatctcaagaagatcctttgat |
| | | cttttctacggggtctgacgctcagtggaacgaaaactcacgttaaggga |
| | | ttttggtcatgcattctaggtactaaaacaattcatccagtaaaatataa |
| | | tattttattttctcccaatcaggcttgatccccagtaagtcaaaaaatag |
| | | ctcgacatactgttcttccccgatatcctccctgatcgaccggacgcaga |
| | | aggcaatgtcataccacttgtccgccctgccgcttctcccaagatcaata |
| | | aagccacttactttgccatctttcacaaagatgttgctgtctcccaggtc |
| | | gccgtgggaaaagacaagttcctcttcgggcttttccgtctttaaaaaat |
| | | catacagctcgcgcggatctttaaatggagtatcttcttcccagttttcg |
| | | caatccacatcggccagatcgttattcagtaagtaatccaattcggctaa |
| | | gcggctgtctaagctattcgtatagggacaatccgatatgtcgatggagt |
| | | gaaagagcctgatgcactccgcatacagctcgataatcttttcagggctt |
| | | tgttcatcttcatactcttccgagcaaaggacgccatcggcctcactcat |
| | | gagcagattgctccagccatcatgccgttcaaagtgcaggacctttggaa |
| | | caggcagctttccttccagccatagcatcatgtccttttcccgttccaca |
| | | tcataggtggtccctttataccggctgtccgtcatttttaaatataggtt |
| | | ttcattttctcccaccagcttatataccttagcctgggcctcatgggcct |
| | | tcctttcactgcccgctttccagtcgggaaacctgtcgtgccaggcccac |
| | | atcaaggcaccctgcctcgcgcgtttcggtgatgacggtgaaaacctctg |
| | | acacatgcagctcccggagacggtcacagcttgtctgtaagcggatgccg |
| | | ggagcagacaagcccgtcagggcgcgtcagcgggtgttggcgggtgtcgg |
| | | ggcgcagccatgacccagtcacgtagcgatagcggagtgtatactggctt |
| | | aactatgcggcatcagagcagattgtactgagagtgcaccatatgcggtg |
| | | tgaaataccgcacagatgcgtaaggagaaaataccgcatcaggcgctctt |
| | | ccgcttcctcgctcactgactcgctgcgctcggtcgttcggctgcggcga |
| | | gcggtatcagctcactcaaaggcggtaatacggttatccacagaatcagg |
| | | ggataacgcaggaaagaacatgtgagcaaaaggccagcaaaaggccagga |
| | | accgtaaaaaggccgcgttgctggcgtttttccataggctccgcccccct |
| | | gacgagcatcacaaaaatcgacgctcaagtcagaggtggcgaaacccgac |
| | | aggactataaagataccaggcgtttccccctggaagctccctcgtgcgct |
| | | ctcctgttccgaccctgccgcttaccggatacctgtccgcctttctccct |
| | | tcgggaagcgtggcgctttctcatagctcacgctgtaggtatctcagttc |
| | | ggtgtaggtcgttcgctccaagctgggctgtgtgcacgaaccccccgttc |
| | | agcccgaccgctgcgccttatccggtaactatcgtcttgagtccaacccg |
| | | gtaagacacgacttatcgccactggcagcagccactggtaacaggattag |
| | | cagagcgaggtatgtaggcggtgctacagagttcttgaagtggtggccta |
| | | actacggctacactagaaggacagtatttggtatctgcgctctgctgaag |
| | | ccagttaccttcggaaaaagagttggtagctcttgatccggcaaacaaac |
| | | caccgctggtagcggtggtttttttgtttgcaagcagcagattacgcgca |
| | | gaaaaaaaggatctcaagaagatcctttgatcttttctacggggtctgac |
| | | gctcagtggaacgaaaactcacgttaagggattttggtcatgcattctag |
| | | gtactaaaacaattcatccagtaaaatataatattttattttctcccaat |
| | | caggcttgatccccagtaagtcaaaaaatagctcgacatactgttcttcc |
| | | ccgatatcctccctgatcgaccggacgcagaaggcaatgtcataccactt |
| | | gtccgccctgccgcttctcccaagatcaataaagccacttactttgccat |
| | | ctttcacaaagatgttgctgtctcccaggtcgccgtgggaaaagacaagt |
| | | tcctcttcgggcttttccgtctttaaaaaatcatacagctcgcgcggatc |
| | | tttaaatggagtatcttcttcccagttttcgcaatccacatcggccagat |
| | | cgttattcagtaagtaatccaattcggctaagcggctgtctaagctattc |
| | | gtatagggacaatccgatatgtcgatggagtgaaagagcctgatgcactc |
| | | cgcatacagctcgataatcttttcagggctttgttcatcttcatactctt |
| | | ccgagcaaaggacgccatcggcctcactcatgagcagattgctccagcca |
| | | tcatgccgttcaaagtgcaggacctttggaacaggcagctttccttccag |
| | | ccatagcatcatgtccttttcccgttccacatcataggtggtccctttat |
| | | accggctgtccgtcatttttaaatataggttttcattttctcccaccagc |
| | | ttatataccttagcaggagacattccttccgtatcttttacgcagcggta |
| | | tttttcgatcagttttttcaattccggtgatattctcattttagccattt |
| | | attatttccttcctcttttctacagtatttaaagataccccaagaagcta |
| | | attataacaagacgaactccaattcactgttccttgcattctaaaacctt |
| | | aaataccagaaaacagctttttcaaagttgttttcaaagttggcgtataa |
| | | catagtatcgacggagccgattttgaaaccgcggtgatcacaggcagcaa |
| | | cgctctgtcatcgttacaatcaacatgctaccctccgcgagatcatccgt |
| | | gtttcaaacccggcagcttagttgccgttcttccgaatagcatcggtaac |
| | | atgagcaaagtctgccgccttacaacggctctcccgctgacgccgtcccg |
| | | gactgatgggctgcctgtatcgagtggtgattttgtgccgagctgccggt |
| | | cggggagctgttggctggctggtggcaggatatattgtggtgtaaacaaa |
| | | ttgacgcttagacaacttaataacacattgcggacgtttttaatgtactg |
| | | aattaacgccgaattaattcgggggatctggattttagtactggattttg |
| | | gttttaggaattagaaattttattgatagaagtattttacaaatacaaat |
| | | acatactaagggtttcttatatgctcaacacatgagcgaaaccctatagg |
| | | aaccctaattcccttatctgggaactactcacacattattatggagaaac |
| | | tcgagcttgtcgatcgacagatcccggtcggcatctactctatttctttg |
| | | ccctcggacgagtgctggggcgtcggtttccactatcggcgagtacttct |
| | | acacagccatcggtccagacggccgcgcttctgcgggcgatttgtgtacg |
| | | cccgacagtcccggctccggatcggacgattgcgtcgcatcgaccctgcg |
| | | cccaagctgcatcatcgaaattgccgtcaaccaagctctgatagagttgg |
| | | tcaagaccaatgcggagcatatacgcccggagtcgtggcgatcctgcaag |
| | | ctccggatgcctccgctcgaagtagcgcgtctgctgctccatacaagcca |
| | | accacggcctccagaagaagatgttggcgacctcgtattgggaatccccg |
| | | aacatcgcctcgctccagtcaatgaccgctgttatgcggccattgtccgt |
| | | caggacattgttggagccgaaatccgcgtgcacgaggtgccggacttcgg |
| | | ggcagtcctcggcccaaagcatcagctcatcgagagcctgcgcgacggac |
| | | gcactgacggtgtcgtccatcacagtttgccagtgatacacatggggatc |
| | | agcaatcgcgcatatgaaatcacgccatgtagtgtattgaccgattcctt |
| | | gcggtccgaatgggccgaacccgctcgtctggctaagatcggccgcagcg |
| | | atcgcatccatagcctccgcgaccggttgtagaacagcgggcagttcggt |
| | | ttcaggcaggtcttgcaacgtgacaccctgtgcacggcgggagatgcaat |
| | | aggtcaggctctcgctaaactccccaatgtcaagcacttccggaatcggg |
| | | agcgcggccgatgcaaagtgccgataaacataacgatctttgtagaaacc |
| | | atcggcgcagctatttacccgcaggacatatccacgccctcctacatcga |
| | | agctgaaagcacgagattcttcgccctccgagagctgcatcaggtcggag |
| | | acgctgtcgaacttttcgatcagaaacttctcgacagacgtcgcggtgag |
| | | ttcaggctttttcatatccggtgcagattatttggattgagagtgaatat |
| | | gagactctaattggataccgaggggaatttatggaacgtcagtggagcat |
| | | ttttgacaagaaatatttgctagctgatagtgaccttaggcgacttttga |
| | | acgcgcaataatggtttctgacgtatgtgcttagctcattaaactccaga |
| | | aacccgcggctgagtggctccttcaacgttgcggttctgtcagttccaaa |
| | | cgtaaaacggcttgtcccgcgtcatcggcgggggtcataacgtgactccc |
| | | ttaattctccgctcatgatcagattgtcgtttcccgccttcagtttaaac |
| | | tatcagtgtccaacatgttggcaagctgctctagccaatacgcaaaccgc |
| | | ctctccccgcgcgttggccgattcattaatgcagctggcacgacaggttt |
| | | cccgactggaaagcgggcagtgagcgcaacgcaattaatgtgagttagct |
| | | cactcattaggcaccccaggctttacactttatgcttccggctcgtatgt |
| | | tgtgtggaattgtgagcggataacaatttcacacaggaaacagctatgac |
| | | catgattacgaattctgccatgtcttcgagctcggtacccggggatccat |
| | | tacaggtgaccagctcgaatttcgaagacatgggaatcgggaattaaact |
| | | atcagtgtttgacaggatatattggcgggtaaacctaagagaaaagagcg |
| | | tttattagaataacggatatttaaaagggcgtgaaaaggtttatccgttc |
| | | gtccatttgtatgtgcatgccaaccacagggttcccctcgggatcaaagt |
| | | actttgatccaacccctccgctgctatagtgcagtcggcttctgacgttc |
| | | agtgcagccgtgttctgaaaacgacatgtcgcacaagtcctaagttacgc |
| | | gacaggctgccgccctgcccttttcctggcgttttcttgtcgcgtgtttt |
| | | agtcgcataaagtagaatacttgcgactagaaccggagacattacgccat |
| | | gaacaagagcgccgccgctggcctgctgggctatgcccgcgtcagcaccg |
| | | acgaccaggacttgaccaaccaacgggccgaactgcacgcggccggctgc |
| | | accaagctgttttccgagaagatcaccggcaccaggcgcgaccgcccgga |
| | | gctggccaggatgcttgaccacctacgccctggcgacgttgtgacagtga |
| | | ccaggctagaccgcctggcccgcagcacccgcgacctactggacattgcc |
| | | gagcgcatccaggaggccggcgcgggcctgcgtagcctggcagagccgtg |
| | | ggccgacaccaccacgccggccggccgcatggtgttgaccgtgttcgccg |
| | | gcattgccgagttcgagcgttccctaatcatcgaccgcacccggagcggg |
| | | cgcgaggccgccaaggcccgaggcgtgaagtttggcccccgccctaccct |
| | | caccccggcacagatcgcgcacgcccgcgagctgatcgaccaggaaggcc |
| | | gcaccgtgaaagaggcggctgcactgcttggcgtgcatcgctcgaccctg |
| | | taccgcgcacttgagcgcagcgaggaagtgacgcccaccgaggccaggcg |
| | | gcgcggtgccttccgtgaggacgcattgaccgaggccgacgccctggcgg |
| | | ccgccgagaatgaacgccaagaggaacaagcatgaaaccgcaccaggacg |
| | | gccaggacgaaccgtttttcattaccgaagagatcgaggcggagatgatc |
| | | gcggccgggtacgtgttcgagccgcccgcgcacgtctcaaccgtgcggct |
| | | gcatgaaatcctggccggtttgtctgatgccaagctggcggcctggccgg |
| | | ccagcttggccgctgaagaaaccgagcgccgccgtctaaaaaggtgatgt |
| | | gtatttgagtaaaacagcttgcgtcatgcggtcgctgcgtatatgatgcg |
| | | atgagtaaataaacaaatacgcaaggggaacgcatgaaggttatcgctgt |
| | | acttaaccagaaaggcgggtcaggcaagacgaccatcgcaacccatctag |
| | | cccgcgccctgcaactcgccggggccgatgttctgttagtcgattc |
| 7 | 912..2018 | atggcactggaacccccaagcccgaggaatcggcgtgacggtcgcaaacc |
| | (Transformation | atccggcccggtacaaatcggcgcggcgctgggtgatgacctggtggaga |
| | plasmid and vector | agttgaaggccgcgcaggccgcccagcggcaacgcatcgaggcagaagca |
| | control) | cgccccggtgaatcgtggcaagcggccgctgatcgaatccgcaaagaatc |
| | | ccggcaaccgccggcagccggtgcgccgtcgattaggaagccgcccaagg |
| | | gcgacgagcaaccagattttttcgttccgatgctctatgacgtgggcacc |
| | | cgcgatagtcgcagcatcatggacgtggccgttttccgtctgtcgaagcg |
| | | tgaccgacgagctggcgaggtgatccgctacgagcttccagacgggcacg |
| | | tagaggtttccgcagggccggccggcatggccagtgtgtgggattacgac |
| | | ctggtactgatggcggtttcccatctaaccgaatccatgaaccgataccg |
| | | ggaagggaagggagacaagcccggccgcgtgttccgtccacacgttgcgg |
| | | acgtactcaagttctgccggcgagccgatggcggaaagcagaaagacgac |
| | | ctggtagaaacctgcattcggttaaacaccacgcacgttgccatgcagcg |
| | | tacgaagaaggccaagaacggccgcctggtgacggtatccgagggtgaag |
| | | ccttgattagccgctacaagatcgtaaagagcgaaaccgggcggccggag |
| | | tacatcgagatcgagctagctgattggatgtaccgcgagatcacagaagg |
| | | caagaacccggacgtgctgacggttcaccccgattactttttgatcgatc |
| | | ccggcatcggccgttttctctaccgcctggcacgccgcgccgcaggcaag |
| | | gcagaagccagatggttgttcaagacgatctacgaacgcagtggcagcgc |
| | | cggagagttcaagaagttctgtttcaccgtgcgcaagctgatcgggtcaa |
| | | atgacctgccggagtacgatttgaaggaggaggcggggcaggctggcccg |
| | | atcctagtcatgcgctaccgcaacctgatcgagggcgaagcatccgccgg |
| | | ttcctaatgtacggagcagatgctagggcaaattgccctagcaggggaaa |
| | | aaggtcgaaaaggtctctttcctgtggatagcacgtacattgggaaccca |
| | | aagccgtacattgggaaccggaacccgtacattgggaacccaaagccgta |
| | | cattgggaaccggtcacacatgtaagtgactgatataaaagagaaaaaag |
| | | gcgatttttccgcctaaaactctttaaaacttattaaaactcttaaaacc |
| | | cgcctggcctgtgcataactgtctggccagcgcacagccgaagagctgca |
| | | aaaagcgcctacccttcggtcgctgcgctccctacgccccgccgcttcgc |
| | | gtcggcctatcgcggccgctggccgctcaaaaatggctggcctacggcca |
| | | ggcaatctaccagggcgcggacaagccgcgccgtcgccactcgaccgccg |
| | | gcgcccacatcaaggcaccctgcctcgcgcgtttcggtgatgacggtgaa |
| | | aacctctgacacatgcagctcccggagacggtcacagcttgtctgtaagc |
| | | ggatgccgggagcagacaagcccgtcagggcgcgtcagcgggtgttggcg |
| | | ggtgtcggggcgcagccatgacccagtcacgtagcgatagcggagtgtat |
| | | actggcttaactatgcggcatcagagcagattgtactgagagtgcaccat |
| | | atgcggtgtgaaataccgcacagatgcgtaaggagaaaataccgcatcag |
| | | gcgctcttccgcttcctcgctcactgactcgctgcgctcggtcgttcggc |
| | | tgcggcgagcggtatcagctcactcaaaggcggtaatacggttatccaca |
| | | gaatcaggggataacgcaggaaagaacatgtgagcaaaaggccagcaaaa |
| | | ggccaggaaccgtaaaaa |
| 8 | 5554..6348 | ctaaaacaattcatccagtaaaatataatattttattttctcccaatcag |
| | (Transformation | gcttgatccccagtaagtcaaaaaatagctcgacatactgttcttccccg |
| | plasmid and vector | atatcctccctgatcgaccggacgcagaaggcaatgtcataccacttgtc |
| | control) | cgccctgccgcttctcccaagatcaataaagccacttactttgccatctt |
| | | tcacaaagatgttgctgtctcccaggtcgccgtgggaaaagacaagttcc |
| | | tcttcgggcttttccgtctttaaaaaatcatacagctcgcgcggatcttt |
| | | aaatggagtatcttcttcccagttttcgcaatccacatcggccagatcgt |
| | | tattcagtaagtaatccaattcggctaagcggctgtctaagctattcgta |
| | | tagggacaatccgatatgtcgatggagtgaaagagcctgatgcactccgc |
| | | atacagctcgataatcttttcagggctttgttcatcttcatactcttccg |
| | | agcaaaggacgccatcggcctcactcatgagcagattgctccagccatca |
| | | tgccgttcaaagtgcaggacctttggaacaggcagctttccttccagcca |
| | | tagcatcatgtccttttcccgttccacatcataggtggtccctttatacc |
| | | ggctgtccgtcatttttaaatataggttttcattttctcccaccagctta |
| | | tataccttagcaggagacattccttccgtatcttttacgcagcggtattt |
| | | ttcgatcagttttttcaattccggtgatattctcattttagccatttatt |
| | | atttccttcctcttttctacagtatttaaagataccccaagaagctaatt |
| | | ataacaagacgaactccaattcactgttccttgcattctaaaaccttaaa |
| | | taccagaaaacagctttttcaaagttgttttcaaagttggcgtataacat |
| | | agtatcgacggagccgattttgaaaccgcggtgatcacaggcagcaacgc |
| | | tctgtcatcgttacaatcaacatgctaccctccgcgagatcatccgtgtt |
| | | tcaaacccggcagcttagttgccgttcttccgaatagcatcggtaacatg |
| | | agcaaagtctgccgccttacaacggctctcccgctgacgccgtcccggac |
| | | tgatgggctgcctgtatcgagtggtgattttgtgccgagctgccggtcgg |
| | | ggagctgttggctggctggtggcaggatatattgtggtgtaaacaaattg |
| | | acgcttagacaacttaataacacattgcggacgtttttaatgtactgaat |
| | | taacgccgaattaattcgggggatctggattttagtactggattttggtt |
| | | ttaggaattagaaattttattgatagaagtattttacaaatacaaataca |
| | | tactaagggtttcttatatgctcaacacatgagcgaaaccctataggaac |
| | | cctaattcccttatctgggaactactcacacattattatggagaaactcg |
| | | agcttgtcgatcgacagatcccggtcggcatctactctatttctttgccc |
| | | tcggacgagtgctggggcgtcggtttccactatcggcgagtacttctaca |
| | | cagccatcggtccagacggccgcgcttctgcgggcgatttgtgtacgccc |
| | | gacagtcccggctccggatcggacgattgcgtcgcatcgaccctgcgccc |
| | | aagctgcatcatcgaaattgccgtcaaccaagctctgatagagttggtca |
| | | agaccaatgcggagcatatacgcccggagtcgtggcgatcctgcaagctc |
| | | cggatgcctccgctcgaagtagcgcgtctgctgctccatacaagccaacc |
| | | acggcctccagaagaagatgttggcgacctcgtattgggaatccccgaac |
| | | atcgcctcgctccagtcaatgaccgctgttatgcggccattgtccgtcag |
| | | gacattgttggagccgaaatccgcgtgcacgaggtgccggacttcggggc |
| | | agtcctcggcccaaagcatcagctcatcgagagcctgcgcgacggacgca |
| | | ctgacggtgtcgtccatcacagtttgccagtgatacacatggggatcagc |
| | | aatcgcgcatatgaaatcacgccatgtagtgtattgaccgattccttgcg |
| | | gtccgaatgggccgaacccgctcgtctggctaagatcggccgcagcgatc |
| | | gcatccatagcctccgcgaccggttgtagaacagcgggcagttcggtttc |
| | | aggcaggtcttgcaacgtgacaccctgtgcacggcgggagatgcaatagg |
| | | tcaggctctcgctaaactccccaatgtcaagcacttccggaatcgggagc |
| | | gcggccgatgcaaagtgccgataaacataacgatctttgtagaaaccatc |
| | | ggcgcagctatttacccgcaggacatatccacgccctcctacatcgaagc |
| | | tgaaagcacgagattcttcgccctccgagagctgcatcaggtcggagacg |
| | | ctgtcgaacttttcgatcagaaacttctcgacagacgtcgcggtgagttc |
| | | aggctttttcatatccggtgcagattatttggattgagagtgaatatgag |
| | | actctaattggataccgaggggaatttatggaacgtcagtggagcatttt |
| | | tgacaagaaatatttgctagctgatagtgaccttaggcgacttttgaacg |
| | | cgcaataatggtttctgacgtatgtgcttagctcattaaactccagaaac |
| | | ccgcggctgagtggctccttcaacgttgcggttctgtcagttccaaacgt |
| | | aaaacggcttgtcccgcgtcatcgggggggtcataacgtgactccctta |
| | | attctccgctcatgatcagattgtcgtttcccgccttcagtttaaactat |
| | | cagtgtccaacatgttggcaagctgctctagccaatacgcaaaccgcctc |
| | | tccccgcgcgttggccgattcattaatgcagctggcacgacaggtttccc |
| | | gactggaaagcgggcagtgagcgcaacgcaattaatgtgagttagctcac |
| | | tcattaggcaccccaggctttacactttatgcttccggctcgtatgttgt |
| | | gtggaattgtgagcggataacaatttcacacaggaaacagctatgaccat |
| | | gattacgaattctgccatgtcttcgagctcggtacccggggatccattac |
| | | aggtgaccagctcgaatttcgaagacatgggaatcgggaattaaactatc |
| | | agtgtttgacaggatatattggcgggtaaacctaagagaaaagagcgttt |
| | | attagaataacggatatttaaaagggcgtgaaaaggtttatccgttcgtc |
| | | catttgtatgtgcatgccaaccacagggttcccctcgggatcaaagtact |
| | | ttgatccaacccctccgctgctatagtgcagtcggcttctgacgttcagt |
| | | gcagccgtgttctgaaaacgacatgtcgcacaagtcctaagttacgcgac |
| | | aggctgccgccctgcccttttcctggcgttttcttgtcgcgtgttttagt |
| | | cgcataaagtagaatacttgcgactagaaccggagacattacgccatgaa |
| | | caagagcgccgccgctggcctgctgggctatgcccgcgtcagcaccgacg |
| | | accaggacttgaccaaccaacgggccgaactgcacgcggccggctgcacc |
| | | aagctgttttccgagaagatcaccggcaccaggcgcgaccgcccggagct |
| | | ggccaggatgcttgaccacctacgccctggcgacgttgtgacagtgacca |
| | | ggctagaccgcctggcccgcagcacccgcgacctactggacattgccgag |
| | | cgcatccaggaggccggcgcgggcctgcgtagcctggcagagccgtgggc |
| | | cgacaccaccacgccggccggccgcatggtgttgaccgtgttcgccggca |
| | | ttgccgagttcgagcgttccctaatcatcgaccgcacccggaggcggcgc |
| | | gaggccgccaaggcccgaggcgtgaagtttggcccccgccctaccctcac |
| | | cccggcacagatcgcgcacgcccgcgagctgatcgaccaggaaggccgca |
| | | ccgtgaaagaggcggctgcactgcttggcgtgcatcgctcgaccctgtac |
| | | cgcgcacttgagcgcagcgaggaagtgacgcccaccgaggccaggcggcg |
| | | cggtgccttccgtgaggacgcattgaccgaggccgacgccctggcggccg |
| | | ccgagaatgaacgccaagaggaacaagcatgaaaccgcaccaggacggcc |
| | | aggacgaaccgtttttcattaccgaagagatcgaggcggagatgatcgcg |
| | | gccgggtacgtgttcgagccgcccgcgcacgtctcaaccgtgcggctgca |
| | | tgaaatcctggccggtttgtctgatgccaagctggcggcctggccggcca |
| | | gcttggccgctgaagaaaccgagcgccgccgtctaaaaaggtgatgtgta |
| | | tttgagtaaaacagcttgcgtcatgcggtcgctgcgtatatgatgcgatg |
| | | agtaaataaacaaatacgcaaggggaacgcatgaaggttatcgctgtact |
| | | taaccagaaaggcgggtcaggcaagacgaccatcgcaacccatctagccc |
| | | gcgccctgcaactcgccggggccgatgttctgttagtcgattc |
| 9 | 7090..8115 | ctatttctttgccctcggacgagtgctggggcgtcggtttccactatcgg |
| | (Transformation | cgagtacttctacacagccatcggtccagacggccgcgcttctgcgggcg |
| | plasmid and vector | atttgtgtacgcccgacagtcccggctccggatcggacgattgcgtcgca |
| | control) | tcgaccctgcgcccaagctgcatcatcgaaattgccgtcaaccaagctct |
| | | gatagagttggtcaagaccaatgcggagcatatacgcccggagtcgtggc |
| | | gatcctgcaagctccggatgcctccgctcgaagtagcgcgtctgctgctc |
| | | catacaagccaaccacggcctccagaagaagatgttggcgacctcgtatt |
| | | gggaatccccgaacatcgcctcgctccagtcaatgaccgctgttatgcgg |
| | | ccattgtccgtcaggacattgttggagccgaaatccgcgtgcacgaggtg |
| | | ccggacttcggggcagtcctcggcccaaagcatcagctcatcgagagcct |
| | | gcgcgacggacgcactgacggtgtcgtccatcacagtttgccagtgatac |
| | | acatggggatcagcaatcgcgcatatgaaatcacgccatgtagtgtattg |
| | | accgattccttgcggtccgaatgggccgaacccgctcgtctggctaagat |
| | | cggccgcagcgatcgcatccatagcctccgcgaccggttgtagaacagcg |
| | | ggcagttcggtttcaggcaggtcttgcaacgtgacaccctgtgcacggcg |
| | | ggagatgcaataggtcaggctctcgctaaactccccaatgtcaagcactt |
| | | ccggaatcgggagcgcggccgatgcaaagtgccgataaacataacgatct |
| | | ttgtagaaaccatcggcgcagctatttacccgcaggacatatccacgccc |
| | | tcctacatcgaagctgaaagcacgagattcttcgccctccgagagctgca |
| | | tcaggtcggagacgctgtcgaacttttcgatcagaaacttctcgacagac |
| | | gtcgcggtgagttcaggctttttcatatccggtgcagattatttggattg |
| | | agagtgaatatgagactctaattggataccgaggggaatttatggaacgt |
| | | cagtggagcatttttgacaagaaatatttgctagctgatagtgaccttag |
| | | gcgacttttgaacgcgcaataatggtttctgacgtatgtgcttagctcat |
| | | taaactccagaaacccgcggctgagtggctccttcaacgttgcggttctg |
| | | tcagttccaaacgtaaaacggcttgtcccgcgtcatcggcgggggtcata |
| | | acgtgactcccttaattctccgctcatgatcagattgtcgtttcccgcct |
| | | tcagtttaaactatcagtgtccaacatgttggcaagctgctctagccaat |
| | | acgcaaaccgcctctccccgcgcgttggccgattcattaatgcagctggc |
| | | acgacaggtttcccgactggaaagcgggcagtgagcgcaacgcaattaat |
| | | gtgagttagctcactcattaggcaccccaggctttacactttatgcttcc |
| | | ggctcgtatgttgtgtggaattgtgagcggataacaatttcacacaggaa |
| | | acagctatgaccatgattacgaattctgccatgtcttcgagctcggtacc |
| | | cggggatccattacaggtgaccagctcgaatttcgaagacatgggaatcg |
| | | ggaattaaactatcagtgtttgacaggatatattggcgggtaaacctaag |
| | | agaaaagagcgtttattagaataacggatatttaaaagggcgtgaaaagg |
| | | tttatccgttcgtccatttgtatgtgcatgccaaccacagggttcccctc |
| | | gggatcaaagtactttgatccaacccctccgctgctatagtgcagtcggc |
| | | ttctgacgttcagtgcagccgtgttctgaaaacgacatgtcgcacaagtc |
| | | ctaagttacgcgacaggctgccgccctgcccttttcctggcgttttcttg |
| | | tcgcgtgttttagtcgcataaagtagaatacttgcgactagaaccggaga |
| | | cattacgccatgaacaagagcgccgccgctggcctgctgggctatgcccg |
| | | cgtcagcaccgacgaccaggacttgaccaaccaacgggccgaactgcacg |
| | | cggccggctgcaccaagctgttttccgagaagatcaccggcaccaggcgc |
| | | gaccgcccggagctggccaggatgcttgaccacctacgccctggcgacgt |
| | | tgtgacagtgaccaggctagaccgcctggcccgcagcacccgcgacctac |
| | | tggacattgccgagcgcatccaggaggccggcgcgggcctgcgtagcctg |
| | | gcagagccgtgggccgacaccaccacgccggccggccgcatggtgttgac |
| | | cgtgttcgccggcattgccgagttcgagcgttccctaatcatcgaccgca |
| | | cccggagcgggcgcgaggccgccaaggcccgaggcgtgaagtttggcccc |
| | | cgccctaccctcaccccggcacagatcgcgcacgcccgcgagctgatcga |
| | | ccaggaaggccgcaccgtgaaagaggcggctgcactgcttggcgtgcatc |
| | | gctcgaccctgtaccgcgcacttgagcgcagcgaggaagtgacgcccacc |
| | | gaggccaggcggcgcggtgccttccgtgaggacgcattgaccgaggccga |
| | | cgccctggcggccgccgagaatgaacgccaagaggaacaagcatgaaacc |
| | | gcaccaggacggccaggacgaaccgtttttcattaccgaagagatcgagg |
| | | cggagatgatcgcggccgggtacgtgttcgagccgcccgcgcacgtctca |
| | | accgtgcggctgcatgaaatcctggccggtttgtctgatgccaagctggc |
| | | ggcctggccggccagcttggccgctgaagaaaccgagcgccgccgtctaa |
| | | aaaggtgatgtgtatttgagtaaaacagcttgcgtcatgcggtcgctgcg |
| | | tatatgatgcgatgagtaaataaacaaatacgcaaggggaacgcatgaag |
| | | gttatcgctgtacttaaccagaaaggcgggtcaggcaagacgaccatcgc |
| | | aacccatctagcccgcgccctgcaactcgccggggccgatgttctgttag |
| | | tcgattc |
| 10 | 8158..8337 | taattggataccgaggggaatttatggaacgtcagtggagcatttttgac |
| | (Transformation | aagaaatatttgctagctgatagtgaccttaggcgacttttgaacgcgca |
| | plasmid and vector | ataatggtttctgacgtatgtgcttagctcattaaactccagaaacccgc |
| | control) | ggctgagtggctccttcaacgttgcggttctgtcagttccaaacgtaaaa |
| | | cggcttgtcccgcgtcatcggcgggggtcataacgtgactcccttaattc |
| | | tccgctcatgatcagattgtcgtttcccgccttcagtttaaactatcagt |
| | | gtccaacatgttggcaagctgctctagccaatacgcaaaccgcctctccc |
| | | cgcgcgttggccgattcattaatgcagctggcacgacaggtttcccgact |
| | | ggaaagcgggcagtgagcgcaacgcaattaatgtgagttagctcactcat |
| | | taggcaccccaggctttacactttatgcttccggctcgtatgttgtgtgg |
| | | aattgtgagcggataacaatttcacacaggaaacagctatgaccatgatt |
| | | acgaattctgccatgtcttcgagctcggtacccggggatccattacaggt |
| | | gaccagctcgaatttcgaagacatgggaatcgggaattaaactatcagtg |
| | | tttgacaggatatattggcgggtaaacctaagagaaaagagcgtttatta |
| | | gaataacggatatttaaaagggcgtgaaaaggtttatccgttcgtccatt |
| | | tgtatgtgcatgccaaccacagggttcccctcgggatcaaagtactttga |
| | | tccaacccctccgctgctatagtgcagtcggcttctgacgttcagtgcag |
| | | ccgtgttctgaaaacgacatgtcgcacaagtcctaagttacgcgacaggc |
| | | tgccgccctgcccttttcctggcgttttcttgtcgcgtgttttagtcgca |
| | | taaagtagaatacttgcgactagaaccggagacattacgccatgaacaag |
| | | agcgccgccgctggcctgctgggctatgcccgcgtcagcaccgacgacca |
| | | ggacttgaccaaccaacgggccgaactgcacgcggccggctgcaccaagc |
| | | tgttttccgagaagatcaccggcaccaggcgcgaccgcccggagctggcc |
| | | aggatgcttgaccacctacgccctggcgacgttgtgacagtgaccaggct |
| | | agaccgcctggcccgcagcacccgcgacctactggacattgccgagcgca |
| | | tccaggaggccggcgcgggcctgcgtagcctggcagagccgtgggccgac |
| | | accaccacgccggccggccgcatggtgttgaccgtgttcgccggcattgc |
| | | cgagttcgagcgttccctaatcatcgaccgcacccggagcgggcgcgagg |
| | | ccgccaaggcccgaggcgtgaagtttggcccccgccctaccctcaccccg |
| | | gcacagatcgcgcacgcccgcgagctgatcgaccaggaaggccgcaccgt |
| | | gaaagaggcggctgcactgcttggcgtgcatcgctcgaccctgtaccgcg |
| | | cacttgagcgcagcgaggaagtgacgcccaccgaggccaggcggcgcggt |
| | | gccttccgtgaggacgcattgaccgaggccgacgccctggcggccgccga |
| | | gaatgaacgccaagaggaacaagcatgaaaccgcaccaggacggccagga |
| | | cgaaccgtttttcattaccgaagagatcgaggcggagatgatcgcggccg |
| | | ggtacgtgttcgagccgcccgcgcacgtctcaaccgtgcggctgcatgaa |
| | | atcctggccggtttgtctgatgccaagctggcggcctggccggccagctt |
| | | ggccgctgaagaaaccgagcgccgccgtctaaaaaggtgatgtgtatttg |
| | | agtaaaacagcttgcgtcatgcggtcgctgcgtatatgatgcgatgagta |
| | | aataaacaaatacgcaaggggaacgcatgaaggttatcgctgtacttaac |
| | | cagaaaggcgggtcaggcaagacgaccatcgcaacccatctagcccgcgc |
| | | cctgcaactcgccggggccgatgttctgttagtcgattc |
| 11 | 8622..8652 | tttacactttatgcttccggctcgtatgttgtgtggaattgtgagcggat |
| | (Transformation | aacaatttcacacaggaaacagctatgaccatgattacgaattctgccat |
| | plasmid and vector | gtcttcgagctcggtacccggggatccattacaggtgaccagctcgaatt |
| | control) | tcgaagacatgggaatcgggaattaaactatcagtgtttgacaggatata |
| | | ttggcgggtaaacctaagagaaaagagcgtttattagaataacggatatt |
| | | taaaagggcgtgaaaaggtttatccgttcgtccatttgtatgtgcatgcc |
| | | aaccacagggttcccctcgggatcaaagtactttgatccaacccctccgc |
| | | tgctatagtgcagtcggcttctgacgttcagtgcagccgtgttctgaaaa |
| | | cgacatgtcgcacaagtcctaagttacgcgacaggctgccgccctgccct |
| | | tttcctggcgttttcttgtcgcgtgttttagtcgcataaagtagaatact |
| | | tgcgactagaaccggagacattacgccatgaacaagagcgccgccgctgg |
| | | cctgctgggctatgcccgcgtcagcaccgacgaccaggacttgaccaacc |
| | | aacgggccgaactgcacgcggccggctgcaccaagctgttttccgagaag |
| | | atcaccggcaccaggcgcgaccgcccggagctggccaggatgcttgacca |
| | | cctacgccctggcgacgttgtgacagtgaccaggctagaccgcctggccc |
| | | gcagcacccgcgacctactggacattgccgagcgcatccaggaggccggc |
| | | gcgggcctgcgtagcctggcagagccgtgggccgacaccaccacgccggc |
| | | cggccgcatggtgttgaccgtgttcgccggcattgccgagttcgagcgtt |
| | | ccctaatcatcgaccgcacccggagcgggcgcgaggccgccaaggcccga |
| | | ggcgtgaagtttggcccccgccctaccctcaccccggcacagatcgcgca |
| | | cgcccgcgagctgatcgaccaggaaggccgcaccgtgaaagaggcggctg |
| | | cactgcttggcgtgcatcgctcgaccctgtaccgcgcacttgagcgcagc |
| | | gaggaagtgacgcccaccgaggccaggcggcgcggtgccttccgtgagga |
| | | cgcattgaccgaggccgacgccctggcggccgccgagaatgaacgccaag |
| | | aggaacaagcatgaaaccgcaccaggacggccaggacgaaccgtttttca |
| | | ttaccgaagagatcgaggcggagatgatcgcggccgggtacgtgttcgag |
| | | ccgcccgcgcacgtctcaaccgtgcggctgcatgaaatcctggccggttt |
| | | gtctgatgccaagctggcggcctggccggccagcttggccgctgaagaaa |
| | | ccgagcgccgccgtctaaaaaggtgatgtgtatttgagtaaaacagcttg |
| | | cgtcatgcggtcgctgcgtatatgatgcgatgagtaaataaacaaatacg |
| | | caaggggaacgcatgaaggttatcgctgtacttaaccagaaaggcgggtc |
| | | aggcaagacgaccatcgcaacccatctagcccgcgccctgcaactcgccg |
| | | gggccgatgttctgttagtcgattc |
| 12 | 10134..517 | atgaaggttatcgctgtacttaaccagaaaggcgggtcaggcaagacgac |
| | (Transformation | catcgcaacccatctagcccgcgccctgcaactcgccggggccgatgttc |
| | plasmid and vector | tgttagtcgattccgatccccagggcagtgcccgcgattgggcggccgtg |
| | control) | cgggaagatcaaccgctaaccgttgtcggcatcgaccgcccgacgattga |
| | | ccgcgacgtgaaggccatcggccggcgcgacttcgtagtgatcgacggag |
| | | cgccccaggcggcggacttggctgtgtccgcgatcaaggcagccgacttc |
| | | gtgctgattccggtgcagccaagcccttacgacatatgggccaccgccga |
| | | cctggtggagctggttaagcagcgcattgaggtcacggatggaaggctac |
| | | aagcggcctttgtcgtgtcgcgggcgatcaaaggcacgcgcatcggcggt |
| | | gaggttgccgaggcgctggccgggtacgagctgcccattcttgagtcccg |
| | | tatcacgcagcgcgtgagctacccaggcactgccgccgccggcacaaccg |
| | | ttcttgaatcagaacccgagggcgacgctgcccgcgaggtccaggcgctg |
| | | gccgctgaaattaaatcaaaactcatttga |
| 13 | AKT2-fwd: 5′- | TGGCTACTAACGGTGCAGAT |
| | TGGCTACTAA | |
| | CGGTGCAGAT-3 | |
| 14 | AKT2-rev: 5′- | ACCCAAACTTCTCTCCTGCA |
| | ACCCAAACTT | |
| | CTCTCCTGCA-3′ | |
| 15 | MeGAPDH-fwd: 5′- | TCTTCGGCGTTAGGAACCCAG |
| | TCTTCGGCG | |
| | TTAGGAACCCAG-3′ | |
| 16 | MeGAPDH-rev: | GCAGCCTTATCCTTGTCGGTG |
| | GCAGCCTTATC | |
| | CTTGTCGGTG | |
| 17 | AtAKT2 | MDLKYSASHCNLSSDMKLRRFHQHRGKGRE |
| | (unmodified)_At4g22200 | EEYDASSLSLNNLSKLILPPLGVASYNQNH |
| | | IRSSGWIISPMDSRYRCWEFYMVLLVAYSA |
| | | WVYPFEVAFLNSSPKRNLCIADNIVDLFFA |
| | | VDIVLTFFVAYIDERTQLLVREPKQIAVRY |
| | | LSTWFLMDVASTIPFDAIGYLITGTSTLNI |
| | | TCNLLGLLRFWRLRRVKHLFTRLEKDIRYS |
| | | YFWIRCFRLLSVTLFLVHCAGCSYYLIADR |
| | | YPHQGKTWTDAIPNFTETSLSIRYIAAIYW |
| | | SITTMTTVGYGDLHASNTIEMVFITVYMLF |
| | | NLGLTAYLIGNMTNLVVEGTRRTMEFRNSI |
| | | EAASNFVNRNRLPPRLKDQILAYMCLRFKA |
| | | ESLNQQHLIDQLPKSIYKSICQHLFLPSVE |
| | | KVYLFKGVSREILLLLVSKMKAEYIPPRED |
| | | VIMQNEAPDDVYIIVSGEVEIIDSEMERES |
| | | VLGTLRCGDIFGEVGALCCRPQSYTFQTKS |
| | | LSQLLRLKTSFLIETMQIKQQDNATMLKNF |
| | | LQHHKKLSNLDIGDLKAQQNGENTDVVPPN |
| | | IASNLIAVVTTGNAALLDELLKAKLSPDIT |
| | | DSKGKTPLHVAASRGYEDCVLVLLKHGCNI |
| | | HIRDVNGNSALWEAIISKHYEIFRILYHFA |
| | | AISDPHIAGDLLCEAAKQNNVEVMKALLKQ |
| | | GLNVDTEDHHGVTALQVAMAEDQMDMVNLL |
| | | ATNGADVVCVNTHNEFTPLEKLRVVEEEEE |
| | | EERGRVSIYRGHPLERRERSCNEAGKLILL |
| | | PPSLDDLKKIAGEKFGFDGSETMVTNEDGA |
| | | EIDSIEVIRDNDKLYFVVNKII |
| 18 | S210N + S329N in | MDLKYSASHCNLSSDMKLRRFHQHRGKGRE |
| | SEQ ID NO: 17 | EEYDASSLSLNNLSKLILPPLGVASYNQNH |
| | | IRSSGWIISPMDSRYRCWEFYMVLLVAYSA |
| | | WVYPFEVAFLNSSPKRNLCIADNIVDLFFA |
| | | VDIVLTFFVAYIDERTQLLVREPKQIAVRY |
| | | LSTWFLMDVASTIPFDAIGYLITGTSTLNI |
| | | TCNLLGLLRFWRLRRVKHLFTRLEKDIRYN |
| | | YFWIRCFRLLSVTLFLVHCAGCSYYLIADR |
| | | YPHQGKTWTDAIPNFTETSLSIRYIAAIYW |
| | | SITTMTTVGYGDLHASNTIEMVFITVYMLF |
| | | NLGLTAYLIGNMTNLVVEGTRRTMEFRNNI |
| | | EAASNFVNRNRLPPRLKDQILAYMCLRFKA |
| | | ESLNQQHLIDQLPKSIYKSICQHLFLPSVE |
| | | KVYLFKGVSREILLLLVSKMKAEYIPPRED |
| | | VIMQNEAPDDVYIIVSGEVEIIDSEMERES |
| | | VLGTLRCGDIFGEVGALCCRPQSYTFQTKS |
| | | LSQLLRLKTSFLIETMQIKQQDNATMLKNF |
| | | LQHHKKLSNLDIGDLKAQQNGENTDVVPPN |
| | | IASNLIAVVTTGNAALLDELLKAKLSPDIT |
| | | DSKGKTPLHVAASRGYEDCVLVLLKHGCNI |
| | | HIRDVNGNSALWEAIISKHYEIFRILYHFA |
| | | AISDPHIAGDLLCEAAKQNNVEVMKALLKQ |
| | | GLNVDTEDHHGVTALQVAMAEDQMDMVNLL |
| | | ATNGADVVCVNTHNEFTPLEKLRVVEEEEE |
| | | EERGRVSIYRGHPLERRERSCNEAGKLILL |
| | | PPSLDDLKKIAGEKFGFDGSETMVTNEDGA |
| | | EIDSIEVIRDNDKLYFVVNKII |
| 19 | >tr\|A0A2C9VK43\| | MEMKSSWENHHEEKKQSNHYEEDDTSLSLS |
| | A0A2C9VK43_MANES | SLSKIILPPLGVSSYNHNPIETKGWIISPM |
| | Potassium channel | NSKYRCWETYMVVLVAYSAWVSPFEVAFLK |
| | OS = Manihot esculenta | SNPNKGLYVADSVVDLFFAIDIVLTFFVAY |
| | OX = 3983 | IDSTTHLMVRDRRKISIRYLSTWFSMDVAS |
| | GN = MANES_07G018900 | TIPFEALGYLFTGKRKMGLSYSLLGMLRFW |
| | PE = 3 SV = 1 (MeAKT2a, | RLRRVKQLFTRLEKDIRFSYFWVRCTRLLF |
| | unmodified) | VTLLLVHCAGCLCYLLADRYPHQGRTWLGS |
| | | VNPNFRETSLRNRYISALYWSVTTMTTVGY |
| | | GDLHAVNTGEMIFIIFYMLFNLGLTAYLIG |
| | | NMTNLVVEGTRRTMEFRNSIEAASNFVCRN |
| | | RLPPRLKEQILAYMCLRFKAESLNQNHLIE |
| | | QLPKSICKCICQHLFLPIAEKVYLFKGVSR |
| | | EILLLLVAEMKAEYIPPREDVIMQNEAPDD |
| | | VYIIVSGEVEIIDSALEKERIFGILQSGDM |
| | | FGEVGALCCKPQSFTFRTKTLSQLLKLKTS |
| | | ALIETMQIKQEDYVAIIKNFLQHHKKLKDF |
| | | KIGEFIAEGGEEDGDPNMAFNLLTAASAGN |
| | | AAFLEELLRAKLDPDIGDSKGRTPLHFAAS |
| | | KGHEDCALALLRHGCNIHLKDVNGNTALWE |
| | | ALSSKHQSVFRILYHFANVSDPHTAGDLLC |
| | | TAAKRNDLTMMNSLLKHGLNVDSKDRQGKT |
| | | AVQIAMAQNYIDMVDLLVMNGADVSAANSS |
| | | EFCSTTLNKMLQRRESGHRITMPDTVTSDE |
| | | VILKMDQEEKQCKSSEKSNELKYTRVSIYR |
| | | GHPLVRKETCCRQAGRLIRLPNSMEELKSI |
| | | AGEKFRFDARNAMVTDEEGSEIDSIEVIRD |
| | | NDKLFIVEDPTPFM |
| 20 | >tr\|A0A2C9V5L9\| | MEMRSTPSNDLYHLPFTMKRSWRNHHGHPQ |
| | A0A2C9V5L9_MANES | TPHHHHHQEDDTSLSVSSLSKIILPPLGVS |
| | Potassium channel | SYNHNPVETKGWIVSPMNSKYRCWETFMVV |
| | OS = Manihot esculenta | LVAYSAWVYPFEVAFLNSSPNKMLYITDNI |
| | OX = 3983 | VDLFFAIDIVLTFFVAYIDSRTQLLVRDRT |
| | GN = MANES_10G122000 | KISIRYLSTWFLMDVASTIPFEALAYFFTG |
| | PE = 3 SV = 1 (MeAKT2b, | KHSMGLSYSLLGMLRFWRLRRVKQLFTRLE |
| | unmodified) | KDIRFSYFWIRCARLIIVTLFLVHCAGCLY |
| | | YLLADRYPHQGRTWIGAVIPNFRETSLWIR |
| | | YISALYWSITTMTTVGYGDLHAVNTMEMIF |
| | | IIFYMLFNLGLTAYLIGNMTNLVVEGTRRT |
| | | MEFRNSIEAASNFVCRNRLPPRLKEQILAY |
| | | MCLRFKAESLNQNHLIEQLPKSICKSICHH |
| | | LFLPTVEKVYLFSGVSREILLLLVAEMKAE |
| | | YIPPREDVIMQNEAPDDVYIIVSGEVEIID |
| | | SDLEKELVVGTLQSGDMFGEVGALCCKVQS |
| | | FTFRTKTLSQLLKLKTSTLIDTMQTKQEDY |
| | | VAIIKNFLQHHKKLKGLKLGESLVDDGEED |
| | | GDPNMAFNLLTVASTGNAAFLEELLRAKLD |
| | | PDIGDSKGRTPLHVAASKGHEDCVLALLRH |
| | | GCNINLRDVNGNTALWEALSSKHQSVFRIL |
| | | YHFSNIDDPHTAGELLCKAAKENDLTMMKE |
| | | LLKHGLNVDAKDRQGKTAVQIAMAQNYVDM |
| | | VDLLVMNGADVSAANTSEFSSTTLNEMLQK |
| | | REIGHRITVPDTVTSDEVILKRNQEEEEGN |
| | | SSGKSNGWECRRVSIYRGHPLIRKETCCLE |
| | | PGRLIRLPNSMEELKSIAGEKFGFDARNAM |
| | | VTDEEGSEIDSIEVIRDNDKLFIVEDPNSSM |
| 21 | AtAKT2 (FIG. 20B) | TGTSTLNITCNLLGLLRFWRLRRVKHLFTR |
| | | LEKDIRYSYFWIRCFRLLSVTLFLVHCAGC |
| | | SYYLIADRYPHQGKTWTDAIPNFTETSLSI |
| | | RYIAAIYWSITTMTTVGYGDLHASNTIEMV |
| | | FITVYMLFNLGLTAYLIGNMTNLVVEGTRR |
| | | TMEFRNSIEAASNFVNRNRLPPRLKDQI |
| 22 | MeAKT2a (FIG. 20B) | TGKRKMGLSYSLLGMLRFWRLRRVKQLFTR |
| | | LEKDIRFSYFWVRCTRLLFVTLLLVHCAGC |
| | | LCYLLADRYPHQGRTWLGSVNPNFRETSLR |
| | | NRYISALYWSVTTMTTVGYGDLHAVNTGEM |
| | | IFIIFYMLFNLGLTAYLIGNMTNLVVEGTR |
| | | RTMEFRNSIEAASNFVCRNRLPPRLKEQI |
| 23 | MeAKT2b (FIG. 20B) | TGKHSMGLSYSLLGMLRFWRLRRVKQLFTR |
| | | LEKDIRFSYFWIRCARLIIVTLFLVHCAGC |
| | | LYYLLADRYPHQGRTWIGAVIPNFRETSLW |
| | | IRYISALYWSITTMTTVGYGDLHAVNTMEM |
| | | IFIIFYMLFNLGLTAYLIGNMTNLVVEGTR |
| | | RTMEFRNSIEAASNFVCRNRLPPRLKEQI |
| 24 | FIG. 20B consensus | TGKXXMGLSYSLLGMLRFWRLRRVKQLFTR |
| | sequence | LEKDIRFSYFWIRCXRLLXVTLFLVHCAGC |
| | | LYYLLADRYPHQGRTWXGAVIPNFRETSLX |
| | | IRYISALYWSITTMTTVGYGDLHAVNTXEM |
| | | IFIIFYMLFNLGLTAYLIGNMTNLVVEGTR |
| | | RTMEFRNSIEAASNFVCRNRLPPRLKEQI |
| 25 | S199N and S319N in | MEMKSSWENHHEEKKQSNHYEEDDTSLSLS |
| | SEQ ID NO: 19 | SLSKIILPPLGVSSYNHNPIETKGWIISPM |
| | | NSKYRCWETYMVVLVAYSAWVSPFEVAFLK |
| | | SNPNKGLYVADSVVDLFFAIDIVLTFFVAY |
| | | IDSTTHLMVRDRRKISIRYLSTWFSMDVAS |
| | | TIPFEALGYLFTGKRKMGLSYSLLGMLRFW |
| | | RLRRVKQLFTRLEKDIRFNYFWVRCTRLLF |
| | | VTLLLVHCAGCLCYLLADRYPHQGRTWLGS |
| | | VNPNFRETSLRNRYISALYWSVTTMTTVGY |
| | | GDLHAVNTGEMIFIIFYMLFNLGLTAYLIG |
| | | NMTNLVVEGTRRTMEFRNNIEAASNFVCRN |
| | | RLPPRLKEQILAYMCLRFKAESLNQNHLIE |
| | | QLPKSICKCICQHLFLPIAEKVYLFKGVSR |
| | | EILLLLVAEMKAEYIPPREDVIMQNEAPDD |
| | | VYIIVSGEVEIIDSALEKERIFGILQSGDM |
| | | FGEVGALCCKPQSFTFRTKTLSQLLKLKTS |
| | | ALIETMQIKQEDYVAIIKNFLQHHKKLKDF |
| | | KIGEFIAEGGEEDGDPNMAFNLLTAASAGN |
| | | AAFLEELLRAKLDPDIGDSKGRTPLHFAAS |
| | | KGHEDCALALLRHGCNIHLKDVNGNTALWE |
| | | ALSSKHQSVFRILYHFANVSDPHTAGDLLC |
| | | TAAKRNDLTMMNSLLKHGLNVDSKDRQGKT |
| | | AVQIAMAQNYIDMVDLLVMNGADVSAANSS |
| | | EFCSTTLNKMLQRRESGHRITMPDTVTSDE |
| | | VILKMDQEEKQCKSSEKSNELKYTRVSIYR |
| | | GHPLVRKETCCRQAGRLIRLPNSMEELKSI |
| | | AGEKFRFDARNAMVTDEEGSEIDSIEVIRD |
| | | NDKLFIVEDPTPFM |
| 26 | S216N and S336N in | MEMRSTPSNDLYHLPFTMKRSWRNHHGHPQ |
| | ID NO: 20 | TPHHHHHQEDDTSLSVSSLSKIILPPLGVS |
| | | SYNHNPVETKGWIVSPMNSKYRCWETFMVV |
| | | LVAYSAWVYPFEVAFLNSSPNKMLYITDNI |
| | | VDLFFAIDIVLTFFVAYIDSRTQLLVRDRT |
| | | KISIRYLSTWFLMDVASTIPFEALAYFFTG |
| | | KHSMGLSYSLLGMLRFWRLRRVKQLFTRLE |
| | | KDIRFNYFWIRCARLIIVTLFLVHCAGCLY |
| | | YLLADRYPHQGRTWIGAVIPNFRETSLWIR |
| | | YISALYWSITTMTTVGYGDLHAVNTMEMIF |
| | | IIFYMLFNLGLTAYLIGNMTNLVVEGTRRT |
| | | MEFRNNIEAASNFVCRNRLPPRLKEQILAY |
| | | MCLRFKAESLNQNHLIEQLPKSICKSICHH |
| | | LFLPTVEKVYLFSGVSREILLLLVAEMKAE |
| | | YIPPREDVIMQNEAPDDVYIIVSGEVEIID |
| | | SDLEKELVVGTLQSGDMFGEVGALCCKVQS |
| | | FTFRTKTLSQLLKLKTSTLIDTMQTKQEDY |
| | | VAIIKNFLQHHKKLKGLKLGESLVDDGEED |
| | | GDPNMAFNLLTVASTGNAAFLEELLRAKLD |
| | | PDIGDSKGRTPLHVAASKGHEDCVLALLRH |
| | | GCNINLRDVNGNTALWEALSSKHQSVFRIL |
| | | YHFSNIDDPHTAGELLCKAAKENDLTMMKE |
| | | LLKHGLNVDAKDRQGKTAVQIAMAQNYVDM |
| | | VDLLVMNGADVSAANTSEFSSTTLNEMLQK |
| | | REIGHRITVPDTVTSDEVILKRNQEEEEGN |
| | | SSGKSNGWECRRVSIYRGHPLIRKETCCLE |
| | | PGRLIRLPNSMEELKSIAGEKFGFDARNAM |
| | | VTDEEGSEIDSIEVIRDNDKLFIVEDPNSSM |
| 27 | MeGAPDH forward | TCTTCGGCGTTAGGAACCCAG |
| 28 | MeGAPDH reverse | GCAGCCTTATCCTTGTCGGTG |
| 29 | AtAKT2 forward | ACAGGGGCTTAACGTCGACAC |
| 30 | AtAKT2 reverse | TGCACCGTTAGTAGCCAGGAGA |
| 31 | AKT2 CDS_RT_F1 | CAGCTTCTTGTCCGTGAACC |
| 32 | AKT2 CDS_RT_R1 | AGGTAAGCAGTGAGGCCAAG |

Having generally described the compositions, methods, and processes of this disclosure, the same will be better understood by reference to certain specific examples, which are included herein to further illustrate the disclosure and are not intended to limit the scope of the invention as defined by the claims.

### EXAMPLES

The present disclosure is described in further detail in the following examples which are not in any way intended to limit the scope of the disclosure as claimed. The attached figures are meant to be considered as integral parts of the specification and description of the disclosure. The following examples are offered to illustrate, but not to limit the claimed disclosure.

### Example 1: Potassium Fertilization LED to an Increase of Storage Root Mass by Shifting Sugar Distribution

The following example describes experiments that showed that the increasing potassium availability resulted in increased growth and yield, as well as altered sugar and ion distribution within the plant.

### Introduction

Potassium, as one of the major plant nutrients, is a key factor for crop yield. The cation is a major active solute in plants with a key function in maintaining turgor pressure and driving changes in cell volume. Potassium is involved in many metabolic processes and also serves as an important enzymatic cofactor. More importantly, potassium is increasingly recognized as a key factor for influencing phloem mass flow. A recent high-impact study in maize for instance showed that plants with compromised phloem sugar loading (knockout of the phloem loader SUT1) can compensate with increased levels of phloem potassium, thereby maintaining phloem pressure and decent sap flow speeds (Babst et al., 2022. Sugar loading is not required for phloem sap flow in maize plants. Nature Plants, 8, 171-180). Another high-impact study has implicated potassium in the energization of phloem membrane transport (Gajdanowicz et al., 2011. Potassium (K⁺) gradients serve as a mobile energy source in plant vascular tissues. Proc Natl Acad Sci USA, 108, 864-9.).

The importance of potassium fertilization for cassava storage root yield has been demonstrated in numerous studies (to cite just a few recent studies: Chua et al., 2020. Potassium Fertilisation Is Required to Sustain Cassava Yield and Soil Fertility. Agronomy [Online], 10; Fernandes et al., 2017. Yield and nutritional requirements of cassava in response to potassium fertilizer in the second cycle. Journal of Plant Nutrition, 40, 2785-2796.; Gazola et al., 2022. Potassium management effects on yield and quality of cassava varieties in tropical sandy soils. Crop and Pasture Science, 73, 285-299.; Sukkaew et al., 2022. Response of cassava (Manihot esculenta Crantz) to calcium and potassium in a humid tropical upland loamy sand soil. Annals of Agricultural Sciences, 67, 204-210.). Van Laere et al. (Van Laere et al., 2023. Carbon allocation in cassava is affected by water deficit and potassium application—A (13) C—CO(2) pulse labelling assessment. Rapid Commun Mass Spectrom, 37, e9426.) recently also showed that potassium application can improve carbon allocation to storage roots.

Improvements to phloem loading and transport through increased AKT2 activity could therefore be a viable way to increase the delivery of assimilates to cassava storage roots, thereby improving storage root yield.

### Methods

### Plant Growth

Unmodified cassava (Manihot esculenta) cultivar TMS60444 (“60444”) was used. Field trials in open field space were conducted at National Chung Hsing University, Taichung, Taiwan.

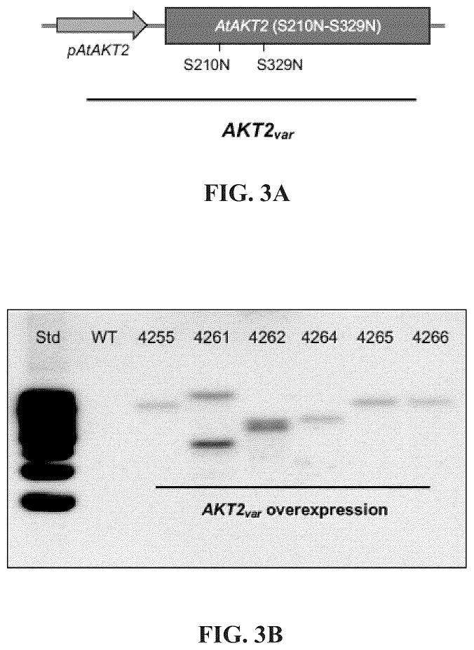

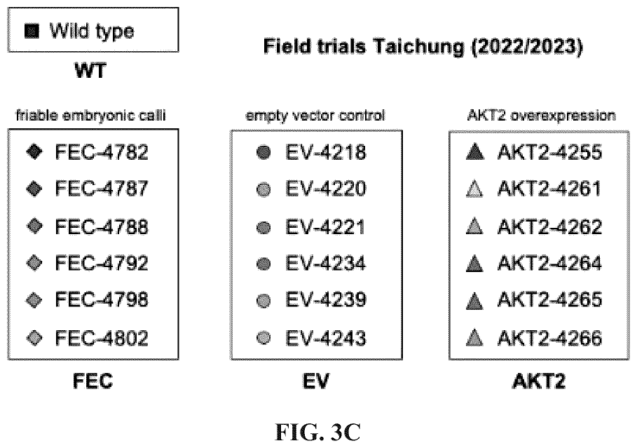

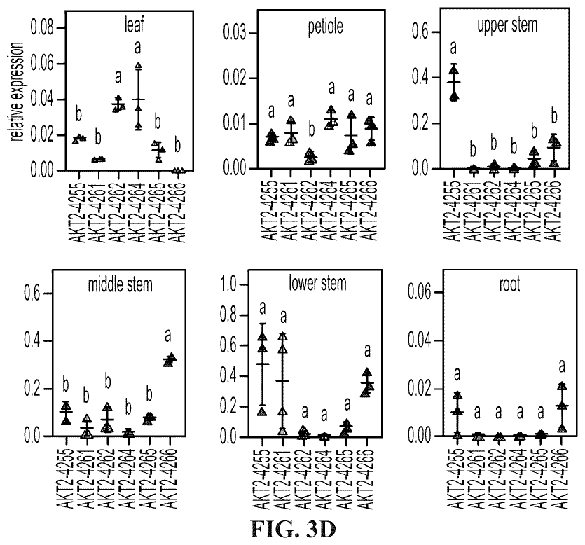

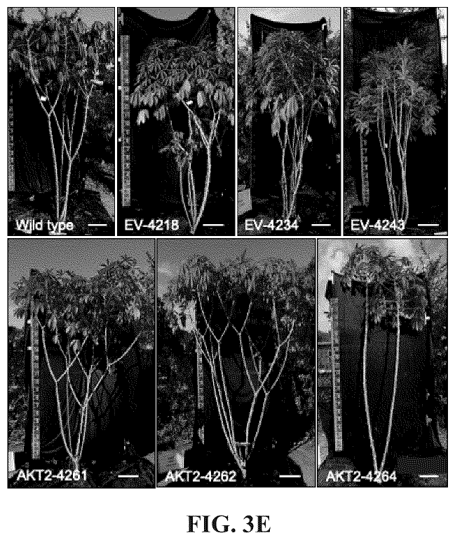

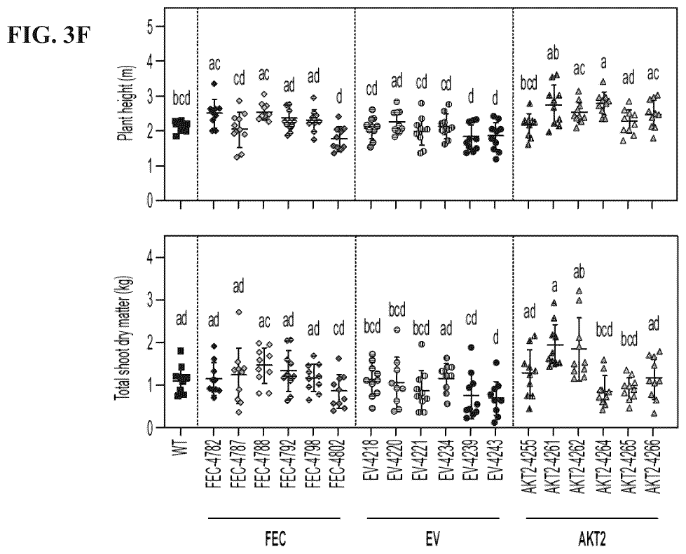

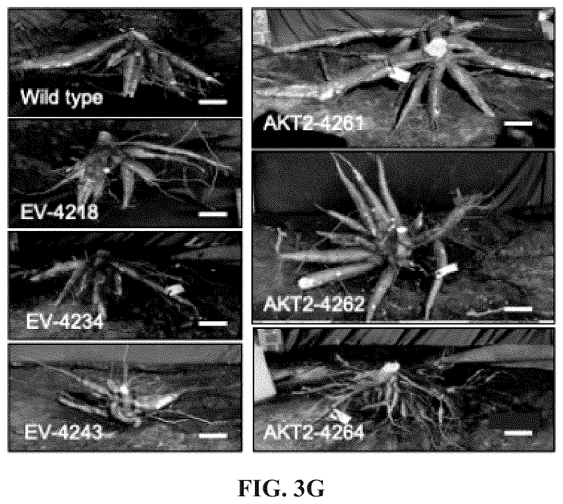

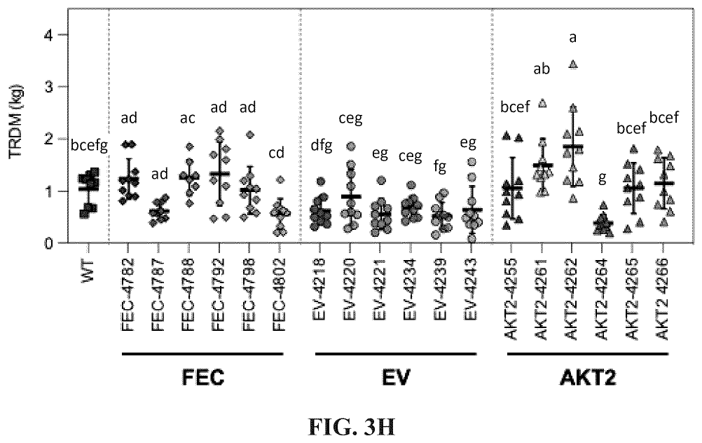

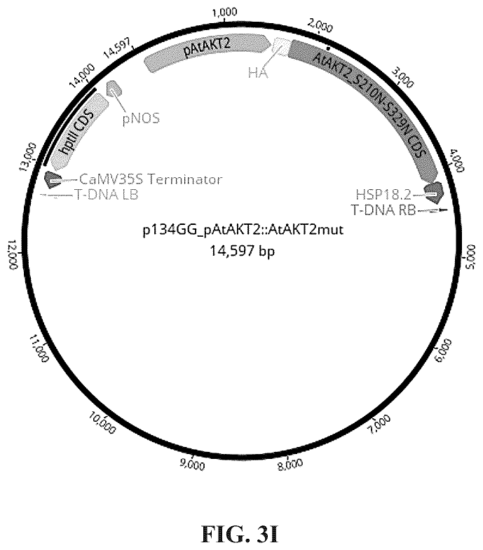

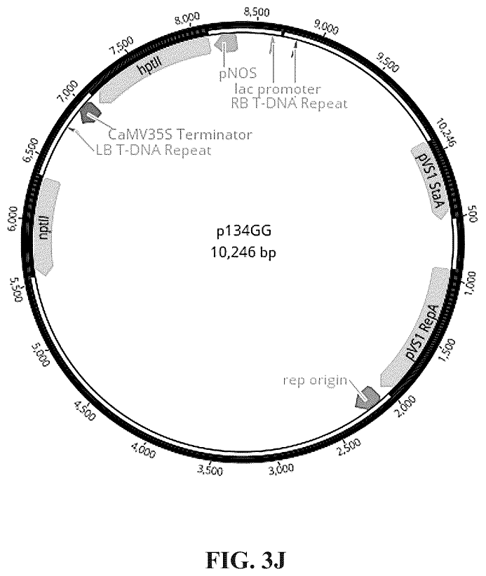

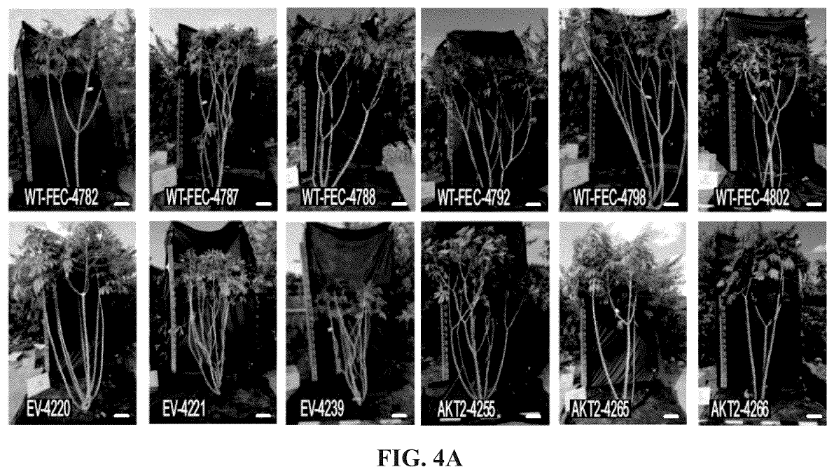

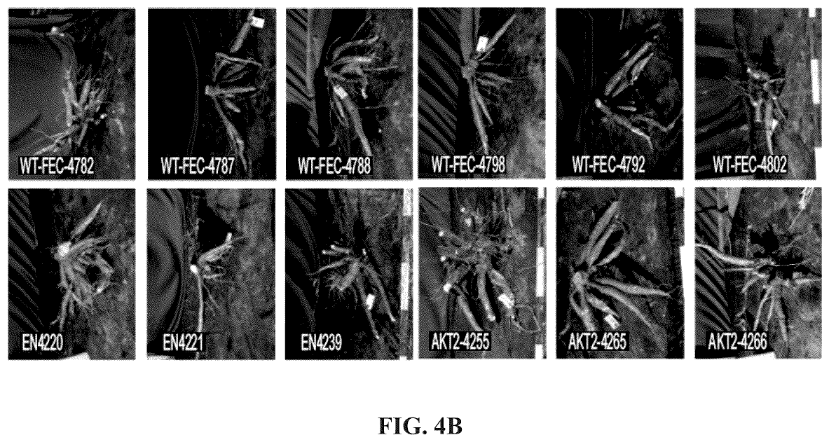

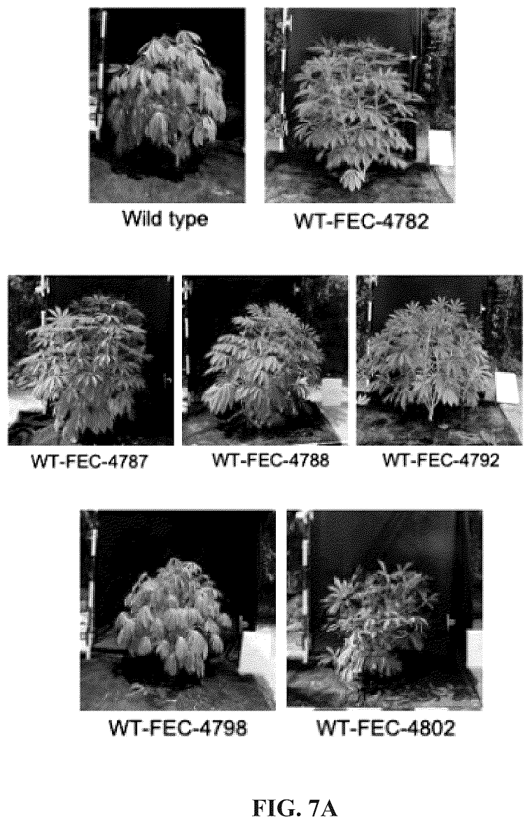

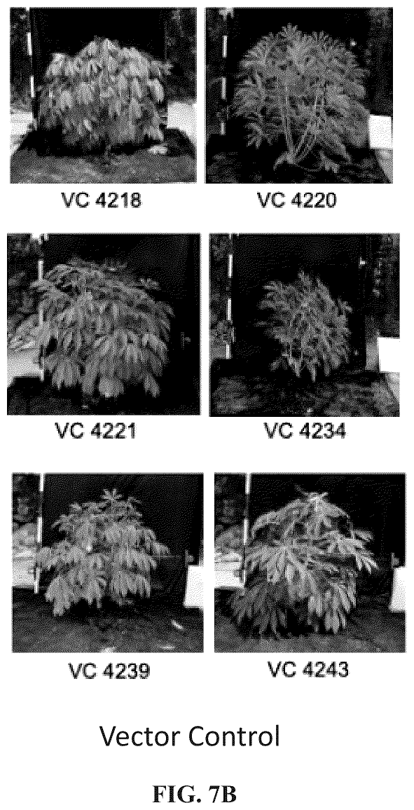

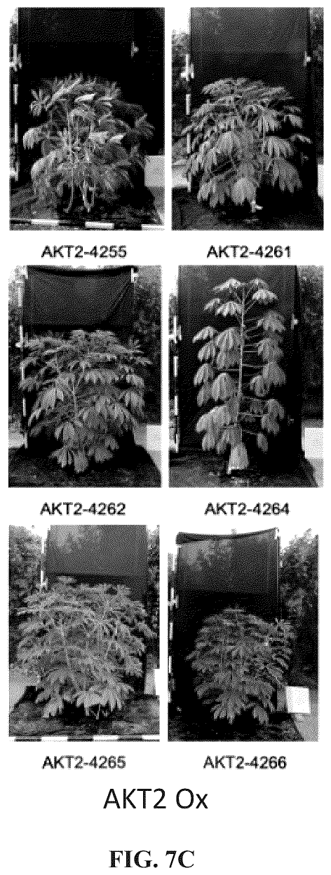

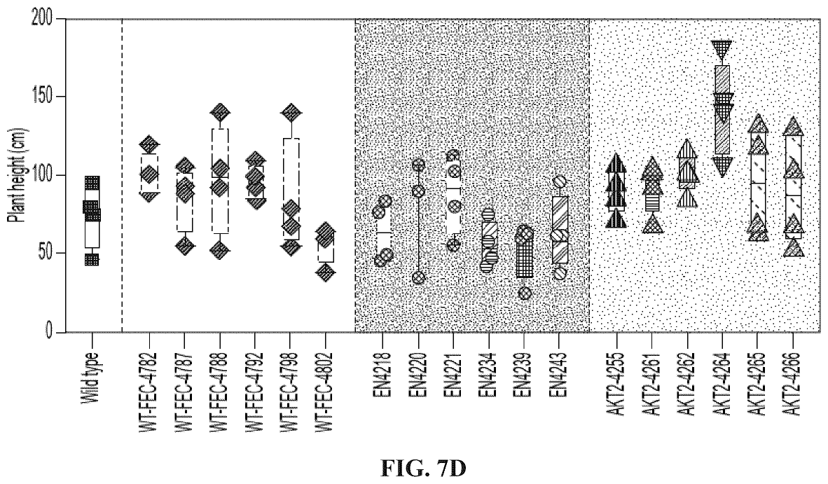

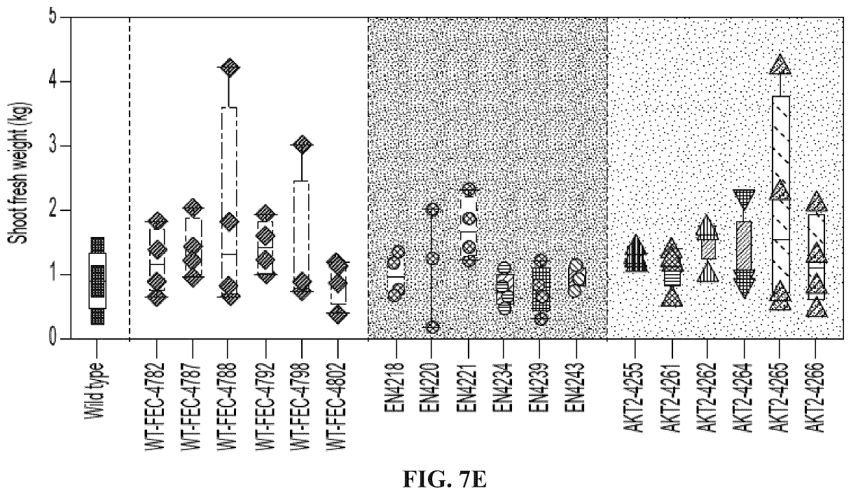

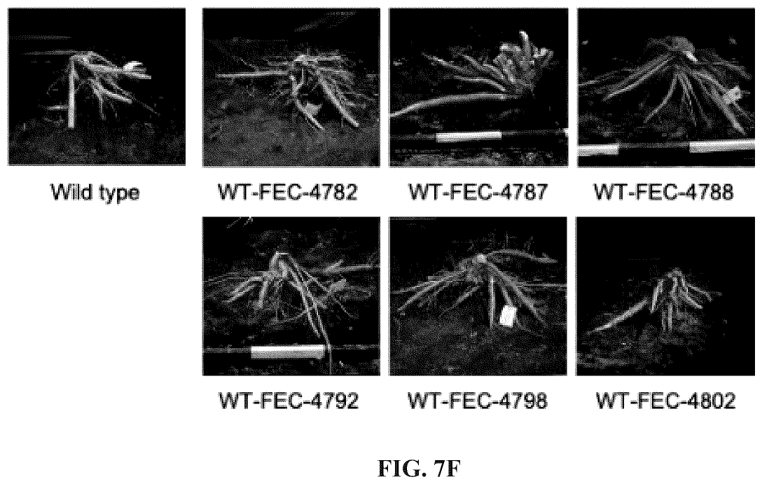

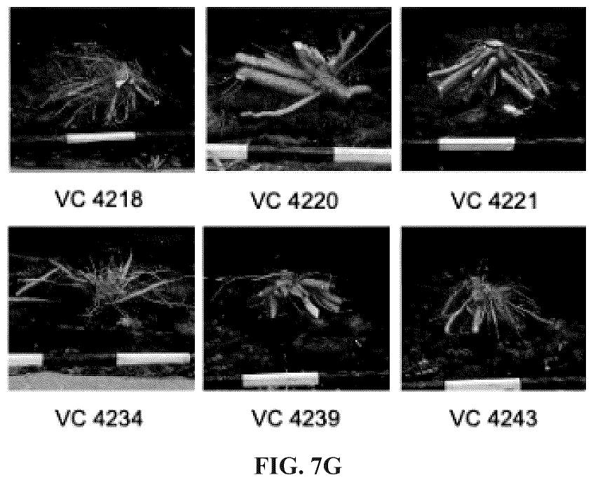

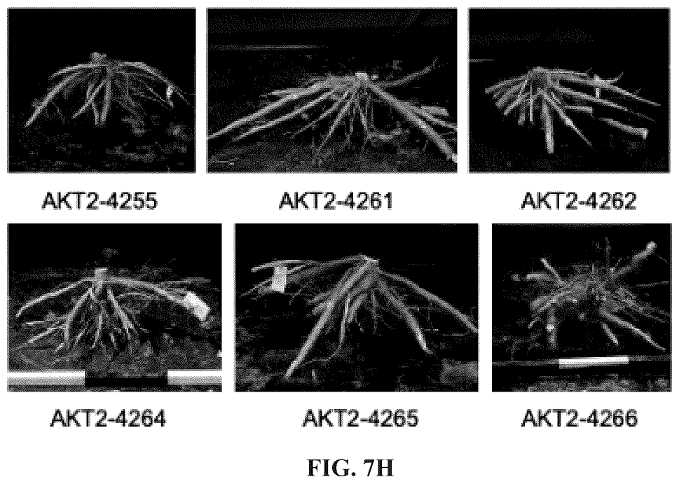

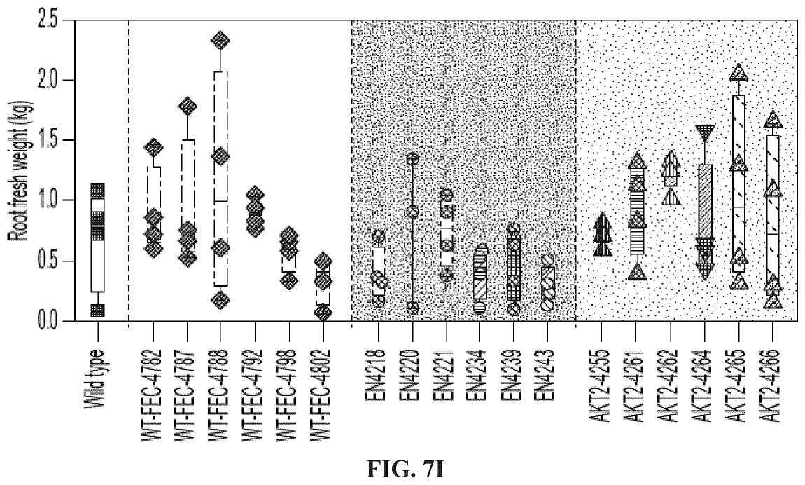

Sterile unmodified cassava 60444 (control) and transgenic 60444 AtAKT2var overexpression (AKT2) in vitro cassava plantlets were transferred from agar growth medium into pots with soil, and grown and hardened in the greenhouse for two months. Soil-grown cassava plants from the greenhouse were then planted in prepared ridged open fields and grown from mid-March to the end of November. Unmodified cassava 60444 (control) and transgenic 60444 (AKT2) plants were grown with sufficient replica numbers to enable robust statistical analysis of agronomic performance and storage root yield data. A selected number (typically 4-6) of unmodified cassava 60444 (control) and transgenic 60444 (AKT2) plant replicas were harvested manually in July (intermediate harvest) for a first assessment for agronomic performance, storage root yield, and biochemical analysis (FIGS. 7A-7C, 7F-7H). The remainder (typically 8-12) of the unmodified cassava 60444 (control) and transgenic 60444 (AKT2) plant replicas were machine-harvested at the end of November (final harvest) for the final assessment of agronomic performance and storage root yield (FIGS. 3E, 3G, 4A, 4B). Environmental data (temperature, sunshine, humidity, rain) were collected regularly during the entire growth period. Field soil parameters (organic matter, nitrogen, phosphate, sulfur, potassium, other iron/micronutrients) were measured before planting of the unmodified cassava 60444 (control) and transgenic 60444 (AKT2) plant replicas into the field in March and after the final harvest at the end of November. Fertilizer was added to the field soil as necessary to provide consistent field soil parameters for the growing season during each year.

### Plant Harvesting and Processing.

All greenhouse-grown plants were harvested after a total growth period of 17 weeks. During harvesting, the height of each plant was measured before the plants were separated into three partitions: leaves, stems, and storage roots. The weight of each partition was determined, and a tissue sample was taken and immediately frozen in liquid nitrogen for further analysis. During sampling of the stem, a part of the stem was separated into peel and core. All samples were frozen in liquid nitrogen for further processing. To prevent thawing of samples the frozen plant material was processed into a fine powder using a mixing mill (Retsch, Haan, Germany). A sample of 70 mg of the frozen plant material was taken for RNA isolation, and the fresh weight was measured using an analytical scale (Sartorius M-pact AX224, Gottingen, Germany). Subsequently the plant material was freeze-dried using a lyophilizer (Alpha 2-4 LDplus, Christ; Osterode am Harz). After freeze-drying, the dry weight of the plant material was determined with an analytical scale, and samples of 10 mg each were taken to analyze the ion, sugar, and starch content.

### Ion, Sugar, and Starch Measurements

Soluble metabolites such as sugars and ions, were extracted from 10 mg of freeze-dried and subsequently dried plant material. For this purpose, sugars were extracted using 800 μl of 80% ethanol. After 5 minutes of centrifugation at 16,000 g, the supernatant was transferred to a new reaction tube, while the remaining pellet containing plant material was retained for subsequent starch extraction. To prepare for measurement, the supernatant was evaporated using a Speedvac concentrator (Eppendorf, Hamburg, Germany). The resulting pellet was resuspended in 300 μl ddH2O.

Before extracting starch, the pellet from the sugar extraction underwent several washing steps with 80% ethanol and water to remove any residual sugars. Once the pellet was washed, 250 μl of ddH2O was added, and the samples were autoclaved at 121° C. for 20 min to hydrolyze the starch. For enzymatic starch digestion, 250 μl of a sodium-acetate-enzyme-mastermix (containing 50 U/ml α-amylase, 6.3 U/ml amyloglucosidase, and 200 mM NaOAc at pH 4.8) was added to the pellet and incubation at 37° C. for 4 hours. The cleavage was terminated by heating the samples to 95° C. for 10 min.

Sugars (glucose, fructose, and sucrose) and hydrolyzed starch concentrations were measured using an enzymatic assay based on the NAD⁺-dependent conversion of glucose-6-phosphate to 6-phosphoglukonolactone as described by Stitt et al. (Stitt et al., 1989. Metabolite levels in specific cells and subcellular compartments of plant leaves. Methods Enzymol. 174, 518-552). Briefly, 5-10 μl of extracted soluble sugars were mixed with 190 μl of Premix [100 mm HEPES (pH 5.7); 10 mm MgCl₂; 2 mm ATP; 1 mm NAD; 0.5 U Glucose-6-phosphate-dehydrogenase] and absorption at 340 nm was measured in a Microplate Reader Infinite® M Nano (Tecan®, Männedorf, Switzerland). Hexokinase, phosphoglucoisomerase, and invertase were added sequentially for measurement, and absorbance was measured after addition of each enzyme until enzymatic saturation was reached.

For isolation of cations and anions, 1 ml of deionized water was added, after which the preparation was thoroughly mixed and kept for 15 minutes at 95° C. After centrifugation (10 minutes, 13,000 rpm, 4° C.) the supernatant was used for ion chromatography quantifications. Anions and cations were measured in a 761 Compact IC system (Metrohm, Herisau, Switzerland). For anion concentration measurements, a Metrosep A Supp 4-250/4.0 column and a Metrosep A Supp 4/5 Guard/4.0 guard column (Metrohm, Herisau, Switzerland) were used. 50 mm H₂SO₄ was used as anti-ion, and 1.8 mm Na₂CO₃ together with 1.7 mm NaHCO₃ dissolved in ultrapure water was used as eluent for anion measurement.

For determination of cation concentrations, a Metrosep C4 150/4.0 column and a Metrosep C4 Guard/4.0 guard column (both Metrohm and Herisau, Switzerland) were used. The eluent consisted of 2 mm HNO₃ and 1.6 mm dipicolinic acid dissolved in ultrapure water. Amino acid concentrations were measured via high performance liquid chromatography in a Dionex™ (Dionex™ Softron, Germering, Germany) system, consisting of a Dionex™ ASI-100™ Automated Sample Injector, a Dionex™ P680 HPLC pump, and a Dionex™ RF2000 fluorescence detector. An AminoPac™ PA1 column (Dionex Softron, Germering, Germany) was used for separation of amino acids. 0.1M NaAc, 7 mm Triethanolamine pH 5.2 was used as eluent. Samples were prepared for measurement by adding 60 μl boric acid buffer (0.2 M; pH 8.8) and 20 μl 6-aminoquinolyl-N-hydroxysuccinimidyl carbamate (3 mg dissolved in 1.5 ml acetonitrile). Samples were vortexed and incubated at 55° C. for 10 minutes. Peaks were analyzed using the CHROMELEON™ software (Thermo Fisher Scientific™, Waltham, Massachusetts, United States).

### Results

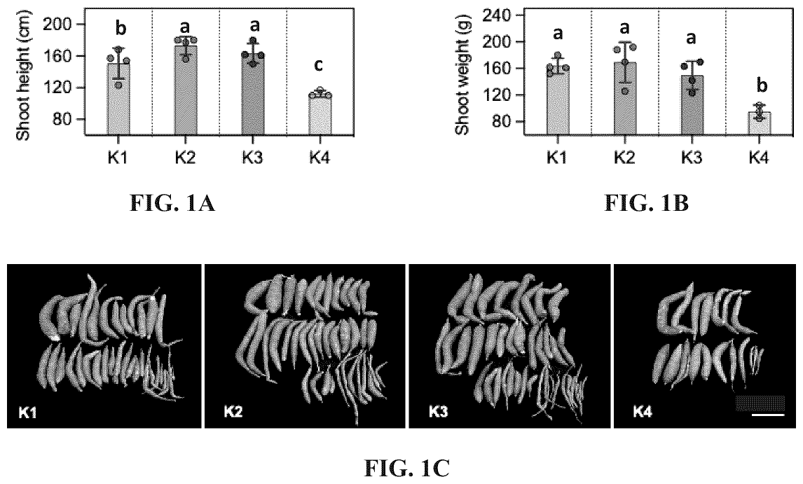

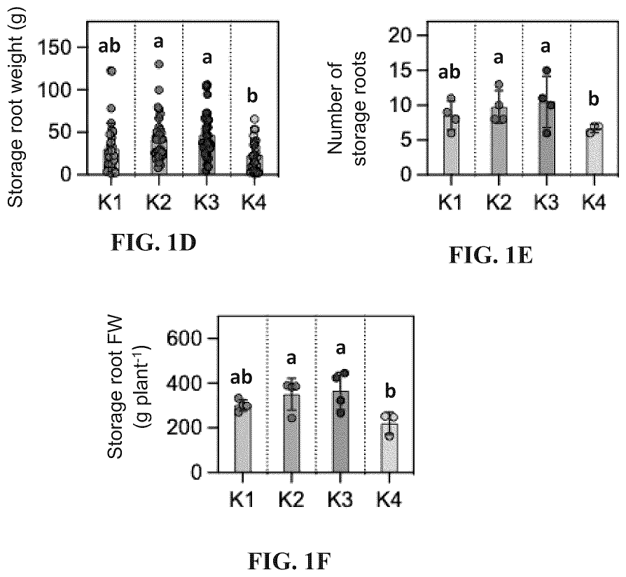

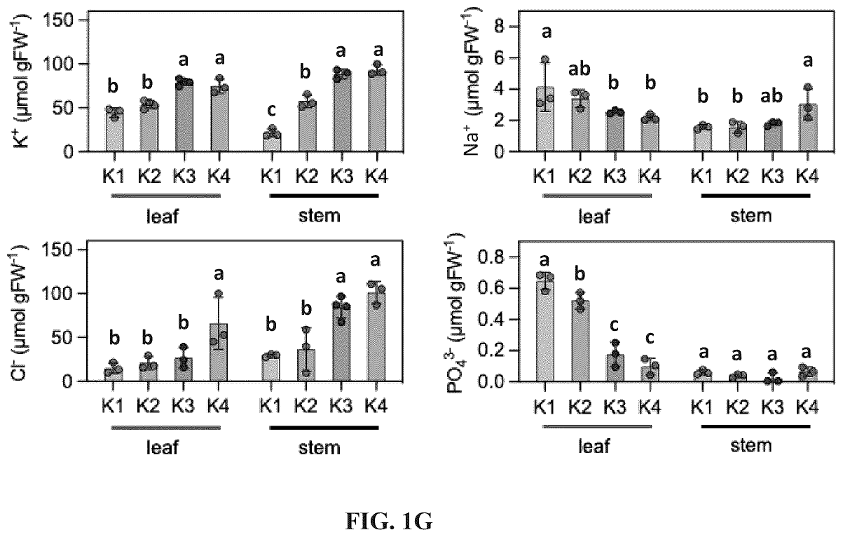

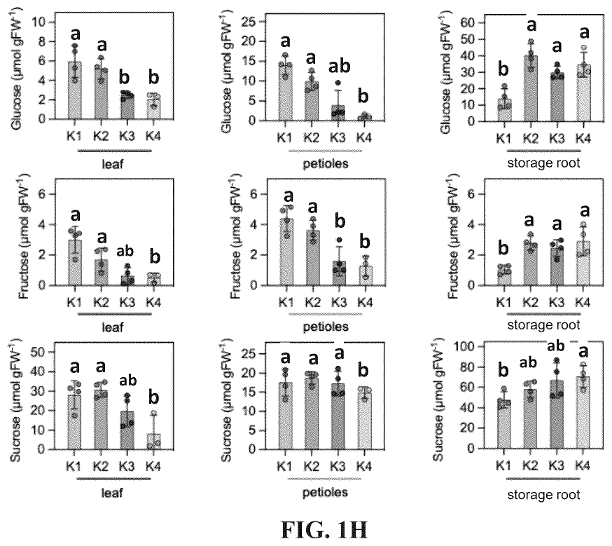

Potassium (K⁺) is not only an essential mineral and thus a growth limiting factor in crops but is also one of the most absorbed nutrients in cassava (Fernandes et al., 2017. Yield and nutritional requirements of cassava in response to potassium fertilizer in the second cycle. Journal of Plant Nutrition, 40, 2785-2796.). To further investigate the effect of potassium on plant growth and storage root formation in cassava, wild type plants were grown under greenhouse conditions on soil fertilized with different concentrations of potassium (K1=27 K⁺/kg soil; K2=142 K⁺/kg soil; K3=500 K⁺/kg soil; K4=2000 mg K⁺/kg soil). While the addition of up to 500 mg K⁺/kg soil (K1 to K3) resulted in improved shoot and storage root growth, addition of 2000 mg K⁺/kg soil (K4) represented a toxic amount that led to reduced plant growth and storage root formation (FIG. 1A-1F). While the fresh weight of the stem tissue increased by only 3.1% for plants grown under K2 conditions in comparison to K1, and even decreased by 8.73% in the case of plants grown under K3 conditions in comparison to K1 (FIG. 1B), interesting differences were found, especially when considering the stem height. Both K2 and K3 conditions resulted in a significantly increased growth height of the cassava plants with 15.15% and 6%, respectively, compared to K1 conditions (FIG. 1A). Further interesting differences were revealed by the examination of the storage roots. While fresh weight of individual storage roots was only slightly increased (FIGS. 1C-1D), K2 and K3 conditions resulted in an increased number of storage roots per plant compared to K1 and K4 (FIG. 1E), resulting in a significantly increased storage root fresh weight per plant (FIG. 1F).

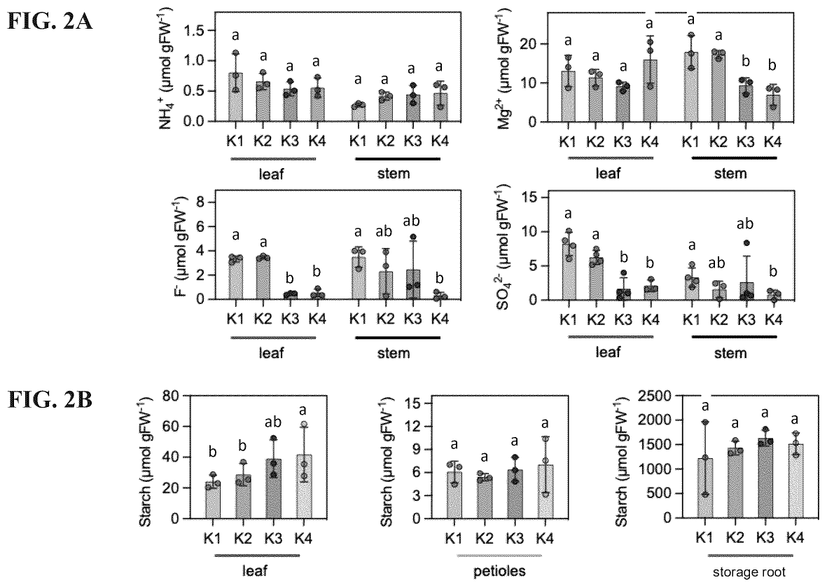

To demonstrate that the supplementation of potassium (K⁺) was successful and to investigate the effects of potassium intake on the ion homeostasis in cassava, the amount of sodium (Na⁺), chloride ions (Cl⁻), hydrogen phosphates (PO₄³⁻) (FIG. 1G), ammonium (NH₄⁺), magnesium (Mg²⁺), fluorine ions (F⁻), and sulphate ions (SO₄²⁻) (FIG. 2A) were determined in addition to potassium. Increased potassium uptake in leaves and stems due to fertilization was clearly shown by the quantification of different cations and anions (FIGS. 1G, 2A). In addition to a decrease in sodium (Na⁺) ions in the leaf, accumulation of K⁺ also resulted in a significant increase in chloride ions (Cl⁻) in leaf and stem and a decrease in hydrogen phosphate (PO₄³⁻) in the leaf (FIG. 1G). While the other cations (ammonium and magnesium) showed only minor changes in leaf and stem, fluoride and sulphate anions in leaf showed a significant decrease with increasing K⁺ and Cl⁻ concentrations (FIGS. 1G, 2A).

In a further step, the influence of potassium supplementation on sugar distribution was also considered in more detail. Glucose, fructose, sucrose, and starch concentrations were determined in the leaf, petioles, and storage roots. Glucose and fructose concentrations decreased in the leaf and petioles as K⁺ levels increased, but accumulated strongly in the roots (FIG. 1H). For sucrose, comparable observations were made in leaf tissue and roots, while the concentration in the petioles showed only minor changes (FIG. 1H). Starch accumulated with increasing potassium concentrations in leaf tissue, but did not show significant changes in petioles and storage roots (FIG. 2B).

Fertilization of cassava with nitrogen, potassium and/or phosphate can increase storage root yield under irrigation conditions. However, most cassava farmers do not use fertilizer and irrigation because fertilizer is expensive and water often limited during the dry season. Other mechanisms to improve storage root yield are therefore needed.

### Example 2: Overexpression of AtAKT2var Resulted in Alteration of Plant Growth in Field Trials

The following example describes experiments that showed that overexpression of mutated Arabidopsis thaliana AKT2 (referred to herein as Atakt2, AtAKT2mut, AtAKT2var, or AtAKT2var) in cassava resulted in increased plant growth, including increased plant height, total shoot dry matter, and total root dry matter.

### Methods

### AtAKT2var Overexpression

Genetic modification of the cassava cultivar 60444 was done following the method described by Bull et al. (Bull et al., 2009. Agrobacterium-mediated transformation of friable embryogenic calli and regeneration of transgenic cassava. Nature Protocols, 4, 1845-54.). In brief, friable embryogenic calli (FEC) were transformed with Agrobacterium tumefaciens containing the binary vector p134GG_pAtAKT2::AtAKT2mut (SEQ ID NO: 1) containing an AKT2 cassette and a selectable marker cassette (FIG. 3I; SEQ ID NO: 1), or p134GG_Vector control that lacked the AKT2 cassette (FIG. 3J, SEQ ID NO: 6). The functional AKT2 cassette included the following: Promoter—Arabidopsis thaliana POTASSIUM TRANSPORTER 2 (pAtAKT2, SEQ ID NO: 2); 5′-UTR—Arabidopsis thaliana POTASSIUM TRANSPORTER 2 (pAtAKT2); N-terminal Tag—6×HA-Tag; CDS—Arabidopsis thaliana POTASSIUM TRANSPORTER 2 (AtAKT2, SEQ ID NO: 3) with the following amino acid exchanges: S210N and S329N; 3′-UTR-terminator—Arabidopsis thaliana HEAT SHOCK PROTEIN 18.2 (HSP18.2) (FIG. 3A). The selectable marker cassette included the following: Promoter—Agrobacterium tumefaciens NOPALINE SYNTHASE (AtuNOS, SEQ ID NO: 5); CDS—HYGROMYCIN PHOSPHOTRANSFERASE (Hpt2, SEQ ID NO: 4); Terminator—CAULIFLOWER MOSAIC VIRUS 35S. Hygromycin-resistant embryos were regenerated and screened to confirm the presence of the transgene.

### Plant Growth

Cassava plants were grown as in Example 1. In addition to wild type “60444” cassava plants, transgenic lines “pAtAKT2::AKT2mut Line 4261,” “pAtAKT2::AKT2mut Line 4262,” “pAtAKT2::AKT2mut Line 4266” and “Vector control Line 4234” were grown. Transgenic and wild type plants arrived in sterile tissue culture jars before pre-hardening and were transferred to pots with soil in preparation for planting in the open field. Cassava empty vector controls (EV), promoter-GUS lines, and AKT2var overexpression lines generated from tissue culture were cultivated in Greiner containers on MS medium at pH 5.8 (Murashige and Skoog Basal Salt Mixture (MS), Duchefa Biochemie, Harleem, Netherlands) supplemented with 0.3% (w/v) gelrite, 2% (v/v) sucrose, and 2 μM CuSO4, under sterile conditions. Plants were maintained in a plant growth chamber under controlled conditions (16 h light/8 h dark; 100-120 mol photons m-2 s-1, 28/26° C.), before being transferred to soil for green house and field trials, respectively.

### qRT Analyses

Leaf, shoot, and root material of soil-grown plants was collected and homogenized in liquid nitrogen prior to extraction of RNA with the Spectrum™ Plant Total RNA Kit (Sigma-Aldrich) according to the manufacturer's specifications. RNA purity and concentration were quantified using a NanoDrop™ spectrophotometer. Total RNA was transcribed into cDNA using the qScript™ cDNA Synthesis Kit (Quantabio, USA). qPCR was performed using the PerfeCTa® SYBR® Green SuperMix with Fluorescein reference color (Quantabio) on CFX96 system (Bio-Rad, Hercules, CA, USA) using specific AKT2 primers (AKT2-fwd: 5′-TGGCTACTAACGGTGCAGAT-3′ (SEQ ID NO: 13), AKT2-rev: 5′-ACCCAAACTTCTCTCCTGCA-3′ (SEQ ID NO: 14)), and Manihot esculenta GAPDH (MeGAPDH-fwd: 5′-TCTTCGGCGTTAGGAACCCAG-3′ (SEQ ID NO: 15), MeGAPDH-rev: GCAGCCTTATCCTTGTCGGTG (SEQ ID NO: 16)) was used as reference gene for transcript normalization.

### Results

Intracellular and intercellular potassium distribution, in addition to increased availability of potassium in the soil, are also critical for improved growth. In Arabidopsis thaliana, the voltage-gated K (+) transporter AKT2 was previously shown to play a supporting role in the maintenance of K (+) gradients. Gajdanowicz et al. (Gajdanowicz et al., 2011. Potassium (K+) gradients serve as a mobile energy source in plant vascular tissues. Proc Natl Acad Sci USA, 108, 864-9.) suggested that AKT2 is involved in phloem companion cell membrane energization, thereby improving phloem loading and transport. Wild type AKT2 has two modes, namely mode 1, where AKT2 acts as an inward-rectifying K⁺ channel (Kᵢₙ), and mode 2, where AKT2 acts as a nonrectifying channel (both Kᵢₙ and Kₒᵤₜ; i.e., mediating both K+ uptake and release). Gajdanowicz et al. showed that the overexpression of AKT2_{S210N-S329N} modified AKT2 such that it was biased toward mode 2 or locked in mode 2. Plants with AKT2_{S210N-S329N} showed improved plant growth, especially under conditions of energy limitations. This gradient is used in vascular tissues as an energy source to (re-)loading processes in the phloem. While knocking out AKT2 impairs sucrose loading into the phloem (Deeken et al., 2002, Loss of the AKT2/3 potassium channel affects sugar loading into the phloem of Arabidopsis. Planta 216: 334-344) the overexpression of a mutagenized version (S210N-S329N) resulted in improved plant growth (Dreyer et al., 2017, The potassium battery: a mobile energy source for transport processes in plant vascular tissues. New Phytologist 216: 1049-1053).

As potassium supplementation has already been shown to influence sugar partitioning and plant growth positively (FIGS. 1A-2B), another approach to growth enhancement of cassava was the overexpression of the Arabidopsis thaliana K⁺ transporter AKT2 in a mutagenized form (S210N-S329N) (FIG. 3A) for cultivation in field trials (National Chung Hsing University, Taichung, Taiwan). In brief, phloem-specific expression, controlled by the pAtAKT2 promoter, of a constitutively activated potassium channel (AKT2_{S210N-S329N}) may result in improved phloem loading and long-distance transport of assimilates. This should result in better resource allocation to storage roots, thereby improving storage root yield. To verify independent transgenic events, Southern blot analyses were conducted, and single insertion events were shown for AKT2var-4255, AKT2var-4264, AKT2var-4265 and AKT2var-4266 whereas AKT2var-4261 and AKT2var-4262 showed at least two insertions (FIG. 3B), using the “AKT2_CDS_RT_F1/R1” primers from Table 2). In addition to wild types (WT) and six different AtAKT2var events (AKT2), six different friable embryonic calli events (FEC), and six different empty vector controls (EV) were examined in this study (FIG. 3C).

TABLE 2
Primer names, sequences, amplifications factors, and efficiency.

| | | SEQ | Amplification | Primer |
|---|---|---|---|---|
| Primer name | Sequence (5′ to 3′) | ID NO | factor | efficiency |
| MeGAPDH fwd | TCTTCGGCGTTAGGAACCCAG | 27 | 1.92 | 92.14 |
| MeGAPDH rev | GCAGCCTTATCCTTGTCGGTG | 28 | | |
| AtAKT2 fwd | ACAGGGGCTTAACGTCGACAC | 29 | 2.07 | 106.59 |
| AtAKT2 rev | TGCACCGTTAGTAGCCAGGAGA | 30 | | |
| AKT2 CDS_RT_F1 | CAGCTTCTTGTCCGTGAACC | 31 | NA | NA |
| AKT2 CDS_RT_R1 | AGGTAAGCAGTGAGGCCAAG | 32 | NA | NA |

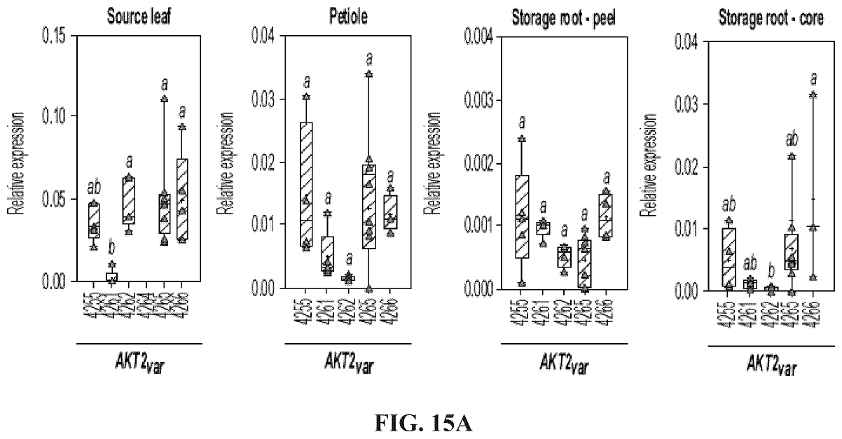

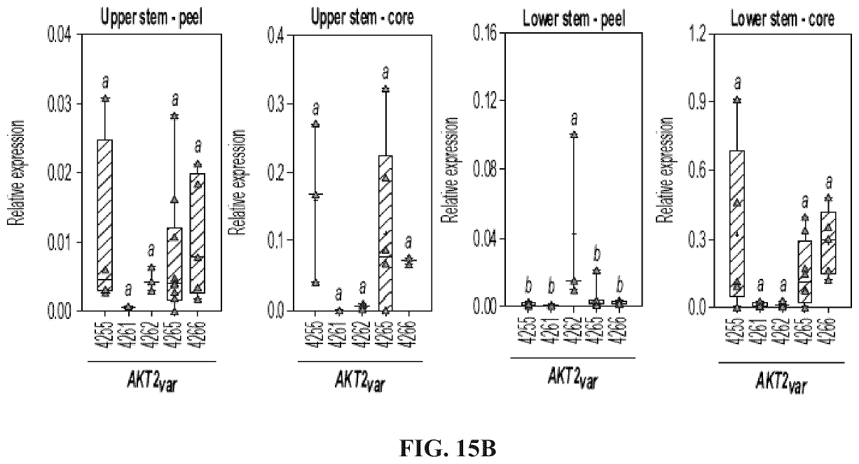

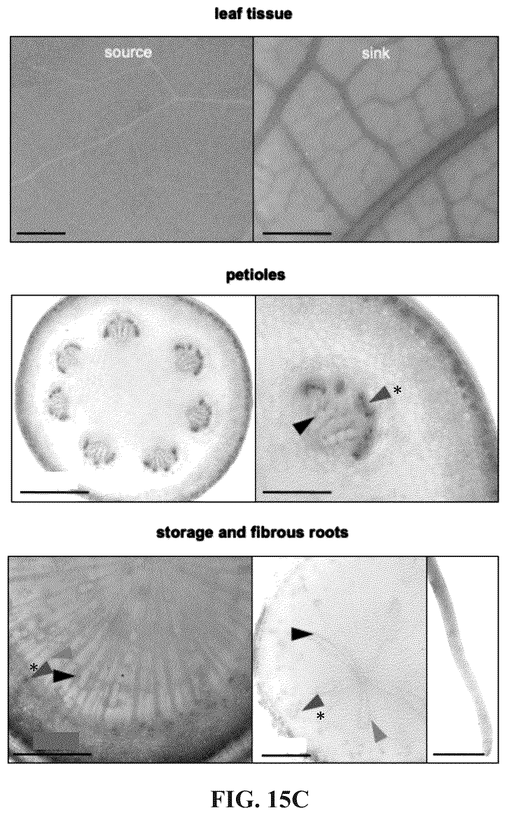

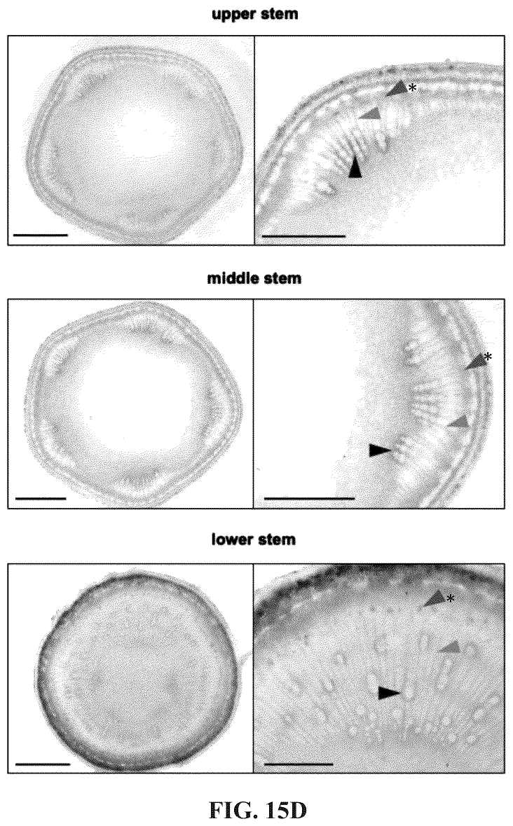

In a further step, qRT analyses were carried out, which showed that no AKT2 from Arabidopsis thaliana was introduced into the empty vector controls (EN4218, EN4220, EN4239, EN4234 and EN4243). In contrast, the AtAKT2var events (AKT2var-4255, AKT2var-4261, AKT2var-4262, AKT2var-4264, AKT2var-4265 and AKT2var-4266) showed different expression patterns in the tissues examined. In these findings, studies of expression in leaves revealed the highest expression levels for AKT2var-4255, AKT2var-4262, and AKT2var-4264 (FIG. 3D). Studies of different stem segments divided into upper-, middle-, and lower stem revealed that in the upper and middle parts of the stem, expression was highest in AKT2var-4255, AKT2var-4265 and AKT2var-4266, respectively, and in the lower stem, expression was highest in AKT2var-4255, AKT2var-4261 and AKT2var-4266 (FIG. 3D). The lowest expression was found by examining the storage root tissue. Here, AKT2var-4255, AKT2var-4265, and AKT2var-4266 exhibited the highest expression, while almost no expression was detectable in AKT2var-4261, AKT2var-4262, and AKT2var-4264 (FIG. 3D). Additional measurements included divisions of the stems and storage tissues into peel and core tissues, with the peel and core representing phloem- and xylem-containing tissues, respectively. For these measurements, leaves and petioles exhibited similarly low AKT2var transcript levels, comparable to those observed in the peel and core of storage roots (FIG. 15A). While the peel of the stem tissue also showed relatively low AKT2var expression, significantly higher transcript levels were detected in the core tissue of upper and lower stems (FIG. 15B).

The growth performance was determined by examining the growth height and determining the total shoot dry matter (TSDM) and total root dry matter (TRDM). The results for the AtAKT2var overexpression lines were comparable to what was observed in potassium fertilization with increasing potassium levels. While the AKT2var-4261, AKT2var-4262, and AKT2var-4264 lines were particularly noticeable in the comparison of growth height (FIGS. 3E-3F), examination of the total shoot dry matter only showed an increase for the AKT2var-4261 and AKT2var-4262 lines, while the AKT2var-4264 line was significantly lower than most controls (FIGS. 3F, 4A). Upon examination of the root tissue, it became clear that not only was shoot growth increased in AKT2-overexpressing plants, but the storage roots also showed improved growth (FIG. 3G). More detailed observations showed that especially AKT2var-4261 and AKT2var-4262 lines had strongly increased TRDM and the AKT2var-4264 line had reduced TRDM, although these lines were the highest growing lines besides AKT2var-4261 and AKT2var-4262 (FIGS. 3G, 3H, 4B).

These observations were consistent with the development of the plant, as shown by studies of the intermediate harvest (approximately 4 months after planting). Here, it was first shown that increased plant growth especially for the AKT2var-4264 line, was present throughout plant development (FIGS. 7A-7C, 7D). In contrast, a determination of the fresh weight of shoots and roots showed that an increase occurred only in the later stages of plant development in AtAKT2var overexpression lines (FIGS. 7E-7I).

Gajdanowicz et al. (Gajdanowicz et al., 2011. Potassium (K+) gradients serve as a mobile energy source in plant vascular tissues. Proc Natl Acad Sci USA, 108, 864-9) hypothesized that K⁺ circulating in the phloem served as a decentralized energy storage that can be used to overcome local energy limitations, and that posttranslational modification of AKT2 allows for access to the “potassium battery” by efficiently assisting the plasma membrane H+-ATPase in energizing the transmembrane phloem loading process. Following this hypothesis, AKT2 and the “potassium battery” would be most relevant in apoplasmic phloem loaders that actively transport assimilates against a concentration gradient. Recent results from cassava, however, suggest that this plant is a largely passive symplasmic phloem loader (Rüscher et al., (2024) Symplasmic phloem loading and subcellular transport in storage roots are key factors for carbon allocation in cassava. bioRxiv.). In a symplasmic phloem loader much less active transport and therefore much less of an impact of AKT2 energizing this active transport would be expected. Therefore, the positive results in cassava are surprising as the overexpression had a positive impact on allocation and yield.

In addition, the positive effects reported by Gajdanowicz et al. (Gajdanowicz et al., 2011. Potassium (K+) gradients serve as a mobile energy source in plant vascular tissues. Proc Nat Acad Sci USA, 108, 864-9) in Arabidopsis plants overexpressing mutant AKT2 seemed mostly related to growth under energy limiting conditions. However, the cassava plants overexpressing AtAKT2var performed better even under standard growth conditions.

### Example 3: Overexpression of AtAKT2var Affected Ion Distributions in Shoot and Root Tissue in Field Trials

The following example describes experiments that showed that overexpression of mutated Arabidopsis thaliana AKT2 in cassava resulted in an alteration of the concentrations of various cations and anions in the shoot and root tissue. For this and subsequent Examples, it is worth noting that in Arabidopsis, the expression of AKT2var enhances stem growth, which is assumed to reflect accelerated sucrose transport within the sieve cells (Gajdanowicz et al., 2011, ibid.). However, such accelerated sucrose transport has so far only been speculated after computational calculations and not previously shown experimentally (Gajdanowicz et al., 2011, ibid.).

### Methods

Cassava plants were grown as in Example 2. Ion quantification was performed as in Example 1.

### Results

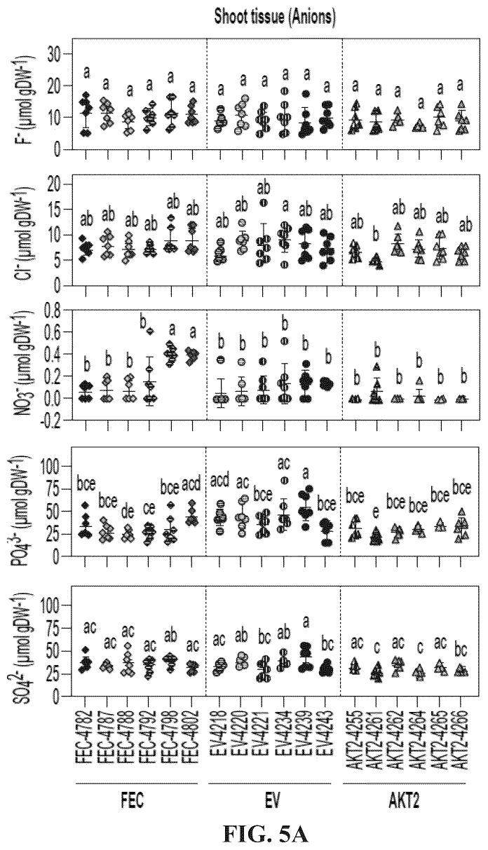

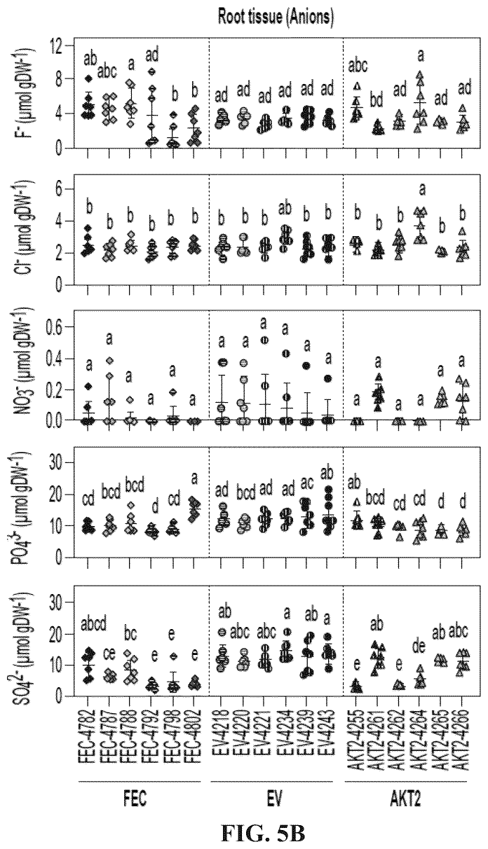

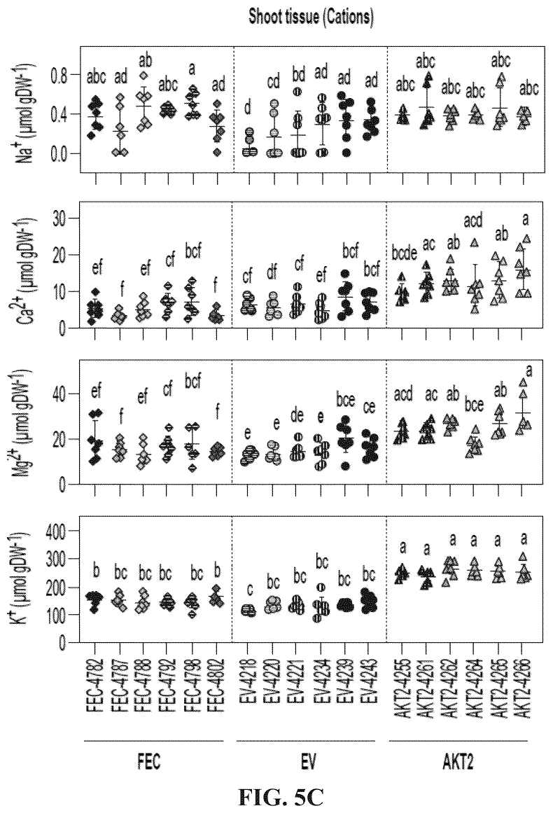

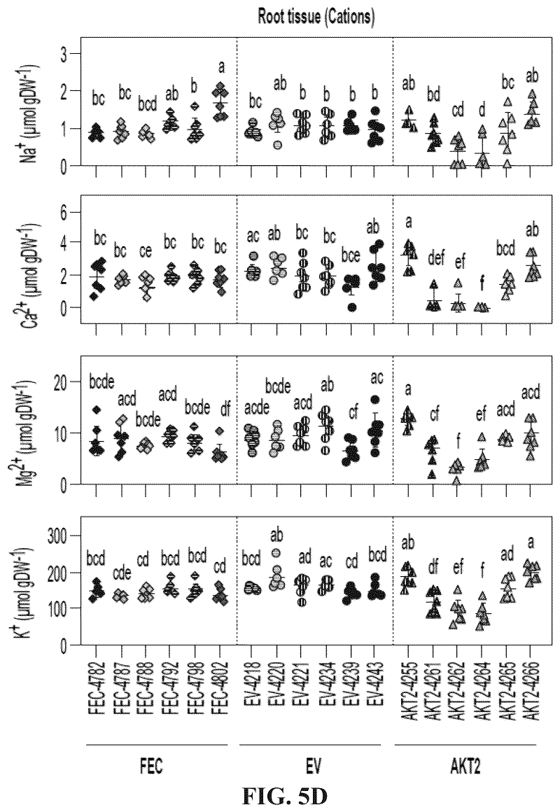

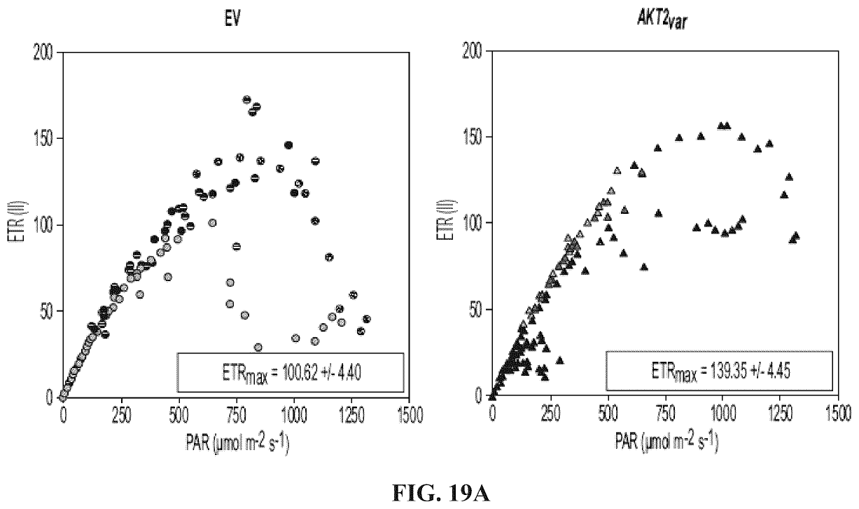

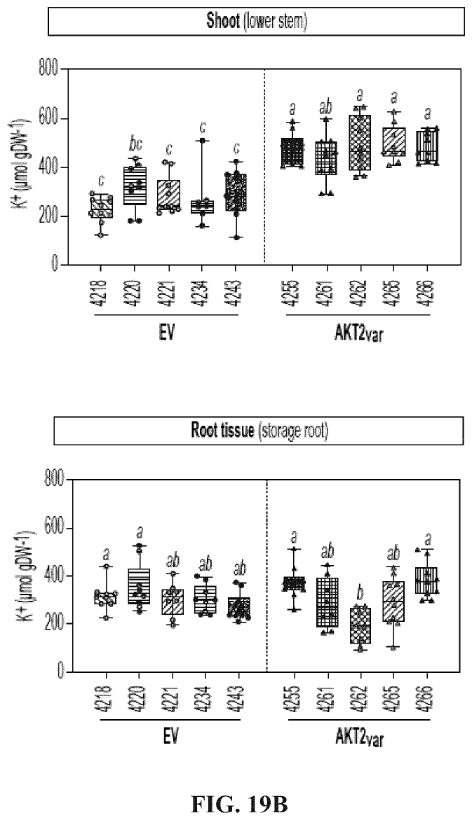

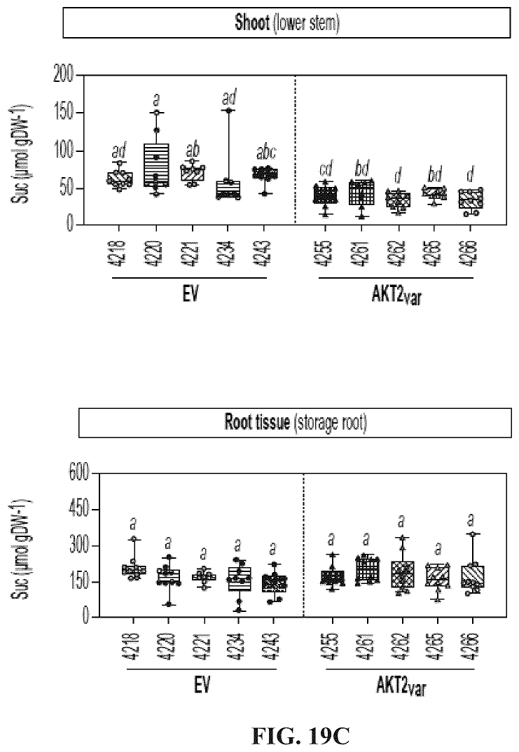

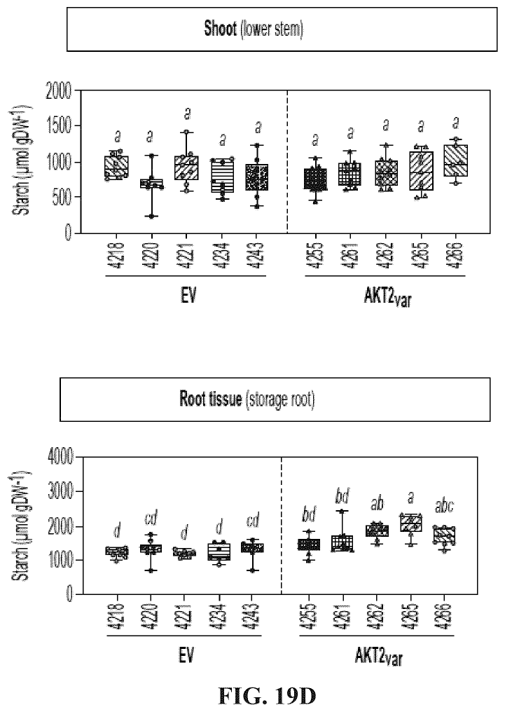

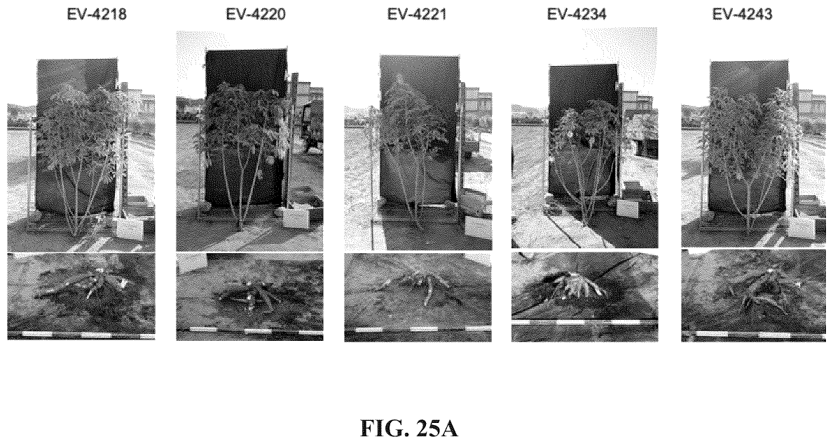

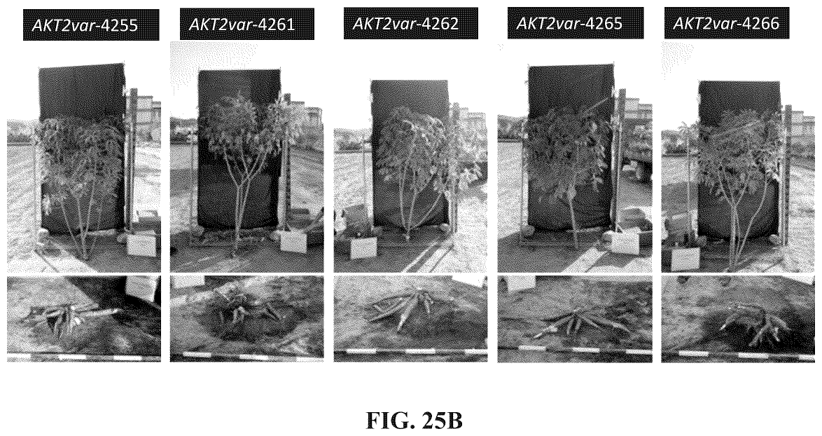

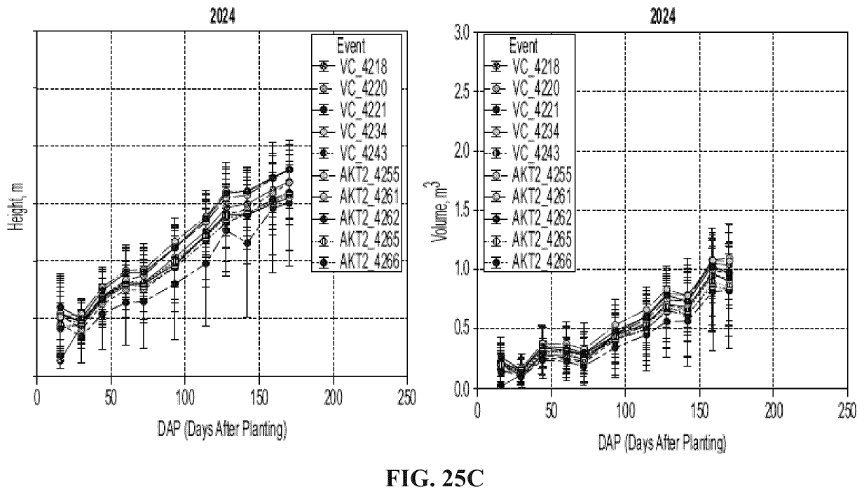

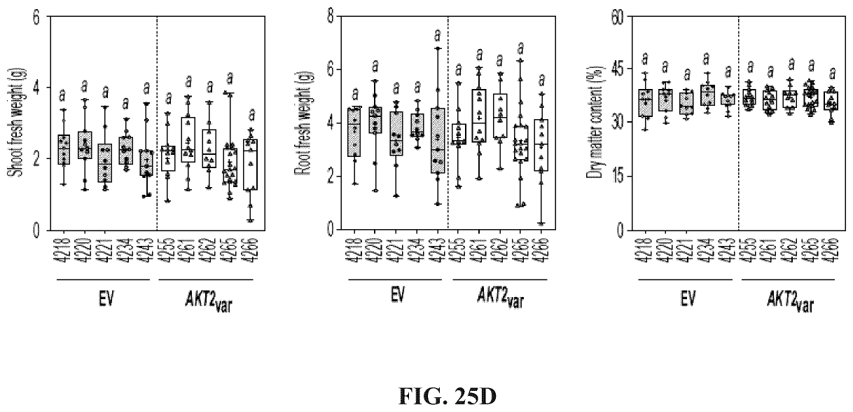

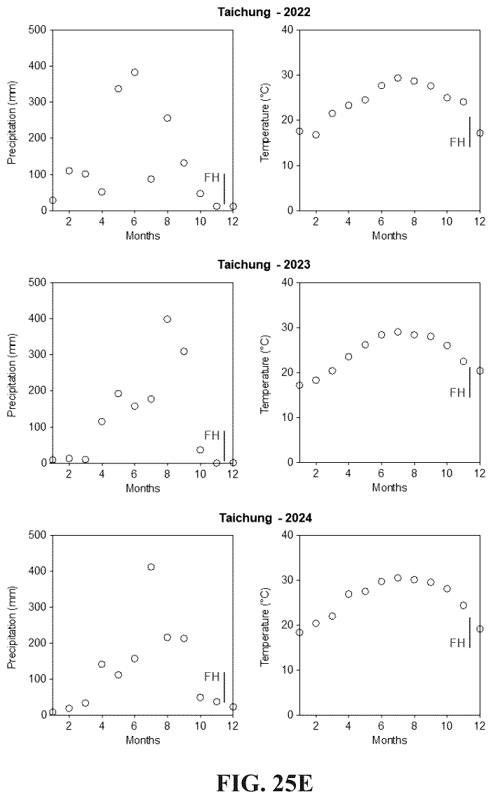

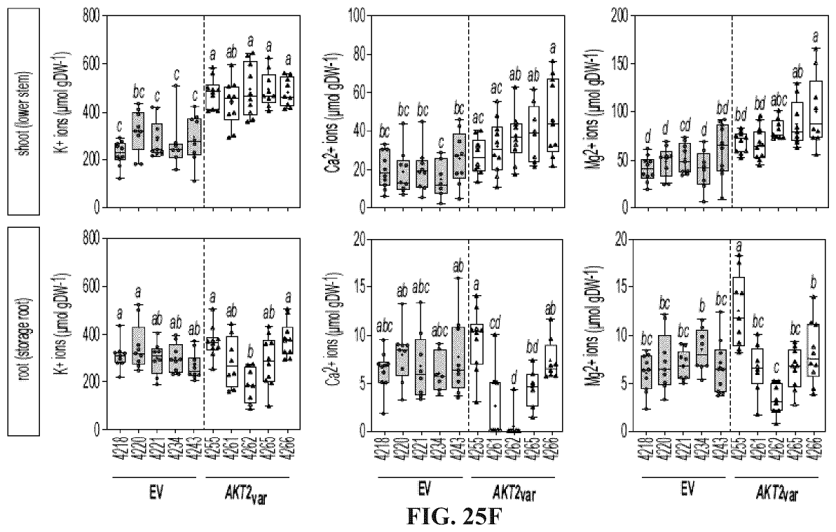

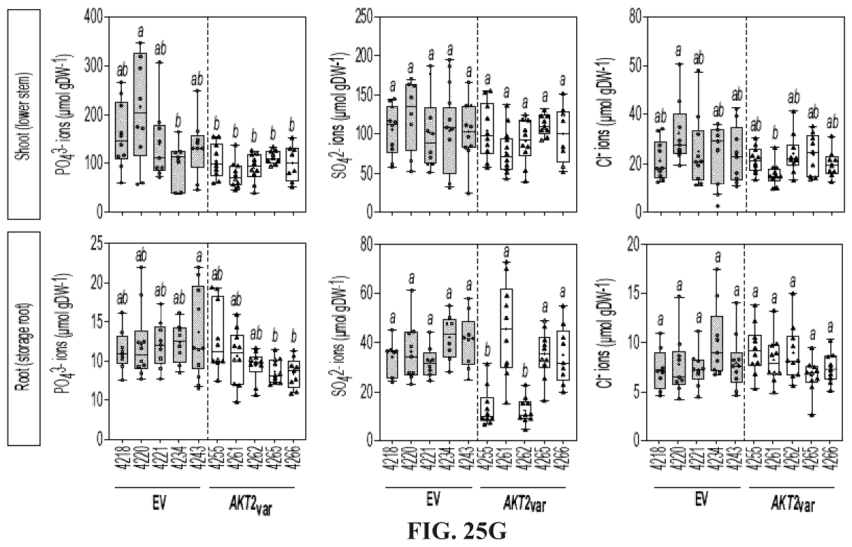

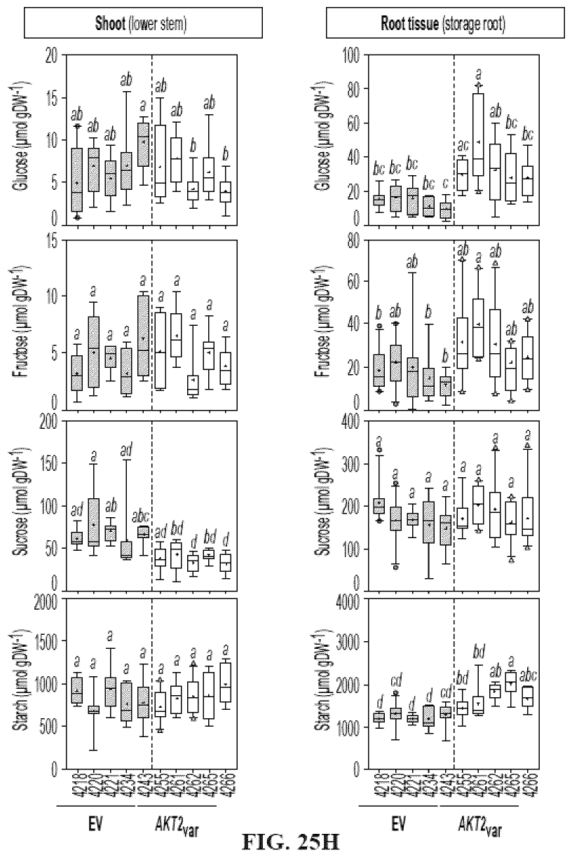

To investigate the influence of AKT2 overexpression on the distribution of ions in cassava, different anions and cations were quantified in stem and root tissue. While determination of the anions fluoride (F⁻), chloride (Cl⁻), nitrate (NO₃⁻), phosphate (PO₄³⁻) and sulphate (SO₄²⁻) in shoot (FIG. 5A) and root tissue (FIG. 5B) showed only slight changes in concentration, the quantification of the cations revealed changes in distribution and concentration. First, it was found that all cations examined (sodium (Na⁺), calcium (Ca²⁺), magnesium (Mg²⁺), and potassium (K⁺)) in all AKT2 overexpression events were elevated in shoot tissue compared to the controls. The most significant change was observed in the potassium concentration, which was uniformly and clearly elevated in all AKT2 events in shoot tissue (FIG. 5C, bottom row). Contrary observations were made in the analysis of cations in the root tissue. While the AKT2var-4255, AKT2var-4265 and AKT2var-4266 lines showed no significant changes when compared to FECs and vector controls, the AKT2var-4261 and AKT2var-4262 lines showed significantly reduced concentrations of all cations investigated (FIG. 5D). Specifically, the K⁺ concentration in the lower stem for AKT2var-expressing lines was 478.2 μmol g DW⁻¹, thus 69.7% higher than in the EV controls (281.9 μmol g DW⁻¹). However, K+ levels in storage roots of the top-performing AKT2var-expressing lines AKT2var-4261 and AKT2var-4262 were significantly lower than those in the EV control lines (FIG. 19B). Ca²⁺ and Mg²⁺ contents followed the same trend as K⁺, while the anions tested accumulated to comparable levels across lines (FIGS. 25F-25G). These data suggest that AKT2var expression not only improves certain growth parameters and photosynthetic efficiency, but also alters the tissue-specific accumulation of cations. Although K⁺ homeostasis may affect the uptake of other nutrients from the soil, no corresponding alterations were observed in the contents of other anions in shoots or roots (FIG. 25G). This selective change in ion homeostasis suggests a K⁺-specific effect of AKT2var expression in cassava.

### Example 4: Overexpression of AtAKT2var Boosted Photosynthetic Performance in Field Trials

The following example describes experiments that showed that overexpression of Arabidopsis thaliana AKT2 in cassava resulted in altered sugar and starch contents, as well as increased photosynthesis, in field trials.

### Methods

### Plant Growth

Cassava plants were grown as in Example 2.

### Measurements of Photosynthetic Performance

Measurements of volume-based seasonal growth were performed with an RGB camera installed on an unmanned aerial vehicle (UAV). Measurements of electron transport rate were performed with a monitoring pulse amplitude modulation (MoniPAM). Raw data downloaded from the MoniPAM was filtered based on the visual (not automated) comparison between the diurnal cycles of PAR and the quantum efficiency of photosystem II, Y (II).

### Results

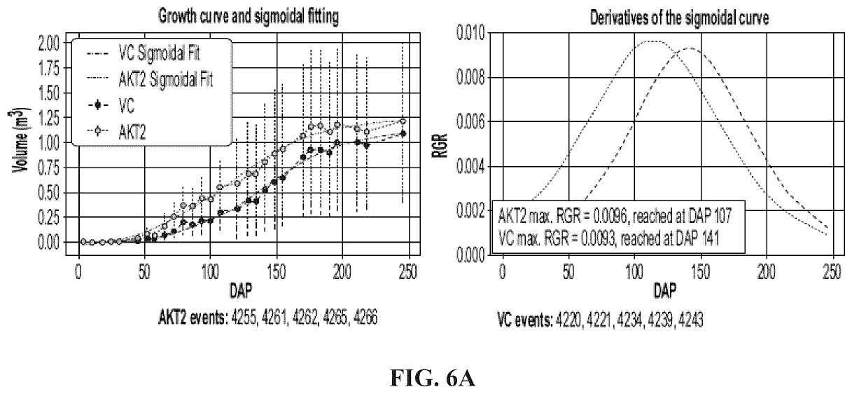

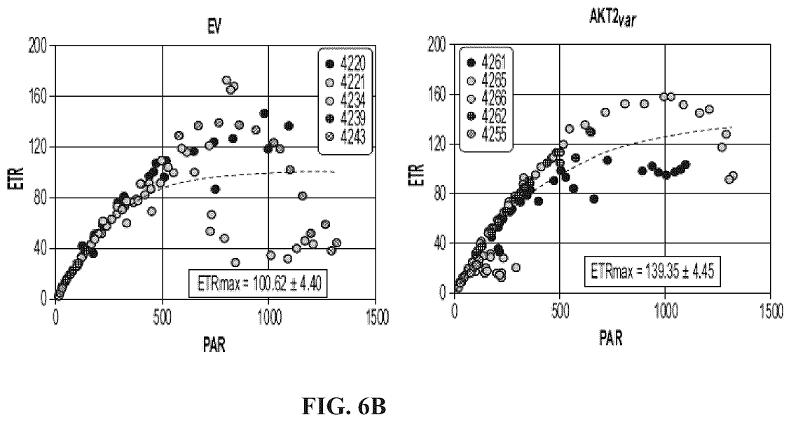

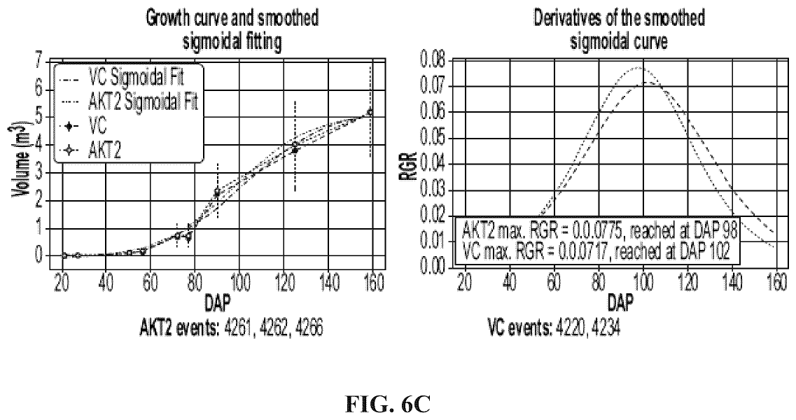

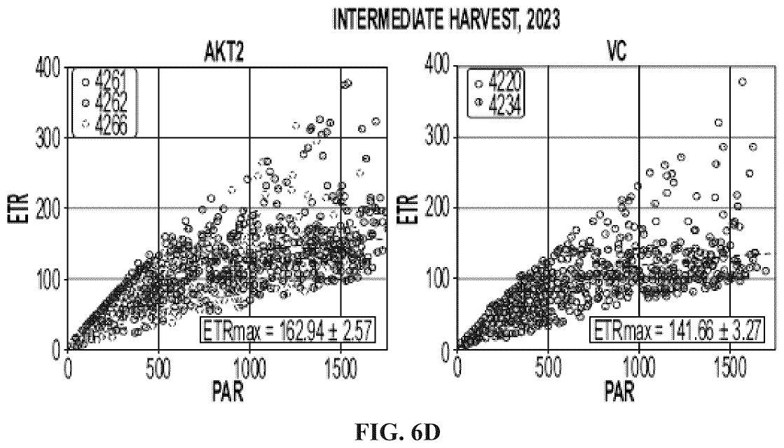

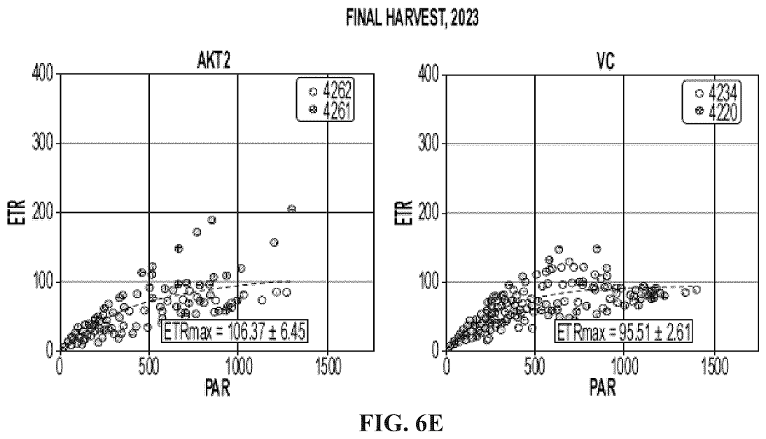

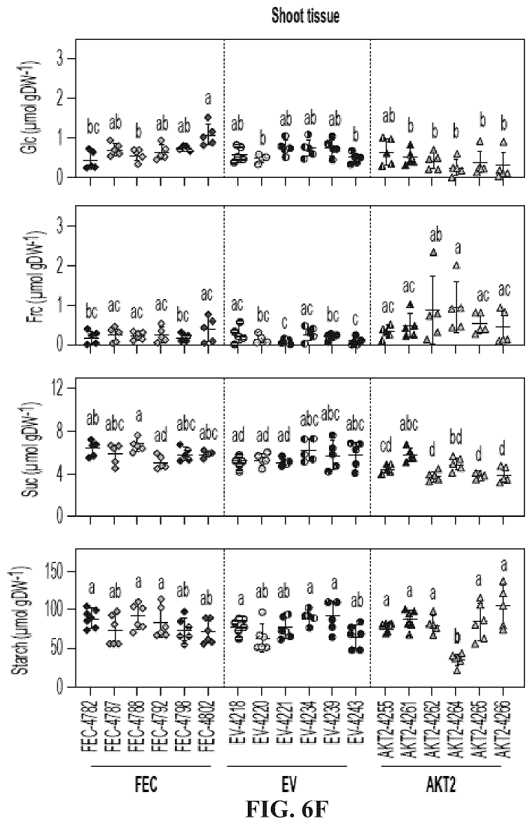

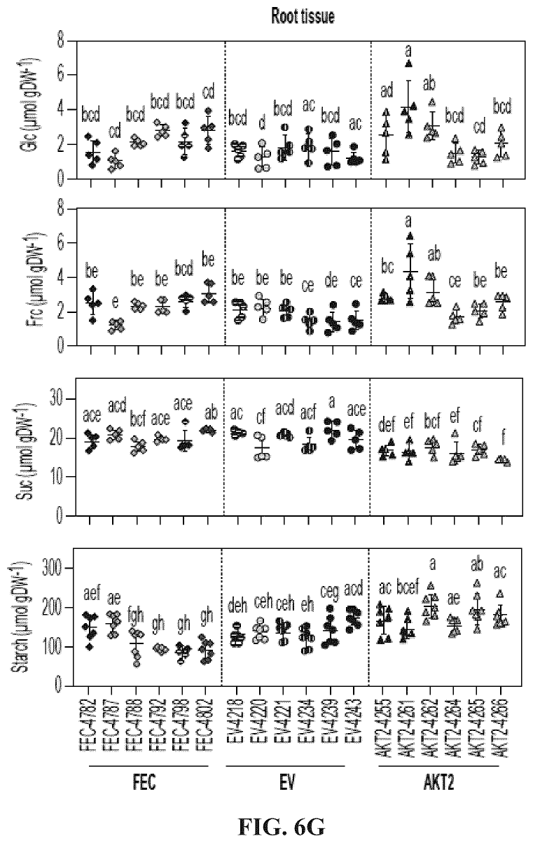

Further evidence for improved plant growth was obtained by measuring various photosynthesis related parameters and altered sugar distribution in AKT2 overexpression events. Field measurements (technical implementation) showed that not only the electron transport rate but also the yield in the investigated AKT2 events were above those of the investigated controls and thus indicated an improved photosynthetic performance (FIGS. 6A-6B). Sugars and starch are the primary carbohydrate products of photosynthesis, and these two carbohydrate products' rates improved in the field-grown AKT2var-expressing lines (FIGS. 19C-19D and FIG. 25H). The sucrose levels in the same AKT2var-expressing lines dropped significantly in the lower stem tissue, accumulating to 38.5 μmol g DW⁻¹ for the AKT2var-expressing lines, against 66.9 μmol g DW⁻¹ for the EV controls (FIG. 19C). By contrast, the starch content in several AKT2var-expressing lines was 1701 μmol g DW⁻¹, or 51.6% higher on average than that in EV control lines with 1122 μmol g DW⁻¹ (FIG. 19D). Glucose and fructose levels were not altered in the lower stem, but glucose reached significantly higher levels in the roots of the AKT2var-expressing lines than in those of the EV controls (FIG. 25H). In summary, expressing AKT2var in cassava shifted K⁺ accumulation toward shoots, resulting in lower K⁺ levels in storage roots. At the same time, AKT2var-expressing lines exhibited lower shoot sucrose levels and a greater phloem transport rate, indicating an enhanced sucrose transport from source to sink tissues, leading to overall higher storage root yield.

AtAKT2var overexpression events reached the maximum relative growth rate about one month before empty vector control events (FIG. 6A). Additionally, using the maximum electron transport rate (ETR) as a reference for photosynthetic efficiency, initial analysis revealed AtAKT2var overexpression events presenting higher photosynthetic efficiency (FIG. 6B, 139.35±4.45 for AKT2, compared to 100.62±4.40 for vector control). Similar, but lesser effects were detected in the harvest of the next year (FIGS. 6C-6E), in which AtAKT2var overexpression events reached the maximum relative growth rate (RGR) four days before vector controls, and presented higher photosynthetic efficiency (162.94±2.57 for AKT2, compared to 141.66±3.27 for vector control at intermediate harvest; 106.37±6.45 for AKT2, compared to 95.51±2.61 for vector control at final harvest).

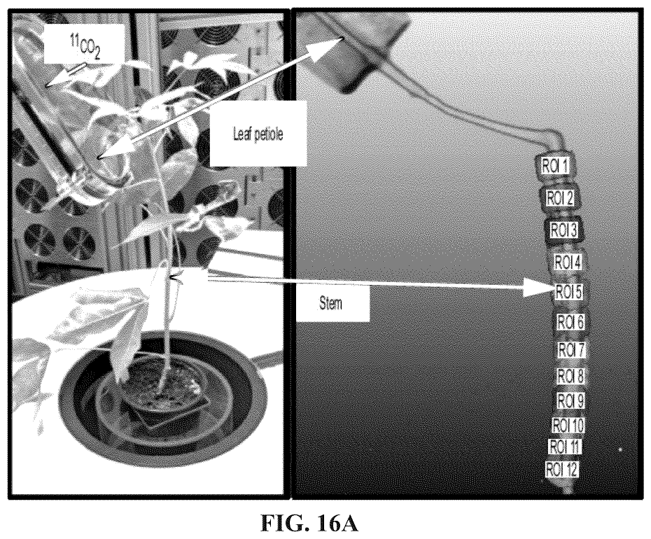

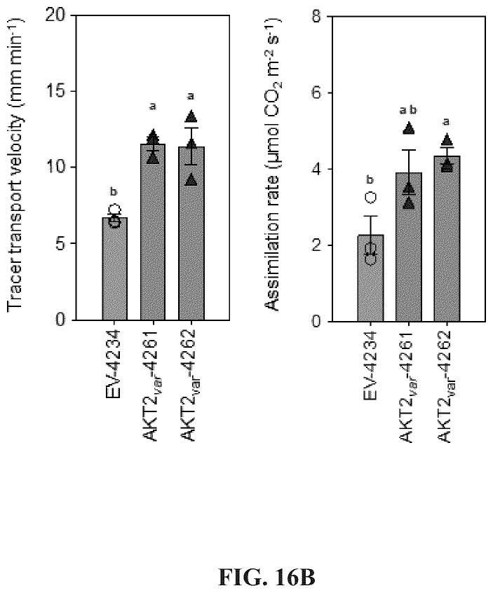

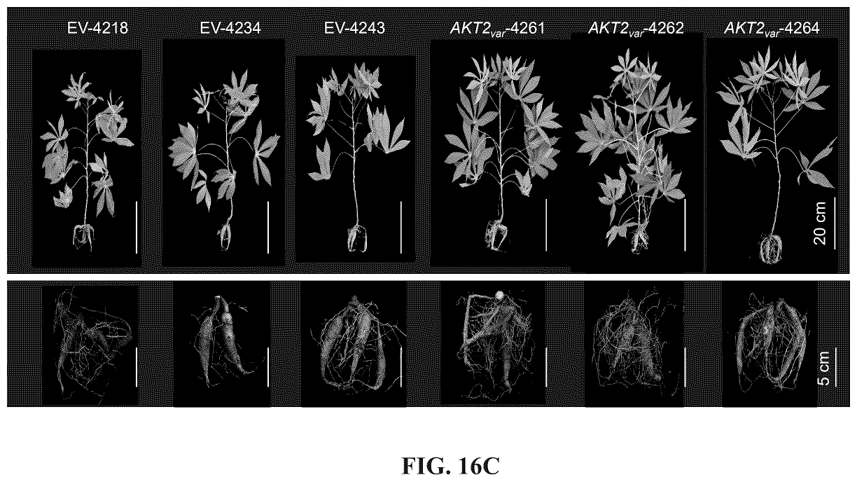

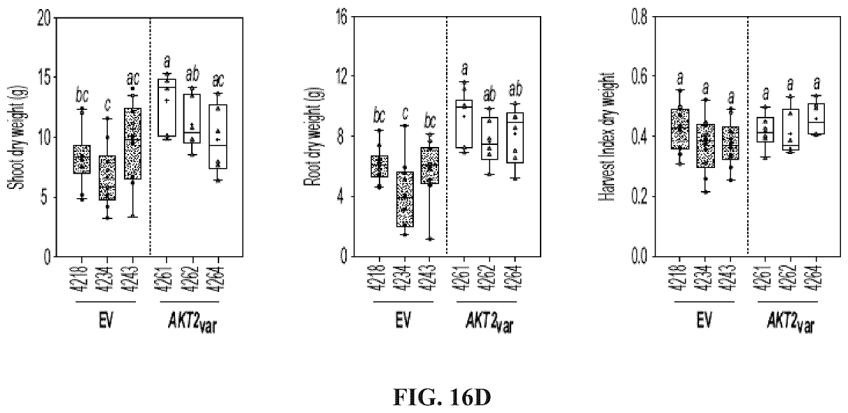

In further analysis of the 2022-2024 results, it was found that the ETR was 8% higher in AKT2var plants compared to EV plants under saturated light conditions, indicating that AKT2var slightly improves photosynthetic efficiency under field conditions. In 2022, the maximum ETR at saturation was 347.7±21.6 for AKT2var plants and 323.9±18.3 for EV plants. In 2023, the maximum ETR at saturation was 106.4±6.4 for AKT2var plants and 95.5±2.4 for EV plants. In 2024, the maximum ETR at saturation was 162.5±1.7 for AKT2var plants and 166.5±2.5 for EV plants. The AKT2var-expressing lines showed a maximal ETR about 38.4% higher on average than that of the EV control lines (FIG. 19A). This enhancement suggested an improved photosynthetic efficiency of AKT2var-expressing lines under field conditions, which was in line with the higher CO₂ fixation rate of plants from representative AKT2var-expressing lines grown under greenhouse conditions (FIG. 16B). Both wild type and wild type friable embryonic calli plants presented higher photosynthetic rates compared to either AtAKT2var overexpression events or empty vector control events (FIGS. 6A-6E).

To check whether improved photosynthetic performance also influenced sugar and starch concentrations, these were investigated in more detail (FIGS. 6F-6G). In the shoot, while only slight changes were observed in the concentrations of glucose, fructose, and starch, sucrose concentrations showed a reduction in almost all AKT2 events (FIG. 6F). The root showed an accumulation of glucose and fructose, especially for the AKT2var-4261 and AKT2var-4262 lines, and a reduced sucrose concentration in almost all AKT2 overexpression events, as in the shoot (FIG. 6G). A comparison of the starch concentration revealed a further change: except for the AKT2var-4261 line, all AKT2 events showed increased starch concentrations in the root, especially in comparison to the FECs but also to the EVs (FIG. 6G).

### Example 5: Overexpression of AtAKT2var Resulted in Alterations of Plant Phenotypes in Greenhouse Trials

The following example describes experiments that showed that overexpression of mutated Arabidopsis thaliana AKT2 in cassava resulted in altered plant growth in greenhouse trials.

### Methods

### Plant Growth

### Initial Trials

Cassava plants were grown as in Example 2, with the following exceptions. Plants from sterile culture were first grown under controlled conditions (14 h light/10 h dark; 180 mol photons m⁻² s⁻¹, 80% humidity, 28° C.) for 3 weeks after transfer to soil. For further cultivation, the plants were transferred to the greenhouse (14 h light/10 h dark; 180 mol photons m⁻² s⁻¹, 26-28° C.) and cultivated there for a further 16 weeks. Plants from cuttings were grown directly in soil and cultivated for 19 weeks in the greenhouse under the same conditions.

### Further Trials

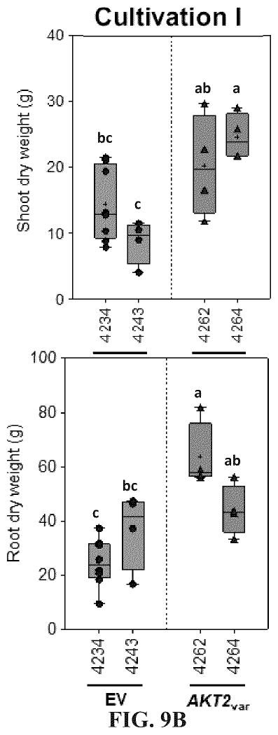

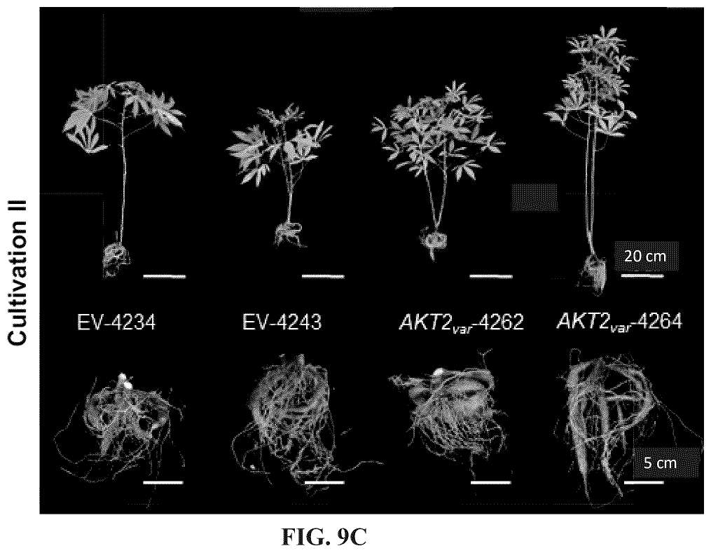

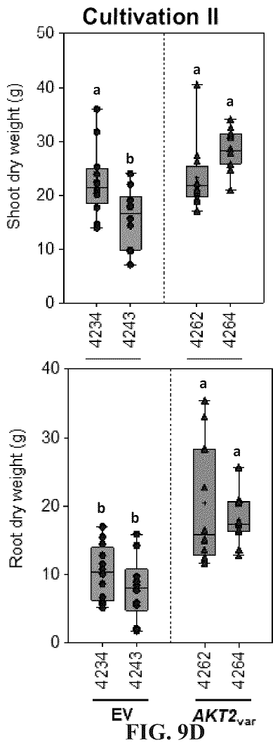

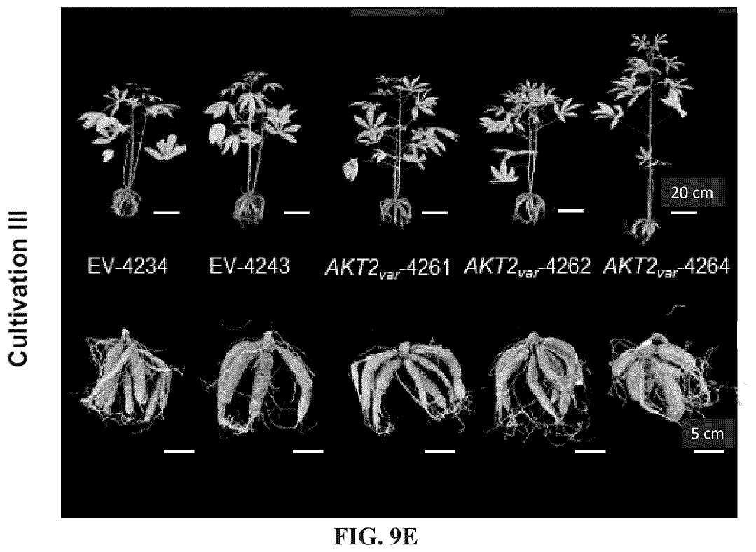

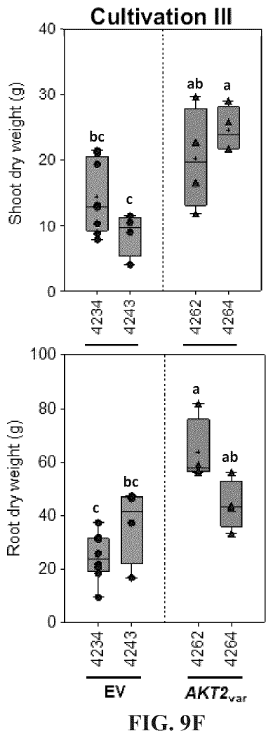

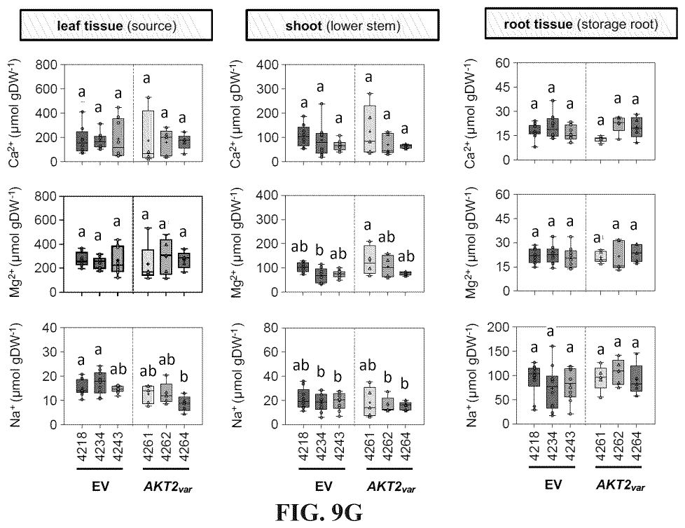

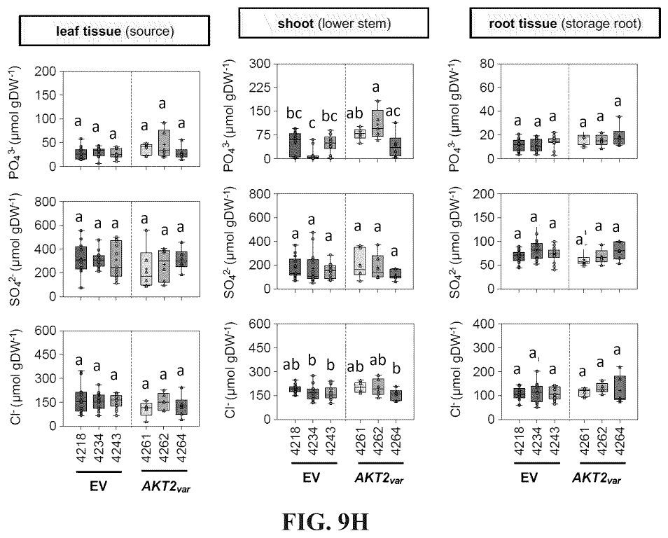

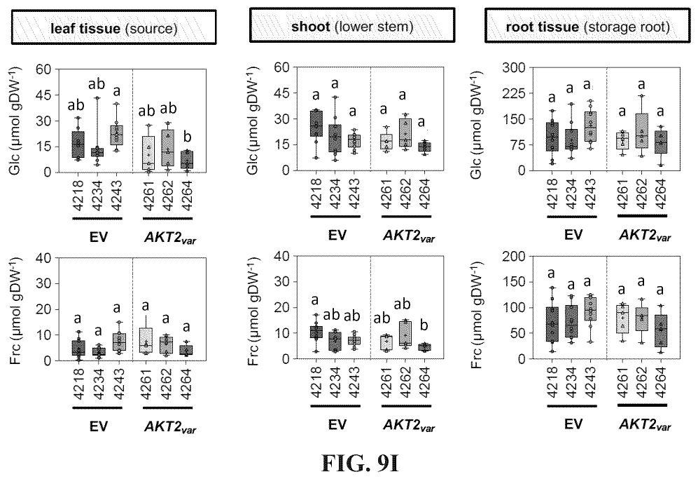

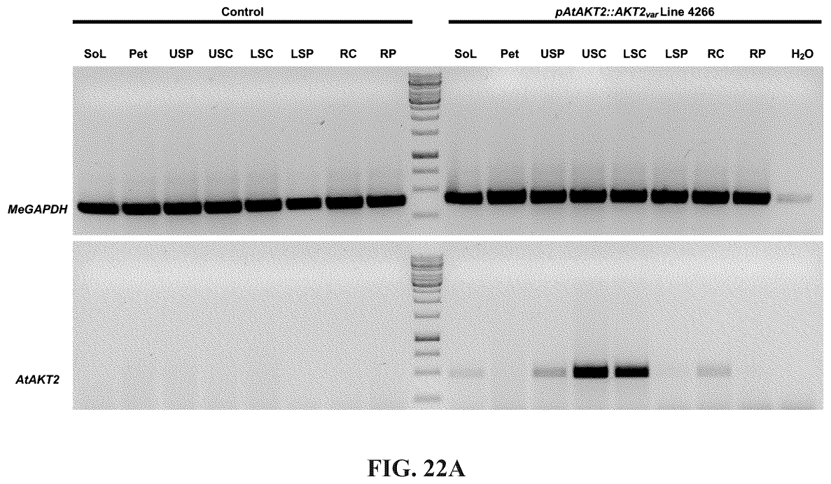

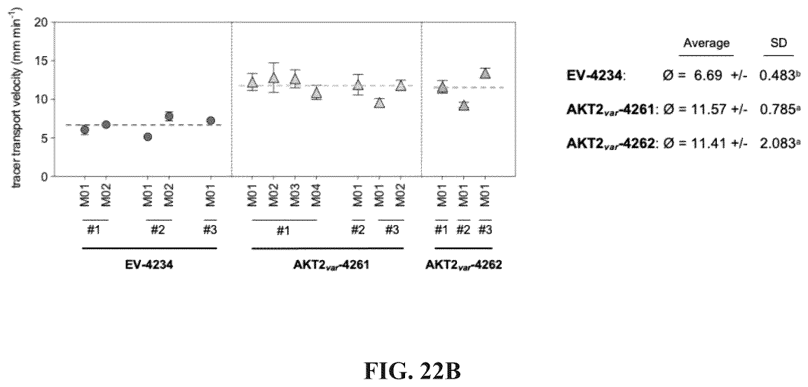

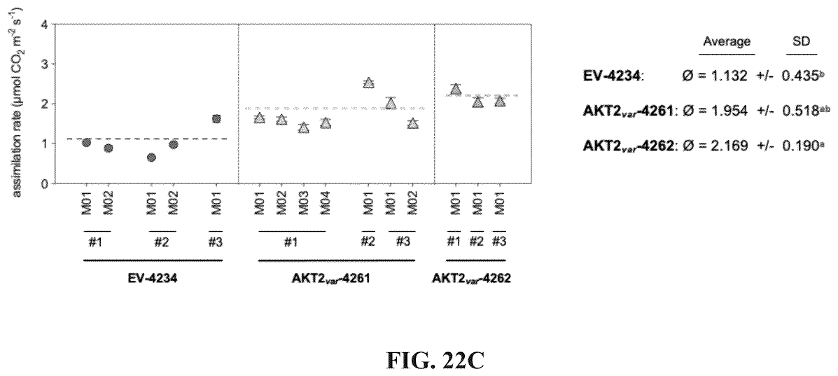

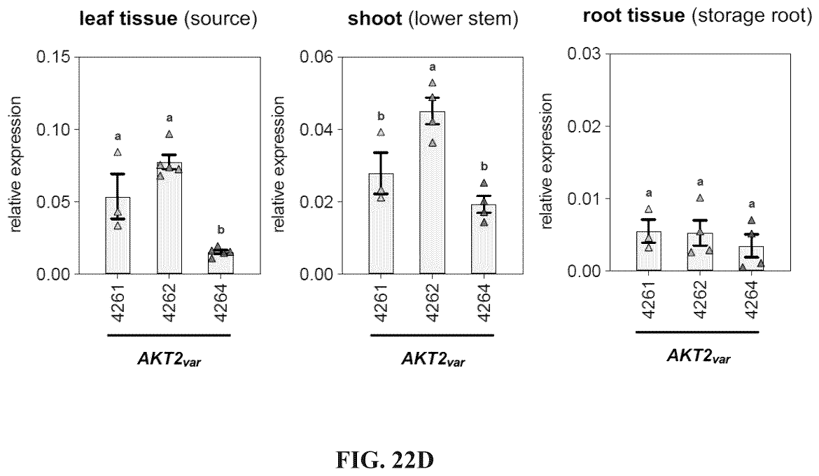

To assess differences in growth performance, additional cassava plants were grown in a greenhouse for about 19 weeks from the three EV control lines EV-4218, EV-4234, and EV-4243 alongside the transgenic AKT2var-expressing lines AKT2var-4261, AKT2var-4262, and a third line, AKT2var-4264, with comparable AKT2var expression levels as AKT2var-4261 and AKT2var-4262 by RT-qPCR (FIG. 22D). Plant total shoot dry matter (TSDM) and total storage root dry matter (TRDM), as well as harvest index dry weight, were determined for all plants (FIGS. 16C-16D and FIGS. 9A-9F).

### Plant Height

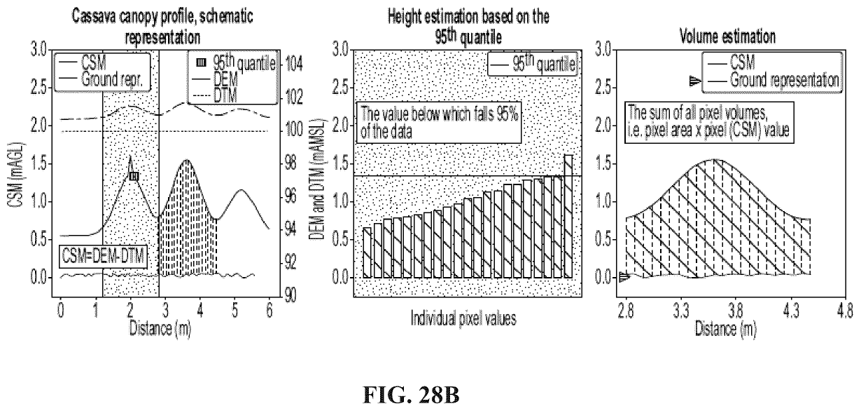

Unmanned aerial vehicle (UAV) flight campaigns were conducted with a Mikrokopter Okto-XL 6S12 (HiSystems GmbH, Moormerland, Germany). A high resolution RGB camera (Sony alpha 6000 with 35 mm lens; Sony Group Corporation, Tokyo, Japan) was used to collect imagery with 80% overlap (side and forward) at 27 meters above ground level (mAGL), resulting in a pixel size of 0.003 m. Nadir images were collected close to solar noon, typically between 11:00 and 13:00 hours local time, on a weekly or bi-weekly basis. A total of 30 ground control points (GCPs) were used to georeference the data based on their known position measured with a real-time kinematics (RTK)-global navigation satellite system (GNSS) system, achieving an accuracy of ˜0.03 m. Individual raw images were further processed with the photogrammetric structure from motion software Metashape (Agisoft LLC, St. Petersburg, Russia), from where georectified mosaic images, and digital elevation models (DEMs, blue continuous line in FIG. 28B) were generated. The DEMs contain information in meters above the mean sea level (mAMSL), and therefore the elevation of the digital terrain model (DTM, blue dashed line in FIG. 28B), also in mAMSL, needed to be subtracted from them in order to obtain the crop surface data. Thus, crop surface models (CSMs, depicted in FIG. 28B) providing plant height information in mAGL were computed for each UAV data acquisition.

Plant height per plant was calculated as the 95th quantile of the CSM values (FIG. 28A) within a ˜0.50 m buffer around each plant center, aiming to reduce noise from outliers (FIG. 28A). Plant volume was estimated as the sum of the CSM pixel values within each buffer, multiplied by the pixel area (FIG. 28A).

### Phenotype Analysis

qRT-PCR was performed as in Example 2.

### Results

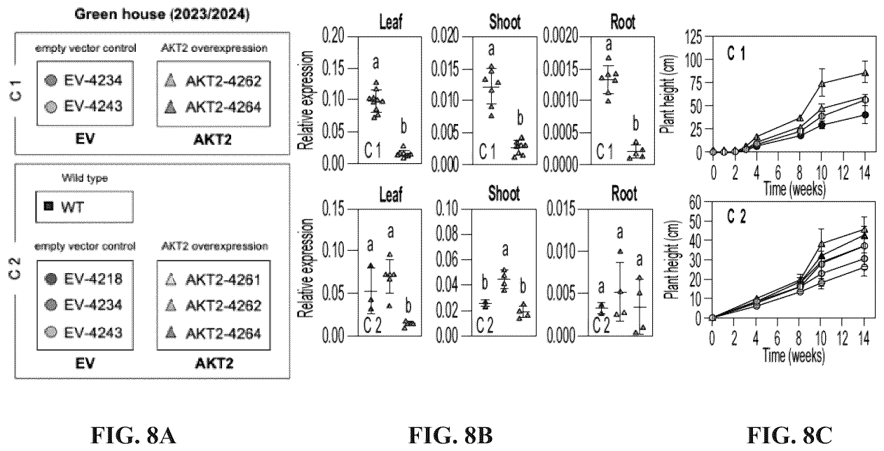

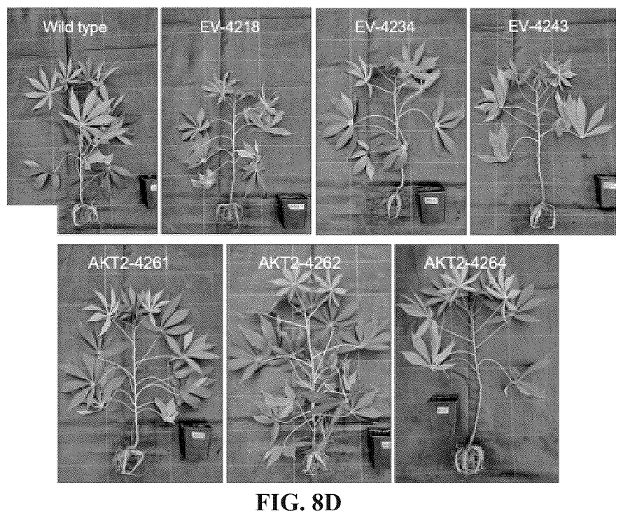

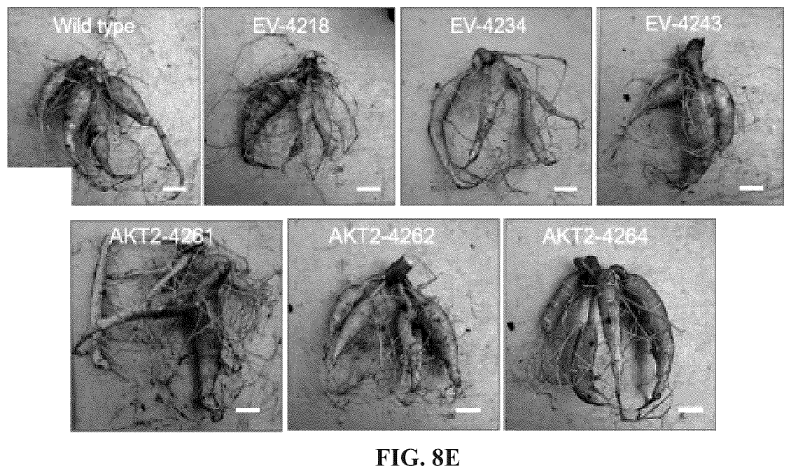

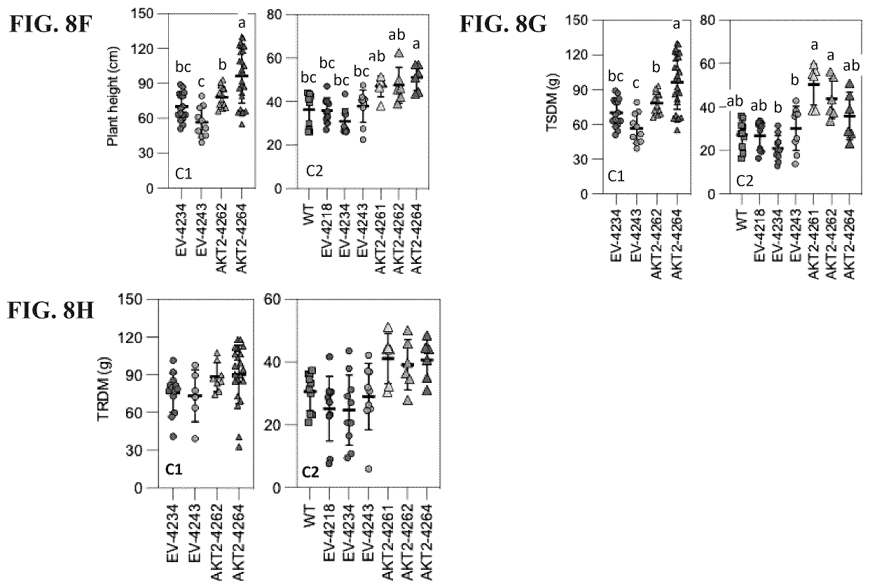

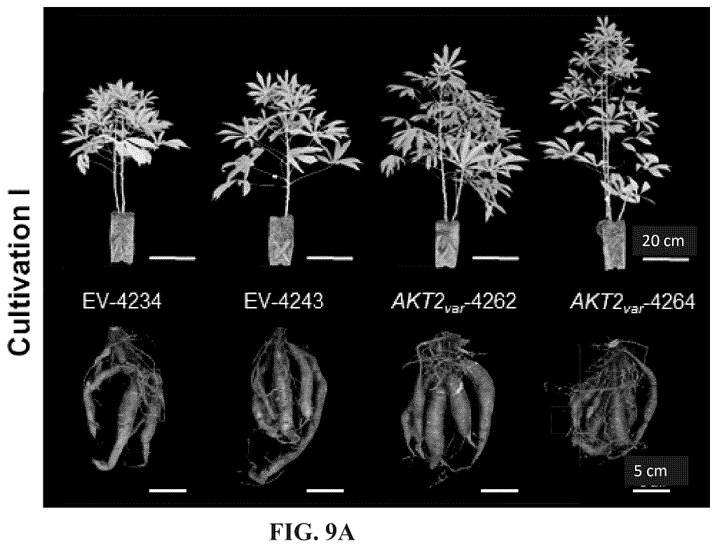

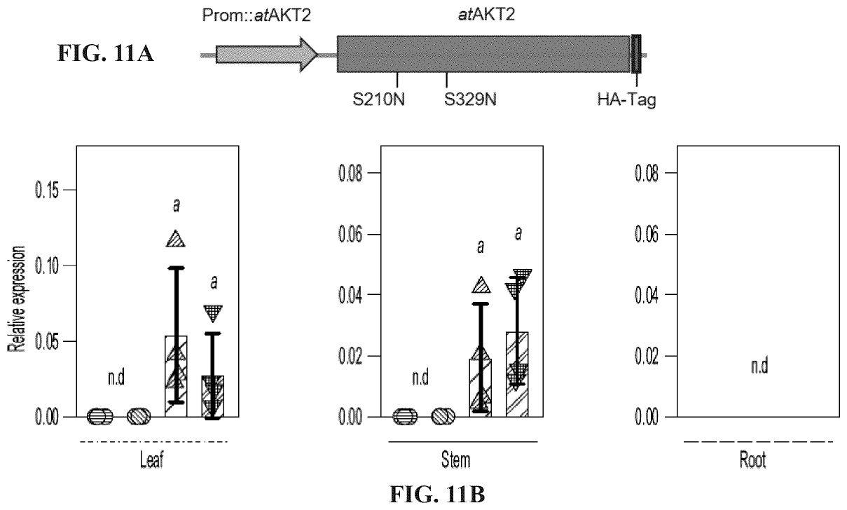

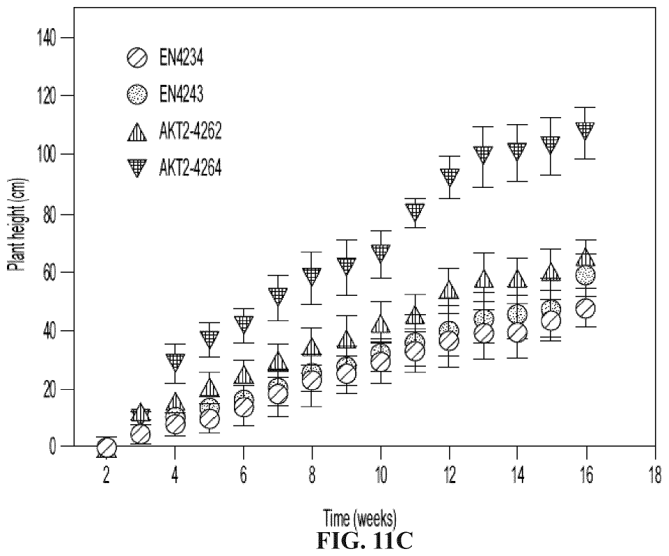

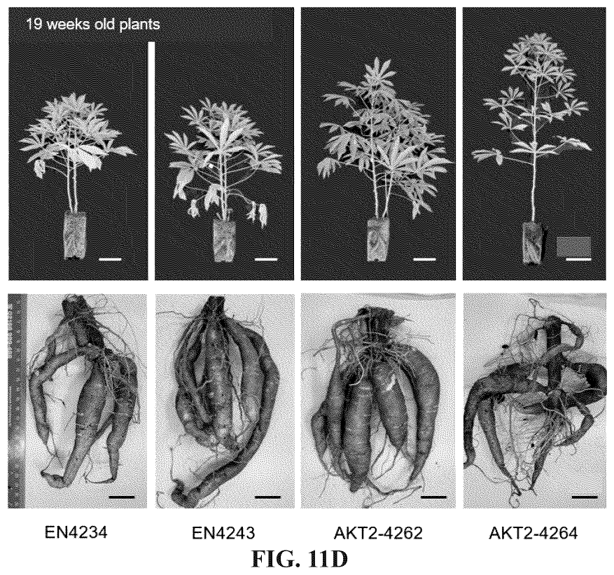

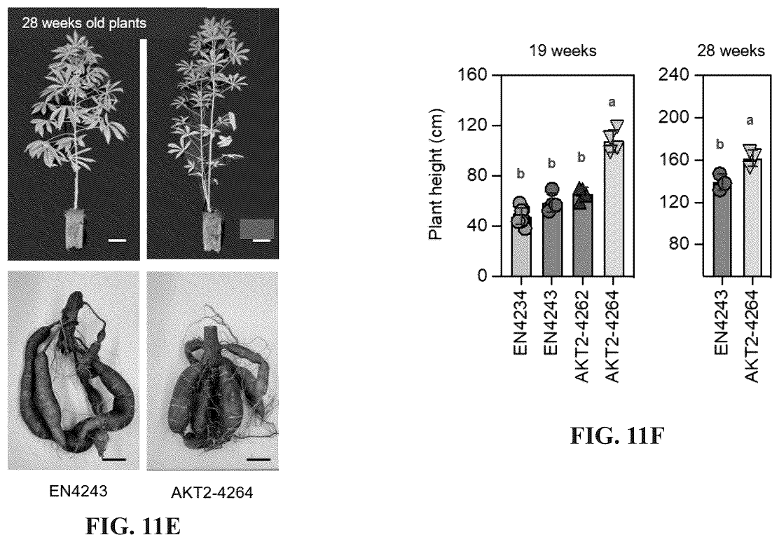

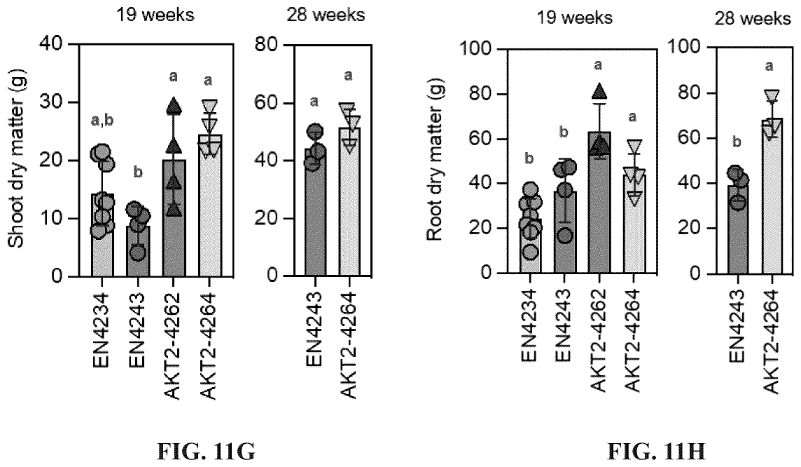

AtAKT2var overexpression events and empty vector controls were also grown under greenhouse conditions. qRT analyses confirmed that AtAKT2var was expressed at detectable levels in leaf, shoot, and root tissue for the AKT2var-4261, AKT2var-4262, and AKT2var-4264 lines (FIGS. 8A-8B, FIGS. 11A-11B).

### Initial Results

Even though the plants grown under greenhouse conditions were significantly smaller than the corresponding plants grown in field trials, they showed comparable results in plant height, shoot weight and storage root growth (FIGS. 8A-8H). As in field trials, the AKT2var-4264 line showed a strongly increased plant height compared to all other lines (FIGS. 8C, FIGS. 8D-8E, FIGS. 11C-11F). A further agreement was shown in the determination of the total shoot dry matter (TSDM). While the AKT2var-4261 and AKT2var-4262 lines showed increased TSDM compared to the other lines, the AKT2var-4264 line did not show a significant increase despite a greatly increased growth height (FIGS. 8F-8G, FIG. 11G). Further similarities with plants from the field trials were found when the storage roots were harvested. As with TSDM, the AKT2var-4261 and AKT2var-4262 lines had significant increases in total root dry matter (TRDM), whereas the other AKT2 overexpression lines had no significant increases over controls (FIG. 8H, FIG. 11H).

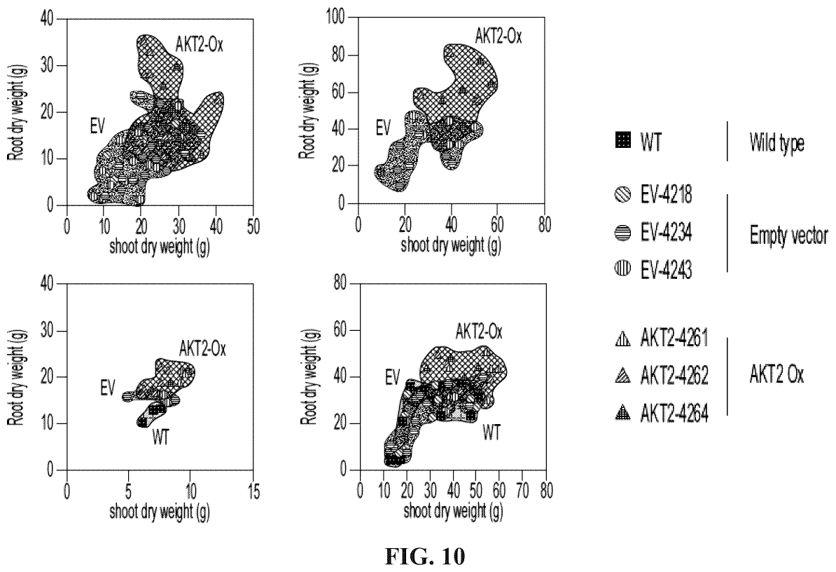

Furthermore, when plotting total shoot dry weight against total root dry weight, AtAKT2var overexpression lines tended to have higher weights than did wildtype or empty vector control plants grown under the same conditions (FIG. 10).

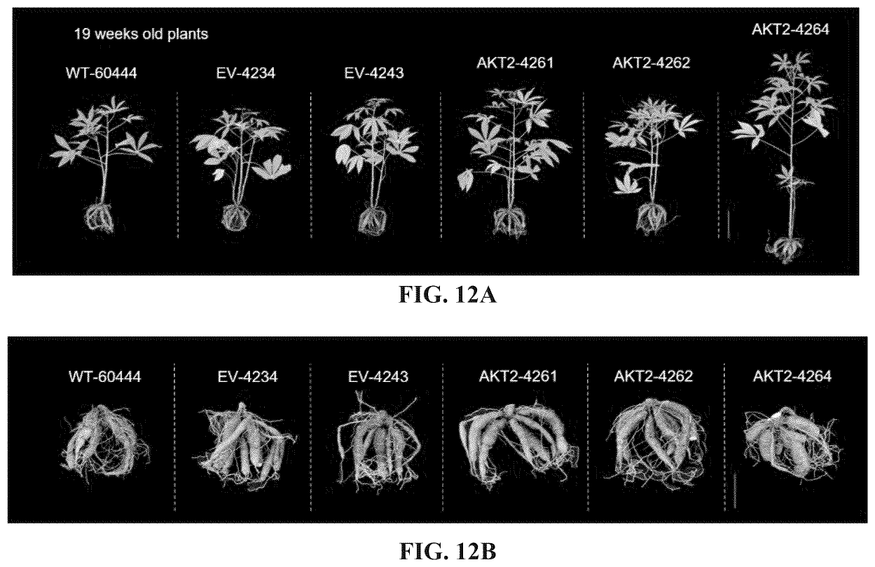

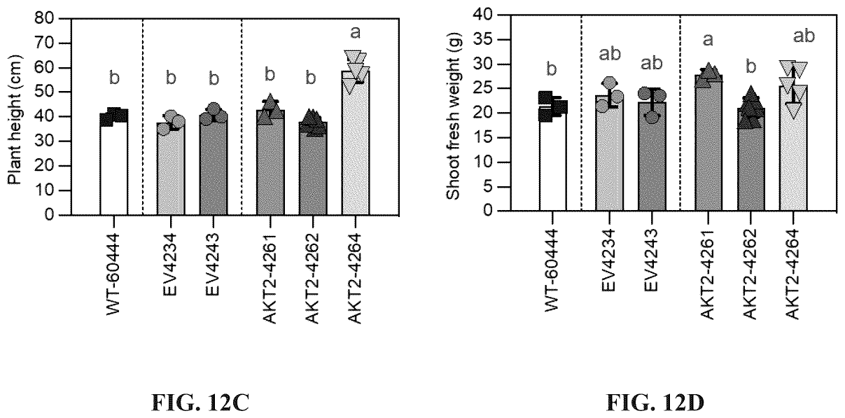

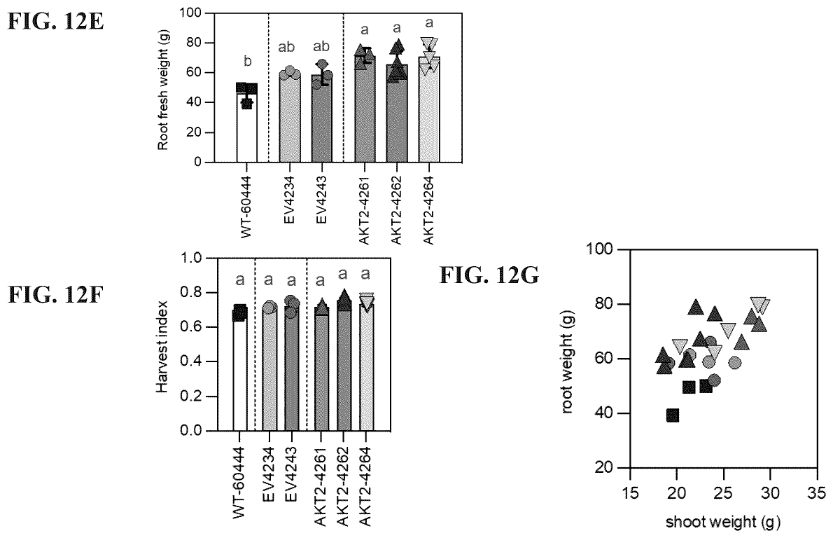

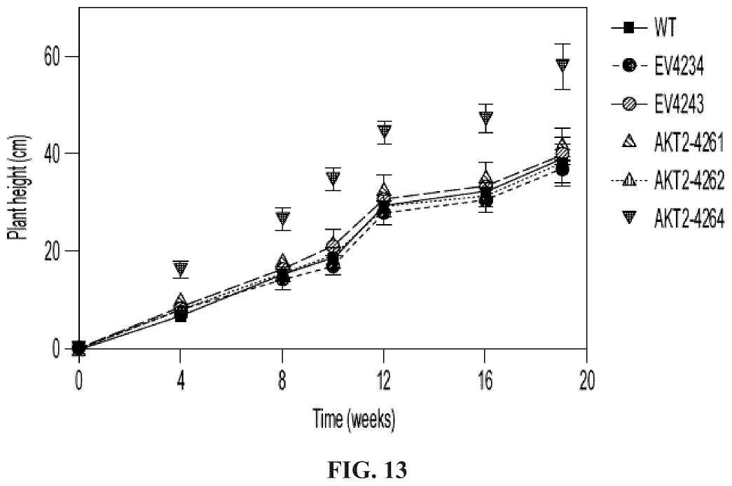

Additional analyses of plants grown under greenhouse conditions were also performed. Images of plants were captured (FIGS. 12A-12B). Phenotypes such as plant height (FIG. 12C), shoot fresh weight (FIG. 12D), root fresh weight (FIG. 12E), and harvest index (FIG. 12F) were analyzed, as was shoot weight in conjunction with root weight (FIG. 12G). In assessing plant height, AKT2 overexpression line ATK2var-4264 exhibited significantly greater plant height than any other tested line (FIG. 12C). In assessing shoot weight in conjunction with root weight, AKT2 overexpression line ATK2var-4262 exhibited a higher root weight for a given shoot weight when compared to other lines (FIG. 12G). While the harvest index was not significantly different when compared between lines (FIG. 12F), all three AKT2 overexpression lines showed significantly higher root fresh weight when compared to wild-type cassava plants (FIG. 12E). Plant height was also tracked for 19 weeks (FIG. 13), during which AKT2 overexpression line ATK2var-4264 continued to exhibit significantly greater growth in terms of plant height.

### Further Results

In further greenhouse testing, AKT2var-expressing plants (48.5 cm on average) were significantly taller than the EV controls (38.4 cm), corresponding to a 26.4% increase relative to EV controls (FIGS. 16C-16D). Similar results were obtained in three additional cultivation trials conducted under greenhouse conditions (FIGS. 9A-9F). All three AKT2var-expressing transgenic lines had significantly higher total shoot and root weights than the EV control plants. Specifically, TSDM was 20.5 g for AKT2var-4261 and 18.6 g for AKT2var-4262, representing a 34.0% and 21.2% increase, respectively, compared to 15.3 g for the EV-4234 control. The TSDM of line AKT2var-4264 rose more modestly, by 24.6%, with a weight of 19.1 g. The AKT2var-expressing plants also produced significantly heavier storage roots, with AKT2var-expressing lines having a mean TRDM of 8.4 g, representing a 56.4% increase relative to the EV controls (5.3 g) (FIG. 16D and FIGS. 9A-9F).

### Example 6: Overexpression of AtAKT2var Only Minorly Affected Ion Distributions in Shoot and Root Tissue in Greenhouse Trials

The following example describes experiments that showed that overexpression of mutated Arabidopsis thaliana AKT2 in cassava resulted in minor alterations of the concentrations of various cations and anions in the shoot and root tissue for greenhouse trials.

### Methods

Cassava plants were grown as in Example 5. Ion quantification was performed as in Example 1.

### Results

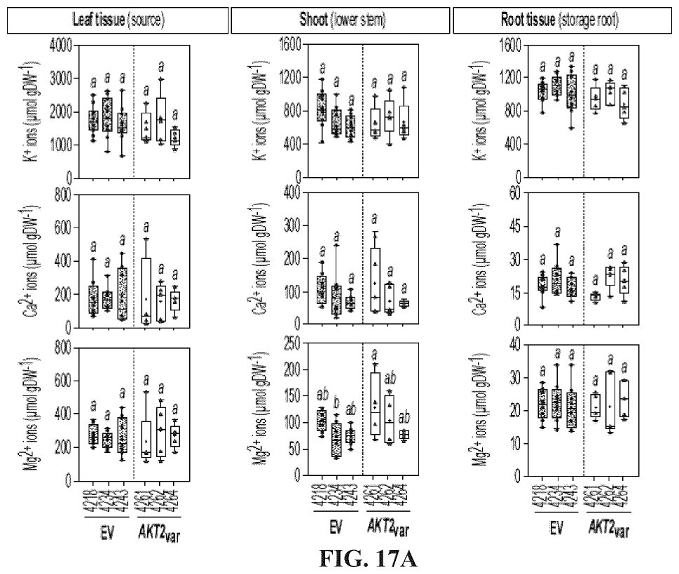

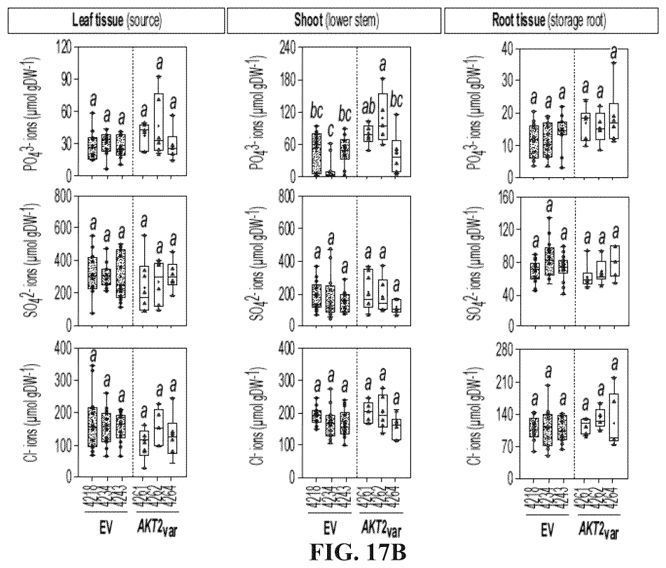

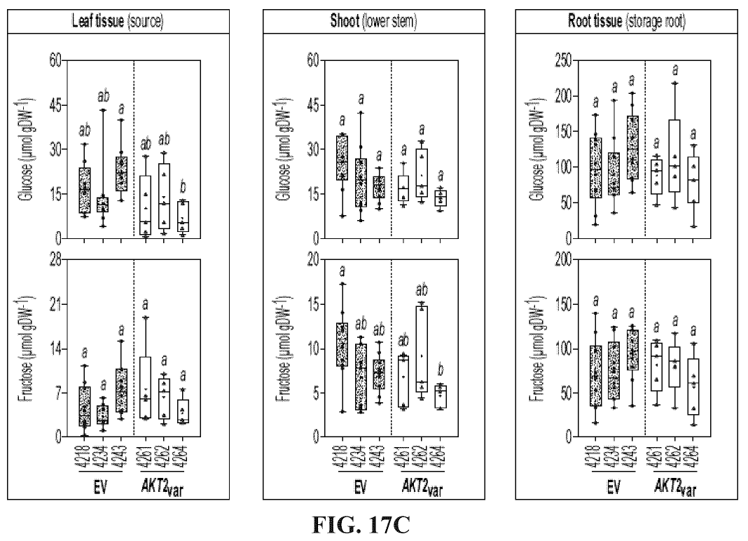

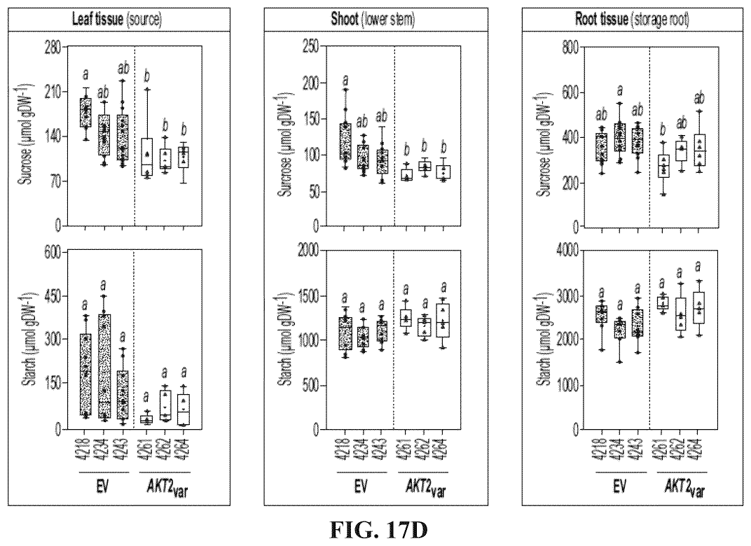

Controlled greenhouse trials revealed only minor changes in ion distributions. The quantification of specific cations and anions per gram dry weight (g DW) revealed only minor changes. The concentrations of the key cations K⁺, calcium (Ca²⁺), and magnesium (Mg²⁺) (FIG. 17A and FIG. 9G); anions phosphate (PO₄³⁻), sulfate (SO₄²⁻), and chloride (Cl⁻) (FIG. 17B and FIG. 9H); and sugar levels for glucose and fructose (FIG. 9I) did not differ significantly in leaves, shoots, and storage roots between EV control and the AKT2var-expressing lines (FIG. 17A and FIGS. 9G-9I).

### Example 7: Overexpression of AtAKT2var Boosted Photosynthetic Performance in Greenhouse Trials

The following example describes experiments that showed that overexpression of Arabidopsis thaliana AKT2 in cassava resulted in increased photosynthesis, as well as altered sugar and starch levels.

### Methods

### Plant Growth

Cassava plants were grown as in Example 5.

### Measurements of Sugar and Starch Levels

Sugar and starch measurements were conducted as described in Example 1.

### Measurements of Photosynthetic Performance

Measurements of volume-based seasonal growth were performed with an RGB camera installed on an unmanned aerial vehicle (UAV). Measurements of electron transport rate were performed with a monitoring pulse amplitude modulation (MoniPAM). Raw data downloaded from MoniPAM was filtered based on the visual (not automated) comparison between the diurnal cycles of PAR and the quantum efficiency of photosystem II, Y (II).

### PAM Measurements

Photosynthesis data of a total of 12 AKT2var and 12 EV plants were acquired in the confined field trial at NCHU Experimental Station Taichung, Taiwan, from November 18 to Nov. 27, 2022. The term “confined field trial” herein means a field experiment of isolated plants (or an isolated field) that allows evaluation of phenotypes and biosafety for transgenic plants. Plants and fields were fenced-in and isolated from wild populations. For this, a monitoring pulse amplitude modulation system (MoniPAM; Heinz Walz, Effeltrich, Germany) was used. The map with the location of the measured plants is shown in FIG. 28A, whereas examples of the instruments attached to samples at varying light conditions are presented in FIG. 28A. The measuring heads were placed southwards in order to ensure maximum exposure to the incoming photosynthetically active radiation (PAR), which was measured using the white panel shown on FIG. 28A. The electron transport rate (ETR) was computed as described by (Maxwell, K. and Johnson, G. N. (2000) Chlorophyll fluorescence—a practical guide. Journal of 1167 Experimental Botany, 51, 659-668), and subsequently fitted to an exponential raise to maximum curve where the maximum ETR (ETRmax) value is calculated and used as a reference of photosynthetic capacity (Rascher, U., Liebig, M. and Lüttge, U. (2000) Evaluation of instant light-response curves of chlorophyll fluorescence parameters obtained with a portable chlorophyll fluorometer on site in the field. Plant, Cell & Environment, 23, 1397-1405.).

### In Vivo Chlorophyll Fluorescence Measurements

In initial greenhouse trials, a MINI Version IMAGING-PAM fluorometer (Walz Instruments, Effeltrich, Germany) was used for in vivo chlorophyll fluorescence measurements (Schreiber, U., Kiihl, M., Klimant, I., & Reising, H. (1996). Measurement of chlorophyll fluorescence within leaves using a modified PAM Fluorometer with a fiber-optic microprobe. Photosynthesis Research, 47(1), 103-109). Seven- and 21-day-old plants were dark adapted for 10 minutes before the measurement. After two saturating light pulses in the dark, actinic light stepwise increased (PAR=1, 21, 41, 76, 134, 205, 249, 298, 371, 456, 581, and 726) every 20 s without additional dark phases. Results were calculated and visualized with Software ImagingWin (v2.41a; Walz Instruments, Effeltrich, Germany).

### Gas Exchange-Related Parameter Measurements

In initial greenhouse trials, gas exchange-related parameters were analyzed with a GFS-3000 system, model 3000-C, with a 3010-M sensor head and a 3055-FL fluorescence unit model (Heinz Walz, Effeltrich, Germany). Individual plants were placed in a 2 cm² gas exchange cuvette, and the following parameters were recorded: CO2-assimilation rate, respiration, leaf CO₂ concentration, and stomatal conductance. The cuvette was set to the conditions for plant growth, including a temperature of 28° C., humidity of 65%, airflow of 650 μmol/s and CO₂ concentrations of 475 ppm. Light respiration was measured for each plant over a period of 1 minute at PAR 125, and dark respiration at PAR 0. Each plant was measured three times with 30 see intervals between measurements to allow the leaves to return to a stable value. The steady-state value was identified automatically by the measured parameters. Stability criteria are provided in Table 3.

TABLE 3
Stability criteria.

| | | Analysis | | |
|---|---|---|---|---|
| Value | Use/ignore | time(s) | Criterion (change smaller than) | |
| CO2abs | = use = | 60 | 4 | ppm/min |
| dCO2ZP | = use = | 20 | 0.4 | ppm/min |
| dCO2MP | = use = | 60 | 0.4 | ppm/min |
| H20abs | ignore rising | 180 | 50 | ppm/min |
| dH20ZP | = use = | 30 | 2 | ppm/min |
| dH20MP | ignore falling | 60 | 20 | ppm/min |
| Pamb | = use = | 30 | 0.1 | kPa/min |
| Flow | = use = | 30 | 2 | μmol/s/min |
| Aux1 | ignore | 0 | 3 | mV/min |
| Aux2 | ignore | 0 | 3 | mV/min |
| Tcuv | = use = | 30 | 0.2 | ° C./min |
| Tleaf | = use = | 30 | 0.2 | ° C./min |
| Ttop | = use = | 30 | 0.2 | ° C./min |
| PARtop | ignore | 30 | 3 | μmol m−2 s−1/min |
| PARbot | ignore | 30 | 3 | μmol m−2 s−1/min |
| PARamb | ignore | 0 | 3 | μmol m−2 s−1/min |
| rh | ignore | 300 | 0.2 | %/min (GWK only) |
| E | ignore falling | 120 | 0.2 | mmol m−2 s−1/min |
| VPD | = use = | 120 | 0.1 | Pa/kPa/min |
| GH20 | ignore falling | 120 | 1 | mmol m−2 s−1/min |
| A | = use = | 120 | 0.2 | μmol m−2 s−1/min |
| ci | ignore falling | 60 | 7 | ppm/min |
| ca | ignore | 60 | 5 | ppm/min |
| wa | = use = | 180 | 30 | ppm/min |
| Ft | = use = | 45 | 3 | mV/min |

Additionally, photosynthetic performance was measured through ¹¹C-PET analysis, ¹¹C-transport velocity, and assessment of CO₂ assimilation.

### ¹¹C-PET Analysis: Tracer Production and Application

Online production of ¹¹CO₂ was achieved via the ¹⁴N(p,a)¹¹C nuclear reaction by irradiation of N in a gas target with 18 MeV protons at the IBA 18/9 MeV cyclotron of the Institute of Plant Sciences “CYPRES” at Forschungszentrum Jülich GmbH. The ¹¹CO2 was collected in specially designed trapping devices similar to the one described in Kim et al. 2014 (Kim, D., Alexof, D. L., Schueller, M., Babst, B., Ferrieri, R., Fowler, J. S. and Schlyer, D. J. (2014) The design and performance of a portable handheld 11CO2 delivery system. Applied Radiation and Isotopes, 94, 338-343.) for transfer to the plant labelling circuit. At the end of the collection period, the activity in the closed trap was measured with a collimated scintillation detector (1″ NaI Scionix detector, Scionix, Bunik, The Netherlands) connected to an Osprey MCA (Mirion Technologies, Rüsselsheim, Germany) before transferring the trap to the labelling system. Plant labelling was performed according to Yu et al. 2024 (Yu P, Li C, Li M, He X, Wang D, Li H, Marcon C, Li Y, Perez-Limón S, Chen X, et al. 2024. Seedling root system adaptation to water availability during maize domestication and global expansion. Nature Genetics 56(6): 1245-1256). Briefly, the activity in the labelling system was circulated in a closed loop until the target activity of 50 MBq was reached, upon which two valves were switched to include the plant leaf-cuvette for 6 minutes into the closed circuit. After 6 minutes had expired, the leaf cuvette was again switched to open mode. In open mode the cuvette was then again supplied with conditioned gas from a gas mixing unit with temperature 26±0.5° C., humidity 66±4% and CO2 390±10 ppm controlled as it was before the measurement. The outflow from the cuvette was passed through a CO2 absorber encased in lead shielding to safely dispose of excess radioactivity.

The in- and outflow of the cuvette was monitored by the following sensors: a differential infrared gas analyser IRGA (LI-7000, LI-COR Biosciences GmbH, Bad Homburg, Germany), a mass flow meter (LowDeltaP, Bronkhorst Deutschland Nord GmbH, 59174 Kamen, Germany), an atmospheric pressure sensor (144SC0811BARO, Sensortechnics, First Sensor 12459 Berlin, Germany) and relative humidity and temperature sensor (AC3001, Rotronic Messgeräte GmbH, 76275 Ettlingen, Germany). The resulting data was used to calculate leaf assimilation rate according to (Jahnke, S. (2001). Atmospheric CO₂ concentration does not directly affect leaf respiration in bean or poplar. Plant, Cell & Environment, 24(10), 1139-1151). Values are given as mean±standard deviation for a period of 2 hours starting 5 minutes after the end of ¹¹CO₂ labelling. Leaf area was measured destructively after harvest (and used to calculate CO₂ uptake per leaf area.). The gas-exchange measurement and labelling system is described in detail in Metzner et al. 2022 (Metzner, R., Chlubek, A., Bühler, J., Pflugfelder, D., Schurr, U., Huber, G., Koller, R. and Jahnke, S. (2022) In Vivo Imaging and Quantification of Carbon Tracer Dynamics in Nodulated Root Systems of Pea Plants. Plants (Basel, Switzerland), 11). Labelling experiments were performed on the 7th or 8th leaf from the top of the plant, which was the youngest source leaf present on the plant.

### Growth and Measurement Conditions

Two days before and between the PET measurements, the plants were kept in a climate chamber with temperature 28° C., humidity 65±3% and 400±10 μmol m⁻¹ s⁻¹ PAR at ambient CO₂ concentration during the 16 h light period. During the 8 h dark period, temperature was dropped to 22±0.5° C. and humidity was kept constant. The climate chamber housing the PET-instrument and the plant during the measurement was set to similar conditions.

### ¹¹C-PET Measurements

The PET system phenoPET used here is a custom built vertical-bore instrument for plant measurements with a field of view of 180 mm diameter and 200 mm height. Details on the instrument and a comparison to other plant-dedicated PET system can be found elsewhere (Hinz, C., Jahnke, S., Metzner, R., Pflugfelder, D., Scheins, J., Streun, M., & Koller, R. (2024). Setup and characterisation according to NEMA NU 4 of the phenoPET scanner, a PET system dedicated for plant sciences. Physics in Medicine & Biology, 69(5), 055001). The system is mounted on a gantry so it can be moved vertically around a potted plant and the whole setup is installed within a climate chamber. Images were reconstructed from the data using the PRESTO toolkit (Scheins, J. J., Herzog, H. and Shah, N.J. (2011) Fully-3D PET Image Reconstruction Using Scanner-Independent, Adaptive Projection Data and Highly Rotation-Symmetric Voxel Assemblies. IEEE Transactions on Medical Imaging, 30, 879-892).

¹¹C-Tracer Data Processing and Analysis of ¹¹C-Transport Velocity

In the reconstructed 3D images of the tracer signal distribution, cylindrical regions of interest (ROI) were placed along the stem. The position of the ROIs in the 3D PET image was determined using anatomical information from visual or imaging observations. The changing activity in these ROI over time, resulting from ¹¹C tracer that was assimilated after ¹¹CO₂ pulse-labelling of a leaf and moving through the stem towards the root, was registered over time as time-activity curves (Bühler, et al. (2014) A class of compartmental models for long-distance tracer transport in plants. J Theor Biol, 341, 131-142; Lanzrath et al. 2025. Analyzing time activity curves from spatio-temporal tracer data to determine tracer transport velocity in plants. Mathematical Biosciences 383:109430). The method used to determine tracer transport velocities from the TAC curves with a compartmental transport model is described in detail in Lanzrath et al. (2025). In the part of the stem directly below the insertion of the petiole (˜5-7 cm) connected to the labelled leaf the data quality was suboptimal, so that only the lower part of the stem in the field of view was used for transport velocity analysis.

### Results

Early computational modelling efforts led to the assumption that expressing AKT2var in Arabidopsis promoted reloading of sucrose leaked out of the phloem system, resulting in higher sucrose concentrations in the sieve element/companion cell (SC/CC) complex (Gajdanowicz et al., 2011, ibid.). However, whether AKT2var expression might lead to higher phloem transport rates had not been experimentally tested. To investigate this aspect, positron emission tomography (PET) analysis was conducted using greenhouse-grown cassava plants using ¹¹C-labelled CO₂. Accordingly, leaves were incubated, collected from the empty vector (EV) control line EV-4234 and the best-performing AKT2var-expressing lines (AKT2var-4261 and AKT2var-4262) in the light with ¹¹C-labelled CO₂, and quantified the resulting phloem flow velocities along the stem (FIG. 16A). For each genotype, three individual plants were analyzed, with 1 to 2 leaves and 1 to 4 individual labelling experiments per plant (FIG. 22B).

Significantly higher tracer transport velocity was measured in the AKT2var-4261 and AKT2var-4262 plants than in the empty vector control EV-4234. Specifically, AKT2var-4261 and AKT2var-4262 exhibited mean tracer velocities of 11.6 mm min⁻¹ and 11.4 mm min⁻¹, respectively, compared to 6.7 mm min-1 for the vector control, corresponding to a 74.5% and 70.7% increase for AKT2var-4261 and AKT2var-4262, respectively (FIG. 16B). Photosynthetic fixation capacity was higher in AKT2var-4261 and AKT2var-4262 than in EV plants, as indicated by quantification of CO₂ assimilation rates. The CO₂ assimilation rate of AKT2var-4261 was 3.9 μmol m-2 s-1, and that of AKT2var-4262 was 4.4 μmol m⁻² s⁻¹, compared to 2.2 μmol m⁻² s⁻¹ for the EV plants, corresponding to a 77.3% and 100% increase for AKT2var-4261 and AKT2var-4262, respectively, relative to the EV control (FIG. 16B and FIG. 22C).

In contrast to ion homeostasis, more pronounced differences appeared for carbohydrate levels. While no significant alterations in glucose or fructose levels were detected (FIG. 9I and FIG. 17C), sucrose and starch levels were notably different (FIG. 17D).

Indeed, sucrose levels were markedly lower in the leaves and stems of all AKT2var-expressing lines than in those of the EV controls (FIG. 17D). In leaves, sucrose levels were 171.8 μmol g DW⁻¹ on average for the EV controls and 111.8 μmol g DW⁻¹ for AKT2var transgenic lines, representing a 34.9% drop in the AKT2var lines. In lower stem tissues, sucrose levels were 118.1 μmol g DW⁻¹ for the EV controls and 81.6 μmol g DW-1 for AKT2var plants, or a 30.9% decline (FIG. 17D). By contrast, starch accumulation was slightly elevated in the lower stems and storage roots of AKT2var-expressing lines. Specifically, starch levels in storage roots were 2361 μmol g DW⁻¹ for the EV controls and 2712 μmol g DW⁻¹ for AKT2var-expressing plants, reflecting a 14.9% increase (FIG. 17D).

### Example 8: Overexpression of AtAKT2var Resulted in Alteration of Plant Growth Across Multiple Years of Confined Field Trials

The following example describes experiments that showed that increased field performance of AKT2var is dependent on the respective environmental conditions.

### Methods

### AtAKT2var Overexpression

Lines overexpressing AtAKT2var and control lines were prepared as in Example 2.

### Multi-Year Field Testing

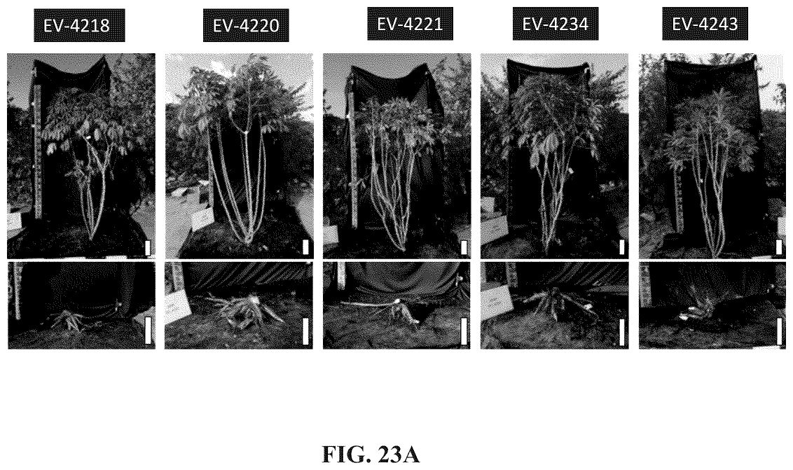

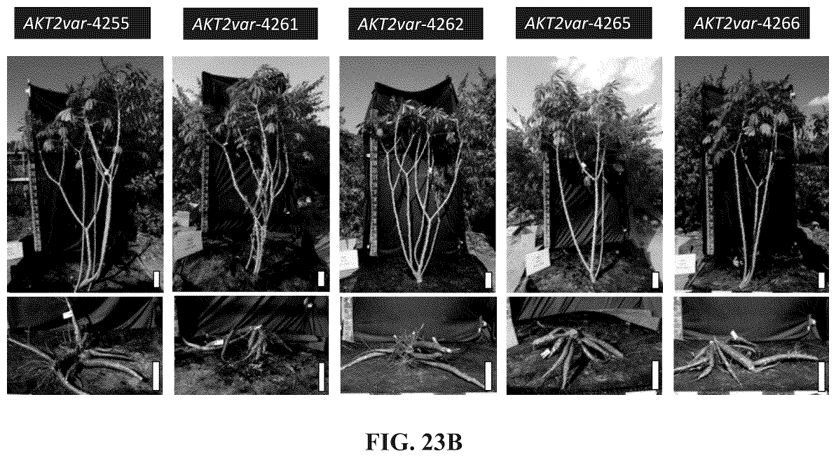

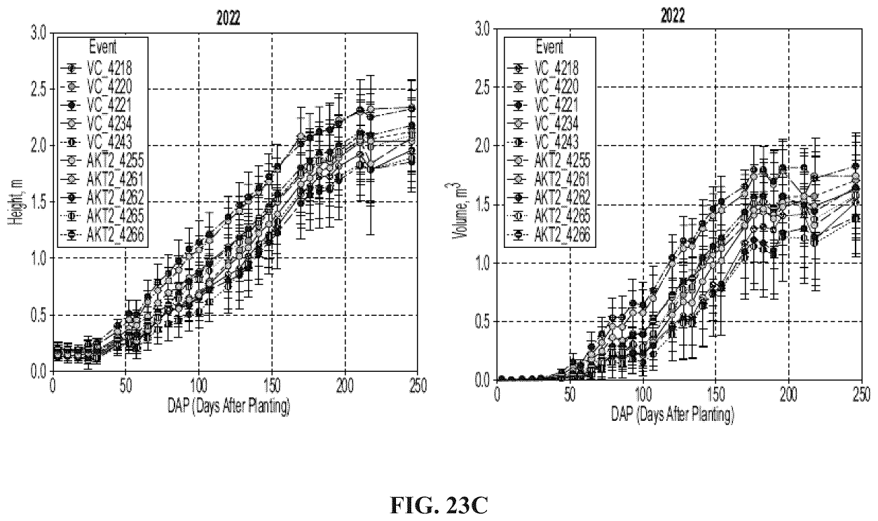

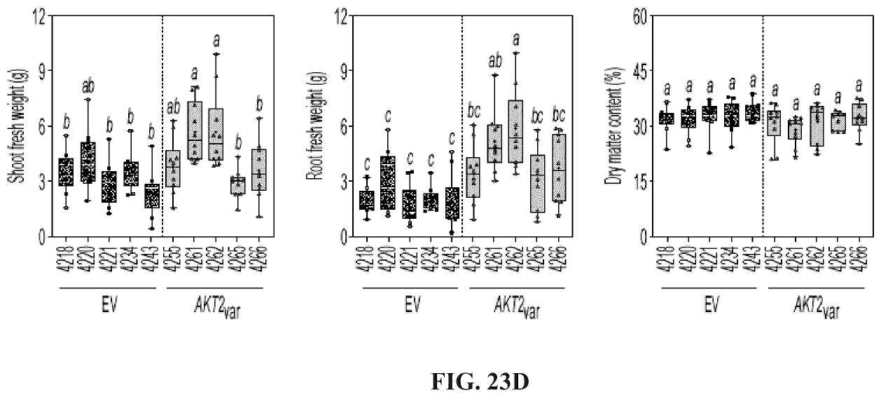

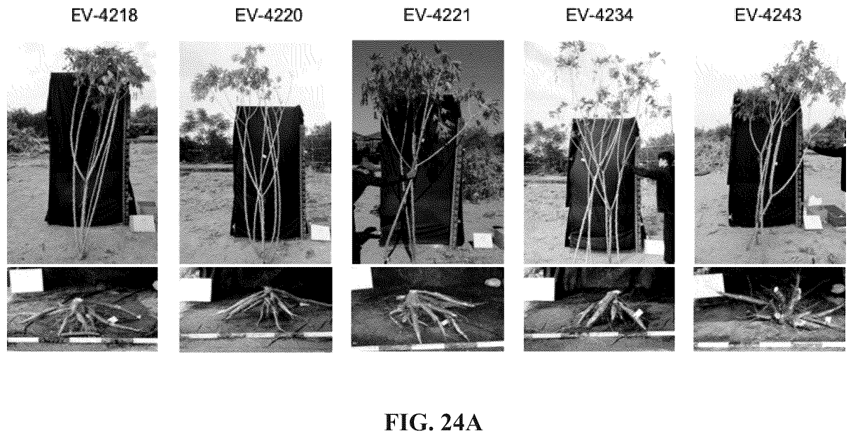

Further, confined field trials were performed at National Chung-Hsing University (NCHU) Experimental Station Taichung (Latitude 240 4′41.50″N; Longitude 120° 42′56.26″E), Taiwan in 2022, 2023, and 2024. To this end, plants from sterile culture were imported, transferred to soil, and subsequently hardened in a greenhouse, for 2-3 weeks, prior to transfer to the field. The fields were ridged and covered with black tarp to avoid the growth of weeds. Field growth was carried out in a randomized serpentine design (FIGS. 23A-23D) with at least 10 replications at final harvest. After a growth phase of 8 to 9 months, plants were harvested and parameters such as plant height, shoot fresh weight, and root fresh weight were recorded. Dry matter content was determined by drying a representative tissue piece. Total dry matter content was calculated by multiplying fresh weight and dry matter content. Samples were taken as described below in plant harvest and processing. Samples were freeze-dried, processed, and sent to RPTU Kaiserslautern, Germany, for further ion, sugar and starch analysis.

### Statistical Field Data Analysis

The data were processed stepwise to account for spatial and temporal variation. First, the data were corrected for design and spatial trends of the field by trait and year using the R package SpATS (Rodríguez-Álvarez, M. X., Boer, M. P., van Eeuwijk, F. A. and Eilers, P. H. C. (2018) Correcting for spatial heterogeneity in plant breeding experiments with P-splines. Spatial Statistics, 23, 52-71) following the formula: Y=f(r, c)+G+R+C, where Y is the phenotypic value, f(r, c) is a smoothed bivariate surface defined over rows and columns, G is the genotype effect, R is the effect of the row, and C the effect of the column. The number of spline points was set to two-thirds of the total number of rows and columns. Based on the spatial correction, outliers were excluded if the residual exceeds 3 standard deviations from the mean.

The best linear unbiased estimates (BLUEs) plus residual error were retained as spatially corrected values (Kronenberg, L., Yates, S., Boer, M. P., Kirchgessner, N., Walter, A. and Hund, A. (2020) Temperature response of wheat aGects final height and the timing of stem elongation under field conditions. Journal of Experimental Botany, 72, 700-717; Pérez-Valencia D M, Rodríguez-Ãlvarez M X, Boer M P, Kronenberg L, Hund A, Cabrera-Bosquet L, Millet E J, Eeuwijk F A V. A two-stage approach for the spatio-temporal analysis of high-throughput phenotyping data. Sci Rep. 2022 Feb. 24; 12(1):3177), which were used for correlation analysis and temporal analysis. In the second step, a mixed linear model was used to account for the temporal variation between the years. The spatially corrected values were used to calculate the genotypic BLUEs across the years for the individual traits using the R package lme4 (Bates, D., Machler, M., Bolker, B. and Walker, S. (2015) Fitting Linear Mixed-EGects Models Using lme4. Journal of Statistical Software, 67, 1-48.) and lmerTest (Kuznetsova, A., Brockhof, P. B. and Christensen, R. H. B. (2017) lmerTest Package: Tests in Linear Mixed EGects Models. Journal of Statistical Software, 82, 1-26.) following the formula: y=p+G+Y+R+F, where y is the spatially corrected value of the respective trait, p is the overall mean, G is the fixed effect of the genotypes, Y is the random effect of years, R is the random effect of the replicates, and F is the residual error. Prior to the modeling, an averaged vector control was calculated from the single vector controls, which functioned as the reference group in the t-test.

### Results

### Further, Multi-Year Testing

In confined field testing, AKT2var-expressing transgenic lines were cultivated at the National Chung-Hsing University (NCHU) Experimental Station in Taichung, Taiwan, from April to December over three consecutive years (2022-2024). In all three confined field trials, the same AKT2var-expressing lines (AKT2var-4255, AKT2var-4261, AKT2var-4262, AKT2var-4265, and AKT2var-4266) and EV control lines (EV-4218, EV-4220, EV-4221, EV-4234, and EV-4243) were planted in a randomized serpentine design. Each year, plants were harvested after about 8 to 9 months of field growth and measured for key agronomic parameters.

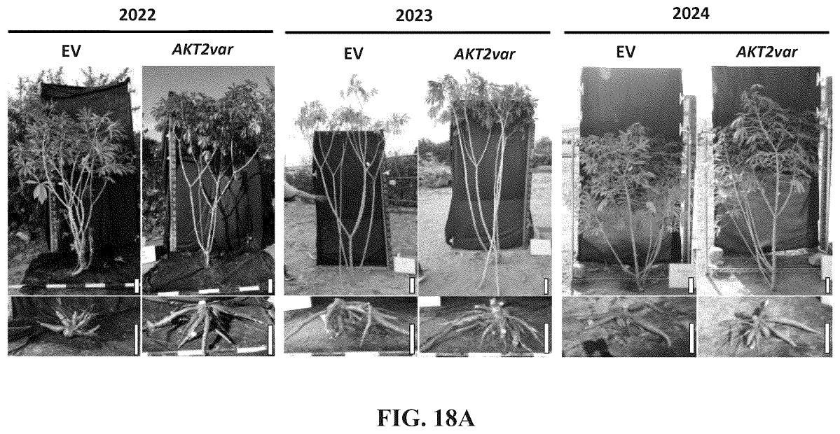

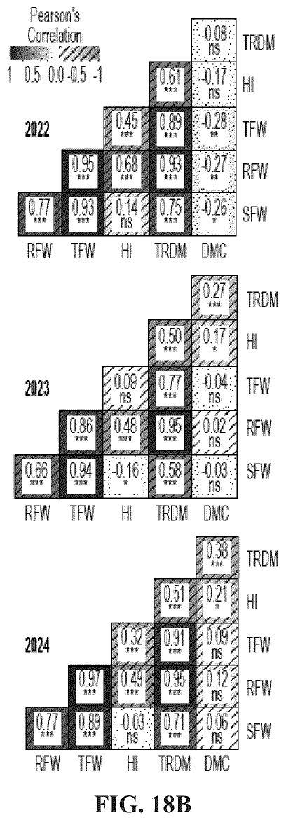

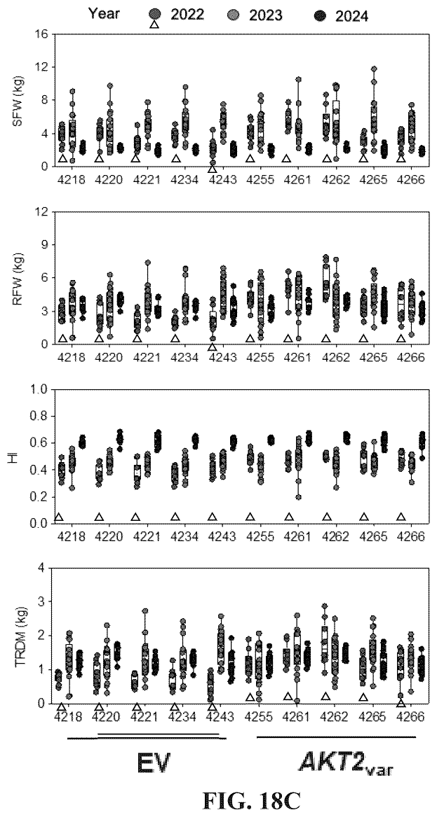

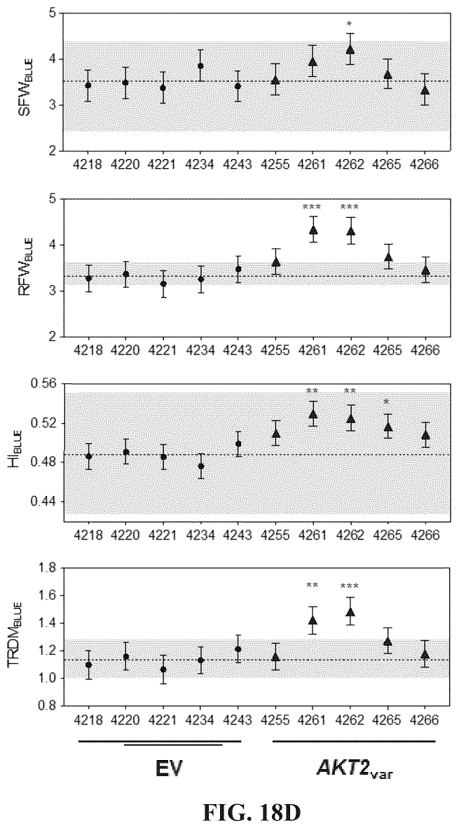

Representative photographs of AKT2var-expressing and EV control lines (FIG. 18A) already showed clear differences among the three growing seasons. Overall, plants grew tallest in 2023, while they grew more compact in 2024, with overall intermediate growth in 2022 (FIGS. 18A, 18C). In all three years, a significant and positive correlation was observed between aboveground and belowground growth (FIG. 18B). However, this correlation was less pronounced in 2023 (R=0.66) than in 2022 or 2024 (R=0.77) (FIG. 18B), with a weakly negative correlation between shoot fresh weight and harvest index in 2023 (R=−0.16) (FIG. 18B), suggesting a slightly higher ratio of aboveground growth in 2023.

Dry matter content (DMC) values showed significant and weakly negative correlations with the other traits in 2022, but not in 2023 or 2024 (FIG. 18B). While overall TRDM values were lower in 2022 than in the other two years for EV control lines, AKT2var-expressing lines did not show this decrease (FIG. 18C).

To evaluate the performance of AKT2var-expressing lines and EV lines over all three years, raw data was spatially corrected using the spATS package (Rodríguez-Álvarez et al., 2018, ibid.) in R, normalized the spatially corrected data to the mean vector control of each year and calculated best linear unbiased estimates (BLUEs) for SFW, RFW, HI, and TRDM (FIG. 18D).

This multi-year statistical analysis revealed a significant increase in SFW values for AKT2var-4262, as well as a significant increase in RFW and TRDM for AKT2var-4261 and AKT2var-4262. A consistent and significant improvement was observed for HI values for AKT2var-4261, AKT2var-4262, and AKT2var-4265 over the EV controls in this integrated analysis (FIG. 18D).

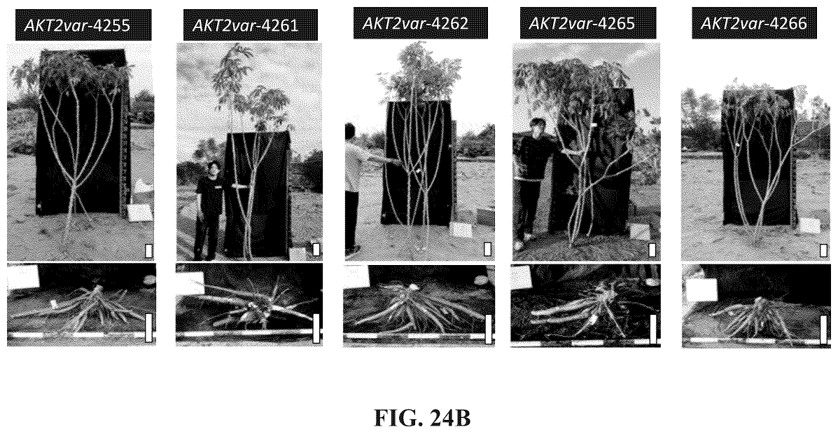

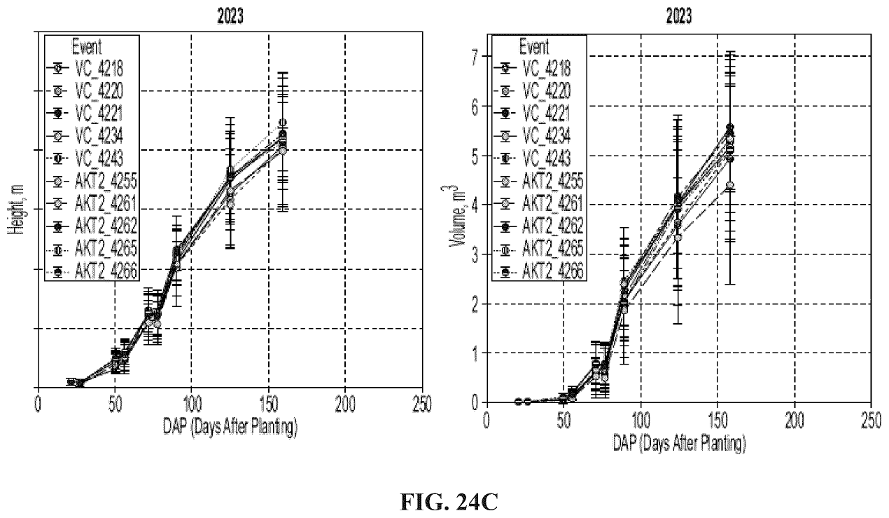

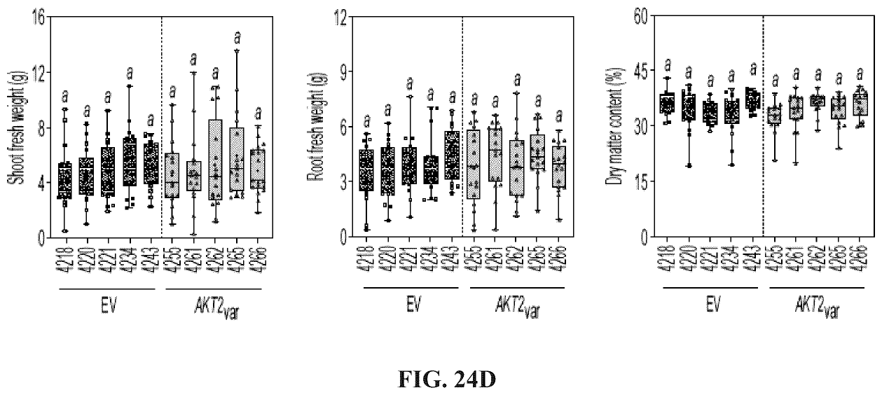

Although AKT2var expression did promote plant growth overall, the changes in performance varied greatly across the three confined field trials. While significantly higher shoot fresh weight was measured, storage root fresh weight, harvest index, and TRDM for select AKT2var-expressing lines in 2022 relative to EV control lines (FIG. 18C, FIGS. 23A-23D) significant changes were not observed in 2023 (FIG. 18C, FIGS. 24A-24D) and 2024 (FIGS. 18A-18D, FIGS. 25A-25H). In 2022, however, shoot fresh weight (SFW) was significantly higher in AKT2var-4261 (5.73 kg) and AKT2var-4262 (5.72 kg) than in the EV controls (3.3 kg), corresponding to a 73.6% and 73.3% increase over the controls, respectively (FIG. 18C, FIGS. 23A-23C). AKT2var lines also grew taller compared to the vector controls (FIGS. 23A-23D). In addition, AKT2var-4261 and AKT2var-4262 lines also had significantly higher storage root fresh weight (RFW) than did the EV controls (FIG. 18C, FIGS. 23A-23D). Specifically, the RFW for AKT2var-4261 was 5.19 kg, or a 48.3% increase compared to the EV controls (2.09 kg), while AKT2var-4262 had an RFW of 5.875 kg, reflecting an 81.1% increase over the EV controls (FIG. 18C, FIGS. 23A-23D). Additionally, AKT2var-4266 displayed a more modest, but still significant, increase compared to EV controls for RFW (FIG. 18C, FIGS. 23A-23D). Importantly, all five AKT2var-expressing lines tested in 2022 had significantly higher harvest index values than the EV control lines (FIG. 18C), indicating an improved assimilate allocation from source to sink tissues in these lines.

The dry matter content values were also calculated for all lines (FIGS. 23A-23D) and the total storage root dry matter content (FIG. 18C), as the most important yield parameter. While the DMC was about 30-35% among all lines and revealed no significant differences between AKT2var-expressing lines and EV controls, TRDM values were significantly higher in the AKT2var-4261, AKT2var-4262, and AKT2var-4266 lines than in the EV controls in 2022 (FIG. 18C).

This suggests that AKT2var improves overall growth rather than relative starch content under the tested field conditions. Precipitation patterns were markedly different in 2022 compared to 2023 and 2024, and while the plants received a comparatively high amount of rain early in the 2022 season, they received less rain for the rest of the season than in 2023 and 2024; the precipitation levels may explain the outperformance of control lines by the AKT2var plants in 2022 (FIG. 25E).

Overall, the results of the field trials are consistent with previous observations from multiple greenhouse trials (FIG. 16D, FIGS. 9A-9F), strongly suggesting a transport- and growth-promoting effect as a result of AKT2var expression.

The three consecutive confined field trials assessed the performance of AKT2var-expressing lines under agronomically relevant conditions. At least two out of six AKT2var-expressing lines exhibited significantly improved shoot and storage root biomass compared to EV controls (FIGS. 18A-18D, FIGS. 23A-23D), accompanied by a higher harvest index for at least three of the six AKT2var-expressing lines (FIGS. 18C-18D). While the differences were pronounced in 2022 and less so for the other two field seasons, it is important to note that field experiments are always subject to environmental influence and that variation in such results is to be expected.

An alternative explanation for the yield variance in confined field trials may derive from the relatively short growth periods in Taiwan. Due to the temperature drops in the winter month, the confined field site in Taichung only allows for a 9-month cassava growth season, while cassava on natural African fields is often grown for 12 months, prior to harvest. Thus, the shorter growing season may have resulted in harvesting storage roots that have not reached full maturity, with changes in shoot-root growth dynamics influencing final storage root yield. Nevertheless, all three confined field trials conducted in this study do support the positive effect of AKT2var expression on cassava performance, as displayed by higher ETRs (FIG. 19A), lower shoot sucrose levels (FIG. 19C), increased harvest index, and improved storage root yields (FIGS. 18A-18D, FIGS. 23A-23D). All of these observations point towards an accelerated assimilate allocation in AKT2var-expressing cassava lines. In sum, these results highlight a role for AKT2var in optimizing source-to-sink transport and support the function of K⁺ gradients as mobile energy sources for transport in the phloem (Gajdanowicz, P., Michard, E., Sandmann, M., Rocha, M., Correa, L. G., Ramirez-Aguilar, S. J., Gomez-Porras, J. L., Gonzalez, W., Thibaud, J. B., van Dongen, J. T. and Dreyer, I. (2011) Potassium (K+) gradients serve as a mobile energy source in plant vascular tissues. Proc Natl Acad Sci USA, 108, 864-869., Dreyer, I., Gomez-Porras, J. L. and Riedelsberger, J. (2017) The potassium battery: a mobile energy source for transport processes in plant vascular tissues. New Phytologist, 216, 1049-1053.).

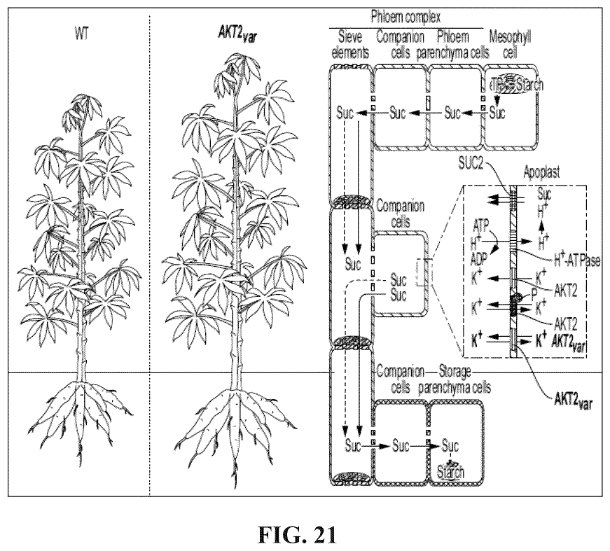

Phloem mass flow under controlled growth conditions is higher in AKT2var-expressing lines than in controls (FIG. 16A). Given that all solutes that enter the phloem are transported simultaneously with the same velocity, the unchanged leaf K⁺ levels in AKT2var-expressing plants (FIG. 17A) led to the assumption that K⁺ cycles between the phloem and xylem, which would maintain high leaf K⁺ levels, although its transport in the phloem is accelerated. Such potassium recycling potentially allows K⁺, subsequent to its export from the transport phloem (Gajdanowicz et al., 2011, ibid.), to be re-imported into the xylem, and from there back to the leaves (for a model, see FIG. 21). With this recycling, leaf K⁺ levels are stabilized, which is of particular importance to allow high rates of photosynthesis, especially under challenging drought conditions, as seen in other species (Jin, S. H., Huang, J. Q., Li, X. Q., Zheng, B. S., Wu, J. S., Wang, Z. J., Liu, G. H. and Chen, M. (2011) Effects of potassium supply on limitations of photosynthesis by mesophyll diffusion conductance in Carya cathayensis. Tree Physiology, 31, 1142-1151; Trankner, M., Tavakol, E. and Jikli, B. (2018) Functioning of potassium and magnesium in photosynthesis, photosynthate translocation and photoprotection. Physiol Plant.). In line with observations is i) the expression of the recombinant AKT2var gene in both phloem and xylem parenchyma (FIGS. 15A-15D), and ii) the fact that K⁺ fertilization generally improves photosynthesis, especially under abiotic stress conditions (Ho, L.-H., Rode, R., Siegel, M., Reinhardt, F., Neuhaus, H. E., Yvin, J.-C., Pluchon, S., Hosseini, S. A. and Pommerrenig, B. (2020) Potassium Application Boosts Photosynthesis and Sorbitol Biosynthesis and Accelerates Cold Acclimation of Common Plantain (Plantago major L.). Plants, 9, 1259.).

### Example 9: Overexpression of AtAKT2var LED to Improved Performance Under Controlled Drought Stress Conditions in Greenhouse Trials

Based on the result described in Example 8, it was hypothesized that AtAKT2var could affect drought tolerance. The following example describes the effect of AtAKT2var on improving drought tolerance in cassava.

### Methods

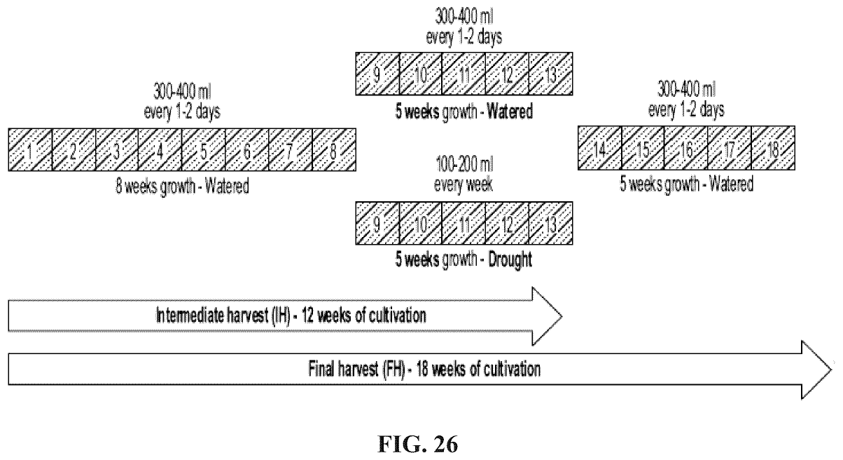

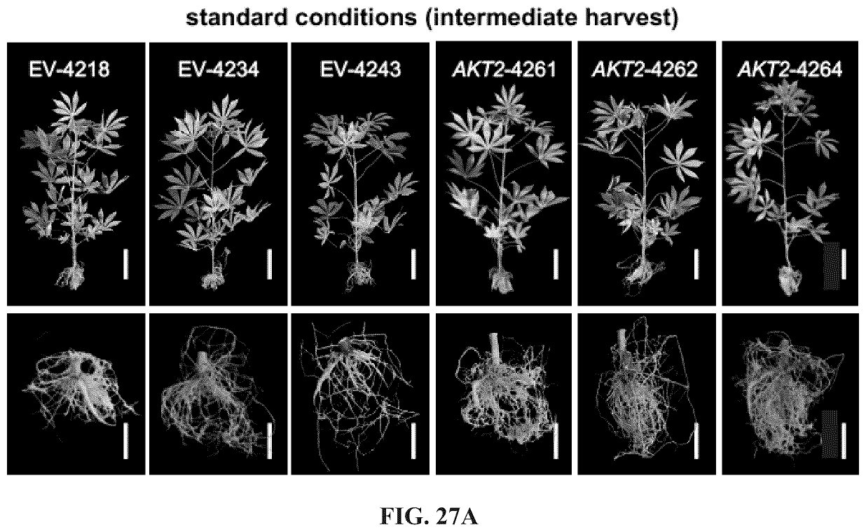

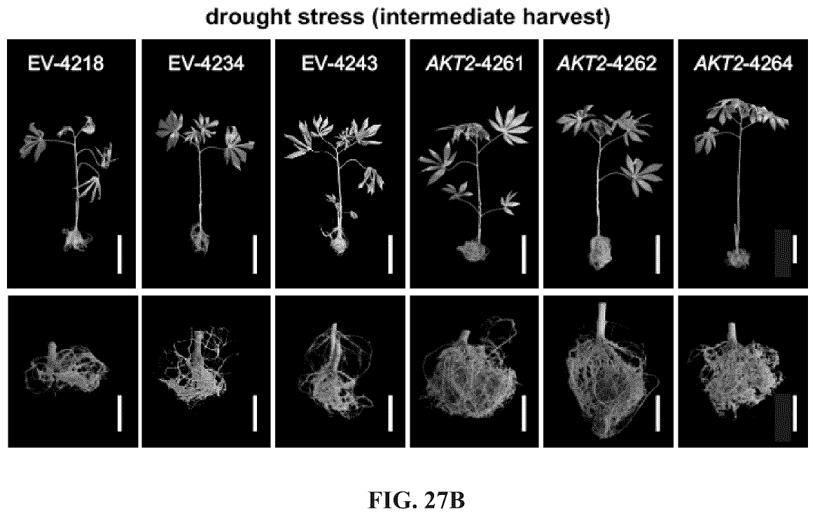

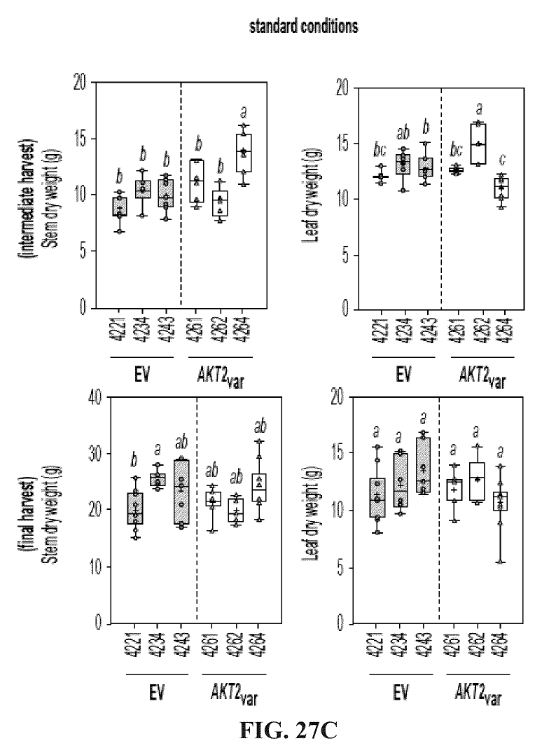

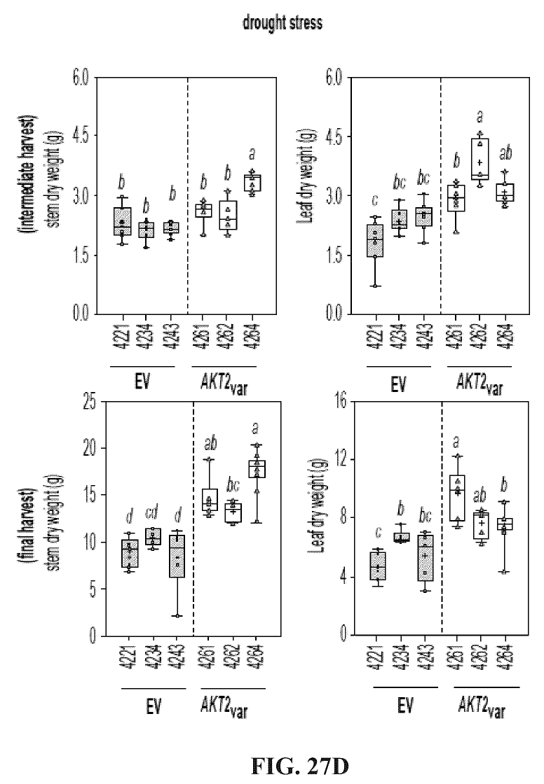

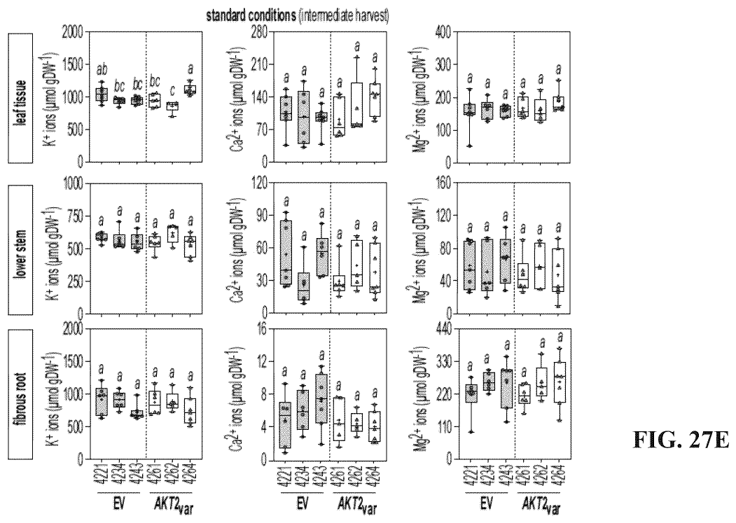

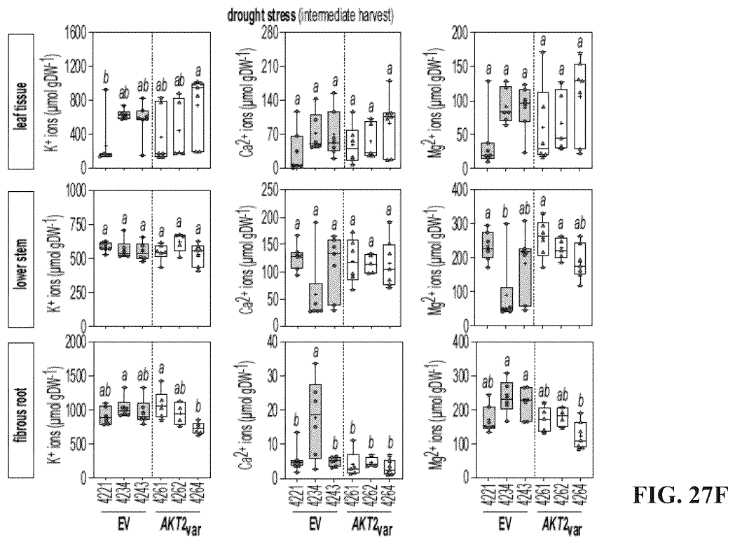

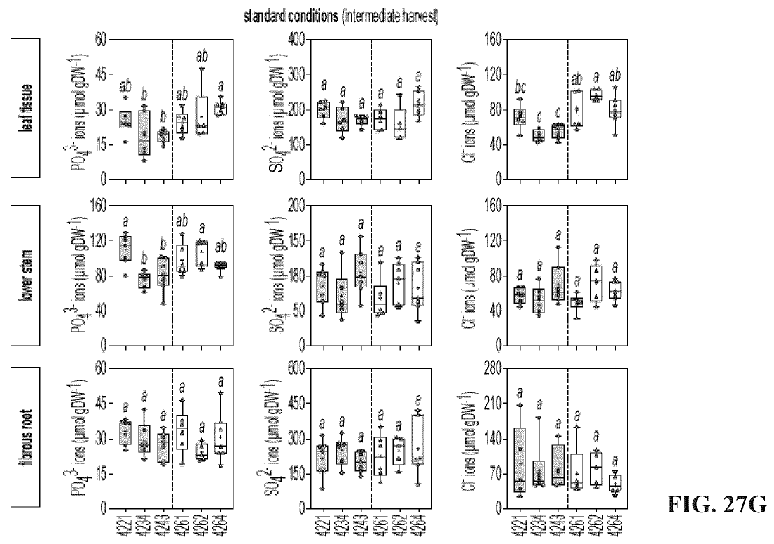

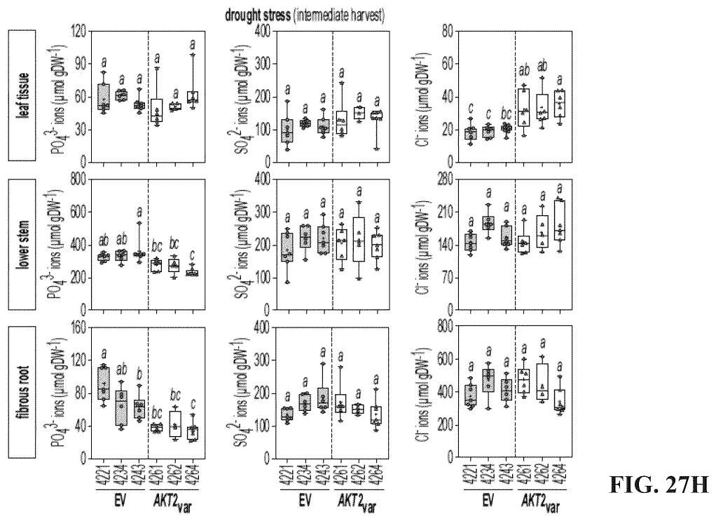

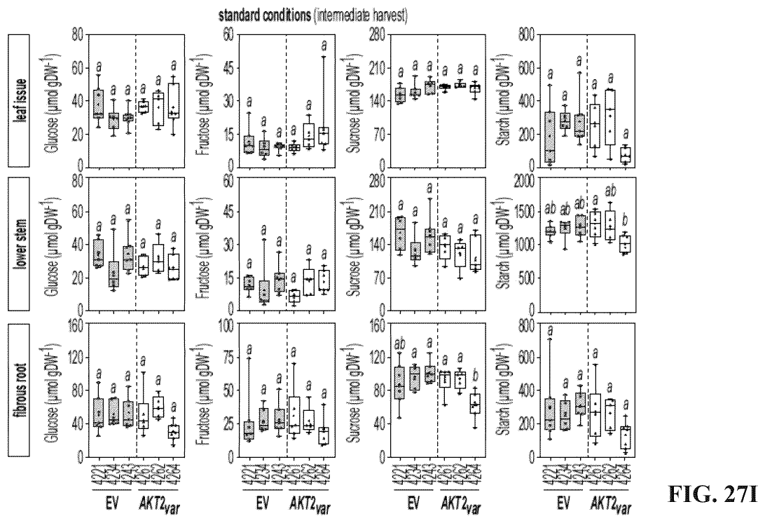

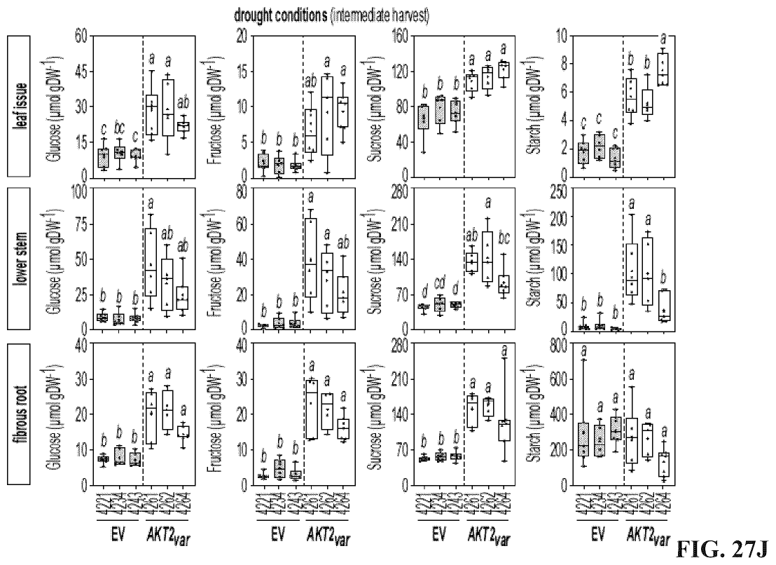

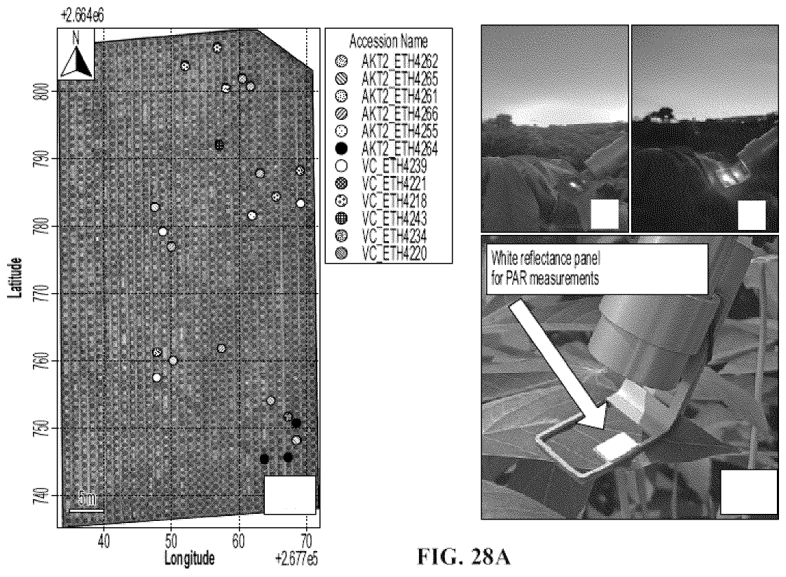

Water availability is closely linked to plant potassium homeostasis (see e.g. Fang, S., Yang, H., Duan, L., Shi, J. and Guo, L. (2023) Potassium fertilizer improves drought stress alleviation potential in sesame by enhancing photosynthesis and hormonal regulation. Plant Physiology and Biochemistry, 200, 107744; Bhardwaj, S., Kapoor, B., Kapoor, D., Thakur, U., Dolma, Y. and Raza, A. (2025) Manifold roles of potassium in mediating drought tolerance in plants and its underlying mechanisms. Plant Science, 351, 112337). To investigate the effect of water availability on AKT2var performance, the EV control lines and three AKT2var-expressing transgenic lines were grown in the greenhouse. All plants were grown for 8 weeks post planting under standard greenhouse conditions with daily watering, followed by 5 weeks of withholding water for drought stress, or daily watering for the control group. Daily watering was then resumed for all plants for 6 weeks under standard greenhouse conditions (FIG. 26). Plants were harvested either after 13 weeks of growth corresponding to the end of the drought stress treatment (for intermediate harvest (“IH”), see FIGS. 20H-20N and FIGS. 27A-27J) or after 19 weeks of growth (“final harvest” [FH]; see FIGS. 20A-20G).

For drought stress experiments, plants were grown from cuttings and initially cultivated under standard conditions with regular water supply for the first 8 weeks. Periodic drought stress was applied from week 9 onwards. For this purpose, watering was stopped until the soil was noticeably dry, and plants exhibited phenotypical signs of drought stress, such as leaf wilting and subsequent shedding. After a 7-day drought period, the plants were watered with a set amount of water (100 ml). This watering schedule was repeated for four weeks to simulate periodic drought stress. After the 13th week of growth, the plants were watered regularly again to initiate recovery (FIG. 26).

For the analysis of free amino acids, such as proline and serine, 20 μl of the ethanol extract was mixed with 60 μl borate buffer (200 mM, pH 8.8) and 20 μl of aminoquinolyl-N-hydroxysuccinimidyl carbamate (AQC) solution (Synchem UG & Co. KG, Felsberg, Germany; 2 mg ml-1 in acetonitrile). The mixture was immediately vortexed and incubated for 10 min to facilitate derivatization. The quantification of the derivatized AQC amino acids was carried out by using a Dionex P680-HPLC system with an RF 2000 fluorescence detector (Dionex, Sunnyvale, CA, USA) and a column system consisting of CC8/4 ND 100-5 C18ec and CC 250/4 ND 100-5 C18ec (Macherey-Nagel, Duren, Germany).

### Results

When always grown under well-watered conditions, the plant height, stem dry weight, and leaf weight of all plants analysed were largely similar (FIGS. 20A, 20C, and 20E; FIGS. 27A and 27C), with only the AKT2var-4264 line producing taller plants than the EV controls. However, all three AKT2var-expressing lines (AKT2var-4261, AKT2var-4262, and AKT2var-4264) exhibited greater root biomass relative to all EV controls (FIG. 20E, FIG. 20H, and FIG. 27A). The average root biomass of EV lines was 11.3 g dry weight (DW), while those of the AKT2var-expressing lines were 14.7 g DW (AKT2var-4261, 30.5% higher), 14.6 g DW (AKT2var-4262, 29.5% higher), and 14.4 g DW (AKT2var-4264, 28% higher) (FIG. 20E).

In marked contrast, all AKT2var-expressing lines showed greater leaf, stem, and root weights, with the AKT2var-4264 line having the tallest plants, compared to EV control plants when grown under drought stress (FIGS. 20B, 20D, 20F, and 20G). Importantly, storage root growth was significantly greater in the AKT2var-expressing lines, with an average of 5.4 g DW compared to the vector controls at 2.6 g DW, representing a 110% increase at final harvest (FIG. 20F and FIG. 20G). The observed differences at FH, following drought stress treatment, were already apparent during IH (FIGS. 20H-20I and 20N; FIGS. 27B-27D).

The levels of the amino acids serine and proline can be taken as a proxy for the degree of drought stress in plants (e.g. Farooq, M., Wahid, A., Kobayashi, N., Fujita, D. and Basra, S. M. A. (2009) Plant drought stress: effects, mechanisms and management. Agronomy for Sustainable Development, 29, 185-212.). When measured in leaves and fibrous roots of well-watered plants at the IH time point, both AKT2var-expressing lines and EV controls displayed similar levels of serine and proline, ranging from 0.87 to 1.42 μmol g DW¹ (FIG. 20J). Similarly, the levels of glutamate in all tissues collected from well-watered plants were highly similar among EV and AKT2var-expressing plants (FIG. 20J). In general, drought stress led to higher levels for all amino acids (FIG. 20K). Under drought stress, however, the concentrations of proline and serine in leaves and roots were significantly lower in AKT2var-expressing lines than in EV lines (FIG. 20K), indicating that AKT2var-expressing transgenic lines experience drought stress to a lesser degree. By contrast, the drought-induced increases in glutamate and glycine (glycine data not shown) in leaves and fibrous roots were similar in both sets of genotypes (FIG. 20K).

Cassava plants expressing AKT2var and grown in confined field trials achieved a higher relative yield than control plants, just as they did when grown under controlled greenhouse conditions (FIGS. 16A-16D and FIGS. 18A-18D). Thus, given the important function of K⁺ homeostasis in plant drought tolerance (Johnson, R. et al. (2022) Potassium in plants: Growth regulation, signaling, and environmental stress tolerance. Plant Physiol Biochem, 172, 56-69.), the responses of all plant lines were tested under controlled drought stress conditions in the greenhouse (FIGS. 20A-20K). The observation that AKT2var-expressing lines exhibited a better root growth performance under drought is in line with diminished molecular symptoms for drought stress in these mutants. This latter conclusion seems justified because proline levels, in general, can serve as a proxy for the degree of drought stress (Szabados, L. and Savouré, A. (2010) Proline: a multifunctional amino acid. Trends Plant Sci, 15, 89-97, Liang, X., Zhang, L., Natarajan, S. K. and Becker, D. F. (2013) Proline mechanisms of stress survival. Antioxid Redox Signal, 19, 998-1011.), while serine levels are indicative of rising rates of drought-induced photorespiration (Siqueira, J. A., Zhang, Y., Nunes-Nesi, A., Fernie, A. R. and Aranjo, W. L. (2023) Beyond photorespiration: the significance of glycine and serine in leaf metabolism. Trends Plant Sci, 28, 1092-1094.). Thus, the lower proline and serine levels observed in AKT2var-expressing plants relative to EV controls suggest that expression of AKT2var led to decreased molecular symptoms for drought. Moreover, the higher sucrose levels in leaves, stems, and roots (FIGS. 20L-20M, FIGS. 27I-27J), as well as higher overall yield, further underscore the increased drought stress tolerance of AKT2var-expressing cassava plants.

There were no significant changes between EV and AKT2var lines in the levels of cations or anions in leaves, stems, and root tissue (FIGS. 27E-27H). However, strong alterations were observed in carbohydrate levels. Specifically, starch levels in leaves were up to 50% higher in AKT2var-expressing lines than in EV lines under drought stress conditions (FIGS. 20L-20M, FIGS. 27I-27J). Sucrose concentrations in the leaves rose from 53 μmol g DW⁻¹ in EV lines to 124.9 μmol g DW⁻¹ in AKT2var lines, while sucrose levels in stems increased from 53.1 μmol g DW⁻¹ in EV lines to 130.2 μmol g DW⁻¹ in AKT2var lines. In storage roots, sucrose levels rose markedly from 53.0 μmol g DW⁻¹ in EV lines to 145.5 μmol g DW⁻¹ in AKT2var lines.

Similar observations were made for leaf and stem starch contents, while root starch levels were similar across genotypes (FIGS. 20L-20M). Glucose and fructose levels were higher in AKT2var-expressing lines in all tissues analysed (FIGS. 27I-27J). The higher levels of all carbohydrates in AKT2var-expressing lines compared to EV lines, together with the greater biomass, clearly indicated that AKT2var-expressing lines maintain a higher metabolism than do control plants under drought conditions.

It is remarkable that AtAKT2var expression can raise yields under both control and drought conditions in cassava, without requiring additional agricultural inputs. Such targeted biotechnological interventions, alongside continued cassava breeding, should help the development of cassava plants that might support smallholder farmers to achieve reasonable yields, even under increasingly challenging growing conditions.

### Example 10: Identification of Relevant Tissues by Assessing Expression Patterns of pAtAKT2 in Cassava

The following example describes determining the expression patterns of the pAtAKT2 promoter in cassava.

### Methods

Constructs were designed with reporter gene GUS being expressed under the pAtAKT2 promoter. Constructs were transformed into cassava as in Example 2. Different cassava tissues were sampled into ice cold 90% acetone solution. Cross-sections were manually prepared with a razor blade. These sections were covered with GUS staining buffer (200 mM NaP pH7, 100 mM K3[Fe(CN₆)], 100 mM K4[Fe(CN₆)], 500 mM EDTA, 0.5% SILWET® gold) and thoroughly vacuum infiltrated for 10 min. The GUS staining buffer was removed and replaced with fresh GUS staining solution containing GUS staining buffer with 0.25 mg/ml 5-bromo-4-chloro-3-indolyl-b-D-glucuronic acid (X-Gluc; pre-dissolved in a small amount of DMSO). The GUS staining solution was thoroughly vacuum infiltrated for 10 min. The infiltrated tissues were incubated for 30 min at 37° C. After removal of the GUS staining solution, 70% ethanol was added to the tissue sections and incubated in 37° C. until the tissues were cleared. Images were taken on a Zeiss STEMI SV11 Stereomicroscope (Zeiss, Wetzlar, Germany). Evaluation of tissue expression patterns was determined by means standard in the art (for example, as in Zierer et al., 2022. A promoter toolbox for tissue-specific expression supporting translational research in cassava (Manihot esculenta). Front Plant Sci 13:1042379.).

### Results

In line with the RT-qPCR analysis (FIGS. 15A-15B), GUS activity was detected, driven by the AtAKT2 promoter most strongly in the stem segments, in the phloem area and xylem parenchyma of the upper stem as well as in the xylem rays spanning the cambial region (FIG. 15D). In these reporter lines, no GUS staining was observed in source leaves, while weak but distinct GUS staining appeared in the minor veins of sink leaves (FIG. 15C). The petioles showed strong staining, particularly in dotted structures, likely representing companion cells, and in the xylem parenchyma located between individual xylem vessels (FIG. 15C). In the lower stem, GUS staining was detected in phloem companion cells and in vascular rays, particularly in their connection to xylem vessels, i.e., xylem vessel-associated cells (VACs; FIG. 15D). In the neck region of storage roots, GUS staining highlighted dotted structures, which likely represent companion cells, together with prominent staining in xylem rays, particularly at their contact points with VACs (FIG. 15C). GUS staining at the early bulking stage of the storage root (characterized by secondary anatomy, but minimal radial growth) displayed a similar but weaker pattern (FIG. 15C). Vascular rays and VACs connect the xylem and phloem in secondary anatomy, where parenchyma cells increase the physical separation between the two transport systems. By contrast, no staining was observed in fibrous roots at initial time points, and faint staining in the vasculature after about 2 h of incubation in GUS-staining solution (FIG. 15C).

Observations of GUS staining were confirmed by end-point PCR. AKT2var expression in the AKT2var-4266 line was highest in the upper and lower stem cores. The leaves, petioles, and upper and lower stem peels only produced weak signals, as did the root core (FIG. 22A). Overall, AKT2var expression appears strongest in the different cell types of the stem vasculature, with weaker expression in the vasculature of other plant parts.

Overall, the AtAKT2 promoter predominantly drove expression in core cassava stem tissues, with additional expression in cassava roots and leaves.

### Example 11: Evaluating AtAKT2 Homologs

The following example describes analyses of the AKT2 family of proteins to evaluate their functionality in enhancing cassava growth.

### Methods

### Phylogenetic Analysis

Sequences of proteins of the Shaker-family of ion channels were obtained from publicly available data. Sequences were aligned, and a phylogenetic tree assembled.

### Results

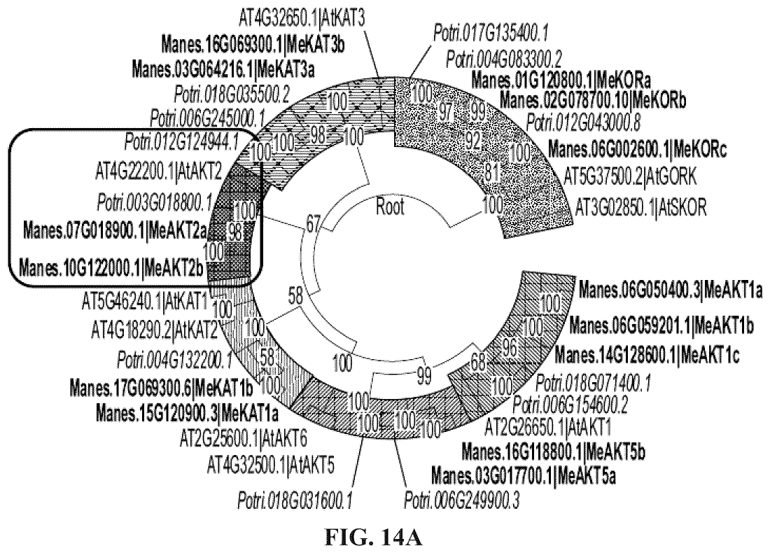

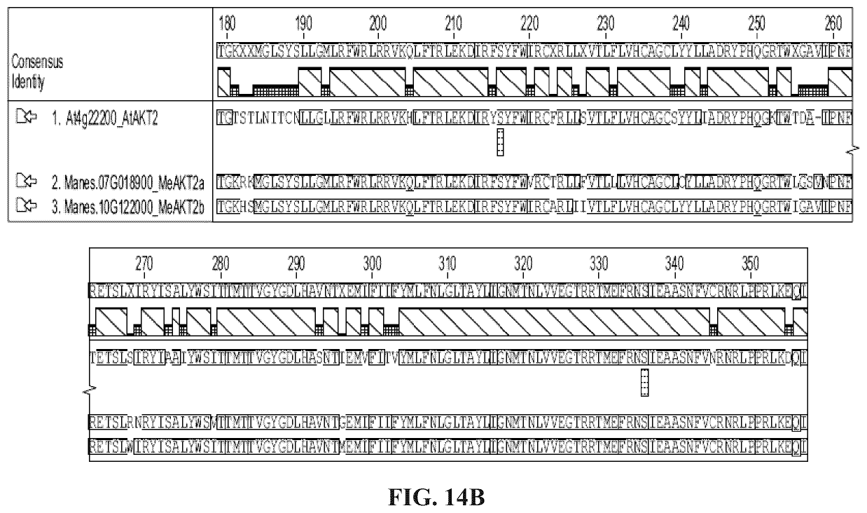

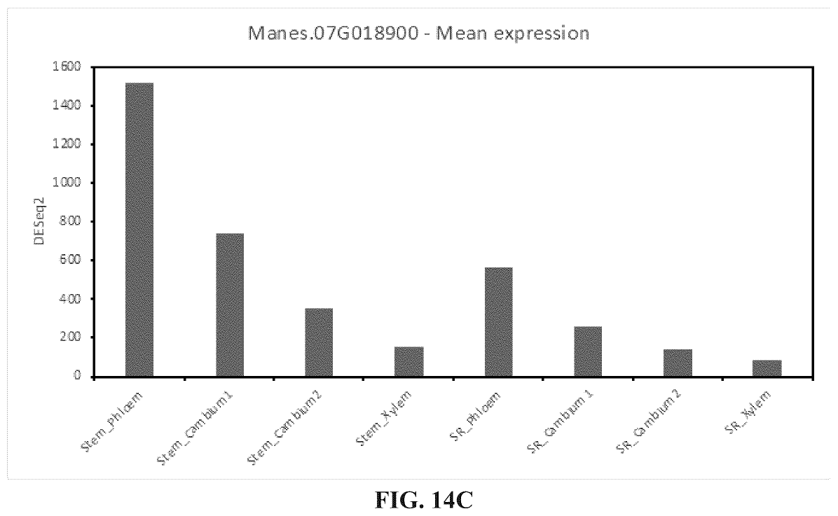

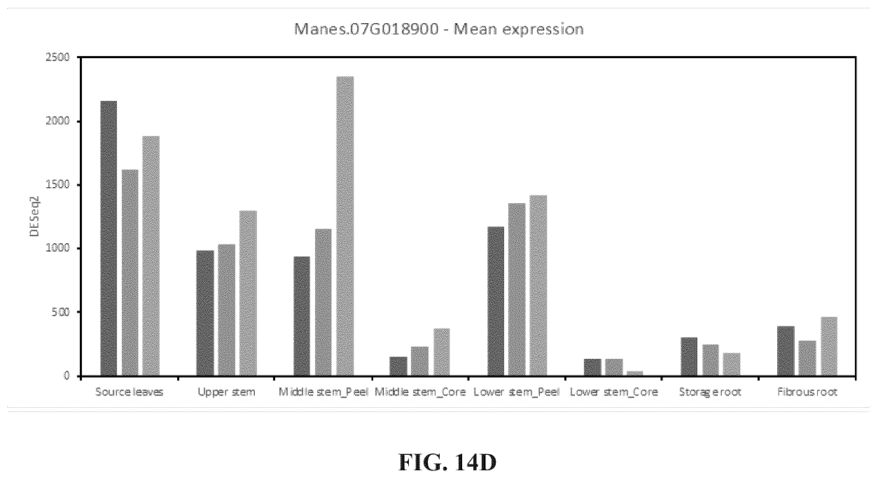

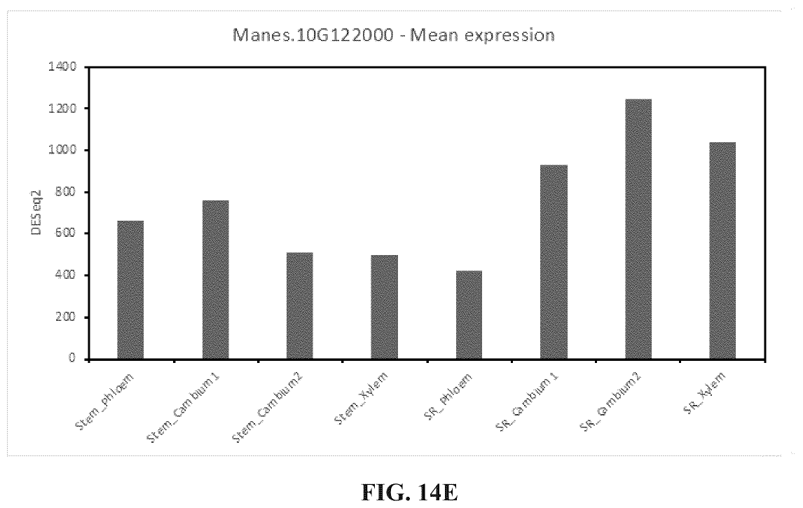

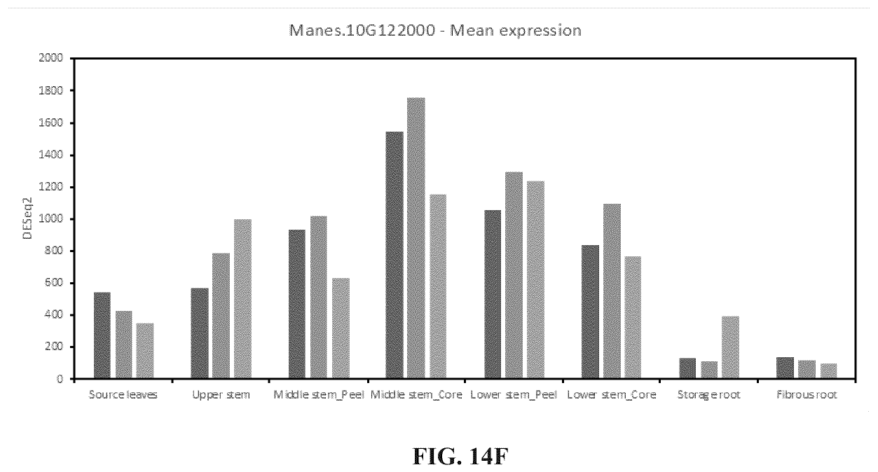

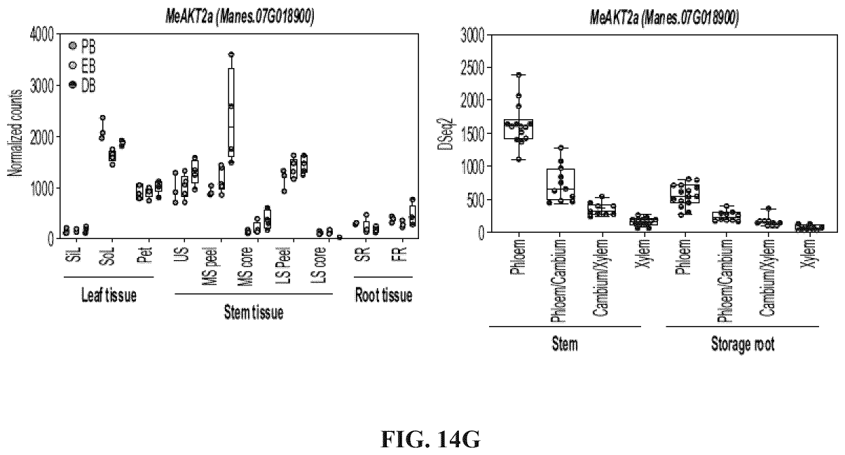

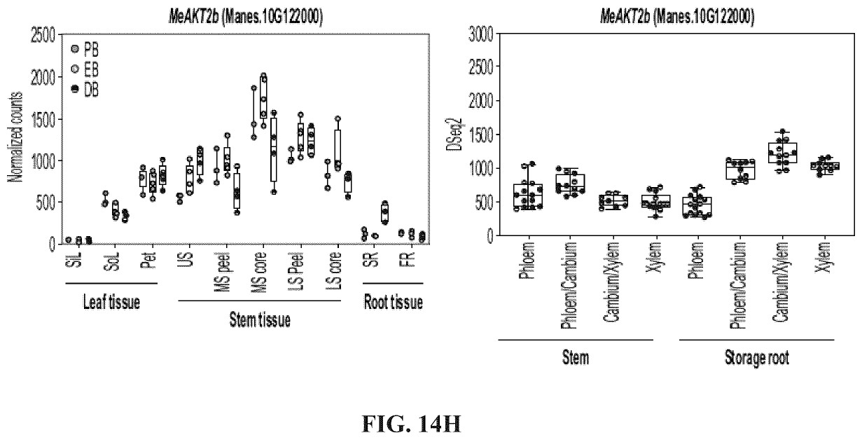

AKT2 belongs to the Shaker-family of ion channels. The gene family has several members, but phylogenetic analysis revealed that two genes in cassava seemed to be the most likely homologs to AtAKT2: MeAKT2a (Manes.07G018900, SEQ ID NO: 19) and MeAKT2b (Manes.10G122000, SEQ ID NO: 20) (FIG. 14A). Sequence alignments of AtAKT2 and the two putative cassava homologs revealed that the AtAKT2 regulatory serines S210 and S329, which were mutated to S210N and S329N in Gajdanowicz et al. (Gajdanowicz et al., 2011. Potassium (K+) gradients serve as a mobile energy source in plant vascular tissues. Proc Natl Acad Sci USA, 108, 864-9.) to turn the channel from an inward-rectifying channel into a non-rectifying channel, are conserved in both MeAKT2a and MeAKT2b (FIG. 14B).

Analysis of gene expression data of the cassava homologs (from Rüscher et al., (2024) Symplasmic phloem loading and subcellular transport in storage roots are key factors for carbon allocation in cassava. doi[DOT]org/10[DOT]1093/plphys/kiae298) showed that MeAKT2a had more of a phloem-sided expression with leaf and stem (peel) dominant expression (FIGS. 14C-14H). MeAKT2b was slightly upregulated during storage root bulking; it also had more xylem-sided expression, and had significant expression in xylem-dominant tissue (for instance, stem “core”) (FIGS. 14E-14F). While not wanting to be limited by hypothesis, MeAKT2a could be a more favorable candidate for gene editing to achieve the same result as AtAKT2.

### Example 12: Overexpression of Mutant MeAKT2a and MeAKT2b Result in Alterations of Plant Phenotypes

The following example describes enhanced cassava growth as a result of overexpression of endogenous cassava homologs of AtAKT2.

### Methods

cDNA of MeAKT2a and MeAKT2b are obtained. Mutant forms of MeAKT2a and MeAKT2b are generated (Meakt2a and Meakt2b) that have the Arabidopsis AKT2 conserved regulatory serines mutated to asparagine.

Constructs are designed to express mutant Meakt2a under the control of the Arabidopsis AKT2 promoter (SEQ ID NO: 2) and mutant Meakt2b under the control of the same promoter. Constructs are transformed into cassava, and cassava is grown as in Example 2. Growth, photosynthesis efficiency, and sugar and ion concentrations are measured as in Examples 2-5.

### Results

Overexpression of mutant endogenous Meakt2a or Meakt2b results in enhanced cassava growth as measured in growth height, total shoot dry matter, and total root dry matter. Phloem transport is improved in plants overexpressing Meakt2a or Meakt2b compared to wild type plants. This is also accompanied by improved photosynthetic performance. Plants overexpressing Meakt2a and Meakt2b also show elevated levels of cations in shoot tissue, and reduced levels of cations in root tissue, as well as increased phloem sugar content and increased starch amounts.

### Example 13: Overexpression of Wild Type AtAKT2, MeAKT2a, and MeAKT2b Result in Alterations of Plant Phenotypes

The following example describes enhanced cassava growth as a result of overexpression of wild type AtAKT2, MeAKT2a, and MeAKT2b.

### Methods

Experiments are performed as in Examples 2-5 and 7, with the following exceptions. Separate constructs are designed and transformed into cassava with each of wild type AtAKT2, wild type MeAKT2a, and wild type MeAKT2b under control of the AtAKT2 promoter. Growth, photosynthesis efficiency, and sugar and ion concentrations are measured as in Examples 2-5.

### Results

Overexpression of AtAKT2var or endogenous MeAKT2a or MeAKT2b results in enhanced cassava growth as measured in growth height, total shoot dry matter, and total root dry matter. Phloem transport is improved in plants overexpressing Meakt2a or Meakt2b compared to wild type plants. This is also accompanied by improved photosynthetic performance, such as increased assimilate delivery and improved growth. Plants overexpressing AtAKT2var, MeAKT2a, or MeAKT2b also show altered levels of cations in shoot tissue and root tissue, as well as increased phloem sugar content and increased starch amounts.

### Example 14: Use of Alternative Promoters to Drive Overexpression

The following example describes enhanced cassava growth as a result of overexpression of wild type or mutant AtAKT2, MeAKT2a, or MeAKT2b under the control of promoters other than pAtAKT2.

### Methods

Constructs are designed as in Examples 2-5 and 7-8, with the following exceptions. The Arabidopsis AKT2 promoter is swapped for a vasculature-specific promoter (e.g., pCoYMV, prolC), a companion cell-specific promoter (e.g., pAtSUC1), a phloem-specific promoter (e.g., prolC, pRTBV (Dutt et al., 2012. Evaluation of four phloem-specific promoters in vegetative tissues of transgenic citrus plants. Tree Physiology. 32(1):83-93), a guard cell-specific promoter (e.g., StKST1), a promoter driving strong expression in the vasculature, a promoter driving strong expression in companion cells, a promoter driving strong expression in phloem, the endogenous MeAKT2a promoter, or the endogenous MeAKT2b promoter. Growth, photosynthesis efficiency, and sugar and ion concentrations are measured as in Examples 2-5.

### Results

Other promoters for driving overexpression of wild type or mutant AtAKT2, MeAKT2a, and MeAKT2b are tested. Promoters to drive expression in companion cells (such as but not limited to pAtSUC1), in vasculature (such as but not limited to pCoYMV), in xylem (such as but not limited to pMeAKT2b) or in phloem (such as but not limited to pRTBV or pMeAKT2a) are tested. Guard-cell specific promoters such as but not limited to StKST1 are also tested.

Similar improvements or alterations of cassava growth, photosynthetic efficiency, ion concentrations, and sugar concentrations are detected in cassava plants overexpressing wild type or mutant AtAKT2, MeAKT2a, or MeAKT2b under control of alternative promoters.

What is claimed is:

1. A genetically modified plant, plant part thereof, or plant cell thereof comprising one or more nucleotide sequences encoding a modified POTASSIUM TRANSPORTER 2 (AKT2) protein, wherein the modified AKT2 protein is selected from the group of a modified plant AKT2 protein, a modified Arabidopsis thaliana AKT2 (AtAKT2) protein, a modified first Manihot esculenta AKT2 protein (MeAKT2a), and/or a modified second Manihot esculenta AKT2 protein (MeAKT2b), or a homolog thereof.

2. The genetically modified plant, plant part thereof, or plant cell thereof of claim 1, wherein a wild-type AKT2 protein has phloem potassium transport activity and the modified AKT2 protein has phloem potassium transport activity.

3. The genetically modified plant or part thereof of claim 2, wherein an AKT2 protein comprises:
(a) mode 1, wherein the AKT2 protein acts as an inward-rectifying K⁺ channel (Kᵢₙ); and
(b) mode 2, wherein the AKT2 protein acts as a nonrectifying channel;
wherein the wild-type AKT2 protein comprises mode 1; and
wherein the modified AKT2 protein comprises modifications that bias the modified AKT2 toward mode 2 or lock the modified AKT2 in mode 2.

4. The genetically modified plant or plant part thereof of claim 1, wherein the modified AKT2 protein, the modified plant AKT2 protein, the modified AtAKT2 protein, the modified MeAKT2a protein, and/or the modified MeAKT2b protein (a) comprises one or more amino acid substitutions, insertions, or deletions in the Ion_trans (PF00520, Ion transport protein) motif (corresponding to amino acids 75-323 of SEQ ID NO: 17) and/or one or more amino acid substitutions, insertions, or deletions in the Ion_trans_2 (PF07885, Ion channel) motif (corresponding to amino acids 225-317 of SEQ ID NO: 17), wherein the one or more substitutions, insertions, or deletions modulate or increase the ion transport activity; (b) comprises one or more amino acid substitutions, insertions, or deletions, wherein the one or more substitutions, insertions, or deletions modulate or increase the ion transport activity; (c) comprises one or both of the amino acid substitutions corresponding to S210N and S329N when aligned to SEQ ID NO: 17; (d) comprises one or both of the amino acid substitutions corresponding to S199N and S139N when aligned to SEQ ID NO: 19; or (e) comprises one or both of the amino acid substitutions corresponding to S216N and S319N when aligned to SEQ ID NO: 20.

5. The genetically modified plant or plant part thereof of claim 1, wherein:
(i) the wild-type plant AKT2 protein comprises a protein comprising at least 70% identity, at least 80% identity, at least 90% identity, at least 95% identity, or at least 99% identity to one of SEQ ID NOs: 17, 19, and 20, the wild-type AtAKT2 protein comprises a protein comprising at least 70% identity, at least 80% identity, at least 90% identity, at least 95% identity, or at least 99% identity to SEQ ID NO: 17, the wild-type MeAKT2a protein comprises a protein comprising at least 70% identity, at least 80% identity, at least 90% identity, at least 95% identity, or at least 99% identity to SEQ ID NO: 19, and the wild-type MeAKT2b protein comprises a protein comprising at least 70% identity, at least 80% identity, at least 90% identity, at least 95% identity, or at least 99% identity to SEQ ID NO: 20; or a functional fragment of one of the foregoing;
(ii) the modified plant AKT2 protein comprises a protein comprising at least 70% identity, at least 80% identity, at least 90% identity, at least 95% identity, or at least 99% identity to one of SEQ ID NOs: 18, 25, and 26 the modified AtAKT2 protein comprises a protein comprising at least 70% identity, at least 80% identity, at least 90% identity, at least 95% identity, or at least 99% identity to SEQ ID NO: 18, wherein the modified MeAKT2a protein comprises a protein comprising at least 70% identity, at least 80% identity, at least 90% identity, at least 95% identity, or at least 99% identity to SEQ ID NO: 25, and wherein the modified MeAKT2b protein comprises a protein comprising at least 70% identity, at least 80% identity, at least 90% identity, at least 95% identity, or at least 99% identity to SEQ ID NO: 26; or a functional fragment of one of the foregoing; and/or
(iii) the one or more nucleotide sequences encoding the modified AtAKT2 protein comprises a nucleotide sequence comprising at least 70% identity, at least 80% identity, at least 90% identity, at least 95% identity, or at least 99% identity to SEQ ID NO: 3.

6. The genetically modified plant or part thereof of claim 5, wherein the one or more nucleotide sequences encoding the modified plant AKT2 protein, the modified AtAKT2 protein, the modified MeAKT2a protein, and/or the modified MeAKT2b protein is operably linked to an expression control sequence; wherein the expression control sequence comprises an overexpression promoter, a phloem-specific promoter, and/or a xylem-specific promoter; and optionally wherein the promoter comprises a plant AKT2 promoter, an Arabidopsis thaliana POTASSIUM TRANSPORTER 2 (pAtAKT2) promoter, an Arabidopsis thaliana SUCROSE TRANSPORTER 1 promoter (pAtSUC1), a cassava MeAKT2a promoter, a cassava MeAKT2b promoter, or a proIC promoter.

7. A genetically modified plant or plant part thereof comprising one or more nucleotide sequences encoding a plant POTASSIUM TRANSPORTER 2 (AKT2) protein operably linked to an expression control sequence, wherein the expression control sequence comprises an overexpression promoter, optionally wherein the plant AKT2 protein is a wild-type protein.

8. The genetically modified plant or plant part thereof of claim 1, wherein:
(i) the plant is a dicot;
(ii) the plant produces storage roots or tubers and/or benefits from potassium fertilization, optionally wherein the plant is selected from the group consisting of cassava, potato, sweet potato, yam, ube, yacón, taro, konjac, ginger, radish, turnip, rutabaga, parsnip, jicama, Jerusalem artichoke, turmeric, horseradish, beet, lotus, maca, celeriac, skirret, wasabi, citrus fruits, bananas, grains, tomatoes, sorghum, cotton, sugar beets, soybeans, cowpea, alfalfa, grapes, peppers, apples, and melons; and/or
(iii) wherein the plant has a large transport distance between a storage organ and a photosynthetic leaf.

9. The genetically modified plant or plant part thereof of claim 1, wherein the genetically modified plant has improved phloem transport, improved phloem mass flow, improved source-sink delivery, increased fibrous root formation, higher photosynthetic efficiency, improved photosynthesis, higher rate of CO₂ fixation and/or electron transport rate, earlier maximum growth rate, increased yield under field conditions, increased yield under drought conditions, increased drought stress resistance, improved drought tolerance, and/or increased yield under potassium deficiency as compared to a control plant grown under the same conditions, optionally wherein the genetically modified plant or a progenitor thereof was selected for improved phloem transport, improved phloem mass flow, improved source-sink delivery, increased fibrous root formation, increased growth, improved photosynthesis, higher rate of CO₂ fixation, and/or electron transport rate when grown under non-limiting energy conditions, earlier maximum growth rate, increased yield, increased plant growth, increased plant height, increased shoot growth, increased total shoot dry matter, increased storage root or tuber biomass, increased number of storage roots or tubers per plant, and/or increased total storage root or tuber dry matter.

10. The genetically modified plant or plant part thereof of claim 1, wherein the genetically modified plant is a cassava plant, wherein the genetically modified cassava plant has improved phloem transport, increased plant growth, increased plant height, increased shoot growth, increased total shoot dry matter (TSDM), increased storage root growth, increased drought stress resistance, increased drought tolerance, improved photosynthetic performance, lower proline and/or serine levels in drought conditions, increased number of storage root per plant, and/or increased total storage root dry matter (TRDM) as compared to a control cassava plant grown under the same conditions.

11. The genetically modified plant or plant part thereof of claim 1, wherein the genetically modified plant comprises
(a) at least one of the following shoot traits: increased height, increased concentrations of sodium (Na⁺), increased concentrations of calcium (Ca²⁺), increased concentrations of magnesium (Mg²⁺), increased concentrations of potassium (K⁺), reduced sucrose concentration or level in aboveground plant parts, increased starch concentration or level, increased shoot fresh weight, increased TSDM, and increased phloem transport rate; and/or
(b) at least one of the following root traits: reduced concentrations of K⁺, reduced sucrose concentration, increased glucose concentration, increased fructose concentration, increased starch concentration, increased root fresh weight, and increased TRDM as compared to a control plant grown under the same conditions.

12. The genetically modified plant or plant part thereof of claim 1, wherein the genetically modified plant
(a) reaches the maximum relative growth rate (RGR) faster,
(b) has an increased harvest index (HI),
(c) has increased yield,
(d) has a higher maximum electron transport rate (ETR),
(e) has an increased tracer transport velocity; and/or
(f) has an increased CO₂ assimilation rate
as compared to a control plant grown under the same conditions.

13. The genetically modified plant or plant part thereof of claim 1, wherein the genetically modified plant grown under drought conditions has
(a) increased relative yield,
(b) elevated sucrose concentrations;
(c) elevated glucose concentrations;
(d) elevated fructose concentrations;
(e) elevated starch concentrations;
(f) increased TSDM;
(g) increased TRDM; and/or
(h) reduced serine and/or proline concentrations
as compared to a control plant grown under drought conditions.

14. The genetically modified plant or plant part thereof of claim 1, wherein the genetically modified plant exhibits increased drought stress resistance and/or increased drought tolerance as compared to a control plant grown under the same conditions, and wherein the increased drought stress resistance and/or increased drought tolerance is indicated by reduced proline concentrations, reduced serine concentrations, and/or increased relative yield as compared to a control plant grown under the same conditions.

15. A method of producing the genetically modified plant or plant part thereof of claim 1, comprising introducing one or more nucleotide sequences encoding the modified plant AKT2 protein, the modified AtAKT2 protein, the modified MeAKT2a protein, and/or the modified MeAKT2b protein, optionally wherein the one or more nucleotide sequences are operably linked to the expression control sequence comprising the overexpression promoter.

16. The method of claim 15, wherein the one or more nucleotide sequences encoding the modified plant AKT2 protein, the modified AtAKT2 protein, the modified MeAKT2a protein, and/or the modified MeAKT2b protein is operably linked to an expression control sequence; wherein the expression control sequence comprises an overexpression promoter and/or a phloem-specific promoter; and optionally wherein the promoter comprises a plant AKT2 promoter, an Arabidopsis thaliana POTASSIUM TRANSPORTER 2 (pAtAKT2) promoter, an Arabidopsis thaliana SUCROSE TRANSPORTER 1 promoter (pAtSUC2), a cassava MeAKT2a promoter, a cassava MeAKT2b promoter, or a proIC promoter.

17. A method of producing the genetically modified plant or plant part thereof of claim 1, comprising
(a) genetically modifying a plant by transforming the plant with one or more gene editing components that target an endogenous nuclear genome sequence encoding the wild-type plant AKT2 protein, the wild-type AtAKT2 protein, the wild-type MeAKT2a protein, and/or the wild-type MeAKT2b protein to produce the modified AKT2 protein, modified AtAKT2 protein, the modified MeAKT2a protein, and/or the modified MeAKT2b protein, wherein the one or more gene editing components comprise a ribonucleoprotein complex that targets the nuclear genome sequence; a vector including a TALEN protein encoding sequence, wherein the TALEN protein targets the nuclear genome sequence; a vector including a ZFN protein encoding sequence, wherein the ZFN protein targets the nuclear genome sequence; an oligonucleotide donor (OND), wherein the OND targets the nuclear genome sequence; or a vector CRISPR/Cas enzyme encoding sequence and a targeting sequence, wherein the targeting sequence targets the nuclear genome sequence; or
(b) genetically modifying a plant by transforming the plant with one or more gene editing components that target an endogenous nuclear genome sequence encoding a promoter of an endogenous plant AKT2 protein, a promoter of an endogenous AtAKT2 protein, a promoter of an endogenous MeAKT2a protein, and/or a promoter of an endogenous MeAKT2b protein to produce a modified AKT2 promoter, a modified AtAKT2 promoter, a modified MeAKT2a promoter, and/or a modified MeAKT2b promoter, wherein the modified AKT2 promoter, the modified AtAKT2 promoter, the modified MeAKT2a promoter, and/or the modified MeAKT2b promoter has increased expression and/or altered tissue-specific expression as compared to the unmodified promoter, wherein the one or more gene editing components comprise a ribonucleoprotein complex that targets the nuclear genome sequence; a vector including a TALEN protein encoding sequence, wherein the TALEN protein targets the nuclear genome sequence; a vector including a ZFN protein encoding sequence, wherein the ZFN protein targets the nuclear genome sequence; an oligonucleotide donor (OND), wherein the OND targets the nuclear genome sequence; or a vector CRISPR/Cas enzyme encoding sequence and a targeting sequence, wherein the targeting sequence targets the nuclear genome sequence.

18. The method of claim 15, wherein the modified plant AKT2 protein, the modified AtAKT2 protein, the modified MeAKT2a protein, and/or the modified MeAKT2b protein (a) comprises one or more amino acid substitutions, insertions, or deletions in the Ion_trans (PF00520, Ion transport protein) motif (corresponding to amino acids 75-323 of SEQ ID NO: 17) and/or one or more amino acid substitutions, insertions, or deletions in the Ion_trans_2 (PF07885, Ion channel) motif (corresponding to amino acids 225-317 of SEQ ID NO: 17), wherein the one or more substitutions, insertions, or deletions modulate or increase the ion transport activity; (b) comprises one or more amino acid substitutions, insertions, or deletions, wherein the one or more substitutions, insertions, or deletions modulate or increase the ion transport activity; or (c) comprises one or both of the amino acid substitutions corresponding to S210N and S329N when aligned to SEQ ID NO: 17.

19. The method of claim 15, wherein:
(i) the wild-type plant AKT2 protein comprises a protein comprising at least 70% identity, at least 80% identity, at least 90% identity, at least 95% identity, or at least 99% identity to one of SEQ ID NOs: 17, 19, and 20, the wild-type AtAKT2 protein comprises a protein comprising at least 70% identity, at least 80% identity, at least 90% identity, at least 95% identity, or at least 99% identity to SEQ ID NO: 17, the wild-type MeAKT2a protein comprises a protein comprising at least 70% identity, at least 80% identity, at least 90% identity, at least 95% identity, or at least 99% identity to SEQ ID NO: 19, and the wild-type MeAKT2b protein comprises a protein comprising at least 70% identity, at least 80% identity, at least 90% identity, at least 95% identity, or at least 99% identity to SEQ ID NO: 20; or a functional fragment of one of the foregoing;
(ii) the modified plant AKT2 protein comprises a protein comprising at least 70% identity, at least 80% identity, at least 90% identity, at least 95% identity, or at least 99% identity to one of SEQ ID NOs: 18, 25, and 26 the modified AtAKT2 protein comprises a protein comprising at least 70% identity, at least 80% identity, at least 90% identity, at least 95% identity, or at least 99% identity to SEQ ID NO: 18, wherein the modified MeAKT2a protein comprises a protein comprising at least 70% identity, at least 80% identity, at least 90% identity, at least 95% identity, or at least 99% identity to SEQ ID NO: 25, and wherein the modified MeAKT2b protein comprises a protein comprising at least 70% identity, at least 80% identity, at least 90% identity, at least 95% identity, or at least 99% identity to SEQ ID NO: 26; or a functional fragment of one of the foregoing; and/or
(iii) the one or more nucleotide sequences encoding the modified AtAKT2 protein comprises a nucleotide sequence comprising at least 70% identity, at least 80% identity, at least 90% identity, at least 95% identity, or at least 99% identity to SEQ ID NO: 3.

20. The method of claim 15, further comprising selecting a genetically modified plant with improved growth, improved photosynthesis, higher rate of CO₂ fixation and/or higher electron transport rate when the genetically modified plant is grown under non-limiting energy conditions, earlier maximum growth rate, increased yield, increased plant growth, increased plant height, increased shoot growth, increased total shoot dry matter, increased storage root or tuber biomass, increased number of storage roots or tubers per plant, and/or increased total storage root or tuber dry matter as compared to a control plant.

21. A genetically modified plant or plant part thereof produced by the method of claim 15.

22. The genetically modified plant or plant part thereof of claim 21, wherein the genetically modified plant has improved phloem transport, improved phloem mass flow, improved source-sink delivery, increased fibrous root formation, higher photosynthetic efficiency, improved photosynthesis, higher rate of CO₂ fixation and/or electron transport rate, earlier maximum growth rate, increased yield under field conditions, increased yield under drought conditions, increased drought stress resistance, increased drought tolerance, and/or increased yield under potassium deficiency as compared to a control plant grown under the same conditions, and wherein:
(i) the plant produces storage roots or tubers and/or benefits from potassium fertilization, optionally wherein the plant is selected from the group consisting of cassava, potato, sweet potato, yam, ube, yacón, taro, konjac, ginger, radish, turnip, rutabaga, parsnip, jicama, Jerusalem artichoke, turmeric, horseradish, beet, lotus, maca, celeriac, skirret, wasabi, citrus fruits, bananas, grains, tomatoes, sorghum, cotton, sugar beets, soybeans, cowpea, alfalfa, grapes, peppers, apples, and melons; and/or
(ii) wherein the plant is a passive symplasmic phloem loader.

23. An expression vector or isolated DNA molecule comprising one or more nucleotide sequences encoding a modified POTASSIUM TRANSPORTER 2 (AKT2) protein operably linked to an expression control sequence, wherein the modified AKT2 protein is selected from the group of a modified plant AKT2 protein, a modified Arabidopsis thaliana POTASSIUM TRANSPORTER 2 (AtAKT2) protein, a modified first Manihot esculenta AKT2 protein (MeAKT2a), and/or a modified second Manihot esculenta AKT2 protein (MeAKT2b); and/or the one or more gene editing components of claim 17.

24. A bacterial cell or an Agrobacterium cell comprising the expression vector or isolated DNA molecule of claim 23.

25. A composition or kit comprising the expression vector or isolated DNA molecule of claim 23 or a bacterial cell or an Agrobacterium cell comprising the expression vector or isolated DNA molecule of claim 23.

26. A genetically modified plant, plant part, plant cell, or seed including the expression vector or isolated DNA molecule of claim 23.

27. A composition or kit comprising the genetically modified plant, plant part, plant cell, or seed of claim 26.

28. A method of improving phloem transport, improving phloem mass flow, improving source-sink delivery, increasing fibrous root formation, increasing photosynthetic efficiency, improving photosynthesis, producing earlier maximum growth rate, increasing yield under field conditions, increasing yield under drought conditions, increasing drought stress resistance, increasing drought tolerance, increasing yield under potassium deficiency, increasing plant growth, increasing plant height, increasing shoot growth, increasing total shoot dry matter, increasing storage root or tuber biomass, increasing number of storage roots or tubers per plant, and/or increasing total storage root or tuber dry matter comprising: introducing a genetic alteration via the expression vector or isolated DNA molecule of claim 23 to a cell, wherein the cell is a plant cell.

29. A cassava plant or plant part thereof comprising
(a) one or more nucleotide sequences encoding a modified AtAKT2 protein, a modified MeAKT2a protein, and/or a modified MeAKT2b protein, and
(b) improved phloem transport, improved phloem mass flow, improved source-sink delivery, increased fibrous root formation, increased growth, improved photosynthesis, higher rate of CO₂ fixation and/or higher electron transport rate when the genetically modified plant is grown under non-limiting energy conditions, earlier maximum growth rate, increased yield, increased plant growth, increased plant height, increased shoot growth, increased total shoot dry matter, increased storage root or tuber biomass, increased number of storage roots or tubers per plant, and/or increased total storage root or tuber dry matter as compared to a control plant.


SEQUENCE LISTING (32 sequences)

SEQ ID NO: 1 - DNA, 14597 bp, synthetic construct
  source 1..14597: mol_type = other DNA
tgccgaattcggatccggagattgagtctaaaatattgaaaatactcactatcacatatgtcaacgataacactaatttttttttttgtataatatacgtaacgacacctgcattagatctaatttttaatcgattagagcttaaatgacatcgttttggtttgactaagagaatcgttaaaaatgttagaaaatctgttaaagcttaataactgtttgtttgttttttttctttctttttttgtcaatcgaaaattagactattaaatgacaattttccgtttaaaatggcaagtcctagatttgaaaaggtcttttgaaatctttttaagagaattaagggtgaattaaatatattcagaaattaaagggtcgaattgcaaactttcttggttcatacttggtctgcctttgtccctttcttatcttcttcgcgcgcttttgaaaaaaatagaaacccgccactgaacgtcagtcatccacgtgtcctctcctgtgctctgtttccgatcgtcggagacatcaatttgcctgcttgtagggtgattcgtcaaatctattatcaggttttaaatatactcgaattgacttccaaattcttagtctctagtgtaatgattttgagaatcacttaactccaaaaatataatccacgatcccgtgttaattattgaagaatcaatcgtttttaatttctcaccaatagatgttgctcttattacttaaaacaaattgtttagacaaatgtagcaagtgtgatacttagtgggatcttaaagacgatttctcctataacagaggacaaacaggtcggtcaattacaatgtcatccctctttaccctgtctttttttttcttcttaaaacctaaccatttgattgtttctaaaggtatttcaagaatatatgatcaatctagatgaatactataccgacgatgactacacacacaaggaaatatatatatcagctttcttttcacctaaaagtggtcccggtttagaatctaattcctttatctctcattttcttctgcttcacattcccgctagtcaaatgttaataagtgcacacaacgttttctcgaagcattagaatgtcctcctcttaattaatctccttctgattagattctcaatagagtttaaatttgttaatggagagatatattgggaccctcaaggcttctaattataccacgtttggcataattctctatcgtttggggccacatctttcacacttcattaccttatcaccaaaacataaaatcaatcaacttttttttgccttattgattgtgttggatccctccaaaattaaaacttgtgttccccacaaaagcttacccaatttcacttcaatcttaacaaataggaccaccactaccacgtacggtttgcatcatacaaaccacaaactccttcttcattacaattattatatcatctactaaaacctctttctccctctctctttcttgttcttagtgctaaattttctttgttcaggagaaatatatactgaattcggatcctctagaccatgtacccatacgatgttcctgactatgcgggctatccctatgacgtcccggactatgcaggattgtatccatatgacgttccagattacgccactagagctgcttacccatacgatgttcctgactatgcgggctatccctatgacgtcccggactatgcaggattgtatccatatgacgttccagattacgcaatggacctcaagtattcagcatctcattgcaacttatcctcagacatgaagctcaggcgttttcatcagcatcgtggaaaaggaagagaagaagagtatgatgcttcttctctcagcttgaacaatctgtcaaaacttattcttcctccacttggtgttgctagctataaccagaatcacatcaggtctagtggatggatcatctcacctatggactcaagatacaggtgctgggaattttatatggtgcttttagtggcatactctgcttgggtttacccttttgaagttgcatttctgaattcatcaccaaagagaaacctttgtatcgctgacaacatcgtagacttgttcttcgctgttgacattgtcttgacttttttcgttgcttacattgacgaaagaacacagcttcttgtccgtgaacctaaacagattgcagtgaggtacctatcaacatggttcttgatggatgttgcatcaactattccatttgacgctattggatacttaatcactggcacatccactttaaatatcacttgtaatctcttgggattacttagattttggagacttcgtagagttaaacacctcttcactaggctcgagaaggacattagatataactatttctggattcgctgctttagacttctatcagtgacattgtttctagtgcactgtgctggatgcagttattacctaattgcagacagatatccacaccaaggaaagacatggactgatgctatccctaatttcacagagacaagtctttccatcagatacattgcagctatttattggtctatcactacaatgaccacagtgggatatggagatcttcatgcaagcaacactattgaaatggtattcattacagtctacatgttattcaatcttggcctcactgcttaccttattggtaacatgactaatttggtcgtggaagggactcgtcgtaccatggaatttaggaataacattgaagcagcttcaaactttgttaacagaaacagattgcctcctagattaaaagaccagattttagcttacatgtgtttaaggtttaaagcagagagcttaaatcagcaacatcttattgaccagctcccaaaatctatctacaaaagcatttgtcaacatctttttcttccatctgttgaaaaagtttacctcttcaaaggcgtctcaagagaaattcttcttcttctggtttcaaaaatgaaggctgagtatattccaccaagagaggatgtcattatgcagaacgaagctccagatgatgtttacattattgtgtcaggagaagttgagatcattgattcagagatggagagagagtctgttttaggcactctacgttgtggagacatttttggagaagttggagcactttgttgcagaccacaaagctacacttttcaaactaagtctttatcacagcttctccgtctcaaaacatctttccttattgagacaatgcagattaaacaacaagacaatgccacaatgctcaagaacttcttgcagcatcacaaaaagctgagtaatttagacattggtgatctaaaggcacaacaaaatggcgaaaacaccgatgttgttcctcctaacattgcctcaaatctcatcgctgtggtgactacaggcaatgcagctcttcttgatgagctacttaaggctaagttaagccctgacattacagattccaaaggaaaaactccattgcatgtagcagcttctagaggatatgaagattgtgttttagtactcttaaagcacggttgcaacatccacattagagatgtgaatggtaatagtgctctatgggaagcaattatttctaagcattacgagattttcagaatcctttatcatttcgcagccatttctgatccacacattgctggagatcttctatgtgaagcagctaaacagaacaatgtagaagtcatgaaggctcttttaaaacaggggcttaacgtcgacacagaggatcaccatggcgtcacagctttacaggtcgctatggctgaggatcagatggacatggtgaatctcctggctactaacggtgcagatgtagtttgtgtgaatacacataatgaattcacaccattggagaagttaagagttgtggaagaagaagaagaagaagaacgtggaagagtgagtatttacagaggacatccattggagaggagagaaagaagttgcaatgaagctgggaagcttattcttcttcctccttcacttgatgacctcaagaaaattgcaggagagaagtttgggtttgatggaagtgagactatggtgactaatgaagatggagctgagattgacagtattgaagtgattagagataatgacaaactctactttgtcgtaaacaagattatttaggcttatatgaagatgaagatgaaatatttggtgtgtcaaataaaaagcttgtgtgcttaagtttgtgtttttttcttggcttgttgtgttatgaatttgtggctttttctaatattaaatgaatgtaagatctcattataatgaataaacaaatgtttctataatccattgtgaatgttttgttggatctcttctgcagcatataactactgtatgtgctatggtatggactatggaattccgctgcaagaggatgcacatgtgaccgagggaatcgggaattaaactatcagtgtttgacaggatatattggcgggtaaacctaagagaaaagagcgtttattagaataacggatatttaaaagggcgtgaaaaggtttatccgttcgtccatttgtatgtgcatgccaaccacagggttcccctcgggatcaaagtactttgatccaacccctccgctgctatagtgcagtcggcttctgacgttcagtgcagccgtgttctgaaaacgacatgtcgcacaagtcctaagttacgcgacaggctgccgccctgcccttttcctggcgttttcttgtcgcgtgttttagtcgcataaagtagaatacttgcgactagaaccggagacattacgccatgaacaagagcgccgccgctggcctgctgggctatgcccgcgtcagcaccgacgaccaggacttgaccaaccaacgggccgaactgcacgcggccggctgcaccaagctgttttccgagaagatcaccggcaccaggcgcgaccgcccggagctggccaggatgcttgaccacctacgccctggcgacgttgtgacagtgaccaggctagaccgcctggcccgcagcacccgcgacctactggacattgccgagcgcatccaggaggccggcgcgggcctgcgtagcctggcagagccgtgggccgacaccaccacgccggccggccgcatggtgttgaccgtgttcgccggcattgccgagttcgagcgttccctaatcatcgaccgcacccggagcgggcgcgaggccgccaaggcccgaggcgtgaagtttggcccccgccctaccctcaccccggcacagatcgcgcacgcccgcgagctgatcgaccaggaaggccgcaccgtgaaagaggcggctgcactgcttggcgtgcatcgctcgaccctgtaccgcgcacttgagcgcagcgaggaagtgacgcccaccgaggccaggcggcgcggtgccttccgtgaggacgcattgaccgaggccgacgccctggcggccgccgagaatgaacgccaagaggaacaagcatgaaaccgcaccaggacggccaggacgaaccgtttttcattaccgaagagatcgaggcggagatgatcgcggccgggtacgtgttcgagccgcccgcgcacgtctcaaccgtgcggctgcatgaaatcctggccggtttgtctgatgccaagctggcggcctggccggccagcttggccgctgaagaaaccgagcgccgccgtctaaaaaggtgatgtgtatttgagtaaaacagcttgcgtcatgcggtcgctgcgtatatgatgcgatgagtaaataaacaaatacgcaaggggaacgcatgaaggttatcgctgtacttaaccagaaaggcgggtcaggcaagacgaccatcgcaacccatctagcccgcgccctgcaactcgccggggccgatgttctgttagtcgattccgatccccagggcagtgcccgcgattgggcggccgtgcgggaagatcaaccgctaaccgttgtcggcatcgaccgcccgacgattgaccgcgacgtgaaggccatcggccggcgcgacttcgtagtgatcgacggagcgccccaggcggcggacttggctgtgtccgcgatcaaggcagccgacttcgtgctgattccggtgcagccaagcccttacgacatatgggccaccgccgacctggtggagctggttaagcagcgcattgaggtcacggatggaaggctacaagcggcctttgtcgtgtcgcgggcgatcaaaggcacgcgcatcggcggtgaggttgccgaggcgctggccgggtacgagctgcccattcttgagtcccgtatcacgcagcgcgtgagctacccaggcactgccgccgccggcacaaccgttcttgaatcagaacccgagggcgacgctgcccgcgaggtccaggcgctggccgctgaaattaaatcaaaactcatttgagttaatgaggtaaagagaaaatgagcaaaagcacaaacacgctaagtgccggccgtccgagcgcacgcagcagcaaggctgcaacgttggccagcctggcagacacgccagccatgaagcgggtcaactttcagttgccggcggaggatcacaccaagctgaagatgtacgcggtacgccaaggcaagaccattaccgagctgctatctgaatacatcgcgcagctaccagagtaaatgagcaaatgaataaatgagtagatgaattttagcggctaaaggaggcggcatggaaaatcaagaacaaccaggcaccgacgccgtggaatgccccatgtgtggaggaacgggcggttggccaggcgtaagcggctgggttgtctgccggccctgcaatggcactggaacccccaagcccgaggaatcggcgtgacggtcgcaaaccatccggcccggtacaaatcggcgcggcgctgggtgatgacctggtggagaagttgaaggccgcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctagctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggtctctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgaagagctgcaaaaagcgcctacccttcggtcgctgcgctccctacgccccgccgcttcgcgtcggcctatcgcggccgctggccgctcaaaaatggctggcctacggccaggcaatctaccagggcgcggacaagccgcgccgtcgccactcgaccgccggcgcccacatcaaggcaccctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgcattctaggtactaaaacaattcatccagtaaaatataatattttattttctcccaatcaggcttgatccccagtaagtcaaaaaatagctcgacatactgttcttccccgatatcctccctgatcgaccggacgcagaaggcaatgtcataccacttgtccgccctgccgcttctcccaagatcaataaagccacttactttgccatctttcacaaagatgttgctgtctcccaggtcgccgtgggaaaagacaagttcctcttcgggcttttccgtctttaaaaaatcatacagctcgcgcggatctttaaatggagtatcttcttcccagttttcgcaatccacatcggccagatcgttattcagtaagtaatccaattcggctaagcggctgtctaagctattcgtatagggacaatccgatatgtcgatggagtgaaagagcctgatgcactccgcatacagctcgataatcttttcagggctttgttcatcttcatactcttccgagcaaaggacgccatcggcctcactcatgagcagattgctccagccatcatgccgttcaaagtgcaggacctttggaacaggcagctttccttccagccatagcatcatgtccttttcccgttccacatcataggtggtccctttataccggctgtccgtcatttttaaatataggttttcattttctcccaccagcttatataccttagcctgggcctcatgggccttcctttcactgcccgctttccagtcgggaaacctgtcgtgccaggcccacatcaaggcaccctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgcattctaggtactaaaacaattcatccagtaaaatataatattttattttctcccaatcaggcttgatccccagtaagtcaaaaaatagctcgacatactgttcttccccgatatcctccctgatcgaccggacgcagaaggcaatgtcataccacttgtccgccctgccgcttctcccaagatcaataaagccacttactttgccatctttcacaaagatgttgctgtctcccaggtcgccgtgggaaaagacaagttcctcttcgggcttttccgtctttaaaaaatcatacagctcgcgcggatctttaaatggagtatcttcttcccagttttcgcaatccacatcggccagatcgttattcagtaagtaatccaattcggctaagcggctgtctaagctattcgtatagggacaatccgatatgtcgatggagtgaaagagcctgatgcactccgcatacagctcgataatcttttcagggctttgttcatcttcatactcttccgagcaaaggacgccatcggcctcactcatgagcagattgctccagccatcatgccgttcaaagtgcaggacctttggaacaggcagctttccttccagccatagcatcatgtccttttcccgttccacatcataggtggtccctttataccggctgtccgtcatttttaaatataggttttcattttctcccaccagcttatataccttagcaggagacattccttccgtatcttttacgcagcggtatttttcgatcagttttttcaattccggtgatattctcattttagccatttattatttccttcctcttttctacagtatttaaagataccccaagaagctaattataacaagacgaactccaattcactgttccttgcattctaaaaccttaaataccagaaaacagctttttcaaagttgttttcaaagttggcgtataacatagtatcgacggagccgattttgaaaccgcggtgatcacaggcagcaacgctctgtcatcgttacaatcaacatgctaccctccgcgagatcatccgtgtttcaaacccggcagcttagttgccgttcttccgaatagcatcggtaacatgagcaaagtctgccgccttacaacggctctcccgctgacgccgtcccggactgatgggctgcctgtatcgagtggtgattttgtgccgagctgccggtcggggagctgttggctggctggtggcaggatatattgtggtgtaaacaaattgacgcttagacaacttaataacacattgcggacgtttttaatgtactgaattaacgccgaattaattcgggggatctggattttagtactggattttggttttaggaattagaaattttattgatagaagtattttacaaatacaaatacatactaagggtttcttatatgctcaacacatgagcgaaaccctataggaaccctaattcccttatctgggaactactcacacattattatggagaaactcgagcttgtcgatcgacagatcccggtcggcatctactctatttctttgccctcggacgagtgctggggcgtcggtttccactatcggcgagtacttctacacagccatcggtccagacggccgcgcttctgcgggcgatttgtgtacgcccgacagtcccggctccggatcggacgattgcgtcgcatcgaccctgcgcccaagctgcatcatcgaaattgccgtcaaccaagctctgatagagttggtcaagaccaatgcggagcatatacgcccggagtcgtggcgatcctgcaagctccggatgcctccgctcgaagtagcgcgtctgctgctccatacaagccaaccacggcctccagaagaagatgttggcgacctcgtattgggaatccccgaacatcgcctcgctccagtcaatgaccgctgttatgcggccattgtccgtcaggacattgttggagccgaaatccgcgtgcacgaggtgccggacttcggggcagtcctcggcccaaagcatcagctcatcgagagcctgcgcgacggacgcactgacggtgtcgtccatcacagtttgccagtgatacacatggggatcagcaatcgcgcatatgaaatcacgccatgtagtgtattgaccgattccttgcggtccgaatgggccgaacccgctcgtctggctaagatcggccgcagcgatcgcatccatagcctccgcgaccggttgtagaacagcgggcagttcggtttcaggcaggtcttgcaacgtgacaccctgtgcacggcgggagatgcaataggtcaggctctcgctaaactccccaatgtcaagcacttccggaatcgggagcgcggccgatgcaaagtgccgataaacataacgatctttgtagaaaccatcggcgcagctatttacccgcaggacatatccacgccctcctacatcgaagctgaaagcacgagattcttcgccctccgagagctgcatcaggtcggagacgctgtcgaacttttcgatcagaaacttctcgacagacgtcgcggtgagttcaggctttttcatatccggtgcagattatttggattgagagtgaatatgagactctaattggataccgaggggaatttatggaacgtcagtggagcatttttgacaagaaatatttgctagctgatagtgaccttaggcgacttttgaacgcgcaataatggtttctgacgtatgtgcttagctcattaaactccagaaacccgcggctgagtggctccttcaacgttgcggttctgtcagttccaaacgtaaaacggcttgtcccgcgtcatcggcgggggtcataacgtgactcccttaattctccgctcatgatcagattgtcgtttcccgccttcagtttaaactatcagtgtccaacatgttggcaagctgctctagccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgaattc

SEQ ID NO: 2 - DNA, 1520 bp, synthetic construct
  source 1..1520: mol_type = other DNA
attgagtctaaaatattgaaaatactcactatcacatatgtcaacgataacactaatttttttttttgtataatatacgtaacgacacctgcattagatctaatttttaatcgattagagcttaaatgacatcgttttggtttgactaagagaatcgttaaaaatgttagaaaatctgttaaagcttaataactgtttgtttgttttttttctttctttttttgtcaatcgaaaattagactattaaatgacaattttccgtttaaaatggcaagtcctagatttgaaaaggtcttttgaaatctttttaagagaattaagggtgaattaaatatattcagaaattaaagggtcgaattgcaaactttcttggttcatacttggtctgcctttgtccctttcttatcttcttcgcgcgcttttgaaaaaaatagaaacccgccactgaacgtcagtcatccacgtgtcctctcctgtgctctgtttccgatcgtcggagacatcaatttgcctgcttgtagggtgattcgtcaaatctattatcaggttttaaatatactcgaattgacttccaaattcttagtctctagtgtaatgattttgagaatcacttaactccaaaaatataatccacgatcccgtgttaattattgaagaatcaatcgtttttaatttctcaccaatagatgttgctcttattacttaaaacaaattgtttagacaaatgtagcaagtgtgatacttagtgggatcttaaagacgatttctcctataacagaggacaaacaggtcggtcaattacaatgtcatccctctttaccctgtctttttttttcttcttaaaacctaaccatttgattgtttctaaaggtatttcaagaatatatgatcaatctagatgaatactataccgacgatgactacacacacaaggaaatatatatatcagctttcttttcacctaaaagtggtcccggtttagaatctaattcctttatctctcattttcttctgcttcacattcccgctagtcaaatgttaataagtgcacacaacgttttctcgaagcattagaatgtcctcctcttaattaatctccttctgattagattctcaatagagtttaaatttgttaatggagagatatattgggaccctcaaggcttctaattataccacgtttggcataattctctatcgtttggggccacatctttcacacttcattaccttatcaccaaaacataaaatcaatcaacttttttttgccttattgattgtgttggatccctccaaaattaaaacttgtgttccccacaaaagcttacccaatttcacttcaatcttaacaaataggaccaccactaccacgtacggtttgcatcatacaaaccacaaactccttcttcattacaattattatatcatctactaaaacctctttctccctctctctttcttgttcttagtgctaaattttctttgttcaggagaaatatatactgaattcggatcctcta

SEQ ID NO: 3 - DNA, 4148 bp, synthetic construct
  source 1..4148: mol_type = other DNA
gacctcaagtattcagcatctcattgcaacttatcctcagacatgaagctcaggcgttttcatcagcatcgtggaaaaggaagagaagaagagtatgatgcttcttctctcagcttgaacaatctgtcaaaacttattcttcctccacttggtgttgctagctataaccagaatcacatcaggtctagtggatggatcatctcacctatggactcaagatacaggtgctgggaattttatatggtgcttttagtggcatactctgcttgggtttacccttttgaagttgcatttctgaattcatcaccaaagagaaacctttgtatcgctgacaacatcgtagacttgttcttcgctgttgacattgtcttgacttttttcgttgcttacattgacgaaagaacacagcttcttgtccgtgaacctaaacagattgcagtgaggtacctatcaacatggttcttgatggatgttgcatcaactattccatttgacgctattggatacttaatcactggcacatccactttaaatatcacttgtaatctcttgggattacttagattttggagacttcgtagagttaaacacctcttcactaggctcgagaaggacattagatataactatttctggattcgctgctttagacttctatcagtgacattgtttctagtgcactgtgctggatgcagttattacctaattgcagacagatatccacaccaaggaaagacatggactgatgctatccctaatttcacagagacaagtctttccatcagatacattgcagctatttattggtctatcactacaatgaccacagtgggatatggagatcttcatgcaagcaacactattgaaatggtattcattacagtctacatgttattcaatcttggcctcactgcttaccttattggtaacatgactaatttggtcgtggaagggactcgtcgtaccatggaatttaggaataacattgaagcagcttcaaactttgttaacagaaacagattgcctcctagattaaaagaccagattttagcttacatgtgtttaaggtttaaagcagagagcttaaatcagcaacatcttattgaccagctcccaaaatctatctacaaaagcatttgtcaacatctttttcttccatctgttgaaaaagtttacctcttcaaaggcgtctcaagagaaattcttcttcttctggtttcaaaaatgaaggctgagtatattccaccaagagaggatgtcattatgcagaacgaagctccagatgatgtttacattattgtgtcaggagaagttgagatcattgattcagagatggagagagagtctgttttaggcactctacgttgtggagacatttttggagaagttggagcactttgttgcagaccacaaagctacacttttcaaactaagtctttatcacagcttctccgtctcaaaacatctttccttattgagacaatgcagattaaacaacaagacaatgccacaatgctcaagaacttcttgcagcatcacaaaaagctgagtaatttagacattggtgatctaaaggcacaacaaaatggcgaaaacaccgatgttgttcctcctaacattgcctcaaatctcatcgctgtggtgactacaggcaatgcagctcttcttgatgagctacttaaggctaagttaagccctgacattacagattccaaaggaaaaactccattgcatgtagcagcttctagaggatatgaagattgtgttttagtactcttaaagcacggttgcaacatccacattagagatgtgaatggtaatagtgctctatgggaagcaattatttctaagcattacgagattttcagaatcctttatcatttcgcagccatttctgatccacacattgctggagatcttctatgtgaagcagctaaacagaacaatgtagaagtcatgaaggctcttttaaaacaggggcttaacgtcgacacagaggatcaccatggcgtcacagctttacaggtcgctatggctgaggatcagatggacatggtgaatctcctggctactaacggtgcagatgtagtttgtgtgaatacacataatgaattcacaccattggagaagttaagagttgtggaagaagaagaagaagaagaacgtggaagagtgagtatttacagaggacatccattggagaggagagaaagaagttgcaatgaagctgggaagcttattcttcttcctccttcacttgatgacctcaagaaaattgcaggagagaagtttgggtttgatggaagtgagactatggtgactaatgaagatggagctgagattgacagtattgaagtgattagagataatgacaaactctactttgtcgtaaacaagattatttaggcttatatgaagatgaagatgaaatatttggtgtgtcaaataaaaagcttgtgtgcttaagtttgtgtttttttcttggcttgttgtgttatgaatttgtggctttttctaatattaaatgaatgtaagatctcattataatgaataaacaaatgtttctataatccattgtgaatgttttgttggatctcttctgcagcatataactactgtatgtgctatggtatggactatggaattccgctgcaagaggatgcacatgtgaccgagggaatcgggaattaaactatcagtgtttgacaggatatattggcgggtaaacctaagagaaaagagcgtttattagaataacggatatttaaaagggcgtgaaaaggtttatccgttcgtccatttgtatgtgcatgccaaccacagggttcccctcgggatcaaagtactttgatccaacccctccgctgctatagtgcagtcggcttctgacgttcagtgcagccgtgttctgaaaacgacatgtcgcacaagtcctaagttacgcgacaggctgccgccctgcccttttcctggcgttttcttgtcgcgtgttttagtcgcataaagtagaatacttgcgactagaaccggagacattacgccatgaacaagagcgccgccgctggcctgctgggctatgcccgcgtcagcaccgacgaccaggacttgaccaaccaacgggccgaactgcacgcggccggctgcaccaagctgttttccgagaagatcaccggcaccaggcgcgaccgcccggagctggccaggatgcttgaccacctacgccctggcgacgttgtgacagtgaccaggctagaccgcctggcccgcagcacccgcgacctactggacattgccgagcgcatccaggaggccggcgcgggcctgcgtagcctggcagagccgtgggccgacaccaccacgccggccggccgcatggtgttgaccgtgttcgccggcattgccgagttcgagcgttccctaatcatcgaccgcacccggagcgggcgcgaggccgccaaggcccgaggcgtgaagtttggcccccgccctaccctcaccccggcacagatcgcgcacgcccgcgagctgatcgaccaggaaggccgcaccgtgaaagaggcggctgcactgcttggcgtgcatcgctcgaccctgtaccgcgcacttgagcgcagcgaggaagtgacgcccaccgaggccaggcggcgcggtgccttccgtgaggacgcattgaccgaggccgacgccctggcggccgccgagaatgaacgccaagaggaacaagcatgaaaccgcaccaggacggccaggacgaaccgtttttcattaccgaagagatcgaggcggagatgatcgcggccgggtacgtgttcgagccgcccgcgcacgtctcaaccgtgcggctgcatgaaatcctggccggtttgtctgatgccaagctggcggcctggccggccagcttggccgctgaagaaaccgagcgccgccgtctaaaaaggtgatgtgtatttgagtaaaacagcttgcgtcatgcggtcgctgcgtatatgatgcgatgagtaaataaacaaatacgcaaggggaacgcatgaaggttatcgctgtacttaaccagaaaggcgggtcaggcaagacgaccatcgcaacccatctagcccgcgccctgcaactcgccggggccgatgttctgttagtcgattccgatcccc

SEQ ID NO: 4 - DNA, 1626 bp, synthetic construct
  source 1..1626: mol_type = other DNA
ctatttctttgccctcggacgagtgctggggcgtcggtttccactatcggcgagtacttctacacagccatcggtccagacggccgcgcttctgcgggcgatttgtgtacgcccgacagtcccggctccggatcggacgattgcgtcgcatcgaccctgcgcccaagctgcatcatcgaaattgccgtcaaccaagctctgatagagttggtcaagaccaatgcggagcatatacgcccggagtcgtggcgatcctgcaagctccggatgcctccgctcgaagtagcgcgtctgctgctccatacaagccaaccacggcctccagaagaagatgttggcgacctcgtattgggaatccccgaacatcgcctcgctccagtcaatgaccgctgttatgcggccattgtccgtcaggacattgttggagccgaaatccgcgtgcacgaggtgccggacttcggggcagtcctcggcccaaagcatcagctcatcgagagcctgcgcgacggacgcactgacggtgtcgtccatcacagtttgccagtgatacacatggggatcagcaatcgcgcatatgaaatcacgccatgtagtgtattgaccgattccttgcggtccgaatgggccgaacccgctcgtctggctaagatcggccgcagcgatcgcatccatagcctccgcgaccggttgtagaacagcgggcagttcggtttcaggcaggtcttgcaacgtgacaccctgtgcacggcgggagatgcaataggtcaggctctcgctaaactccccaatgtcaagcacttccggaatcgggagcgcggccgatgcaaagtgccgataaacataacgatctttgtagaaaccatcggcgcagctatttacccgcaggacatatccacgccctcctacatcgaagctgaaagcacgagattcttcgccctccgagagctgcatcaggtcggagacgctgtcgaacttttcgatcagaaacttctcgacagacgtcgcggtgagttcaggctttttcatatccggtgcagattatttggattgagagtgaatatgagactctaattggataccgaggggaatttatggaacgtcagtggagcatttttgacaagaaatatttgctagctgatagtgaccttaggcgacttttgaacgcgcaataatggtttctgacgtatgtgcttagctcattaaactccagaaacccgcggctgagtggctccttcaacgttgcggttctgtcagttccaaacgtaaaacggcttgtcccgcgtcatcggcgggggtcataacgtgactcccttaattctccgctcatgatcagattgtcgtttcccgccttcagtttaaactatcagtgtccaacatgttggcaagctgctctagccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgaattc

SEQ ID NO: 5 - DNA, 558 bp, synthetic construct
  source 1..558: mol_type = other DNA
taattggataccgaggggaatttatggaacgtcagtggagcatttttgacaagaaatatttgctagctgatagtgaccttaggcgacttttgaacgcgcaataatggtttctgacgtatgtgcttagctcattaaactccagaaacccgcggctgagtggctccttcaacgttgcggttctgtcagttccaaacgtaaaacggcttgtcccgcgtcatcggcgggggtcataacgtgactcccttaattctccgctcatgatcagattgtcgtttcccgccttcagtttaaactatcagtgtccaacatgttggcaagctgctctagccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgaattc

SEQ ID NO: 6 - DNA, 10246 bp, synthetic construct
  source 1..10246: mol_type = other DNA
cgatccccagggcagtgcccgcgattgggcggccgtgcgggaagatcaaccgctaaccgttgtcggcatcgaccgcccgacgattgaccgcgacgtgaaggccatcggccggcgcgacttcgtagtgatcgacggagcgccccaggcggcggacttggctgtgtccgcgatcaaggcagccgacttcgtgctgattccggtgcagccaagcccttacgacatatgggccaccgccgacctggtggagctggttaagcagcgcattgaggtcacggatggaaggctacaagcggcctttgtcgtgtcgcgggcgatcaaaggcacgcgcatcggcggtgaggttgccgaggcgctggccgggtacgagctgcccattcttgagtcccgtatcacgcagcgcgtgagctacccaggcactgccgccgccggcacaaccgttcttgaatcagaacccgagggcgacgctgcccgcgaggtccaggcgctggccgctgaaattaaatcaaaactcatttgagttaatgaggtaaagagaaaatgagcaaaagcacaaacacgctaagtgccggccgtccgagcgcacgcagcagcaaggctgcaacgttggccagcctggcagacacgccagccatgaagcgggtcaactttcagttgccggcggaggatcacaccaagctgaagatgtacgcggtacgccaaggcaagaccattaccgagctgctatctgaatacatcgcgcagctaccagagtaaatgagcaaatgaataaatgagtagatgaattttagcggctaaaggaggcggcatggaaaatcaagaacaaccaggcaccgacgccgtggaatgccccatgtgtggaggaacgggcggttggccaggcgtaagcggctgggttgtctgccggccctgcaatggcactggaacccccaagcccgaggaatcggcgtgacggtcgcaaaccatccggcccggtacaaatcggcgcggcgctgggtgatgacctggtggagaagttgaaggccgcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctagctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggtctctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgaagagctgcaaaaagcgcctacccttcggtcgctgcgctccctacgccccgccgcttcgcgtcggcctatcgcggccgctggccgctcaaaaatggctggcctacggccaggcaatctaccagggcgcggacaagccgcgccgtcgccactcgaccgccggcgcccacatcaaggcaccctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgcattctaggtactaaaacaattcatccagtaaaatataatattttattttctcccaatcaggcttgatccccagtaagtcaaaaaatagctcgacatactgttcttccccgatatcctccctgatcgaccggacgcagaaggcaatgtcataccacttgtccgccctgccgcttctcccaagatcaataaagccacttactttgccatctttcacaaagatgttgctgtctcccaggtcgccgtgggaaaagacaagttcctcttcgggcttttccgtctttaaaaaatcatacagctcgcgcggatctttaaatggagtatcttcttcccagttttcgcaatccacatcggccagatcgttattcagtaagtaatccaattcggctaagcggctgtctaagctattcgtatagggacaatccgatatgtcgatggagtgaaagagcctgatgcactccgcatacagctcgataatcttttcagggctttgttcatcttcatactcttccgagcaaaggacgccatcggcctcactcatgagcagattgctccagccatcatgccgttcaaagtgcaggacctttggaacaggcagctttccttccagccatagcatcatgtccttttcccgttccacatcataggtggtccctttataccggctgtccgtcatttttaaatataggttttcattttctcccaccagcttatataccttagcctgggcctcatgggccttcctttcactgcccgctttccagtcgggaaacctgtcgtgccaggcccacatcaaggcaccctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgcattctaggtactaaaacaattcatccagtaaaatataatattttattttctcccaatcaggcttgatccccagtaagtcaaaaaatagctcgacatactgttcttccccgatatcctccctgatcgaccggacgcagaaggcaatgtcataccacttgtccgccctgccgcttctcccaagatcaataaagccacttactttgccatctttcacaaagatgttgctgtctcccaggtcgccgtgggaaaagacaagttcctcttcgggcttttccgtctttaaaaaatcatacagctcgcgcggatctttaaatggagtatcttcttcccagttttcgcaatccacatcggccagatcgttattcagtaagtaatccaattcggctaagcggctgtctaagctattcgtatagggacaatccgatatgtcgatggagtgaaagagcctgatgcactccgcatacagctcgataatcttttcagggctttgttcatcttcatactcttccgagcaaaggacgccatcggcctcactcatgagcagattgctccagccatcatgccgttcaaagtgcaggacctttggaacaggcagctttccttccagccatagcatcatgtccttttcccgttccacatcataggtggtccctttataccggctgtccgtcatttttaaatataggttttcattttctcccaccagcttatataccttagcaggagacattccttccgtatcttttacgcagcggtatttttcgatcagttttttcaattccggtgatattctcattttagccatttattatttccttcctcttttctacagtatttaaagataccccaagaagctaattataacaagacgaactccaattcactgttccttgcattctaaaaccttaaataccagaaaacagctttttcaaagttgttttcaaagttggcgtataacatagtatcgacggagccgattttgaaaccgcggtgatcacaggcagcaacgctctgtcatcgttacaatcaacatgctaccctccgcgagatcatccgtgtttcaaacccggcagcttagttgccgttcttccgaatagcatcggtaacatgagcaaagtctgccgccttacaacggctctcccgctgacgccgtcccggactgatgggctgcctgtatcgagtggtgattttgtgccgagctgccggtcggggagctgttggctggctggtggcaggatatattgtggtgtaaacaaattgacgcttagacaacttaataacacattgcggacgtttttaatgtactgaattaacgccgaattaattcgggggatctggattttagtactggattttggttttaggaattagaaattttattgatagaagtattttacaaatacaaatacatactaagggtttcttatatgctcaacacatgagcgaaaccctataggaaccctaattcccttatctgggaactactcacacattattatggagaaactcgagcttgtcgatcgacagatcccggtcggcatctactctatttctttgccctcggacgagtgctggggcgtcggtttccactatcggcgagtacttctacacagccatcggtccagacggccgcgcttctgcgggcgatttgtgtacgcccgacagtcccggctccggatcggacgattgcgtcgcatcgaccctgcgcccaagctgcatcatcgaaattgccgtcaaccaagctctgatagagttggtcaagaccaatgcggagcatatacgcccggagtcgtggcgatcctgcaagctccggatgcctccgctcgaagtagcgcgtctgctgctccatacaagccaaccacggcctccagaagaagatgttggcgacctcgtattgggaatccccgaacatcgcctcgctccagtcaatgaccgctgttatgcggccattgtccgtcaggacattgttggagccgaaatccgcgtgcacgaggtgccggacttcggggcagtcctcggcccaaagcatcagctcatcgagagcctgcgcgacggacgcactgacggtgtcgtccatcacagtttgccagtgatacacatggggatcagcaatcgcgcatatgaaatcacgccatgtagtgtattgaccgattccttgcggtccgaatgggccgaacccgctcgtctggctaagatcggccgcagcgatcgcatccatagcctccgcgaccggttgtagaacagcgggcagttcggtttcaggcaggtcttgcaacgtgacaccctgtgcacggcgggagatgcaataggtcaggctctcgctaaactccccaatgtcaagcacttccggaatcgggagcgcggccgatgcaaagtgccgataaacataacgatctttgtagaaaccatcggcgcagctatttacccgcaggacatatccacgccctcctacatcgaagctgaaagcacgagattcttcgccctccgagagctgcatcaggtcggagacgctgtcgaacttttcgatcagaaacttctcgacagacgtcgcggtgagttcaggctttttcatatccggtgcagattatttggattgagagtgaatatgagactctaattggataccgaggggaatttatggaacgtcagtggagcatttttgacaagaaatatttgctagctgatagtgaccttaggcgacttttgaacgcgcaataatggtttctgacgtatgtgcttagctcattaaactccagaaacccgcggctgagtggctccttcaacgttgcggttctgtcagttccaaacgtaaaacggcttgtcccgcgtcatcggcgggggtcataacgtgactcccttaattctccgctcatgatcagattgtcgtttcccgccttcagtttaaactatcagtgtccaacatgttggcaagctgctctagccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgaattctgccatgtcttcgagctcggtacccggggatccattacaggtgaccagctcgaatttcgaagacatgggaatcgggaattaaactatcagtgtttgacaggatatattggcgggtaaacctaagagaaaagagcgtttattagaataacggatatttaaaagggcgtgaaaaggtttatccgttcgtccatttgtatgtgcatgccaaccacagggttcccctcgggatcaaagtactttgatccaacccctccgctgctatagtgcagtcggcttctgacgttcagtgcagccgtgttctgaaaacgacatgtcgcacaagtcctaagttacgcgacaggctgccgccctgcccttttcctggcgttttcttgtcgcgtgttttagtcgcataaagtagaatacttgcgactagaaccggagacattacgccatgaacaagagcgccgccgctggcctgctgggctatgcccgcgtcagcaccgacgaccaggacttgaccaaccaacgggccgaactgcacgcggccggctgcaccaagctgttttccgagaagatcaccggcaccaggcgcgaccgcccggagctggccaggatgcttgaccacctacgccctggcgacgttgtgacagtgaccaggctagaccgcctggcccgcagcacccgcgacctactggacattgccgagcgcatccaggaggccggcgcgggcctgcgtagcctggcagagccgtgggccgacaccaccacgccggccggccgcatggtgttgaccgtgttcgccggcattgccgagttcgagcgttccctaatcatcgaccgcacccggagcgggcgcgaggccgccaaggcccgaggcgtgaagtttggcccccgccctaccctcaccccggcacagatcgcgcacgcccgcgagctgatcgaccaggaaggccgcaccgtgaaagaggcggctgcactgcttggcgtgcatcgctcgaccctgtaccgcgcacttgagcgcagcgaggaagtgacgcccaccgaggccaggcggcgcggtgccttccgtgaggacgcattgaccgaggccgacgccctggcggccgccgagaatgaacgccaagaggaacaagcatgaaaccgcaccaggacggccaggacgaaccgtttttcattaccgaagagatcgaggcggagatgatcgcggccgggtacgtgttcgagccgcccgcgcacgtctcaaccgtgcggctgcatgaaatcctggccggtttgtctgatgccaagctggcggcctggccggccagcttggccgctgaagaaaccgagcgccgccgtctaaaaaggtgatgtgtatttgagtaaaacagcttgcgtcatgcggtcgctgcgtatatgatgcgatgagtaaataaacaaatacgcaaggggaacgcatgaaggttatcgctgtacttaaccagaaaggcgggtcaggcaagacgaccatcgcaacccatctagcccgcgccctgcaactcgccggggccgatgttctgttagtcgattc

SEQ ID NO: 7 - DNA, 2018 bp, synthetic construct
  source 1..2018: mol_type = other DNA
atggcactggaacccccaagcccgaggaatcggcgtgacggtcgcaaaccatccggcccggtacaaatcggcgcggcgctgggtgatgacctggtggagaagttgaaggccgcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctagctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggtctctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgaagagctgcaaaaagcgcctacccttcggtcgctgcgctccctacgccccgccgcttcgcgtcggcctatcgcggccgctggccgctcaaaaatggctggcctacggccaggcaatctaccagggcgcggacaagccgcgccgtcgccactcgaccgccggcgcccacatcaaggcaccctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaa

SEQ ID NO: 8 - DNA, 4693 bp, synthetic construct
  source 1..4693: mol_type = other DNA
ctaaaacaattcatccagtaaaatataatattttattttctcccaatcaggcttgatccccagtaagtcaaaaaatagctcgacatactgttcttccccgatatcctccctgatcgaccggacgcagaaggcaatgtcataccacttgtccgccctgccgcttctcccaagatcaataaagccacttactttgccatctttcacaaagatgttgctgtctcccaggtcgccgtgggaaaagacaagttcctcttcgggcttttccgtctttaaaaaatcatacagctcgcgcggatctttaaatggagtatcttcttcccagttttcgcaatccacatcggccagatcgttattcagtaagtaatccaattcggctaagcggctgtctaagctattcgtatagggacaatccgatatgtcgatggagtgaaagagcctgatgcactccgcatacagctcgataatcttttcagggctttgttcatcttcatactcttccgagcaaaggacgccatcggcctcactcatgagcagattgctccagccatcatgccgttcaaagtgcaggacctttggaacaggcagctttccttccagccatagcatcatgtccttttcccgttccacatcataggtggtccctttataccggctgtccgtcatttttaaatataggttttcattttctcccaccagcttatataccttagcaggagacattccttccgtatcttttacgcagcggtatttttcgatcagttttttcaattccggtgatattctcattttagccatttattatttccttcctcttttctacagtatttaaagataccccaagaagctaattataacaagacgaactccaattcactgttccttgcattctaaaaccttaaataccagaaaacagctttttcaaagttgttttcaaagttggcgtataacatagtatcgacggagccgattttgaaaccgcggtgatcacaggcagcaacgctctgtcatcgttacaatcaacatgctaccctccgcgagatcatccgtgtttcaaacccggcagcttagttgccgttcttccgaatagcatcggtaacatgagcaaagtctgccgccttacaacggctctcccgctgacgccgtcccggactgatgggctgcctgtatcgagtggtgattttgtgccgagctgccggtcggggagctgttggctggctggtggcaggatatattgtggtgtaaacaaattgacgcttagacaacttaataacacattgcggacgtttttaatgtactgaattaacgccgaattaattcgggggatctggattttagtactggattttggttttaggaattagaaattttattgatagaagtattttacaaatacaaatacatactaagggtttcttatatgctcaacacatgagcgaaaccctataggaaccctaattcccttatctgggaactactcacacattattatggagaaactcgagcttgtcgatcgacagatcccggtcggcatctactctatttctttgccctcggacgagtgctggggcgtcggtttccactatcggcgagtacttctacacagccatcggtccagacggccgcgcttctgcgggcgatttgtgtacgcccgacagtcccggctccggatcggacgattgcgtcgcatcgaccctgcgcccaagctgcatcatcgaaattgccgtcaaccaagctctgatagagttggtcaagaccaatgcggagcatatacgcccggagtcgtggcgatcctgcaagctccggatgcctccgctcgaagtagcgcgtctgctgctccatacaagccaaccacggcctccagaagaagatgttggcgacctcgtattgggaatccccgaacatcgcctcgctccagtcaatgaccgctgttatgcggccattgtccgtcaggacattgttggagccgaaatccgcgtgcacgaggtgccggacttcggggcagtcctcggcccaaagcatcagctcatcgagagcctgcgcgacggacgcactgacggtgtcgtccatcacagtttgccagtgatacacatggggatcagcaatcgcgcatatgaaatcacgccatgtagtgtattgaccgattccttgcggtccgaatgggccgaacccgctcgtctggctaagatcggccgcagcgatcgcatccatagcctccgcgaccggttgtagaacagcgggcagttcggtttcaggcaggtcttgcaacgtgacaccctgtgcacggcgggagatgcaataggtcaggctctcgctaaactccccaatgtcaagcacttccggaatcgggagcgcggccgatgcaaagtgccgataaacataacgatctttgtagaaaccatcggcgcagctatttacccgcaggacatatccacgccctcctacatcgaagctgaaagcacgagattcttcgccctccgagagctgcatcaggtcggagacgctgtcgaacttttcgatcagaaacttctcgacagacgtcgcggtgagttcaggctttttcatatccggtgcagattatttggattgagagtgaatatgagactctaattggataccgaggggaatttatggaacgtcagtggagcatttttgacaagaaatatttgctagctgatagtgaccttaggcgacttttgaacgcgcaataatggtttctgacgtatgtgcttagctcattaaactccagaaacccgcggctgagtggctccttcaacgttgcggttctgtcagttccaaacgtaaaacggcttgtcccgcgtcatcggcgggggtcataacgtgactcccttaattctccgctcatgatcagattgtcgtttcccgccttcagtttaaactatcagtgtccaacatgttggcaagctgctctagccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgaattctgccatgtcttcgagctcggtacccggggatccattacaggtgaccagctcgaatttcgaagacatgggaatcgggaattaaactatcagtgtttgacaggatatattggcgggtaaacctaagagaaaagagcgtttattagaataacggatatttaaaagggcgtgaaaaggtttatccgttcgtccatttgtatgtgcatgccaaccacagggttcccctcgggatcaaagtactttgatccaacccctccgctgctatagtgcagtcggcttctgacgttcagtgcagccgtgttctgaaaacgacatgtcgcacaagtcctaagttacgcgacaggctgccgccctgcccttttcctggcgttttcttgtcgcgtgttttagtcgcataaagtagaatacttgcgactagaaccggagacattacgccatgaacaagagcgccgccgctggcctgctgggctatgcccgcgtcagcaccgacgaccaggacttgaccaaccaacgggccgaactgcacgcggccggctgcaccaagctgttttccgagaagatcaccggcaccaggcgcgaccgcccggagctggccaggatgcttgaccacctacgccctggcgacgttgtgacagtgaccaggctagaccgcctggcccgcagcacccgcgacctactggacattgccgagcgcatccaggaggccggcgcgggcctgcgtagcctggcagagccgtgggccgacaccaccacgccggccggccgcatggtgttgaccgtgttcgccggcattgccgagttcgagcgttccctaatcatcgaccgcacccggagcgggcgcgaggccgccaaggcccgaggcgtgaagtttggcccccgccctaccctcaccccggcacagatcgcgcacgcccgcgagctgatcgaccaggaaggccgcaccgtgaaagaggcggctgcactgcttggcgtgcatcgctcgaccctgtaccgcgcacttgagcgcagcgaggaagtgacgcccaccgaggccaggcggcgcggtgccttccgtgaggacgcattgaccgaggccgacgccctggcggccgccgagaatgaacgccaagaggaacaagcatgaaaccgcaccaggacggccaggacgaaccgtttttcattaccgaagagatcgaggcggagatgatcgcggccgggtacgtgttcgagccgcccgcgcacgtctcaaccgtgcggctgcatgaaatcctggccggtttgtctgatgccaagctggcggcctggccggccagcttggccgctgaagaaaccgagcgccgccgtctaaaaaggtgatgtgtatttgagtaaaacagcttgcgtcatgcggtcgctgcgtatatgatgcgatgagtaaataaacaaatacgcaaggggaacgcatgaaggttatcgctgtacttaaccagaaaggcgggtcaggcaagacgaccatcgcaacccatctagcccgcgccctgcaactcgccggggccgatgttctgttagtcgattc

SEQ ID NO: 9 - DNA, 3157 bp, synthetic construct
  source 1..3157: mol_type = other DNA
ctatttctttgccctcggacgagtgctggggcgtcggtttccactatcggcgagtacttctacacagccatcggtccagacggccgcgcttctgcgggcgatttgtgtacgcccgacagtcccggctccggatcggacgattgcgtcgcatcgaccctgcgcccaagctgcatcatcgaaattgccgtcaaccaagctctgatagagttggtcaagaccaatgcggagcatatacgcccggagtcgtggcgatcctgcaagctccggatgcctccgctcgaagtagcgcgtctgctgctccatacaagccaaccacggcctccagaagaagatgttggcgacctcgtattgggaatccccgaacatcgcctcgctccagtcaatgaccgctgttatgcggccattgtccgtcaggacattgttggagccgaaatccgcgtgcacgaggtgccggacttcggggcagtcctcggcccaaagcatcagctcatcgagagcctgcgcgacggacgcactgacggtgtcgtccatcacagtttgccagtgatacacatggggatcagcaatcgcgcatatgaaatcacgccatgtagtgtattgaccgattccttgcggtccgaatgggccgaacccgctcgtctggctaagatcggccgcagcgatcgcatccatagcctccgcgaccggttgtagaacagcgggcagttcggtttcaggcaggtcttgcaacgtgacaccctgtgcacggcgggagatgcaataggtcaggctctcgctaaactccccaatgtcaagcacttccggaatcgggagcgcggccgatgcaaagtgccgataaacataacgatctttgtagaaaccatcggcgcagctatttacccgcaggacatatccacgccctcctacatcgaagctgaaagcacgagattcttcgccctccgagagctgcatcaggtcggagacgctgtcgaacttttcgatcagaaacttctcgacagacgtcgcggtgagttcaggctttttcatatccggtgcagattatttggattgagagtgaatatgagactctaattggataccgaggggaatttatggaacgtcagtggagcatttttgacaagaaatatttgctagctgatagtgaccttaggcgacttttgaacgcgcaataatggtttctgacgtatgtgcttagctcattaaactccagaaacccgcggctgagtggctccttcaacgttgcggttctgtcagttccaaacgtaaaacggcttgtcccgcgtcatcggcgggggtcataacgtgactcccttaattctccgctcatgatcagattgtcgtttcccgccttcagtttaaactatcagtgtccaacatgttggcaagctgctctagccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgaattctgccatgtcttcgagctcggtacccggggatccattacaggtgaccagctcgaatttcgaagacatgggaatcgggaattaaactatcagtgtttgacaggatatattggcgggtaaacctaagagaaaagagcgtttattagaataacggatatttaaaagggcgtgaaaaggtttatccgttcgtccatttgtatgtgcatgccaaccacagggttcccctcgggatcaaagtactttgatccaacccctccgctgctatagtgcagtcggcttctgacgttcagtgcagccgtgttctgaaaacgacatgtcgcacaagtcctaagttacgcgacaggctgccgccctgcccttttcctggcgttttcttgtcgcgtgttttagtcgcataaagtagaatacttgcgactagaaccggagacattacgccatgaacaagagcgccgccgctggcctgctgggctatgcccgcgtcagcaccgacgaccaggacttgaccaaccaacgggccgaactgcacgcggccggctgcaccaagctgttttccgagaagatcaccggcaccaggcgcgaccgcccggagctggccaggatgcttgaccacctacgccctggcgacgttgtgacagtgaccaggctagaccgcctggcccgcagcacccgcgacctactggacattgccgagcgcatccaggaggccggcgcgggcctgcgtagcctggcagagccgtgggccgacaccaccacgccggccggccgcatggtgttgaccgtgttcgccggcattgccgagttcgagcgttccctaatcatcgaccgcacccggagcgggcgcgaggccgccaaggcccgaggcgtgaagtttggcccccgccctaccctcaccccggcacagatcgcgcacgcccgcgagctgatcgaccaggaaggccgcaccgtgaaagaggcggctgcactgcttggcgtgcatcgctcgaccctgtaccgcgcacttgagcgcagcgaggaagtgacgcccaccgaggccaggcggcgcggtgccttccgtgaggacgcattgaccgaggccgacgccctggcggccgccgagaatgaacgccaagaggaacaagcatgaaaccgcaccaggacggccaggacgaaccgtttttcattaccgaagagatcgaggcggagatgatcgcggccgggtacgtgttcgagccgcccgcgcacgtctcaaccgtgcggctgcatgaaatcctggccggtttgtctgatgccaagctggcggcctggccggccagcttggccgctgaagaaaccgagcgccgccgtctaaaaaggtgatgtgtatttgagtaaaacagcttgcgtcatgcggtcgctgcgtatatgatgcgatgagtaaataaacaaatacgcaaggggaacgcatgaaggttatcgctgtacttaaccagaaaggcgggtcaggcaagacgaccatcgcaacccatctagcccgcgccctgcaactcgccggggccgatgttctgttagtcgattc

SEQ ID NO: 10 - DNA, 2089 bp, synthetic construct
  source 1..2089: mol_type = other DNA
taattggataccgaggggaatttatggaacgtcagtggagcatttttgacaagaaatatttgctagctgatagtgaccttaggcgacttttgaacgcgcaataatggtttctgacgtatgtgcttagctcattaaactccagaaacccgcggctgagtggctccttcaacgttgcggttctgtcagttccaaacgtaaaacggcttgtcccgcgtcatcggcgggggtcataacgtgactcccttaattctccgctcatgatcagattgtcgtttcccgccttcagtttaaactatcagtgtccaacatgttggcaagctgctctagccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgaattctgccatgtcttcgagctcggtacccggggatccattacaggtgaccagctcgaatttcgaagacatgggaatcgggaattaaactatcagtgtttgacaggatatattggcgggtaaacctaagagaaaagagcgtttattagaataacggatatttaaaagggcgtgaaaaggtttatccgttcgtccatttgtatgtgcatgccaaccacagggttcccctcgggatcaaagtactttgatccaacccctccgctgctatagtgcagtcggcttctgacgttcagtgcagccgtgttctgaaaacgacatgtcgcacaagtcctaagttacgcgacaggctgccgccctgcccttttcctggcgttttcttgtcgcgtgttttagtcgcataaagtagaatacttgcgactagaaccggagacattacgccatgaacaagagcgccgccgctggcctgctgggctatgcccgcgtcagcaccgacgaccaggacttgaccaaccaacgggccgaactgcacgcggccggctgcaccaagctgttttccgagaagatcaccggcaccaggcgcgaccgcccggagctggccaggatgcttgaccacctacgccctggcgacgttgtgacagtgaccaggctagaccgcctggcccgcagcacccgcgacctactggacattgccgagcgcatccaggaggccggcgcgggcctgcgtagcctggcagagccgtgggccgacaccaccacgccggccggccgcatggtgttgaccgtgttcgccggcattgccgagttcgagcgttccctaatcatcgaccgcacccggagcgggcgcgaggccgccaaggcccgaggcgtgaagtttggcccccgccctaccctcaccccggcacagatcgcgcacgcccgcgagctgatcgaccaggaaggccgcaccgtgaaagaggcggctgcactgcttggcgtgcatcgctcgaccctgtaccgcgcacttgagcgcagcgaggaagtgacgcccaccgaggccaggcggcgcggtgccttccgtgaggacgcattgaccgaggccgacgccctggcggccgccgagaatgaacgccaagaggaacaagcatgaaaccgcaccaggacggccaggacgaaccgtttttcattaccgaagagatcgaggcggagatgatcgcggccgggtacgtgttcgagccgcccgcgcacgtctcaaccgtgcggctgcatgaaatcctggccggtttgtctgatgccaagctggcggcctggccggccagcttggccgctgaagaaaccgagcgccgccgtctaaaaaggtgatgtgtatttgagtaaaacagcttgcgtcatgcggtcgctgcgtatatgatgcgatgagtaaataaacaaatacgcaaggggaacgcatgaaggttatcgctgtacttaaccagaaaggcgggtcaggcaagacgaccatcgcaacccatctagcccgcgccctgcaactcgccggggccgatgttctgttagtcgattc

SEQ ID NO: 11 - DNA, 1625 bp, synthetic construct
  source 1..1625: mol_type = other DNA
tttacactttatgcttccggctcgtatgttgtgtggaattgtgagcggataacaatttcacacaggaaacagctatgaccatgattacgaattctgccatgtcttcgagctcggtacccggggatccattacaggtgaccagctcgaatttcgaagacatgggaatcgggaattaaactatcagtgtttgacaggatatattggcgggtaaacctaagagaaaagagcgtttattagaataacggatatttaaaagggcgtgaaaaggtttatccgttcgtccatttgtatgtgcatgccaaccacagggttcccctcgggatcaaagtactttgatccaacccctccgctgctatagtgcagtcggcttctgacgttcagtgcagccgtgttctgaaaacgacatgtcgcacaagtcctaagttacgcgacaggctgccgccctgcccttttcctggcgttttcttgtcgcgtgttttagtcgcataaagtagaatacttgcgactagaaccggagacattacgccatgaacaagagcgccgccgctggcctgctgggctatgcccgcgtcagcaccgacgaccaggacttgaccaaccaacgggccgaactgcacgcggccggctgcaccaagctgttttccgagaagatcaccggcaccaggcgcgaccgcccggagctggccaggatgcttgaccacctacgccctggcgacgttgtgacagtgaccaggctagaccgcctggcccgcagcacccgcgacctactggacattgccgagcgcatccaggaggccggcgcgggcctgcgtagcctggcagagccgtgggccgacaccaccacgccggccggccgcatggtgttgaccgtgttcgccggcattgccgagttcgagcgttccctaatcatcgaccgcacccggagcgggcgcgaggccgccaaggcccgaggcgtgaagtttggcccccgccctaccctcaccccggcacagatcgcgcacgcccgcgagctgatcgaccaggaaggccgcaccgtgaaagaggcggctgcactgcttggcgtgcatcgctcgaccctgtaccgcgcacttgagcgcagcgaggaagtgacgcccaccgaggccaggcggcgcggtgccttccgtgaggacgcattgaccgaggccgacgccctggcggccgccgagaatgaacgccaagaggaacaagcatgaaaccgcaccaggacggccaggacgaaccgtttttcattaccgaagagatcgaggcggagatgatcgcggccgggtacgtgttcgagccgcccgcgcacgtctcaaccgtgcggctgcatgaaatcctggccggtttgtctgatgccaagctggcggcctggccggccagcttggccgctgaagaaaccgagcgccgccgtctaaaaaggtgatgtgtatttgagtaaaacagcttgcgtcatgcggtcgctgcgtatatgatgcgatgagtaaataaacaaatacgcaaggggaacgcatgaaggttatcgctgtacttaaccagaaaggcgggtcaggcaagacgaccatcgcaacccatctagcccgcgccctgcaactcgccggggccgatgttctgttagtcgattc

SEQ ID NO: 12 - DNA, 630 bp, synthetic construct
  source 1..630: mol_type = other DNA
atgaaggttatcgctgtacttaaccagaaaggcgggtcaggcaagacgaccatcgcaacccatctagcccgcgccctgcaactcgccggggccgatgttctgttagtcgattccgatccccagggcagtgcccgcgattgggcggccgtgcgggaagatcaaccgctaaccgttgtcggcatcgaccgcccgacgattgaccgcgacgtgaaggccatcggccggcgcgacttcgtagtgatcgacggagcgccccaggcggcggacttggctgtgtccgcgatcaaggcagccgacttcgtgctgattccggtgcagccaagcccttacgacatatgggccaccgccgacctggtggagctggttaagcagcgcattgaggtcacggatggaaggctacaagcggcctttgtcgtgtcgcgggcgatcaaaggcacgcgcatcggcggtgaggttgccgaggcgctggccgggtacgagctgcccattcttgagtcccgtatcacgcagcgcgtgagctacccaggcactgccgccgccggcacaaccgttcttgaatcagaacccgagggcgacgctgcccgcgaggtccaggcgctggccgctgaaattaaatcaaaactcatttga

SEQ ID NO: 13 - DNA, 20 bp, synthetic construct
  source 1..20: mol_type = other DNA
tggctactaacggtgcagat

SEQ ID NO: 14 - DNA, 20 bp, synthetic construct
  source 1..20: mol_type = other DNA
acccaaacttctctcctgca

SEQ ID NO: 15 - DNA, 21 bp, synthetic construct
  source 1..21: mol_type = other DNA
tcttcggcgttaggaacccag

SEQ ID NO: 16 - DNA, 21 bp, synthetic construct
  source 1..21: mol_type = other DNA
gcagccttatccttgtcggtg

SEQ ID NO: 17 - protein, 802 aa, Arabidopsis thaliana
MDLKYSASHCNLSSDMKLRRFHQHRGKGREEEYDASSLSLNNLSKLILPPLGVASYNQNHIRSSGWIISPMDSRYRCWEFYMVLLVAYSAWVYPFEVAFLNSSPKRNLCIADNIVDLFFAVDIVLTFFVAYIDERTQLLVREPKQIAVRYLSTWFLMDVASTIPFDAIGYLITGTSTLNITCNLLGLLRFWRLRRVKHLFTRLEKDIRYSYFWIRCFRLLSVTLFLVHCAGCSYYLIADRYPHQGKTWTDAIPNFTETSLSIRYIAAIYWSITTMTTVGYGDLHASNTIEMVFITVYMLFNLGLTAYLIGNMTNLVVEGTRRTMEFRNSIEAASNFVNRNRLPPRLKDQILAYMCLRFKAESLNQQHLIDQLPKSIYKSICQHLFLPSVEKVYLFKGVSREILLLLVSKMKAEYIPPREDVIMQNEAPDDVYIIVSGEVEIIDSEMERESVLGTLRCGDIFGEVGALCCRPQSYTFQTKSLSQLLRLKTSFLIETMQIKQQDNATMLKNFLQHHKKLSNLDIGDLKAQQNGENTDVVPPNIASNLIAVVTTGNAALLDELLKAKLSPDITDSKGKTPLHVAASRGYEDCVLVLLKHGCNIHIRDVNGNSALWEAIISKHYEIFRILYHFAAISDPHIAGDLLCEAAKQNNVEVMKALLKQGLNVDTEDHHGVTALQVAMAEDQMDMVNLLATNGADVVCVNTHNEFTPLEKLRVVEEEEEEERGRVSIYRGHPLERRERSCNEAGKLILLPPSLDDLKKIAGEKFGFDGSETMVTNEDGAEIDSIEVIRDNDKLYFVVNKII

SEQ ID NO: 18 - protein, 802 aa, Arabidopsis thaliana
MDLKYSASHCNLSSDMKLRRFHQHRGKGREEEYDASSLSLNNLSKLILPPLGVASYNQNHIRSSGWIISPMDSRYRCWEFYMVLLVAYSAWVYPFEVAFLNSSPKRNLCIADNIVDLFFAVDIVLTFFVAYIDERTQLLVREPKQIAVRYLSTWFLMDVASTIPFDAIGYLITGTSTLNITCNLLGLLRFWRLRRVKHLFTRLEKDIRYNYFWIRCFRLLSVTLFLVHCAGCSYYLIADRYPHQGKTWTDAIPNFTETSLSIRYIAAIYWSITTMTTVGYGDLHASNTIEMVFITVYMLFNLGLTAYLIGNMTNLVVEGTRRTMEFRNNIEAASNFVNRNRLPPRLKDQILAYMCLRFKAESLNQQHLIDQLPKSIYKSICQHLFLPSVEKVYLFKGVSREILLLLVSKMKAEYIPPREDVIMQNEAPDDVYIIVSGEVEIIDSEMERESVLGTLRCGDIFGEVGALCCRPQSYTFQTKSLSQLLRLKTSFLIETMQIKQQDNATMLKNFLQHHKKLSNLDIGDLKAQQNGENTDVVPPNIASNLIAVVTTGNAALLDELLKAKLSPDITDSKGKTPLHVAASRGYEDCVLVLLKHGCNIHIRDVNGNSALWEAIISKHYEIFRILYHFAAISDPHIAGDLLCEAAKQNNVEVMKALLKQGLNVDTEDHHGVTALQVAMAEDQMDMVNLLATNGADVVCVNTHNEFTPLEKLRVVEEEEEEERGRVSIYRGHPLERRERSCNEAGKLILLPPSLDDLKKIAGEKFGFDGSETMVTNEDGAEIDSIEVIRDNDKLYFVVNKII

SEQ ID NO: 19 - protein, 824 aa, Manihot esculenta
MEMKSSWENHHEEKKQSNHYEEDDTSLSLSSLSKIILPPLGVSSYNHNPIETKGWIISPMNSKYRCWETYMVVLVAYSAWVSPFEVAFLKSNPNKGLYVADSVVDLFFAIDIVLTFFVAYIDSTTHLMVRDRRKISIRYLSTWFSMDVASTIPFEALGYLFTGKRKMGLSYSLLGMLRFWRLRRVKQLFTRLEKDIRFSYFWVRCTRLLFVTLLLVHCAGCLCYLLADRYPHQGRTWLGSVNPNFRETSLRNRYISALYWSVTTMTTVGYGDLHAVNTGEMIFIIFYMLFNLGLTAYLIGNMTNLVVEGTRRTMEFRNSIEAASNFVCRNRLPPRLKEQILAYMCLRFKAESLNQNHLIEQLPKSICKCICQHLFLPIAEKVYLFKGVSREILLLLVAEMKAEYIPPREDVIMQNEAPDDVYIIVSGEVEIIDSALEKERIFGILQSGDMFGEVGALCCKPQSFTFRTKTLSQLLKLKTSALIETMQIKQEDYVAIIKNFLQHHKKLKDFKIGEFIAEGGEEDGDPNMAFNLLTAASAGNAAFLEELLRAKLDPDIGDSKGRTPLHFAASKGHEDCALALLRHGCNIHLKDVNGNTALWEALSSKHQSVFRILYHFANVSDPHTAGDLLCTAAKRNDLTMMNSLLKHGLNVDSKDRQGKTAVQIAMAQNYIDMVDLLVMNGADVSAANSSEFCSTTLNKMLQRRESGHRITMPDTVTSDEVILKMDQEEKQCKSSEKSNELKYTRVSIYRGHPLVRKETCCRQAGRLIRLPNSMEELKSIAGEKFRFDARNAMVTDEEGSEIDSIEVIRDNDKLFIVEDPTPFM

SEQ ID NO: 20 - protein, 841 aa, Manihot esculenta
MEMRSTPSNDLYHLPFTMKRSWRNHHGHPQTPHHHHHQEDDTSLSVSSLSKIILPPLGVSSYNHNPVETKGWIVSPMNSKYRCWETFMVVLVAYSAWVYPFEVAFLNSSPNKMLYITDNIVDLFFAIDIVLTFFVAYIDSRTQLLVRDRTKISIRYLSTWFLMDVASTIPFEALAYFFTGKHSMGLSYSLLGMLRFWRLRRVKQLFTRLEKDIRFSYFWIRCARLIIVTLFLVHCAGCLYYLLADRYPHQGRTWIGAVIPNFRETSLWIRYISALYWSITTMTTVGYGDLHAVNTMEMIFIIFYMLFNLGLTAYLIGNMTNLVVEGTRRTMEFRNSIEAASNFVCRNRLPPRLKEQILAYMCLRFKAESLNQNHLIEQLPKSICKSICHHLFLPTVEKVYLFSGVSREILLLLVAEMKAEYIPPREDVIMQNEAPDDVYIIVSGEVEIIDSDLEKELVVGTLQSGDMFGEVGALCCKVQSFTFRTKTLSQLLKLKTSTLIDTMQTKQEDYVAIIKNFLQHHKKLKGLKLGESLVDDGEEDGDPNMAFNLLTVASTGNAAFLEELLRAKLDPDIGDSKGRTPLHVAASKGHEDCVLALLRHGCNINLRDVNGNTALWEALSSKHQSVFRILYHFSNIDDPHTAGELLCKAAKENDLTMMKELLKHGLNVDAKDRQGKTAVQIAMAQNYVDMVDLLVMNGADVSAANTSEFSSTTLNEMLQKREIGHRITVPDTVTSDEVILKRNQEEEEGNSSGKSNGWECRRVSIYRGHPLIRKETCCLEPGRLIRLPNSMEELKSIAGEKFGFDARNAMVTDEEGSEIDSIEVIRDNDKLFIVEDPNSSM

SEQ ID NO: 21 - protein, 178 aa, Arabidopsis thaliana
TGTSTLNITCNLLGLLRFWRLRRVKHLFTRLEKDIRYSYFWIRCFRLLSVTLFLVHCAGCSYYLIADRYPHQGKTWTDAIPNFTETSLSIRYIAAIYWSITTMTTVGYGDLHASNTIEMVFITVYMLFNLGLTAYLIGNMTNLVVEGTRRTMEFRNSIEAASNFVNRNRLPPRLKDQI

SEQ ID NO: 22 - protein, 179 aa, Manihot esculenta
TGKRKMGLSYSLLGMLRFWRLRRVKQLFTRLEKDIRFSYFWVRCTRLLFVTLLLVHCAGCLCYLLADRYPHQGRTWLGSVNPNFRETSLRNRYISALYWSVTTMTTVGYGDLHAVNTGEMIFIIFYMLFNLGLTAYLIGNMTNLVVEGTRRTMEFRNSIEAASNFVCRNRLPPRLKEQI

SEQ ID NO: 23 - protein, 179 aa, Manihot esculenta
TGKHSMGLSYSLLGMLRFWRLRRVKQLFTRLEKDIRFSYFWIRCARLIIVTLFLVHCAGCLYYLLADRYPHQGRTWIGAVIPNFRETSLWIRYISALYWSITTMTTVGYGDLHAVNTMEMIFIIFYMLFNLGLTAYLIGNMTNLVVEGTRRTMEFRNSIEAASNFVCRNRLPPRLKEQI

SEQ ID NO: 24 - protein, 179 aa, synthetic construct
  source 1..179: note = Consensus
TGKXXMGLSYSLLGMLRFWRLRRVKQLFTRLEKDIRFSYFWIRCXRLLXVTLFLVHCAGCLYYLLADRYPHQGRTWXGAVIPNFRETSLXIRYISALYWSITTMTTVGYGDLHAVNTXEMIFIIFYMLFNLGLTAYLIGNMTNLVVEGTRRTMEFRNSIEAASNFVCRNRLPPRLKEQI

SEQ ID NO: 25 - protein, 824 aa, Manihot esculenta
MEMKSSWENHHEEKKQSNHYEEDDTSLSLSSLSKIILPPLGVSSYNHNPIETKGWIISPMNSKYRCWETYMVVLVAYSAWVSPFEVAFLKSNPNKGLYVADSVVDLFFAIDIVLTFFVAYIDSTTHLMVRDRRKISIRYLSTWFSMDVASTIPFEALGYLFTGKRKMGLSYSLLGMLRFWRLRRVKQLFTRLEKDIRFNYFWVRCTRLLFVTLLLVHCAGCLCYLLADRYPHQGRTWLGSVNPNFRETSLRNRYISALYWSVTTMTTVGYGDLHAVNTGEMIFIIFYMLFNLGLTAYLIGNMTNLVVEGTRRTMEFRNNIEAASNFVCRNRLPPRLKEQILAYMCLRFKAESLNQNHLIEQLPKSICKCICQHLFLPIAEKVYLFKGVSREILLLLVAEMKAEYIPPREDVIMQNEAPDDVYIIVSGEVEIIDSALEKERIFGILQSGDMFGEVGALCCKPQSFTFRTKTLSQLLKLKTSALIETMQIKQEDYVAIIKNFLQHHKKLKDFKIGEFIAEGGEEDGDPNMAFNLLTAASAGNAAFLEELLRAKLDPDIGDSKGRTPLHFAASKGHEDCALALLRHGCNIHLKDVNGNTALWEALSSKHQSVFRILYHFANVSDPHTAGDLLCTAAKRNDLTMMNSLLKHGLNVDSKDRQGKTAVQIAMAQNYIDMVDLLVMNGADVSAANSSEFCSTTLNKMLQRRESGHRITMPDTVTSDEVILKMDQEEKQCKSSEKSNELKYTRVSIYRGHPLVRKETCCRQAGRLIRLPNSMEELKSIAGEKFRFDARNAMVTDEEGSEIDSIEVIRDNDKLFIVEDPTPFM

SEQ ID NO: 26 - protein, 841 aa, Manihot esculenta
MEMRSTPSNDLYHLPFTMKRSWRNHHGHPQTPHHHHHQEDDTSLSVSSLSKIILPPLGVSSYNHNPVETKGWIVSPMNSKYRCWETFMVVLVAYSAWVYPFEVAFLNSSPNKMLYITDNIVDLFFAIDIVLTFFVAYIDSRTQLLVRDRTKISIRYLSTWFLMDVASTIPFEALAYFFTGKHSMGLSYSLLGMLRFWRLRRVKQLFTRLEKDIRFNYFWIRCARLIIVTLFLVHCAGCLYYLLADRYPHQGRTWIGAVIPNFRETSLWIRYISALYWSITTMTTVGYGDLHAVNTMEMIFIIFYMLFNLGLTAYLIGNMTNLVVEGTRRTMEFRNNIEAASNFVCRNRLPPRLKEQILAYMCLRFKAESLNQNHLIEQLPKSICKSICHHLFLPTVEKVYLFSGVSREILLLLVAEMKAEYIPPREDVIMQNEAPDDVYIIVSGEVEIIDSDLEKELVVGTLQSGDMFGEVGALCCKVQSFTFRTKTLSQLLKLKTSTLIDTMQTKQEDYVAIIKNFLQHHKKLKGLKLGESLVDDGEEDGDPNMAFNLLTVASTGNAAFLEELLRAKLDPDIGDSKGRTPLHVAASKGHEDCVLALLRHGCNINLRDVNGNTALWEALSSKHQSVFRILYHFSNIDDPHTAGELLCKAAKENDLTMMKELLKHGLNVDAKDRQGKTAVQIAMAQNYVDMVDLLVMNGADVSAANTSEFSSTTLNEMLQKREIGHRITVPDTVTSDEVILKRNQEEEEGNSSGKSNGWECRRVSIYRGHPLIRKETCCLEPGRLIRLPNSMEELKSIAGEKFGFDARNAMVTDEEGSEIDSIEVIRDNDKLFIVEDPNSSM

SEQ ID NO: 27 - DNA, 21 bp, synthetic construct
  source 1..21: mol_type = other DNA
tcttcggcgttaggaacccag

SEQ ID NO: 28 - DNA, 21 bp, synthetic construct
  source 1..21: mol_type = other DNA
gcagccttatccttgtcggtg

SEQ ID NO: 29 - DNA, 21 bp, synthetic construct
  source 1..21: mol_type = other DNA
acaggggcttaacgtcgacac

SEQ ID NO: 30 - DNA, 22 bp, synthetic construct
  source 1..22: mol_type = other DNA
tgcaccgttagtagccaggaga

SEQ ID NO: 31 - DNA, 20 bp, synthetic construct
  source 1..20: mol_type = other DNA
cagcttcttgtccgtgaacc

SEQ ID NO: 32 - DNA, 20 bp, synthetic construct
  source 1..20: mol_type = other DNA
aggtaagcagtgaggccaag